(19) United States
(12) Patent Application Publication
(10) Pub. No.: US 2013/0345215 A1
(43) Pub. Date: Dec. 26, 2013

(54) PYRAZOLOPYRIMIDONE AND PYRAZOLOPYRIDONE INHIBITORS OF TANKYRASE

(71) Applicant: Genentech, Inc., South San Francisco, CA (US)

(72) Inventors: Jianwen Feng, Millbrae, CA (US); Nancy-Ellen Haynes, Cranford, NJ (US); Johannes Cornelius Hermann, Jersey City, NJ (US); Kyungjin Kim, Livingston, NJ (US); Jin-Jun Liu, Warren, NJ (US); Nathan Robert Scott, Livingston, NJ (US); Lin Yi, Basking Ridge, NJ (US); Mark Edward Zak, San Mateo, CA (US); Guiling Zhao, Palo Alto, CA (US)

(21) Appl. No.: 13/911,585

(22) Filed: Jun. 6, 2013

Related U.S. Application Data
(60) Provisional application No. 61/656,644, filed on Jun. 7, 2012.

(51) Int. Cl. C07D 487/04; C07D 471/04

(52) U.S. Cl. CPC C07D 487/04; C07D 471/04
USPC 514/234.2; 544/262; 514/252.16; 514/262.1; 544/362; 514/253.04; 546/119; 514/303; 514/252.11; 544/118; 435/184

(57) ABSTRACT

There are provided compounds of the formula

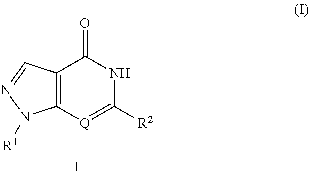

or a pharmaceutically acceptable salt thereof, wherein Q, R₁ and R₂ are as defined herein.

The compounds of formula I are useful in the treatment of cancer.

### CROSS REFERENCE TO PRIOR APPLICATIONS

This application claims the benefit of priority to U.S. Ser. No. 61/656,644 filed Jun. 7, 2012 which is hereby incorporated by reference in its entirety.

### FIELD OF THE INVENTION

The present invention relates to pyrazolopyrimidinones and pyrazolopyridones which act as inhibitors of tankyrase and are useful in the amelioration or treatment of cancer.

### BACKGROUND OF THE INVENTION

Cancer is a disease characterized by the loss of appropriate control for cell growth. The American Cancer Society has estimated that there were in excess of 1.5 million new cases of cancer within the United Stated of America in 2010 and approximately 570,000 deaths that year estimated to be attributable to cancer. The World Health Organization has estimated that cancer was the leading cause of death globally in 2010, with the number of deaths caused by cancer growing to 12 million per year by 2030.

It has been suggested that there are 6 capabilities which need to be developed by cells in order to lead to the formation of cancerous lesions. These traits are self-sufficiency in growth signals, insensitivity to anti-growth signals, tissue invasion and metastasis, limitless replication potential, sustained angiogenesis and evasion of apoptosis. Growth signaling is required for cells to transition from a quiescent state into an active proliferative state. These signals are typically transmitted from transmembrane receptors, through signal transduction cascades involving numerous intracellular kinases, eventually resulting in changes in gene expression at the nuclear level within the cell. In recent years there has been much interest in the area of signal transduction inhibitors, particularly kinase inhibitors, and their use for the treatment of cancer. Several examples from this class of compounds have been successfully evaluated in clinical settings and are now commercially available and marketed for the treatment of specific forms of cancer e.g. imatinib tosylate (marketed as Gleevec® by Novartis for the treatment of Philadelphia chromosome-positive chronic myeloid leukemia), lapatinib ditosylate (marketed as Tykerb® by GlaxoSmithKline for the treatment of HER² positive breast cancer in combination with other chemotherapeutic agents), sunitinib malate (marketed as Sutent® by Pfizer and approved for the treatment of renal cancer) and sorafenib (marketed as Nexavar by Bayer for the treatment of renal cancer).

In addition to the growth factor associated signaling pathways, which predominantly utilize kinase catalyzed transfer of phosphate groups as the key component of the signaling pathway, numerous other signaling pathways also exist within cells and their proper regulation is critical for maintaining correct levels of cell growth and replication. In the emerging area of cancer stem cell inhibition the Wnt, Notch and Hedgehog pathways have received much interest as potential ways in which to avoid tumor relapse and metastasis. The Wnt pathway is instrumental in embryonic development and in tissue maintenance in adults with the activity of individual components within the pathway under tight regulation. In cancer and other diseases cell signaling pathways no longer exhibit the appropriate level of control. In the case of the Wnt pathway, signal transduction is controlled by the relative stabilities of 2 proteins, axin and β-catenin. An overabundance of β-catenin leads to increased Wnt signaling and activation of associated nuclear transcription factors while excess axin results in the degradation of intracellular β-catenin and decreased signaling. Dysregulation of the canonical Wnt signaling pathway has been implicated in a range of human carcinomas such as colon cancer, hepatocellular carcinoma, endometrial ovarian cancer, pilomatricoma skin cancer, prostate cancer, melanoma and Wilms tumor.

In the canonical Wnt signaling pathway signaling is initiated by interaction of a Wnt ligand with a receptor complex containing a Frizzled family member and low-density lipoprotein receptor-related protein. This leads to the formation of a disheveled-frizzled complex and relocation of axin from the destruction complex to the cell membrane. Axin is the concentration limiting component of the destruction complex, and it is this complex which is formed with adenomatous polyposis coli proteins, casein-kinase 1α and glycogen synthase kinase 3β which is responsible for controlling intracellular levels of β-catenin. In the presence of functional destruction complex, β-catenin is sequentially phosphorylated by casein-kinase 1α and glycogen synthase kinase 3β on a conserved set of serine and threonine residues at the amino-terminus. Phosphorylation facilitates binding of β-catenin to β-transducin repeat-containing protein which then mediates ubiquitination and subsequent proteasomal degradation of β-catenin. In the absence of sufficiently elevated concentrations of the destruction complex, un-phosphorylated β-catenin is able to migrate to the cell nucleus and interact with T-cell factor proteins and convert them into potent transcriptional activators through the recruitment of co-activator proteins.

It has recently been reported that intracellular axin levels are influenced by the poly(ADP-ribose) polymerase enzyme family members tankyrase-1 and tankyrase-2 (also known as PARP5a and PARP5b) (Nature Chemical Biology 2009, 5, 100 and Nature 2009, 461, 614). Tankyrase enzymes are able to poly-ADP ribosylate (PARsylate) axin, which marks this protein for subsequent ubiquitination and proteasomal degradation. Thus, it would be expected that in the presence of an inhibitor of tankyrase catalytic activity, axin protein concentration would be increased, resulting in higher concentration of the destruction complex and decreased concentrations of unphosphorylated intracellular β-catenin and decreased Wnt signaling. An inhibitor of tankyrase-1 and -2 would also be expected to have an effect on other biological functions of the tankyrase proteins e.g. chromosome end protection (telomeres), insulin responsiveness and spindle assembly during mitosis (Biochimie 2009, 5, 100).

Therapeutics which are directed at and can correct dysregulation of the Wnt signaling pathway have been implicated in conditions such as bone density defects, coronary disease, late onset Alzheimer's disease, familial exudative vitreoretinopathy, retinal angiogenesis, tetra-amelia, Mullerian-duct regression and virilization, SERKAL syndrome, type 2 diabetes, Fuhrmann syndrome, skeletal dysplasia, focal dermal hypoplasia and neural tube defects. Although the above introduction has focused on the relevance of Wnt signaling in cancer, the Wnt signaling pathway is of fundamental importance and has potential implication in a broad range of human diseases, not necessarily limited to the examples provided above for illustrative purposes.

### SUMMARY OF THE INVENTION

There is a continuing need for new and novel therapeutic agents that can be used for cancer and hyperproliferative conditions. The tankyrase enzymes, which modulate Wnt activity, are members of the PARP family. Design and development of new pharmaceutical compounds that inhibit or modulate their activity is essential. In one aspect of the present invention there is provided a compound according to formula I

One aspect of the invention is a compound of formula I wherein:

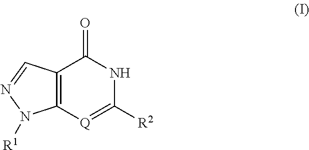

Q and X are independently in each occurrences N or CH;

R¹ is selected from the group consisting of hydrogen, C₁₋₆ alkyl, C₁₋₆ hydroxyalkyl, C₁₋₆-dihydroxyalkyl, 1,1-dioxothian-4-yl or tetrahydropyran-4-yl;

R² is

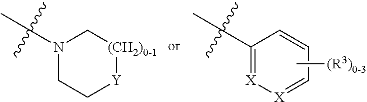

Y is selected from the group consisting of CR⁴R⁵ or NR⁴ wherein R⁵ is hydrogen, C₁₋₆ alkyl, —OH or —CN;

R³ is selected from the group consisting of (i) hydrogen, (ii) C₁₋₆ alkyl, (iii) C₁₋₆ haloalkyl, (iv) halogen, (v) C₁₋₆ alkoxy, (vi) S(O)₂R^{3a} wherein R^{1a} is C₁₋₆ alkyl, C₃₋₆ cycloalkyl, C₁₋₃ alkyl-C₃₋₆ cycloalkyl or NH₂ or (vii) CONR^{3b}R^{3c} wherein R^{3b} and R^{1c} are independently hydrogen, C₁₋₃ alkyl or R^{3b} and R^{3c} together with the nitrogen to which they are attached form a cyclic amine

R⁴ is selected from the group consisting of: (i) hydrogen, (ii) C₁₋₆ alkyl, (iii) C₁₋₆ haloalkyl optionally substituted with hydroxyl, (iv) C₃₋₇ cycloalkyl (v) C₃₋₇cycloalkyl-C₁₋₃ alkyl, (vi) C₅₋₁₀ bicycloalkyl,

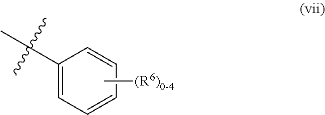

(viii) heteroaryl, (ix) heteroaryl-C₁₋₃ alkyl, (x) heterocyclyl; (xi) heterocyclyl C₁₋₃ alkyl;

each R⁶ is independently selected from the group consisting of: (a) C₁₋₆ alkyl, (b) C₁₋₆ haloalkyl optionally substituted with hydroxyl, (c) C₁₋₆ hydroxyalkyl, (d) C₁₋₆-dihydroxyalkyl, (e) C₁₋₃ alkoxy-C₁₋₃ alkyl, (f) C₃₋₇ cycloalkyl, -(g) C₁₋₆ acyl, (h) halo, (i) cyano, (j) NO₂, (k) carboxyl, (1) C₁₋₆ alkoxycarbonyl, (m) CO NR^{4b}R^{4c} wherein R^{4b} and R^{4c} are independently hydrogen, C₁₋₆ alkyl or R^{4b} and R^{4c} together with the nitrogen atom to which they are attached are a cyclic amine, (n) —S(O)₂R^{4a} wherein R^{4a} is C₁₋₆ alkyl, C₃₋₆ cycloalkyl, C₁₋₃ alkyl-C₃₋₆ cycloalkyl or NH₂, (o) NR^{4b}R^{4c}, (p), OR^{4d} wherein R^{4d} is selected from the group consisting of (i) hydrogen, (ii) C₁₋₆ alkyl, (iii) C₁₋₃ alkoxy-C₁₋₃ alkyl, (iv) C₁₋₆ hydroxyalkyl said hydroxyalkyl further optionally substituted with halogen, (v) C₁₋₆ dihydroxyalkyl, (vi) (alkylene)₂₋₆NR^{4e}R^{4f} wherein R^{4e} and R^{4f} are independently hydrogen or C₁₋₆ alkyl or R^{4e} and R^{4f} together with the nitrogen to which they are attached form a cyclic amine optionally containing another heteroatom selected from NR^{4g}, O or S(O)₀₋₂ wherein R^{4g} is hydrogen or C₁₋₃ alkyl, (vii) oxetanyl, (viii) tetrahydropyranyl, (ix) 1,1-dioxothianyl, (x) (1-oxothietan-3-yl)methyl and (xi) (alkylene)₂₋₆OR^{4b} wherein R^{4b} is C(O)CH(NH₂)R⁴ⁱ wherein R⁴ⁱ C₁₋₆ alkyl or P(═O)(OH)₂; (q) heterocyclyl-C₁₋₃ alkyl wherein said heterocycle is piperidine, morpholine, piperazine or 4-methyl-piperazine; (r) 1H-tetrazol-5-yl, and (s) 1,1-dioxothiolan-3-yl; and wherein:

each said cycloalkyl is optionally substituted by one to three hydroxyl or C₁₋₃ alkoxy-C₁₋₆ alkoxy;

each said heteroaryl is optionally further substituted with C₁₋₆ alkyl, C₁₋₃ hydroxyalkyl, C₁₋₆ haloalkyl, halogen or C₁₋₆ alkylsulfonyl;

each said heterocycle is selected from tetrahydropyran-4-yl, tetrahydrofuran-2-yl, oxetan-3-yl, 1,1-dioxo-tetrahydrothiophenyl, 1-Boc-piperidinyl, piperidin-4-yl, 1-methyl-piperidin-4-yl, 1-Boc-piperazin-4-yl; 1-methyl-piperazin-4-yl or piperazin-4-yl; or a pharmaceutically acceptable salt thereof.

The present invention additionally relates to pharmaceutical compositions comprising one or more compounds of the invention, or a pharmaceutically acceptable salt, and a pharmaceutically acceptable carrier or excipient.

The present invention further relates to a method of treating, ameliorating or preventing cancer in a mammal, preferably a human, comprising administering to said mammal a therapeutically effective amount of a compound according to the invention or a pharmaceutically acceptable salt thereof.

### DETAILED DESCRIPTION OF THE INVENTION

The phrase “a” or “an” entity as used herein refers to one or more of that entity; for example, a compound refers to one or more compounds or at least one compound. As such, the terms “a” (or “an”), “one or more”, and “at least one” can be used interchangeably herein.

The phrase “as defined herein above” refers to the broadest definition for each group as provided in the Summary of the Invention or the broadest claim. In all other embodiments provided below, substituents which can be present in each embodiment and which are not explicitly defined retain the broadest definition provided in the Summary of the Invention.

As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least”. When used in the context of a process, the term “comprising” means that the process includes at least the recited steps, but may include additional steps. When used in the context of a compound or composition, the term “comprising” means that the compound or composition includes at least the recited features or components, but may also include additional features or components.

The term “independently” is used herein to indicate that a variable is applied in any one instance without regard to the presence or absence of a variable having that same or a different definition within the same compound. Thus, in a compound in which R″ appears twice and is defined as “independently carbon or nitrogen”, both R″s can be carbon, both R″s can be nitrogen, or one R″ can be carbon and the other nitrogen.

When any variable (e.g., R¹, R^{4a}, Ar, X¹ or Het) occurs more than one time in any moiety or formula depicting and describing compounds employed or claimed in the present invention, its definition on each occurrence is independent of its definition at every other occurrence. Also, combinations of substituents and/or variables are permissible only if such compounds result in stable compounds.

The symbols “*” at the end of a bond or “------” drawn through a bond each refer to the point of attachment of a functional group or other chemical moiety to the rest of the molecule of which it is a part. Thus, for example:

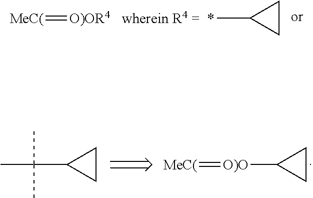

A bond drawn into ring system (as opposed to connected at a distinct vertex) indicates that the bond may be attached to any of the suitable ring atoms.

The term “optional” or “optionally” as used herein means that a subsequently described event or circumstance may, but need not, occur, and that the description includes instances where the event or circumstance occurs and instances in which it does not. For example, “optionally substituted” means that the optionally substituted moiety may incorporate a hydrogen or a substituent.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

As used herein, the recitation of a numerical range for a variable is intended to convey that the invention may be practiced with the variable equal to any of the values within that range. Thus, for a variable which is inherently discrete, the variable can be equal to any integer value of the numerical range, including the end-points of the range. Similarly, for a variable which is inherently continuous, the variable can be equal to any real value of the numerical range, including the end-points of the range. As an example, a variable which is described as having values between 0 and 2, can be 0, 1 or 2 for variables which are inherently discrete, and can be 0.0, 0.1, 0.01, 0.001, or any other real value for variables which are inherently continuous.

The present invention relates to a compound of the formula (I)

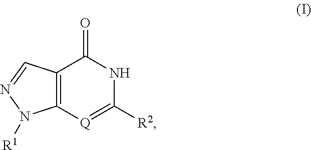

wherein

Q and X are independently in each occurrences N or CH;

R¹ is selected from the group consisting of hydrogen, C₁₋₆ alkyl, C₁₋₆ hydroxyalkyl, C₁₋₆-dihydroxyalkyl, 1,1-dioxothian-4-yl or tetrahydropyran-4-yl;

R² is

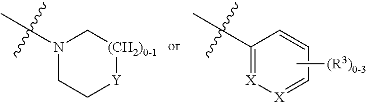

Y is selected from the group consisting of CR⁴R⁵ or NR⁴ wherein R⁵ is hydrogen, C₁₋₆ alkyl, —OH or —CN;

R³ is selected from the group consisting of (i) hydrogen, (ii) C₁₋₆ alkyl, (iii) C₁₋₆ haloalkyl, (iv) halogen, (v) C₁₋₆ alkoxy, (vi) S(O)₂R^{3a} wherein R^{1a} is C₁₋₆ alkyl, C₃₋₆ cycloalkyl, C₁₋₃ alkyl-C₃₋₆ cycloalkyl or NH₂ or (vii) CONR^{3b}R^{3e} wherein R^{3b} and R^{3c} are independently hydrogen, C₁₋₃ alkyl or R^{3b} and R^{3c} together with the nitrogen to which they are attached form a cyclic amine

R⁴ is selected from the group consisting of:
(i) hydrogen,
(ii) C₁₋₆ alkyl,
(iii) C₁₋₆ haloalkyl optionally substituted with hydroxyl,
(iv) C₃₋₇ cycloalkyl
(v) C₃₋₇cycloalkyl-C₁₋₃ alkyl,
(vi) C₅₋₁₀ bicycloalkyl,

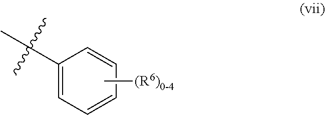

wherein each R⁶ is independently selected from the group consisting of:
   (a) C₁₋₆ alkyl,
   (b) C₁₋₆ haloalkyl optionally substituted with hydroxyl,
   (c) C₁₋₆ hydroxyalkyl,
   (d) C₁₋₆-dihydroxyalkyl,
   (e)₁₋₃ alkoxy-C₁₋₃ alkyl,
   (f) C₃₋₇ cycloalkyl,
   (g) C₁₋₆ acyl,
   (h) halo,
   (i) cyano,
   (j) NO₂,
   (k) carboxyl,
   (l) C₁₋₆ alkoxycarbonyl,
   (m) CO NR^{4b}R^{4c} wherein R^{4b} and R^{4c} are independently hydrogen, C₁₋₆ alkyl or R^{4b} and R^{4c} together with the nitrogen atom to which they are attached are a cyclic amine,
   (n) —S(O)₂R^{4a} wherein R^{4a} is C₁₋₆ alkyl, C₃₋₆ cycloalkyl, C₁₋₃ alkyl-C₃₋₆ cycloalkyl or NH₂,
   (o) NR^{4b}R^{4c}, wherein R^{4b} and R^{4c} are independently C₁₋₆ alkyl or hydrogen
   (p) OR^{4d} wherein R^{4d} is selected from the group consisting of (i) hydrogen, (ii) C₁₋₆ alkyl, (iii) C₁₋₃ alkoxy-C₁₋₃ alkyl, (iv) C₁₋₆ hydroxyalkyl said hydroxyalkyl further optionally substituted with halogen, (v) C₁₋₆ dihydroxyalkyl, (vi) (alkylene)₂₋₆NR^{4e}R^{f4} wherein R^{4e} and R^{4f} are independently hydrogen or C₁₋₆ alkyl or R^{4e} and R^{4f} together with the nitrogen to which they are attached form a cyclic amine optionally containing another heteroatom selected from NR^{4g}, O or S(O)₀₋₂ wherein R^{4g} is hydrogen or C₁₋₃ alkyl, (vii) oxetanyl, (viii) tetrahydropyranyl, (ix) 1,1-dioxothianyl, (x) (1-oxothietan-3-yl)methyl and (xi) (alkylene)₂₋₆OR^{4h} wherein R⁴ⁱ is C(O)CH(NH₂)R^{4h} wherein R⁴ⁱ is C₁₋₆ alkyl or P(═O)(OH)₂;
   (q) heterocyclyl-C₁₋₃ alkyl wherein said heterocycle is piperidine, morpholine, piperazine or 4-methyl-piperazine;
   (r) 1H-tetrazol-5-yl, and,
   (s) 1,1-dioxothiolan-3-yl;
(viii) heteroaryl
(ix) heteroaryl-C₁₋₃ alkyl
(x) heterocyclyl;
(xi) heterocyclyl C₁₋₃ alkyl;
(xii) —S(O)R^{4a} wherein R^{4a} is C₃₋₆ alkyl, C₃₋₆ cycloalkyl, C₁₋₃ alkyl-C₃₋₆ cycloalkyl or NH₂;
(xiii) 1,1-dioxothiolan-3-yl;
and wherein:
each said cycloalkyl is optionally substituted by one to three hydroxyl or C₁₋₃ alkoxy-C₁₋₆ alkoxy;
each said heteroaryl is optionally further substituted with C₁₋₆ alkyl, C₁₋₆ alkoxy, C₁₋₃ hydroxyalkyl, C₁₋₆ haloalkyl, halogen, CN, pyrazinyl or C₁₋₆ alkylsulfonyl;
each said heterocycle is selected from tetrahydropyran-4-yl, tetrahydrofuran-2-yl, oxetan-3-yl, 1,1-dioxo-tetrahydrothiophenyl, 1,1-dioxothiolan-3-yl, 1-Boc-piperidinyl, piperidin-4-yl, 1-methyl-piperidin-4-yl, 1-Boc-piperazin-4-yl; 1-methyl-piperazin-4-yl or piperazin-4-yl;

or a pharmaceutically acceptable salt thereof.

In one embodiment of the present invention there is provided a compound of formula I wherein

Q and X are independently in each occurrences N or CH;

R¹ is selected from the group consisting of hydrogen, C₁₋₆ alkyl, C₁₋₆ hydroxyalkyl, C₁₋₆-dihydroxyalkyl or 1,1-dioxothian-4-yl;

R² is

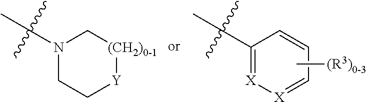

Y is selected from the group consisting of CR⁴R⁵ or NR⁴ wherein R⁵ is hydrogen, C₁₋₆ alkyl, —OH or —CN;

R³ is selected from the group consisting of haloalkyl, S(O)₂R^{3a} wherein R^{1a} is C₁₋₆ alkyl, C₃₋₆ cycloalkyl or C₁₋₃ alkyl-C₃₋₆ cycloalkyl;

R⁴ is selected from the group consisting of C₃₋₇ cycloalkyl, C₃₋₇cycloalkyl-C₁₋₃ alkyl, C₅₋₁₀ bicycloalkyl, heteroaryl, heteroaryl-Cl_{—}3 alkyl, heterocyclyl C₁₋₃ alkyl, —S(O)₂R^{4a} wherein R^{4a} is C₁₋₆ alkyl and

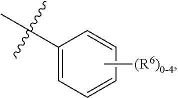

wherein each R⁶ is independently selected from the group consisting of:
   (a) C₁₋₆ haloalkyl optionally substituted with hydroxyl,
   (b) C₁₋₆ hydroxyalkyl,
   (c) C₁₋₆-dihydroxyalkyl,
   (d) C₁₋₃ alkoxy-C₁₋₃ alkyl,
   (e) halo,
   (f) cyano
   (g) NO₂,
   (h) carboxyl,
   (i) C₁₋₆ alkoxycarbonyl,
   (j) CO NR^{4b}R^{4c} wherein R^{4b} and R^{4c} are independently hydrogen, C₁₋₆ alkyl or R^{4b} and R^{4c} together with the nitrogen atom to which they are attached are a cyclic amine,
   (k) —S(O)₂R^{4a} wherein R^{4a} is C₁₋₆ alkyl, or NH₂,
   (l) NR^{4b}R^{4c} wherein R^{4b} and R^{4c} are independently C₁₋₆ alkyl or hydrogen,
   (m), OR^{4d} wherein R^{4d} is selected from the group consisting of hydrogen, C₁₋₃ alkoxy-C₁₋₃ alkyl, C₁₋₆ hydroxyalkyl said hydroxyalkyl further optionally substituted with halogen, (alkylene)₂₋₆NR^{4e}R^{4f} wherein R^{4e} and R^{4f} are independently hydrogen or C₁₋₆ alkyl or R^{4e} and R^{4f} together with the nitrogen to which they are attached form a cyclic amine, oxetanyl, 1,1-dioxothianyl, (1-oxothietan-3-yl)methyl and (alkylene)₂₋₆OR^{4h} wherein R^{4h} is C(O)CH(NH₂)R⁴ⁱ wherein R⁴ⁱ C₁₋₆ alkyl or P(═O)(OH)₂,
   (n) heterocyclyl-C₁₋₃ alkyl wherein said heterocycle is morpholine, or 4-methyl-piperazine, and
   (o) 1H-tetrazol-5-yl;
and wherein:
each said cycloalkyl is optionally substituted by one to three hydroxyl or C₁₋₃ alkoxy-C₁₋₆ alkoxy;
each said heteroaryl is optionally further substituted with C₁₋₆ alkyl, C₁₋₆ alkoxy, C₁₋₃ hydroxyalkyl, C₁₋₆ haloalkyl, halogen, CN, pyrazinyl or C₁₋₆ alkylsulfonyl;
each said heterocycle is selected from tetrahydropyran-4-yl, tetrahydrofuran-2-yl, oxetan-3-yl, 1,1-dioxo-tetrahydrothiophenyl, 1,1-dioxothiolan-3-yl, 1-Boc-piperidinyl, piperidin-4-yl, 1-methyl-piperidin-4-yl, 1-Boc-piperazin-4-yl; 1-methyl-piperazin-4-yl or piperazin-4-yl.

In one embodiment of the present invention there is provided a compound of formula I wherein 0 is N, R₁ is 1,1-dioxothian-4-yl, R₁ is

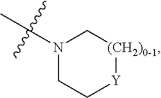

### Y is NR₄, R₄

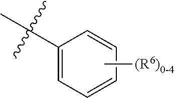

and R⁶ is independently selected from halogen and —O(CH₂)₂OCH₃.

In one embodiment of the present invention there is provided a compound according to formula Ia: wherein:

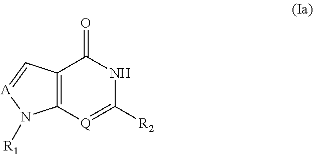

A is N,

Q is N or CH,

R₁ is selected from the group consisting of hydrogen and alkyl,

R₂ is

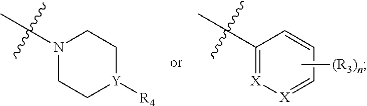

R₄ is selected from the group consisting of:

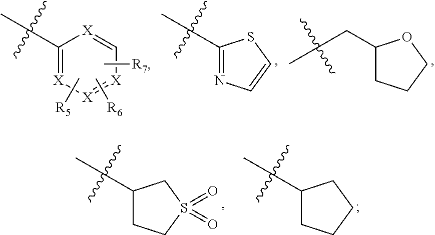

X is CH or N,

Y is selected from the group consisting of nitrogen, carbon, COH and CCN,

R₅ is halogen,

R₆ is halogen or hydrogen,

R₃ and R₇ is selected from the group consisting, of hydrogen, alkyl, substituted alkyl, haloalkyl, halogen, O-alkyl, O-substituted alkyl, CN, trifluoromethyl, nitro, carboxyalkyl, alkylsulfonyl, hydroxyl, —NH₂, hydroxyalkyl, carboxylic acid, sulfonamide, tetrazole and alkyl ketone and

n is 0 to 3; or a pharmaceutically acceptable salt thereof.

Also provided are compounds of formula Ia wherein

Q is N or CH,

A is N,

R₁ is selected from hydrogen or alkyl and

R₂ is

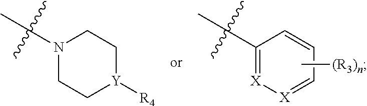

Also provided are compounds of formula I wherein

R₂ is

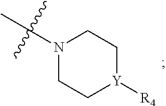

Y is N and

R₄ is

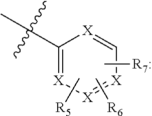

wherein X is CH,

R₅ is chloro or fluoro,

R₆ is chloro, fluoro or hydrogen, and

R₇ is hydrogen, substituted alkyl, O— alkyl or O— substituted alkyl.

Also provided are compounds of formula Ia wherein

R₂ is

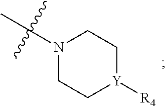

wherein Y is N and

R₄ is

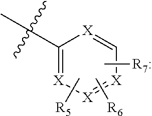

wherein

R₅ is chloro or fluoro,

R₆ is chloro, fluoro or hydrogen and

R₇ is hydrogen, substituted alkyl, O— alkyl or O— substituted alkyl.

Also provided are compounds of formula Ia wherein

R₂ is

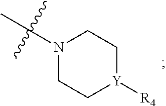

wherein Y is CH and

R₄ is

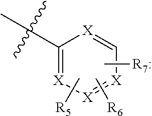

wherein X is CH or one of the X atoms is nitrogen and the remaining X atoms are carbon,

R₅ is chloro or fluoro,

R₆ is chloro, fluoro or hydrogen and

R₇ is hydrogen, substituted alkyl, O— alkyl or O— substituted alkyl.

Also provided are compounds of formula Ia wherein Q is N.

Also provided are compounds of formula Ia wherein Q is CH.

Also provided are compounds of the formula I-1 to I-58 in TABLE 1. Also provided are compounds I-59 to I-144 in TABLE 1.

In one embodiment of the present invention there is provided a compound according to formula I where in R¹, R², R³, R⁴, R⁵, R⁶, R^{3a}, R^{3b}, R^{3c}, R^{4a}, R^{4b}, R^{4c}, R^{4d}, R^{4e}, R^{4f}, Q, X and Y are as defined herein above. In all other embodiments provided below, substituents which can be present in each embodiment and which are not explicitly limited retain the broadest definition provided in the Summary of the Invention.

In another embodiment of the present invention there is provided a compound of formula I wherein R¹ is hydrogen or C₁₋₆ alkyl; R² is formula (II); and Y is NR⁴ or CR⁵R⁴.

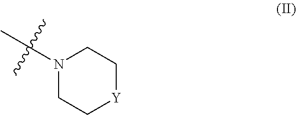

In another embodiment of the present invention there is provided a compound of formula I wherein R¹ is hydrogen or C₁₋₆ alkyl; R² is formula (II); and Y is NR⁴.

In another embodiment of the present invention there is provided a compound of formula I wherein R¹ is hydrogen or C₁₋₆ alkyl; R² is formula (II); and Y is CR⁵R⁴.

In another embodiment of the present invention there is provided a compound of formula I wherein Q is N; R¹ is hydrogen or C₁₋₆ alkyl; R² is formula (II); and Y is NR⁴.

In another embodiment of the present invention there is provided a compound of formula I wherein Q is N; R¹ is C₁₋₆ hydroxyalkyl or C₁₋₆ dihydroxyalkyl; R² is formula (II); and Y is NR⁴.

In another embodiment of the present invention there is provided a compound of formula I wherein Q is CH; R¹ is hydrogen or C₁₋₆ alkyl; R² is formula (II); and Y is NR⁴.

In another embodiment of the present invention there is provided a compound of formula I wherein Q is N; R¹ is hydrogen or C₁₋₆ alkyl; R² is formula (II); and Y is CR⁵R⁴.

In another embodiment of the present invention there is provided a compound of formula I wherein Q is CH; R¹ is hydrogen or C₁₋₆ alkyl; R² is formula (II); and Y is CR⁵R⁴.

In another embodiment of the present invention there is provided a compound of formula I wherein Q is N; R¹ is hydrogen or C₁₋₆ alkyl; R² is formula (II); Y is NR⁴; and R⁴ is optionally substituted phenyl.

In another embodiment of the present invention there is provided a compound of formula I wherein Q is CH; R¹ is hydrogen or C₁₋₆ alkyl; R² is formula (II); Y is NR⁴; and R⁴ is optionally substituted phenyl.

In another embodiment of the present invention there is provided a compound of formula I wherein Q is N; R¹ is hydrogen or C₁₋₆ alkyl; R² is formula (II); Y is CR⁵R⁴ and R⁴ is optionally substituted phenyl.

In another embodiment of the present invention there is provided a compound of formula I wherein Q is CH; R¹ is hydrogen or C₁₋₆ alkyl; R² is formula (II); Y is CR⁵R⁴; and R⁴ is optionally substituted phenyl.

In another embodiment of the present invention there is provided a compound of formula I wherein Q is N; R¹ is hydrogen or C₁₋₆ alkyl; R² is formula (II); Y is Nle; and R⁴ is phenyl substituted at least by one R⁶ selected from the group consisting of (c) C₁₋₆ hydroxyalkyl, (d) C₁₋₆-dihydroxyalkyl, (q) heterocyclyl-C₁₋₃ alkyl and (p) OR^{4d} wherein R^{4d} is selected from the group consisting of (iii) C₁₋₃ alkoxy-C₁₋₃ alkyl, (iv) C₁₋₆ hydroxyalkyl, (v) C₁₋₆ dihydroxyalkyl, (vi) (alkylene)₂₋₆NR^{4e}R^{4f} wherein R^{4e} and R^{4f} are independently hydrogen or C₁₋₆ alkyl or R^{4e} and R^{4f} together with the nitrogen to which they are attached form a cyclic amine optionally containing another heteroatom selected from NO, O or S(O)₀₋₂ wherein R^{4g} is hydrogen or C₁₋₃ alkyl, (vii) oxetanyl, (viii) tetrahydropyranyl, (ix) 1,1-dioxothianyl, (x) (1-oxothietan-3-yl)methyl and (xi) (alkylene)₂₋₆OR^{4h} wherein R^{4h} is C(O)CH(NH₂)R⁴ⁱ or P(═O)(OH)₂ wherein R⁴ⁱ C₁₋₆ alkyl and wherein said phenyl is optionally further substituted by one or two halogens. In a subembodiment there is provided a compound wherein one R⁶ is OR^{4d} and R^{4d} is selected from the group consisting of (iii) C₁₋₃ alkoxy-C₁₋₃ alkyl, (iv) C₁₋₆ hydroxyalkyl, (v) C₁₋₆ dihydroxyalkyl, (vi) (alkylene)₂₋₆NR^{4e}R^{4f} wherein R^{4e} and R^{4f} are independently hydrogen or C₁₋₆ alkyl or R^{4e} and R^{4f} together with the nitrogen to which they are attached form a cyclic amine optionally containing another heteroatom selected from NR^{4g}, O or S(O)₀₋₂ wherein R^{4g} is hydrogen or C₁₋₃ alkyl and wherein said phenyl is optionally further substituted by one or two halogens. In another subembodiment there is provided a compound wherein R⁶ is OR^{4d} wherein R^{4d} is (xi) (alkylene)₂₋₆OR^{4h} wherein R^{4h} is C(O)CH(NH₂)R⁴ⁱ or P(═O)(OH)₂ wherein R⁴ⁱ C₁₋₆ alkyl and wherein said phenyl is optionally further substituted by one or two halogens.

In another embodiment of the present invention there is provided a compound of formula I wherein Q is CH; R¹ is hydrogen or C₁₋₆ alkyl; R² is formula (II); Y is NR⁴; and R⁴ is phenyl substituted at least by one R⁶ selected from the group consisting of (c) C₁₋₆ hydroxyalkyl, (d) C₁₋₆-dihydroxyalkyl, (q) heterocyclyl-C₁₋₃ alkyl and (p) OR^{4d} wherein R^{4d} is selected from the group consisting of (iii) C₁₋₃ alkoxy-C₁₋₃ alkyl, (iv) C₁₋₆ hydroxyalkyl, (v) C₁₋₆ dihydroxyalkyl, (vi) (alkylene)₂₋₆NR^{4e}R^{4f} wherein R^{4e} and R^{4f} are independently hydrogen or C₁₋₆ alkyl or R^{4e} and R^{4f} together with the nitrogen to which they are attached form a cyclic amine optionally containing another heteroatom selected from NR^{4g}, O or S(O)₀₋₂ wherein R^{4g} is hydrogen or C₁₋₃ alkyl, (vii) oxetanyl, (viii) tetrahydropyranyl, (ix) 1,1-dioxothianyl, (x) (1-oxothietan-3-yl)methyl and (xi) (alkylene)₂₋₆OR^{4h} wherein R^{4h} is C(O)CH(NH₂)R⁴ⁱ or P(═O)(OH)₂ wherein R⁴ⁱ C₁₋₆ alkyl and wherein said phenyl is optionally further substituted by one or two halogens. In a subembodiment there is provided a compound wherein one R⁶ is OR^{4d} and R^{4d} is selected from the group consisting of (iii) C₁₋₃ alkoxy-C₁₋₃ alkyl, (iv) C₁₋₆ hydroxyalkyl, (v) C₁₋₆ dihydroxyalkyl, (vi) (alkylene)₂₋₆NR^{4e}R^{4f} wherein R^{4e} and R^{4f} are independently hydrogen or C₁₋₆ alkyl or R^{4e} and R^{4f} together with the nitrogen to which they are attached form a cyclic amine optionally containing another heteroatom selected from NR^{4g}, O or S(O)₀₋₂ wherein R^{4g} is hydrogen or C₁₋₃ alkyl and wherein said phenyl is optionally further substituted by one or two halogens. In another subembodiment there is provided a compound wherein R⁶ is OR^{4d} wherein R^{4d} is (xi) (alkylene)₂₋₆OR^{4h} wherein R^{4h} is C(O)CH(NH₂)R⁴ⁱ or P(═O)(OH)₂ wherein R⁴ⁱ C₁₋₆ alkyl and wherein said phenyl is optionally further substituted by one or two halogens.

In another embodiment of the present invention there is provided a compound of formula I wherein Q is N; R¹ is hydrogen or C₁₋₆ alkyl; R² is formula (II); Y is CR⁵R⁴; and R⁴ is phenyl substituted at least by one R⁶ selected from the group consisting of (c) C₁₋₆ hydroxyalkyl, (d) C₁₋₆-dihydroxyalkyl, (q) heterocyclyl-C₁₋₃ alkyl and (p) OR^{4d} wherein R^{4d} is selected from the group consisting of (iii) C₁₋₃ alkoxy-C₁₋₃ alkyl, (iv) C₁₋₆ hydroxyalkyl, (v) C₁₋₆ dihydroxyalkyl, (vi) (alkylene)₂₋₆NR^{4e}R^{4f} wherein R^{4e} and R^{4f} are independently hydrogen or C₁₋₆ alkyl or R^{4e} and R^{4f} together with the nitrogen to which they are attached form a cyclic amine optionally containing another heteroatom selected from NR^{4g}, O or S(O)₀₋₂ wherein R^{4g} is hydrogen or C₁₋₃ alkyl, (vii) oxetanyl, (viii) tetrahydropyranyl, (ix) 1,1-dioxothianyl, (x) (1-oxothietan-3-yl)methyl and (xi) (alkylene)₂₋₆OR^{4h} wherein R^{4h} is C(O)CH(NH₂)R⁴ⁱ or P(═O)(OH)₂ wherein R⁴ⁱ C₁₋₆ alkyl and wherein said phenyl is further optionally substituted by one or two halogens and wherein said phenyl is further optionally substituted by one or two halogens. In a subembodiment there is provided a compound wherein one R⁶ is OR^{4d} and R^{4d} is selected from the group consisting of (iii) C₁₋₃ alkoxy-C₁₋₃ alkyl, (iv) C₁₋₆ hydroxyalkyl, (v) C₁₋₆ dihydroxyalkyl, (vi) (alkylene)₂₋₆NR^{4e}R^{4f} wherein R^{4e} and R^{4f} are independently hydrogen or C₁₋₆ alkyl or Rae and R^{4f} together with the nitrogen to which they are attached form a cyclic amine optionally containing another heteroatom selected from NR^{4g}, O or S(O)₀₋₂ wherein R^{4g} is hydrogen or C₁₋₃ alkyl and wherein said phenyl is optionally further substituted by one or two halogens. In another subembodiment there is provided a compound wherein R⁶ is OR^{4d} wherein R^{4d} is (xi) (alkylene)₂₋₆OR^{4h} wherein R⁴ⁱ is C(O)CH(NH₂)R⁴ⁱ or P(═O)(OH)₂ wherein R⁴ⁱ C₁₋₆ alkyl and wherein said phenyl is optionally further substituted by one or two halogens. In another embodiment of the present invention there is provided a compound of formula I wherein Q is CH; R¹ is hydrogen or C₁₋₆ alkyl; R² is formula (II); Y is CR⁵R⁴; and R⁴ is phenyl substituted at least by one R⁶ selected from the group consisting of (c) C₁₋₆ hydroxyalkyl, (d) C₁₋₆-dihydroxyalkyl, (q) heterocyclyl C₁₋₃ alkyl and (p) OR^{4d} wherein R^{4d} is selected from the group consisting of (iii) C₁₋₃ alkoxy-C₁₋₃ alkyl, (iv) C₁₋₆ hydroxyalkyl said hydroxyalkyl further optionally substituted with halogen, (v) C₁₋₆ dihydroxyalkyl, (vi) (alkylene)₂₋₆NR^{4e}R^{4f} wherein R^{4e} and R^{4f} are independently hydrogen or C₁₋₆ alkyl or R^{4e} and R^{4f} together with the nitrogen to which they are attached form a cyclic amine optionally containing another heteroatom selected from NR^{4g}, O or S(O)₀₋₂ wherein R^{4g} is hydrogen or C₁₋₃ alkyl, (vii) oxetanyl, (viii) tetrahydropyranyl, (ix) 1,1-dioxothianyl, (x) (1-oxothietan-3-yl)methyl and (xi) (alkylene)₂₋₆OR^{4h} wherein R⁴ⁱ is C(O)CH(NH₂)R⁴ⁱ wherein R⁴ⁱ C₁₋₆ alkyl or P(═O)(OH)₂ and wherein said phenyl is further optionally substituted by one or two halogens. In a subembodiment there is provided a compound wherein one R⁶ is OR^{4d} and R^{4d} is selected from the group consisting of (iii) C₁₋₃ alkoxy-C₁₋₃ alkyl, (iv) C₁₋₆ hydroxyalkyl, (v) C₁₋₆ dihydroxyalkyl, (vi) (alkylene)₂₋₆NR^{4e}R^{4f} wherein R^{4e} and R^{4f} are independently hydrogen or C₁₋₆ alkyl or R^{4e} and R^{4f} together with the nitrogen to which they are attached form a cyclic amine optionally containing another heteroatom selected from NR^{4g}, O or S(O)₀₋₂ wherein R^{4g} is hydrogen or C₁₋₃ alkyl and wherein said phenyl is optionally further substituted by one or two halogens. In another subembodiment there is provided a compound wherein R⁶ is OR^{4d} wherein R^{4d} is (xi) (alkylene)₂₋₆OR^{4h} wherein R^{4h} is C(O)CH(NH₂)R⁴ⁱ or P(═O)(OH)₂ wherein R⁴ⁱ C₁₋₆ alkyl and wherein said phenyl is optionally further substituted by one or two halogens.

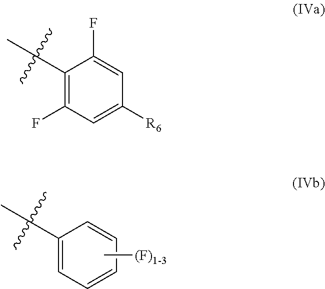

In another embodiment of the present invention there is provided a compound of formula I wherein Q is N, Y is Nle; R⁴ is IVa; and R⁶ is selected from the group consisting of (c) C₁₋₆ hydroxyalkyl, (d) C₁₋₆-dihydroxyalkyl, (q) heterocyclyl-C₁₋₃ alkyl and (p) OR^{4d} wherein R^{4d} is selected from the group consisting of (iii) C₁₋₃ alkoxy-C₁₋₃ alkyl, (iv) C₁₋₆ hydroxyalkyl, (v) C₁₋₆ dihydroxyalkyl, (vi) (alkylene)₂₋₆NR^{4e}R^{4f} wherein R^{4e} and R^{4f} are independently hydrogen or C₁₋₆ alkyl or R^{4e} and R^{4f} together with the nitrogen to which they are attached form a cyclic amine optionally containing another heteroatom selected from NR^{4g}, O or S(O)₀₋₂ wherein R^{4g} is hydrogen or C₁₋₃ alkyl, (vii) oxetanyl, (viii) tetrahydropyranyl, (ix) 1,1-dioxothianyl, (x) (1-oxothietan-3-yl)methyl and (xi) (alkylene)₂₋₆OR⁴⁶ wherein R⁴ⁱ is C(O)CH(NH₂)R⁴ⁱ or P(═O)(OH)₂ wherein R⁴ⁱ C₁₋₆ alkyl. In a subembodiment there is provided a compound wherein one R⁶ is OR^{4d} and R^{4d} is selected from the group consisting of (iii) C₁₋₃ alkoxy-C₁₋₃ alkyl, (iv) C₁₋₆ hydroxyalkyl, (v) C₁₋₆ dihydroxyalkyl, (vi) (alkylene)₂₋₆NR^{4e}R^{4f} wherein R^{4e} and R^{4f} are independently hydrogen or C₁₋₆ alkyl or R^{4e} and R^{4f} together with the nitrogen to which they are attached form a cyclic amine optionally containing another heteroatom selected from NR^{4g}, O or S(O)₀₋₂ wherein R^{4g} is hydrogen or C₁₋₃ alkyl and wherein said phenyl is optionally further substituted by one or two halogens. In another subembodiment there is provided a compound wherein R⁶ is OR^{4d} wherein R^{4d} is (xi) (alkylene)₂₋₆OR^{4h} wherein R⁴ⁱ is C(O)CH(NH₂)R⁴ⁱ or P(═O)(OH)₂ wherein R⁴ⁱ C₁₋₆ alkyl and wherein said phenyl is optionally further substituted by one or two halogens.

In another embodiment of the present invention there is provided a compound of formula I wherein Q is N, Y is NR⁴; R⁴ is IVa; and R⁶ is selected from the group consisting of (c) C₁₋₆ hydroxyalkyl, (d) C₁₋₆-dihydroxyalkyl, (q) heterocyclyl-C₁₋₃ alkyl and (p) OR^{4d} wherein R^{4d} is selected from the group consisting of (iii) C₁₋₃ alkoxy-C₁₋₃ alkyl, (iv) C₁₋₆ hydroxyalkyl, (v) C₁₋₆ dihydroxyalkyl, (vi) (alkylene)₂₋₆NR^{4e}R^{4f} wherein R^{4g} and R^{4f} are independently hydrogen or C₁₋₆ alkyl or R^{4e} and R^{4f} together with the nitrogen to which they are attached form a cyclic amine optionally containing another heteroatom selected from NR^{4g}, O or S(O)₀₋₂ wherein R^{4g} is hydrogen or C₁₋₃ alkyl and (xi) (alkylene)₂₋₆OR^{4h} wherein R^{4h} is C(O)CH(NH₂)R⁴ⁱ or P(═O)(OH)₂ wherein R⁴ⁱ C₁₋₆ alkyl. In a subembodiment there is provided a compound wherein one R⁶ is OR^{4d} and R^{4d} is selected from the group consisting of (iii) C₁₋₃ alkoxy-C₁₋₃ alkyl, (iv) C₁₋₆ hydroxyalkyl, (v) C₁₋₆ dihydroxyalkyl, (vi) (alkylene)₂₋₆NR^{4e}R^{4f} wherein R^{4e} and R^{4f} are independently hydrogen or C₁₋₆ alkyl or R^{4e} and R^{4f} together with the nitrogen to which they are attached form a cyclic amine optionally containing another heteroatom selected from NR^{4g}, O or S(O)₀₋₂ wherein R^{4g} is hydrogen or C₁₋₃ alkyl and wherein said phenyl is optionally further substituted by one or two halogens. In another subembodiment there is provided a compound wherein R⁶ is OR^{4d} wherein R^{4d} is (xi) (alkylene)₂₋₆OR^{4h} wherein R^{4h} is C(O)CH(NH₂)R⁴ⁱ or P(═O)(OH)₂ wherein R⁴ⁱ C₁₋₆ alkyl and wherein said phenyl is optionally further substituted by one or two halogens.

In another embodiment of the present invention there is provided a compound of formula I wherein Q is N; R¹ is hydrogen or C₁₋₆ alkyl; R² is II; Y is NR⁴; and, R⁴ is IVb.

In another embodiment of the present invention there is provided a compound of formula I wherein Q is CH; R¹ is hydrogen or C₁₋₆ alkyl; R² is II; Y is NR⁴; and, R⁴ is IVb.

In another embodiment of the present invention there is provided a compound of formula I wherein Q is N; R¹ is hydrogen or C₁₋₆ alkyl; R² is II; Y is CR⁵R⁴; R⁵ is hydrogen; and, R⁴ is IVb.

In another embodiment of the present invention there is provided a compound of formula I wherein Q is CH; R¹ is hydrogen or C₁₋₆ alkyl; R² is II; Y is CR⁵R⁴; R⁵ is hydrogen; and, R⁴ is IVb

In another embodiment of the present invention there is provided a compound of formula I wherein Q is N; R¹ is hydrogen or C₁₋₆ alkyl; R² is II; Y is NR⁴; and, R⁴ is optionally substituted pyridinyl.

In another embodiment of the present invention there is provided a compound of formula I wherein Q is CH; R¹ is hydrogen or C₁₋₆ alkyl; R² is II; Y is Nle and, R⁴ is optionally substituted pyridinyl.

In another embodiment of the present invention there is provided a compound of formula I wherein Q is N; R¹ is hydrogen or C₁₋₆ alkyl; R² is II; Y is CR⁵R⁴; R⁴ is optionally substituted pyridinyl; and R⁵ is hydrogen or C₁₋₆ alkyl.

In another embodiment of the present invention there is provided a compound of formula I wherein Q is CH; R¹ is hydrogen or C₁₋₆ alkyl; R² is II; Y is CR⁵R⁴; R⁴ is optionally substituted pyridinyl; and R⁵ is hydrogen or C₁₋₆ alkyl.

In another embodiment of the present invention there is provided a compound of formula I wherein Q is N; R¹ is hydrogen or C₁₋₆ alkyl; R² is formula (II); Y is NR⁴ and R⁴ is optionally substituted heteroaryl.

In another embodiment of the present invention there is provided a compound of formula I wherein Q is CH; R¹ is hydrogen or C₁₋₆ alkyl; R² is formula (II); Y is NR⁴ and R⁴ is optionally substituted heteroaryl.

In another embodiment of the present invention there is provided a compound of formula I wherein Q is N; R¹ is hydrogen or C₁₋₆ alkyl; R² is formula (II); Y is CR⁵R⁴; R⁴ is optionally substituted heteroaryl; and, R⁵ is hydrogen or C₁₋₆ alkyl.

In another embodiment of the present invention there is provided a compound of formula I wherein Q is CH; R¹ is hydrogen or C₁₋₆ alkyl; R² is formula (II); Y is CR⁵R⁴; R⁴ is optionally substituted heteroaryl; and, R⁵ is hydrogen or C₁₋₆ alkyl.

In another embodiment of the present invention there is provided a compound of formula I wherein Q is N; R¹ is hydrogen or C₁₋₆ alkyl; R² is formula (II); Y is NR⁴; and, R⁴ is optionally substituted heteroaryl selected from the group consisting of (a) pyridinyl, (b) pyrimidinyl, (c) thiazolyl, (d) isothiazolyl, (e) oxazolyl, (f) isoxazolyl, (g) imidazolyl, (h) pyrazolyl, (i) 1,2,4-triazolyl, (j) 3-(pyrazinyl)-1,2,4-oxadiazolyl and (k) 1,2,4-oxadiazolyl.

In another embodiment of the present invention there is provided a compound of formula I wherein Q is CH; R¹ is hydrogen or C₁₋₆ alkyl; R² is formula (II); Y is NR⁴; and, R⁴ is optionally substituted heteroaryl selected from the group consisting of (a) pyridinyl, (b) pyrimidinyl, (c) thiazolyl, (d) isothiazolyl, (e) oxazolyl, (f) isoxazolyl, (g) imidazolyl, (h) pyrazolyl, (i) 1,2,4-triazolyl, (j) 3-(pyrazinyl)-1,2,4-oxadiazolyl and (k) 1,2,4-oxadiazolyl.

In another embodiment of the present invention there is provided a compound of formula I wherein Q is N; R¹ is hydrogen or C₁₋₆ alkyl; R² is formula (II); Y is CR⁵R⁴; R⁴ is optionally substituted heteroaryl selected from the group consisting of (a) pyridinyl, (b) pyrimidinyl, (c) thiazolyl, (d) isothiazolyl, (e) oxazolyl, (f) isoxazolyl, (g) imidazolyl, (h) pyrazolyl, (i) 1,2,4-triazolyl, (j) 3-(pyrazinyl)-1,2,4-oxadiazolyl and (k) 1,2,4-oxadiazolyl; and, R⁵ is hydrogen or C₁₋₆ alkyl.

In another embodiment of the present invention there is provided a compound of formula I wherein Q is CH; R¹ is hydrogen or C₁₋₆ alkyl; R² is formula (II); Y is CR⁵R⁴ and R⁴ is optionally substituted heteroaryl selected from the group consisting of (a) pyridinyl, (b) pyrimidinyl, (c) thiazolyl, (d) isothiazolyl, (e) oxazolyl, (f) isoxazolyl, (g) imidazolyl, (h) pyrazolyl, (i) 1,2,4-triazolyl, (j) 3-(pyrazinyl)-1,2,4-oxadiazolyl and (k) 1,2,4-oxadiazolyl; and,
R⁵ is hydrogen or C₁₋₆ alkyl.

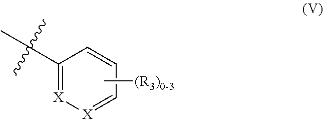

In another embodiment of the present invention there is afforded a compound of formula I R¹ is hydrogen or C₁₋₆ alkyl and R² is (V).

In another embodiment of the present invention there is afforded a compound of formula I wherein Q is N, R¹ is hydrogen or C₁₋₆ alkyl and R² is V.

In another embodiment of the present invention there is afforded a compound of formula I wherein Q is CH, R¹ is hydrogen or C₁₋₆ alkyl and R² is V.

In another embodiment of the present invention there is afforded a compound of formula I wherein Q is N, R¹ is C₁₋₆ alkyl and R² is V.

In another embodiment of the present invention there is afforded a compound of formula I wherein Q is CH, R¹ is C₁₋₆ alkyl and R² is V.

In another embodiment of the present invention there is afforded a compound of formula I wherein R⁴ is hydrogen or C₁₋₆ alkyl; R² is V; and, each X is CH.

In another embodiment of the present invention there is afforded a compound of formula I wherein Q is N, R¹ is hydrogen or C₁₋₆ alkyl; R² is V; and, each X is CH.

In another embodiment of the present invention there is afforded a compound of formula I wherein Q is CH, R¹ is hydrogen or C₁₋₆ alkyl; R² is V; and, each X is CH.

In another embodiment of the present invention there is afforded a compound of formula I wherein Q is N, R¹ is C₁₋₆ alkyl; R² is V; and, each X is CH.

In another embodiment of the present invention there is afforded a compound of formula I wherein Q is CH, R¹ is C₁₋₆ alkyl; R² is V; and, each X is CH.

In another embodiment of the present invention there is afforded a compound of formula I wherein R¹ is hydrogen or C₁₋₆ alkyl; R² is V; and, one X is N and the other X is CH.

In another embodiment of the present invention there is afforded a compound of formula I wherein Q is N, R¹ is hydrogen or C₁₋₆ alkyl; R² is V; and; one X is N and the other X is CH.

In another embodiment of the present invention there is afforded a compound of formula I wherein Q is CH, R¹ is hydrogen or C₁₋₆ alkyl; R² is V; and, one X is N and the other X is CH.

In another embodiment of the present invention there is afforded a compound of formula I wherein Q is N, R¹ is C₁₋₆ alkyl; R² is V; and, one X is N and the other X is CH.

In another embodiment of the present invention there is afforded a compound of formula I wherein Q is CH, R¹ is C₁₋₆ alkyl; R² is V; and, one X is N and the other X is CH.

In another embodiment of the present invention there is afforded a compound of formula I wherein R¹ is hydrogen or C₁₋₆ alkyl; R² is (V); each X is CH; and, each R³ is independently selected from the group consisting of C₁₋₆ alkyl, C₁₋₆ haloalkyl, halo, cyano, C₁₋₆ alkylsulfonyl, and OR^{4d} wherein R^{4d} is selected from the group consisting of (i) C₁₋₆ alkyl (ii) C₁₋₃ alkoxy-C₁₋₃ alkyl, (iii) C₁₋₆ hydroxyalkyl and (iv) C₁₋₆ dihydroxyalkyl.

In another embodiment of the present invention there is afforded a compound of formula I wherein Q is N, R¹ is hydrogen or C₁₋₆ alkyl; R² is V; each X is CH; and, each R³ is independently selected from the group consisting of C₁₋₆ alkyl, C₁₋₆ haloalkyl, halo, cyano, C₁₋₆ alkylsulfonyl, and OR^{4d} wherein R^{4d} is selected from the group consisting of (i) C₁₋₆ alkyl (ii) C₁₋₃ alkoxy-C₁₋₃ alkyl, (iii) C₁₋₆ hydroxyalkyl and (iv) C₁₋₆ dihydroxyalkyl.

In another embodiment of the present invention there is afforded a compound of formula I wherein Q is CH; R¹ is hydrogen or C₁₋₆ alkyl; R² is V); each X is CH; and, each R³ is independently selected from the group consisting of C₁₋₆ alkyl, C₁₋₆ haloalkyl, halo, cyano, C₁₋₆ alkylsulfonyl, and OR^{4d} wherein R^{4d} is selected from the group consisting of (i) C₁₋₆ alkyl (ii) C₁₋₃ alkoxy-C₁₋₃ alkyl, (iii) C₁₋₆ hydroxyalkyl and (iv) C₁₋₆ dihydroxyalkyl.

In another embodiment of the present invention there is afforded a compound of formula I wherein Q is N; R¹ is C₁₋₆ alkyl; R² is V; each X is CH; and, each R³ is independently selected from the group consisting of C₁₋₆ alkyl, C₁₋₆ haloalkyl, halo, cyano, C₁₋₆ alkylsulfonyl, and OR^{4d} wherein R^{4d} is selected from the group consisting of (i) C₁₋₆ alkyl (ii) C₁₋₃ alkoxy-C₁₋₃ alkyl, (iii) C₁₋₆ hydroxyalkyl and (iv) C₁₋₆ dihydroxyalkyl.

In another embodiment of the present invention there is afforded a compound of formula I wherein Q is CH; R¹ is C₁₋₆ alkyl; R² is V; each X is CH; and, each R³ is independently selected from the group consisting of C₁₋₆ alkyl, C₁₋₆ haloalkyl, halo, cyano, C₁₋₆ alkylsulfonyl, and OR^{4d} wherein R^{4d} is selected from the group consisting of (i) C₁₋₆ alkyl (ii) C₁₋₃ alkoxy-C₁₋₃ alkyl, (iii) C₁₋₆ hydroxyalkyl and (iv) C₁₋₆ dihydroxyalkyl.

In another embodiment of the present invention there is afforded a compound of formula I wherein R¹ is hydrogen or C₁₋₆ alkyl; R² is V; each X is CH; and, each R³ is independently selected from the group consisting of C₁₋₆ alkyl, C₁₋₆ haloalkyl, halo, cyano, C₁₋₆ alkylsulfonyl, and OR^{4d} wherein R^{4d} is selected from the group consisting of (i) C₁₋₆ alkyl (ii) C₁₋₃ alkoxy-C₁₋₃ alkyl, (iii) C₁₋₆ hydroxyalkyl and (iv) C₁₋₆ dihydroxyalkyl.

In another embodiment of the present invention there is afforded a compound of formula I wherein Q is N; R¹ is hydrogen or C₁₋₆ alkyl; R² is V; one X is N and the other X is CH; and, each R³ is independently selected from the group consisting of C₁₋₆ alkyl, C₁₋₆ haloalkyl, halo, cyano, C₁₋₆ alkylsulfonyl, and OR^{4d} wherein R^{4D} is selected from the group consisting of (i) C₁₋₆ alkyl (ii) C₁₋₃ alkoxy-C₁₋₃ alkyl, (iii) C₁₋₆ hydroxyalkyl and (iv) C₁₋₆ dihydroxyalkyl.

In another embodiment of the present invention there is afforded a compound of formula I wherein Q is CH; R¹ is hydrogen or C₁₋₆ alkyl; R² is. V; one X is N and the other X is CH and, each R³ is independently selected from the group consisting of C₁₋₆ alkyl, C₁₋₆ haloalkyl, halo, cyano, C₁₋₆ alkylsulfonyl, and OR^{4d} wherein R^{4d} is selected from the group consisting of (i) C₁₋₆ alkyl (ii) C₁₋₃ alkoxy-C₁₋₃ alkyl, (iii) C₁₋₆ hydroxyalkyl and (iv) C₁₋₆ dihydroxyalkyl.

In another embodiment of the present invention there is afforded a compound of formula I wherein Q is N; R¹ is C₁₋₆ alkyl; R² is V; one X is N and the other X is C; and, each R³ is independently selected from the group consisting of C₁₋₆ alkyl, C₁₋₆ haloalkyl, halo, cyano, C₁₋₆ alkylsulfonyl, and OR^{4d} wherein R^{4d} is selected from the group consisting of (i) C₁₋₆ alkyl (ii) C₁₋₃ alkoxy-C₁₋₃ alkyl, (iii) C₁₋₆ hydroxyalkyl and (iv) C₁₋₆ dihydroxyalkyl.

In another embodiment of the present invention there is afforded a compound of formula I wherein Q is CH; R¹ is C₁₋₆ alkyl; R² is V; one X is N and the other X is CH and, each R³ is independently selected from the group consisting of C₁₋₆ alkyl, C₁₋₆ haloalkyl, halo, cyano, C₁₋₆ alkylsulfonyl, and OR^{4d} wherein R^{4d} is selected from the group consisting of (i) C₁₋₆ alkyl (ii) C₁₋₃ alkoxy-C₁₋₃ alkyl, (iii) C₁₋₆ hydroxyalkyl and (iv) C₁₋₆ dihydroxyalkyl.

In another embodiment of the present invention there is provided a method of inhibiting tankyrase 1 and/or tankyrase 2 by contacting either or both with a compound for formula I wherein R¹, R², R³, R⁴, R⁵, R⁶, R^{3a}, R^{3b}, R^{3c}, R^{4a}, R^{4b}, R^{4c}, R^{4d}, R^{4e}, R^{4f}, Q, X and Y are as defined hereinabove.

In another embodiment of the present invention there is provided a method for treating cancer by administering to a patient in need thereof a therapeutically active amount of a compound according to formula I wherein R¹, R², R³, R⁴, R⁵, R⁶, R^{3a}, R^{3b}, R^{3c}, R^{4a}, R^{4ab}, R^{4c}, R^{4d}, R^{4e}, R^{4f}, Q, X and Y are as defined hereinabove.

In another embodiment of the present invention there is provided a method for treating colorectal cancer by administering to a patient in need thereof a therapeutically active amount of a compound according to formula I wherein R¹, R², R³, R⁴, R⁵, R⁶, R^{3a}, R^{3b}, R^{3c}, R^{4a}, R^{4ab}, R^{4c}, R^{4d}, R^{4e}, R^{4f}, Q, X and Y are as defined hereinabove.

In another embodiment of the present invention there is provided a compound according to formula I for the preparation of a medicament for the treatment of cancer wherein R¹, R², R³, R⁴, R⁵, R⁶, R^{3a}, R^{3b}, R^{3c}, R^{4a}, R^{4ab}, R^{4c}, R^{4d}, R^{4e}, R^{4f}, Q, X and Y are as defined hereinabove.

In another embodiment of the present invention there is provided a pharmaceutical composition containing a compound according to formula I wherein R¹, R², R³, R⁴, R⁵, R⁶, R^{3a}, R^{3b}, R^{3c}, R^{4a}, R^{4ab}, R^{4c}, R^{4d}, R^{4e}, R^{4f}, Q, X and Y are as defined hereinabove and at least one pharmaceutically acceptable carrier, diluent or excipient.

In another embodiment of the present invention there is provided a compound of the formula I′

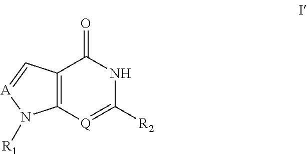

wherein

Q is N or CH,

A is N,

R₁ is selected from the group consisting of hydrogen and alkyl,

R₂ is

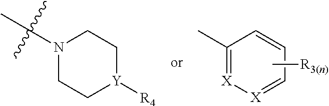

R₄ is selected from the group consisting of

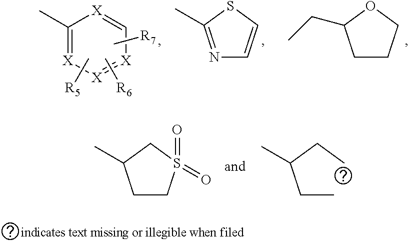

### X is CH or N,

Y is selected from the group consisting of nitrogen, carbon, COH and CCN,

R₅ is halogen,

R₆ is halogen or hydrogen,

R₃ and R₇ is selected from the group consisting of hydrogen, alkyl, substituted alkyl, haloalkyl, halogen, O-alkyl, O-substituted alkyl, CN, trifluoromethyl, nitro, carboxyalkyl, alkylsulfonyl, hydroxyl, —NH₂, hydroxyalkyl, carboxylic acid, sulfonamide, tetrazole and alkyl ketone and

n is 0 to 3

or a pharmaceutically acceptable salt thereof.

In another embodiment of the present invention there is provided a compound of formula I′ as described herein wherein

R₁ is selected from hydrogen or alkyl,

R₂ is

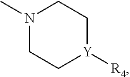

R₄ is

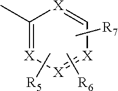

X is CH or N,

Y is selected from the group consisting of nitrogen, carbon, COH and CCN,

R₅ is halogen,

R₆ is halogen or hydrogen and

R₇ is selected from the group consisting of hydrogen, alkyl, substituted alkyl, haloalkyl, halogen, O-alkyl, O-substituted alkyl, CN, trifluoromethyl, nitro, carboxyalkyl, alkylsulfonyl, hydroxyl, —NH₂, hydroxyalkyl, carboxylic acid, sulfonamide, tetrazole and alkyl ketone.

In another embodiment of the present invention there is provided a compound of formula I′ as described herein wherein

X is CH

R₂ is

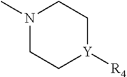

wherein Y is N

R₄ is

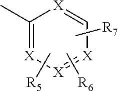

R₅ is chloro or fluoro,

R₆ is chloro, fluoro or hydrogen and

R₇ is hydrogen, substituted alkyl, O— alkyl or O— substituted alkyl.

In another embodiment of the present invention there is provided a compound of formula I′ as described herein wherein

R₂ is

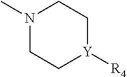

wherein Y is N and

R₄ is

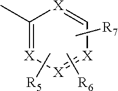

wherein for R₄ one of X atoms is nitrogen and the remaining X atoms are CH

R₅ is chloro or fluoro,

R₆ is chloro, fluoro or hydrogen and

R₇ is hydrogen, substituted alkyl, O— alkyl or O— substituted alkyl.

In another embodiment of the present invention there is provided a compound of formula I′ as described herein wherein

R₂ is

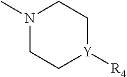

wherein Y is CH and

R₄ is

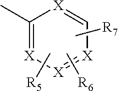

wherein for R₄ one of the X atoms is nitrogen and the remaining X atoms are carbon

R₅ is chloro or fluoro,

R₆ is chloro, fluoro or hydrogen and

R₇ is hydrogen, substituted alkyl, O— alkyl or O— substituted alkyl.

In another embodiment of the present invention there is provided a compound of formula I′ as described herein wherein

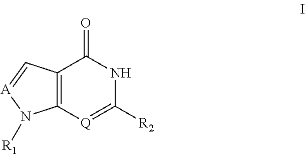

wherein Q is CH,

A is N,

R₁ is selected from the group consisting of hydrogen and alkyl,

R₂ is

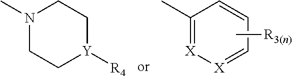

R₄ is selected from the group consisting of

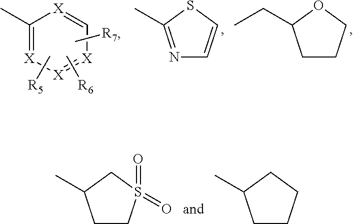

X is CH or N,

Y is selected from the group consisting of nitrogen, carbon, COH and CCN,

R₅ is halogen,

R₆ is halogen or hydrogen,

R₃ and R₇ is selected from the group consisting of hydrogen, alkyl, substituted alkyl, haloalkyl, halogen, O-alkyl, O-substituted alkyl, CN, trifluoromethyl, nitro, carboxyalkyl, alkylsulfonyl, hydroxyl, —NH₂, hydroxyalkyl, carboxylic acid, sulfonamide, tetrazole and alkyl ketone and

n is 0 to 3.

In another embodiment of the present invention there is provided a compound of formula I′ as described herein wherein

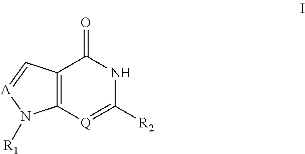

wherein Q is N,

A is N,

R₁ is selected from the group consisting of hydrogen and alkyl,

R₂ is

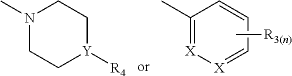

R₄ is selected from the group consisting of

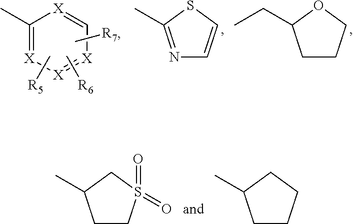

X is CH or N,

Y is selected from the group consisting of nitrogen, carbon, COH and CCN,

R₅ is halogen,

R₆ is halogen or hydrogen,

R₃ and R₇ is selected from the group consisting of hydrogen, alkyl, substituted alkyl, haloalkyl, halogen, O-alkyl, O-substituted alkyl, CN, trifluoromethyl, nitro, carboxyalkyl, alkylsulfonyl, hydroxyl, —NH₂, hydroxyalkyl, carboxylic acid, sulfonamide, tetrazole and alkyl ketone and

n is 0 to 3.

In another embodiment of the present invention there is provided a compound of formula I′ as described herein wherein the compound is selected from the group consisting of
1-Methyl-6-(4-thiazol-2-yl-piperazin-1-yl)-1,5-dihydro-pyrazolo[3,4-d]pyrimidin-4-one,
6-[4-(4-Fluoro-3-trifluoromethyl-phenyl)-piperazin-1-yl]-1-methyl-1,5-dihydropyrazolo[3,4-d]pyrimidin-4-one,
4-[4-(1-Methyl-4-oxo-4,5-dihydro-1H-pyrazolo[3,4-d]pyrimidin-6-yl)-piperazin-1-yl]-benzoic acid ethyl ester,
6-[4-(2,4-Difluoro-phenyl)-piperazin-1-yl]-1-methyl-1,5-dihydro-pyrazolo[3,4-d]pyrimidin-4-one,
6-[4-(2-Fluoro-phenyl)-piperazin-1-yl]-1-methyl-1,5-dihydro-pyrazolo[3,4-d]pyrimidin-4-one,
2-[4-(1-Methyl-4-oxo-4,5-dihydro-1H-pyrazolo[3,4-d]pyrimidin-6-yl)-piperazin-1-yl]-benzonitrile,
3-Fluoro-4-[4-(1-methyl-4-oxo-4,5-dihydro-1H-pyrazolo[3,4-d]pyrimidin-6-yl)-piperazin-1-yl]-benzonitrile,
6-{4-[2-Fluoro-4-(2-methoxy-ethoxy)-phenyl]-piperazin-1-yl}-1-methyl-1,5-dihydropyrazolo[3,4-d]pyrimidin-4-one,
6-[4-(3-Methoxy-pyridin-2-yl)-piperazin-1-yl]-1-methyl-1,5-dihydro-pyrazolo[3 d]pyrimidin-4-one,
6-{4-[2,6-Difluoro-4-(2-methoxy-ethoxy)-phenyl]-piperazin-1-yl}-1-methyl-1,5-dihydropyrazolo[3,4-d]pyrimidin-4-one and
3-Fluoro-4-[4-(1-methyl-4-oxo-4,5-dihydro-1H-pyrazolo[3,4-d]pyrimidin-6-yl]-piperazin-1-A-benzenesulfonamide.

In another embodiment of the present invention there is provided a compound of formula I′ as described herein wherein the compound is selected from the group consisting of
6-[4-(4-Fluoro-phenyl)-piperidin-1-yl]-1-methyl-1,5-dihydro-pyrazolo[3,4-d]pyrimidin-4-one,
1-Methyl-6-[4-(4-trifluoromethyl-phenyl)-piperidin-1-yl]-1,5-dihydro-pyrazolo[3,4-d]pyrimidin-4-one,
4-(4-Fluoro-phenyl)-1-(1-methyl-4-oxo-4,5-dihydro-1H-pyrazolo[3,4-d]pyrimidin-6-yl)-piperidine-4-carbonitrile,
1′-(1-Methyl-4-oxo-4,5-dihydro-1H-pyrazolo[3,3-d]pyrimidin-6-yl)-2′,3′,5′,6′-tetrahydro-1′H-[2,4′]bipyridinyl-4′-carbonitrile,
1-Methyl-6-[4-(tetrahydro-furan-2-ylmethyl)-piperazin-1-yl]-1,5-dihydro-pyrazolo[3,4-d]pyrimidin-4-one,
6-[4-(3,5-Dichloro-pyridin-4-yl)-piperazin-1-yl]-1-methyl-1,5-dihydro-pyrazolo[3,4-d]pyrimidin-4-one,
3-Fluoro-4-[4-(1-methyl-4-oxo-4,5-dihydro-1H-pyrazolo[3,4-d]pyrimidin-6-yl)-piperazin-1-yl]-benzoic acid,
6-[4-(2,6-Dichloro-phenyl)-piperazin-1-yl]-1-methyl-1,5-dihydro-pyrazolo[3,4-d]pyrimidin-4-one,
6-[4-(2,6-Difluoro-4-propionyl-phenyl)-piperazin-1-yl]-1-methyl-1,5-dihydro-pyrazolo[3,4-d]pyrimidin-4-one,
6-[4-(2,3-Dichloro-pyridin-4-yl)-piperazin-1-yl]-1-methyl-1,5-dihydro-pyrazolo[3,4-d]pyrimidin-4-one and
2-[4-(1-Methyl-4-oxo-4,5-dihydro-1H-pyrazolo[3,4-d]pyrimidin-6-yl)-piperazin-1-yl]-nicotinonitrile.

In another embodiment of the present invention there is provided a compound of formula I′ as described herein wherein the compound is selected from the group consisting of
1-Methyl-6-[4-(3-trifluoromethyl-pyridin-2-yl)-piperazin-1-yl]-1,5-dihydro-pyrazolo[3,4-d]pyrimidin-4-one,
6-[4-(2-Fluoro-4-methanesulfonyl-phenyl)-piperazin-1-yl]-1-methyl-1,5-dihydro-pyrazolo[3,4-d]pyrimidin-4-one,
6-[4-(4-Fluoro-2-methanesulfonyl-phenyl)-piperazin-1-yl]-1-methyl-1,5-dihydro-pyrazolo[3,4-d]pyrimidin-4-one,
6-(4′-Hydroxy-3′,4′,5′,6′-tetrahydro-2′H-[2,4]bipyridinyl-1′-yl)-1-methyl-1,5-dihydro-pyrazolo[3,4-d]pyrimidin-4-one,
6-[4-(1,1-Dioxo-tetrahydro-1×,*6*-thiophen-3-yl)-piperazin-1-yl]-1-methyl-1,5-dihydro-pyrazolo[3,4-d]pyrimidin-4-one,
6-(4-Cyclopentyl-piperazin-1-yl)-1-methyl-1,5-dihydro-pyrazolo[3,4-d]pyrimidin-4-one,
6-[4-(2,6-Difluoro-phenyl)-piperazin-1-yl]-1-methyl-1,5-dihydro-pyrazolo[3,4-d]pyrimidin-4-one,
6-[4-(2,6-Difluoro-4-nitro-phenyl)-piperazin-1-yl]-1-methyl-1,5-dihydro-pyrazolo[3,4-d]pyrimidin-4-one,
1-Methyl-6-[4-(2-trifluoromethyl-phenyl)-piperazin-1-yl]-1,5-dihydro-pyrazolo[3,4-d]pyrimidin-4-one,
6-(4-Hydroxy-4-phenyl-piperidin-1-yl)-1-methyl-1,5-dihydro-pyrazolo[3,4-d]pyrimidin-4-one and
1-(1-Methyl-4-oxo-4,5-dihydro-1H-pyrazolo[3,4-d]pyrimidin-6-yl)-4-phenyl-piperidine-4-carbonitrile.

In another embodiment of the present invention there is provided a compound of formula I′ as described herein wherein the compound is selected from the group consisting of
3,5-Difluoro-4-[4-(1-methyl-4-oxo-4,5-dihydro-1H-pyrazolo[3,4-d]pyrimidin-6-yl)-piperazin-1-yl]-benzonitrile,
6-{4-[3-Fluoro-5-(2-methoxy-ethoxy)-phenyl]-piperazin-1-yl}-1-methyl-1,5-dihydro pyrazolo[3,4-d]pyrimidin-4-one,
6-[4-(2,3-Difluoro-5-hydroxy-phenyl)-piperazin-1-yl]-1-methyl-1,5-dihydro-pyrazolo[3,4-d]pyrimidin-4-one,
6-[4-(4-Amino-2,6-difluoro-phenyl)-piperazin-1-yl]-1-methyl-1,5-dihydro-pyrazolo[3,4-d]pyrimidin-4-one,
6-[4-(4-Fluoro-phenyl)-piperidin-1-yl]-1,5-dihydro-pyrazolo[3,4-d]pyrimidin-4-one,
6-[4-(2-Fluoro-phenyl)-piperazin-1-yl]-1,5-dihydro-pyrazolo[3,4-d]pyrimidin-4-one,
6-[4-(4-Fluoro-phenyl)-piperidin-1-yl]-1-methyl-1,5-dihydro-pyrazolo[4,3-c]pyridin-4-one,
6-[4-(2-Fluoro-phenyl)-piperazin-1-yl]-1-methyl-1,5-dihydro-pyrazolo[4,3-c]pyridin-4-one,
6-[4-(2,4-Difluoro-phenyl)-piperazin-1-yl]-1-methyl-1,5-dihydro-pyrazolo[4,3-c]pyridin-4-one,
2-[4-(1-Methyl-4-oxo-4,5-dihydro-1H-pyrazolo[4,3-c]pyridin-6-yl)-piperazin-1-yl]-benzonitrile and
6-[4-(4-Fluoro-2-methylsulfonyl-phenyl)-piperazin-1-yl]-1-methyl-1,5-dihydro-pyrazolo[4,3-c]pyridin-4-one.

In another embodiment of the present invention there is provided a compound of formula I′ as described herein wherein the compound is selected from the group consisting of
1-Methyl-6-[4-(3-trifluoromethyl-pyridin-2-yl)-piperazin-1-yl]-1,5-dihydro-pyrazolo[4,3-c]pyridin-4-one,
6-[4-(3,5-Dichloro-pyridin-4-yl)-piperazin-1-yl]-1-methyl-1,5-dihydro-pyrazolo[4,3-c]pyridin-4-one,
6-[4-(2,6-Fluoro-phenyl)-piperazin-1-yl]-1-methyl-1,5-dihydro-pyrazolo[4,3-c]pyridin-4-one,
6-{4-[2,6-Difluoro-4-(2-methoxy-ethoxy)-phenyl]-piperazin-1-yl}-1-methyl-1,5-dihydro-pyrazolo[4,3-c]pyridin-4-one,
6-[4-(4-Fluoro-phenyl)-piperidin-1-yl]-1,5-dihydro-pyrazolo[4,3-c]pyridin-4-one,
6-[4-(2-Fluoro-phenyl)-piperazin-1-yl]-1,5-dihydro-pyrazolo[4,3-c]pyridin-4-one,
1-Methyl-6-(6-trifluoromethyl-pyridin-3-yl)-1,5-dihydro-pyrazolo[3,4-d]pyrimidin-4-one,
1-Methyl-6-(4-trifluoromethyl-phenyl)-1,5-dihydro-pyrazolo[4,3-c]pyridin-4-one,
1-Methyl-6-(6-trifluoromethyl-pyridin-3-yl)-1,5-dihydro-pyrazolo[4,3-c]pyridin-4-one,
1-Methyl-6-(4-trifluoromethyl-phenyl)-1,5-dihydro-pyrazolo[3,4-d]pyrimidin-4-one,
6-{4-[2-Fluoro-4-(1-hydroxy-1-methyl-ethyl)-phenyl]-piperazin-1-yl}-1-methyl-1,5-dihydro-pyrazolo[3,4-d]pyrimidin-4-one,
1-Methyl-6-(5-trifluoromethyl-pyridin-2-yl)-1,5-dihydro-pyrazolo[3,4-d]pyrimidin-4-one and
6-{4-[2-Fluoro-4-(1H-tetrazol-5-yl)-phenyl]-piperazin-1-yl}-1-methyl-1,5-dihydro-pyrazolo[3,4-d]pyrimidin-4-one.

In another embodiment the invention relates to a compound of formula I as described herein for use as therapeutically active substance.

In another embodiment the invention relates to a compound of formula I as described herein for the use as therapeutically active substance for the therapeutic and/or prophylactic treatment of cancer.

In another embodiment the invention relates to the use of a compound of formula I as described herein for the therapeutically active substance for the therapeutic and/or prophylactic treatment of cancer.

### DEFINITIONS

As used herein, the following terms shall have the following definitions.

The term “alkyl” refers to straight- or branched-chain saturated hydrocarbon groups having from 1 to about 12 carbon atoms, including groups having from 1 to about 7 carbon atoms. In certain embodiments, alkyl substituents may be lower alkyl substituents. The term “lower alkyl” refers to alkyl groups having from 1 to 6 carbon atoms, preferably from 1 to 4 carbon atoms. Examples of alkyl groups include, but are not limited to, methyl, ethyl, n-propyl, i-propyl, n-butyl, s-butyl, t-butyl, n-pentyl, and s-pentyl.

The term “alkenyl” as used herein means an unsaturated straight-chain or branched aliphatic hydrocarbon group containing at least one double bond and having 2 to 6, preferably 2 to 4 carbon atoms. Examples of such “alkenyl group” are vinyl, ethenyl, allyl, isopropenyl, 1-propenyl, 2-methyl-1-propenyl, 1-butenyl, 2-butenyl, 3-butenyl, 2-ethyl-1-butenyl, 3-methyl-2-butenyl, 1-pentenyl, 2-pentenyl, 3-pentenyl, 4-pentenyl, 4-methyl-3-pentenyl, 1-hexenyl, 2-hexenyl, 3-hexenyl, 4-hexenyl and 5-hexenyl.

The terms “alkoxy, alkoxyl or lower alkoxy” refer to any of the above lower alkyl groups which is attached to the remainder of the molecule by an oxygen atom (RO—). Typical lower alkoxy groups include methoxy, ethoxy, isopropoxy or propoxy, butyloxy and the like. Further included within the meaning of alkoxy are multiple alkoxy side chains, e.g. ethoxy ethoxy, methoxy ethoxy, methoxy ethoxy ethoxy and the like and substituted alkoxy side chains, e.g., dimethylamino ethoxy, diethylamino ethoxy, dimethoxy-phosphoryl methoxy and the like.

The term “alkynyl” as used herein means an unsaturated straight-chain or branched aliphatic hydrocarbon group containing one triple bond and having 2 to 6, preferably 2 to 4 carbon atoms. Examples of such “alkynyl group” are ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl and 5-hexynyl.

The term “alkylene” as used herein denotes a divalent saturated linear hydrocarbon radical of 1 to 10 carbon atoms (e.g., (CH₂)ₙ) or a branched saturated divalent hydrocarbon radical of 2 to 10 carbon atoms (e.g., —CHMe- or —CH₂CH(i-Pr)CH₂—), unless otherwise indicated. C₀₋₄ alkylene or (alkylene)₀₄ refers to a linear or branched saturated divalent hydrocarbon radical comprising 1-4 carbon atoms or, in the case of C₀, the alkylene radical is omitted. Except in the case of methylene, the open valences of an alkylene group are not attached to the same atom. Examples of alkylene radicals include, but are not limited to, methylene, ethylene, propylene, 2-methyl-propylene, 1,1-dimethyl-ethylene, butylene, 2-ethylbutylene.

The term “acyl”, “alkanoyl” or “alkylcarbonyl” as used herein denotes a group of formula —C(═O)R wherein R is hydrogen or lower alkyl as defined herein. The term or “alkylcarbonyl” as used herein denotes a group of formula C(═O)R wherein R is alkyl as defined herein. The term C₁₋₆ acyl refers to a group —C(═O)R contain 1 to 6 carbon atoms. The C₁ acyl group is the formyl group wherein R═H and a C₆ acyl group refers to hexanoyl when the alkyl chain is unbranched. The term “arylcarbonyl” or “aroyl” as used herein means a group of formula C(═O)R wherein R is an aryl group; the term “benzoyl” as used herein an “arylcarbonyl” or “aroyl” group wherein R is phenyl.

The terms “alkoxycarbonyl” and “aryloxycarbonyl” as used herein denotes a group of formula —C(═O)OR wherein R is alkyl or aryl respectively and alkyl and aryl are as defined herein.

Amino means the group NH₂.

“Aryl” means a monovalent, monocyclic or bicyclic, aromatic hydrocarbon radical, preferably a 6-10 member aromatic ring system. Preferred aryl groups include, but are not limited to, phenyl, naphthyl, tolyl, and xylyl.

Carboxyl or carboxy means the monovalent group COOH. Carboxy lower alkyl or lower alkoxycarbonyl means COOR, wherein R is lower alkyl. Carbonyl means the group R′—C(═O)—R″, where R′ and R″ independently can be any of a number of chemical groups including alkyl.

The term “cycloalkyl” as used herein means any stable monocyclic or polycyclic system which consists of carbon atoms only, any ring of which being saturated, and the term “cycloalkenyl” is intended to refer to any stable monocyclic or polycyclic system which consists of carbon atoms only, with at least one ring thereof being partially unsaturated. Examples of cycloalkyls include, but are not limited to, cyclopropyl, cyclobutyl, cyclopentyl, cyclohexyl, cycloheptyl, adamantyl, cyclooctyl, bicycloalkyls, including bicyclooctanes such as [2.2.2]bicyclooctane or [3.3.0]bicyclooctane, bicyclononanes such as [4.3.0]bicyclononane, and bicyclodecanes such as [4.4.0]bicyclodecane (decalin), or spino compounds. Examples of cycloalkenyls include, but are not limited to, cyclopentenyl or cyclohexenyl.

The term “cycloalkylalkyl” as used herein refers to the radical R′R″, wherein R′ is a cycloalkyl radical, and R″ is an alkylene radical as both are defined herein with the understanding that the attachment point of the cycloalkylalkyl moiety will be on the alkylene radical. Examples of cycloalkylalkyl radicals include, but are not limited to, cyclopropylmethyl, cyclohexylmethyl, cyclopentylethyl. C₃₋₇ cycloalkyl-C₁₋₃ alkyl refers to the radical R′R″ where R′ is C₃₋₇ cycloalkyl and R″ is C₁₋₃ alkylene as defined herein.

The term “cyclic amine” denotes a saturated carbon ring, containing from 3 to 6 carbon atoms as defined above, and wherein at least one of the carbon atoms is replaced by a nitrogen atom and one or more other carbon atoms can be replaced by a heteroatom selected from the group consisting of N, O or S(O)₀₋₂, for example, piperidine, piperazine, morpholine, thiomorpholine, di-oxo-thiomorpholine, pyrrolidine, pyrazoline, imidazolidine, azetidine wherein the cyclic carbon atoms are optionally substituted by one or more substituents, selected from the group consisting of halogen, hydroxy, phenyl, lower alkyl, lower alkoxy or 2-hydrogen atoms on a carbon are both replace by oxo (═O). When the cyclic amine is a piperazine, one nitrogen atom can be optionally substituted by C₁₋₆ alkyl, C₁₋₆ acyl, C₁₋₆ alkylsulfonyl.

The term “haloalkyl” as used herein denotes an alkyl group as defined above wherein at least one hydrogen atom is substituted by a halogen. Examples are 1-fluoromethyl, 1-chloromethyl, 1-bromomethyl, 1-iodomethyl, difluoromethyl, trifluoromethyl, trichloromethyl, 1-fluoroethyl, 1-chloroethyl, 2-fluoroethyl, 2-chloroethyl, 2-bromoethyl, 2,2-dichloroethyl, 3-bromopropyl or 2,2,2-trifluoroethyl.

The term “halogen” as used herein means fluorine, chlorine, bromine, or iodine, preferably fluorine and chlorine.

The term “heteroaryl” means an aromatic heterocyclic ring system containing up to two rings. Preferred heteroaryl groups include, but are not limited to, thienyl, furyl, indolyl, pyrrolyl, pyridinyl, pyrazinyl, oxazolyl, thiaxolyl, quinolinyl, pyrimidinyl, imidazole substituted or unsubstituted triazolyl and substituted or unsubstituted tetrazolyl. In some embodiments heteroaryl is pyridinyl.

The term “heteroarylalkyl” or “heteroaralkyl” means the radical of the formula R′R″, wherein R′ is an optionally substituted heteroaryl radical as defined herein, and R″ is an alkylene radical as defined herein with the understanding that the attachment point of the heteroaryl radical will be on the alkylene radical. Examples of heteroarylalkyl radicals include, but are not limited to, 2-imidazolylmethyl, 3-pyrrolylethyl, 4-pyridinylmethyl and 5-pyrimidinylmethyl.

In the case of aryl or heteroaryl which are bicyclic it should be understood that one ring may be aryl while the other is heteroaryl and both being substituted or unsubstituted and the point of attachment is on the aryl or heteroaryl ring respectively.

The term “hetero atom” means an atom selected from N, O and S.

The terms “heterocycle” or “heterocyclic ring” means a substituted or unsubstituted 5 to 8 membered, mono- or bicyclic, non-aromatic hydrocarbon, wherein 1 to 3 carbon atoms are replaced by a hetero atom selected from nitrogen, oxygen or sulfur atom. Examples include pyrrolidin-2-yl; pyrrolidin-3-yl; piperidinyl; morpholin-4-yl and the like which in turn can be substituted.

The term “heterocycloalkyl” (or “heterocyclylalkyl”) denotes the radical of the formula R′R″, wherein R′ is a heterocyclic radical as defined herein, and R″ is an alkylene radical as defined herein and the attachment point of the heterocycloalkyl radical will be on the alkylene radical. Examples of heterocycloalkyl radicals include, but are not limited to, 1-piperazinylmethyl, 2-morpholinomethyl, and the like.

Hydroxy or hydroxyl is a prefix indicating the presence of a monovalent OH group.

The terms “hydroxyalkyl” or “alkoxyalkyl” as used herein denotes alkyl radical as herein defined wherein one to three hydrogen atoms on different carbon atoms is/are replaced by hydroxyl or alkoxy groups respectively. A C₁₋₃ alkoxy-C₁₋₆ alkyl moiety refers to a C₁₋₆ alkyl substituent in which 1 to 3 hydrogen atoms are replaced by a C₁₋₃ alkoxy and the point of attachment of the alkoxy is the oxygen atom.

“Lower” as in “lower alkenyl” means a group having 1 to 6 carbon atoms (“C₁₋₆alkyl”).

The term “nitro” means NO₂.

The term “cyano” means CN.

The term “oxo” means the group ═O.

“Substituted,” as in substituted alkyl, means that the substitution can occur at one or more positions and, unless otherwise indicated, that the substituents at each substitution site are independently selected from the specified options. The term “optionally substituted” refers to the fact that one or more hydrogen atoms of a chemical group (with one or more hydrogen atoms) can be, but does not necessarily have to be, substituted with another substituent. In the specification where indicated the various groups may be substituted by preferably, 1-3 substituents independently selected from the group consisting of H, carboxyl, amido, hydroxyl, alkoxy, substituted alkoxy, sulfide, sulfone, sulfonamide, sulfoxide, halogen, nitro, amino, substituted amino, lower alkyl, substituted lower alkyl, lower cycloalkyl, substituted lower cycloalkyl, lower alkenyl, substituted lower alkenyl, lower cycloalkenyl, substituted lower cycloalkenyl, aryl, substituted aryl, heteroaryl, substituted heteroaryl, heterocycle or substituted heterocycle.

“Pharmaceutically acceptable,” such as pharmaceutically acceptable carrier, excipient, etc., means pharmacologically acceptable and substantially non-toxic to the subject to which the particular compound is administered.

“Pharmaceutically acceptable salt” refers to conventional acid-addition salts or base-addition salts that retain the biological effectiveness and properties of the compounds of the present invention and are formed from suitable non-toxic organic or inorganic acids or organic or inorganic bases. Sample acid-addition salts include those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, trifluoro acetic acid and the like. Sample base-addition salts include those derived from ammonium, potassium, sodium and, quaternary ammonium hydroxides, such as for example, tetramethylammonium hydroxide. Chemical modification of a pharmaceutical compound (i.e. drug) into a salt is a technique well known to pharmaceutical chemists to obtain improved physical and chemical stability, hygroscopicity, flowability and solubility of compounds. See, e.g., Ansel et al., Pharmaceutical Dosage Forms and Drug Delivery Systems (1995) at pgs. 456-457.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems, Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

An embodiment, therefore, includes a pharmaceutical composition comprising a compound of Formula I, or a stereoisomer or pharmaceutically acceptable salt thereof. In a further embodiment includes a pharmaceutical composition comprising a compound of Formula I, or a stereoisomer or pharmaceutically acceptable salt thereof, together with a pharmaceutically acceptable carrier or excipient.

Another embodiment includes a pharmaceutical composition comprising a compound of Formula I for use in the treatment of a hyperproliferative disease. Another embodiment includes a pharmaceutical composition comprising a compound of Formula I for use in the treatment of cancer.

Commonly used abbreviations include: acetyl (Ac), aqueous (aq.), atmospheres (Atm), tert-butoxycarbonyl (Boc), di-tert-butyl pyrocarbonate or boc anhydride (BOC₂O), benzyl (Bn), benzotriazol-1-yloxy-tris-(dimethylamino)phosphonium hexafluorophosphate (BOP), butyl (Bu), benzoyl (Bz), Chemical Abstracts Registration Number (CASRN), benzyloxycarbonyl (CBZ or Z), carbonyl diimidazole (CDI), dibenzylideneacetone (DBA), 1,5-diazabicyclo[4.3.0]non-5-ene (DBN), 1,8-diazabicyclo[5.4.0]undec-7-ene (DBU), N,N′-dicyclohexylcarbodiimide (DCC), 1,2-dichloroethane (DCE), dichloromethane (DCM), diethyl azodicarboxylate (DEAD), di-iso-propylazodicarboxylate (DIAD), di-iso-butylaluminumhydride (DIBAL or DIBAL-H), di-iso-propylethylamine (DIPEA), N,N-dimethyl acetamide (DMA), 4-N,N-dimethylaminopyridine (DMAP), DMF(DMF), dimethyl sulfoxide (DMSO), 1,1′-bis-(diphenylphosphino)ethane (dppe), 1,1′-bis-(diphenylphosphino)ferrocene (dppf), 1-(3-dimethylaminopropyl)-3-ethylcarbodiimide hydrochloride (EDCI), ethyl (Et), ethyl acetate (EtOAc), ethanol (EtOH), 2-ethoxy-2H-quinoline-1-carboxylic acid ethyl ester (EEDQ), diethyl ether (Et₂O), O-(7-azabenzotriazole-1-yl)-N,N,N′N′-tetramethyluronium hexafluorophosphate acetic acid (HATU), acetic acid (HOAc), 1-N-hydroxybenzotriazole (HOBt), high pressure liquid chromatography (HPLC), iso-propanol (IPA), lithium hexamethyldisilazide (LiHMDS), methanol (MeOH), melting point (mp), MeSO₂— (mesyl or Ms), methyl (Me), acetonitrile (MeCN), m-chloroperbenzoic acid (MCPBA), mass spectrum (ms), methyl tert-butyl ether (MTBE), N-methylmorpholine (NMM), N-methylpyrrolidone (NMP), petroleum ether (pet ether, i.e. hydrocarbons),)phenyl (Ph), propyl (Pr), iso-propyl (i-Pr), pounds per square inch (psi), bromo-tris-pyrrolidinophosphonium hexafluorophosphate (PyBrOP), pyridine (pyr), room temperature (rt or RT), satd. (saturated), tert-butymethyl ether (TBME), tert-butyldimethylsilyl or t-BuMe₂Si (TBDMS or TBS), triethylamine (TEA or Et₃N), triflate or CF₃SO₂— (Tf), trifluoroacetic acid (TFA), O-b enzotriazol-1-yl-N,N,N′,N′-tetramethyluronium tetrafluoroborate (TBTU), thin layer chromatography (TLC), tetrahydrofuran (THF), tetramethylethylenediamine (TMEDA), trimethylsilyl or Me₃Si (TMS), 2-(trimethylsilyl)ethoxymethyl (SEM), p-toluenesulfonic acid monohydrate (TsOH or pTsOH), 4-Me-C₆H₄SO₂— or tosyl (Ts), N-urethane-N-carboxyanhydride (UNCA). Conventional nomenclature including the prefixes normal (n), iso (i-), secondary (sec-), tertiary (tert- or -t) and neo- have their customary meaning when used with an alkyl moiety. (J. Rigaudy and D. P. Klesney, Nomenclature in Organic Chemistry, IUPAC 1979 Pergamon Press, Oxford.). IWR2 refers to 4-((1S,2R,6S,7R)-3,5-dioxo-4-aza-tricyclo[5.2.1.0*2,6*]dec-8-en-4-yl)-N-(4-methyl-quinolin-8-yl)-benzamide and XAV9392 refers to-(4-trifluoromethyl-phenyl)-3,5,7,8-tetrahydro-thiopyrano[4,3-d]pyrimidin-4-one

Compounds and Preparation

Examples of representative compounds within the scope of the invention are provided in the following Table. These examples and preparations which follow are provided to enable those skilled in the art to more clearly understand and to practice the present invention. They should not be considered as limiting the scope of the invention, but merely as being illustrative and representative thereof.

If there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it. The following numbering system is used herein.

In general, the nomenclature used in this Application is based on AUTONOM™ v.4.0, a Beilstein Institute computerized system for the generation of IUPAC systematic nomenclature. If there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

TABLE 1

| | | TNKS1 | TNKS2 | |
|---|---|---|---|---|
| CMPD. | | ¹ (IC₅₀) | (IC₅₀) | |
| No. | STRUCTURE | (μM) | (μM) | IUPAC NAME |
| I-1 | 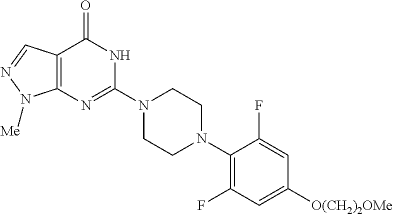 | 0.0076 | 0.0244 | 6-[4-[2 6-difluoro-4-(2- methoxyethoxy)phenyl] piperazin-1-yl]-1-methyl-5H- pyrazolo[3 4-d]pyrimidin- 4-one |
| I-2 | 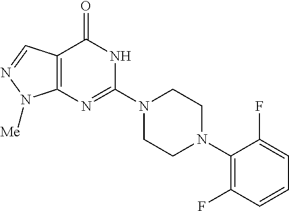 [SMILES: Cn1ncc2c(=O)[nH]c(N3CCN(c4c(F)cccc4F)CC3)nc21] | 0.0109 | 0.0187 | 6-[4-(2 6- difluorophenyl)piperazin- 1-yl]-1-methyl-5H- pyrazolo[3 4-d]pyrimidin- 4-one |
| I-3 | 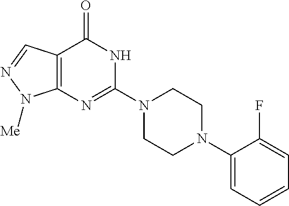 [SMILES: Cn1ncc2c(=O)[nH]c(N3CCN(c4ccccc4F)CC3)nc21] | 0.027 | 0.0344 | 6-[4-(2- fluorophenyl)piperazin-1- yl]-1-methyl-5H- pyrazolo[3 4-d]pyrimidin- 4-one |
| I-4 | 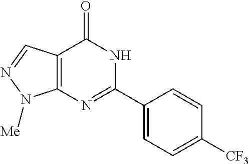 [SMILES: Cn1ncc2c(=O)[nH]c(-c3ccc(C(F)(F)F)cc3)nc21] | 0.025 | 0.0345 | 1-methyl-6-[4- (trifluoromethyl)phenyl]- 5H-pyrazolo[3 4- d]pyrimidin-4-one |
| I-5 | 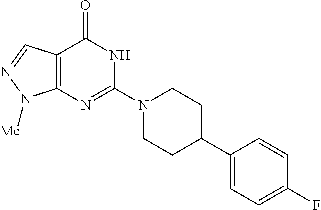 [SMILES: Cn1ncc2c(=O)[nH]c(N3CCC(c4ccc(F)cc4)CC3)nc21] | 0.031 | 0.0376 | 6-[4-(4-fluorophenyl)-1- piperidyl]-1-methyl-5H- pyrazolo[3 4-d]pyrimidin- 4-one |
| I-6 | 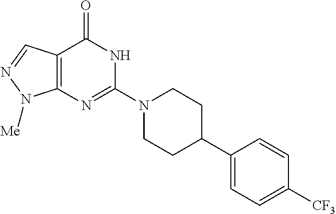 [SMILES: Cn1ncc2c(=O)[nH]c(N3CCC(c4ccc(C(F)(F)F)cc4)CC3)nc21] | 0.066 | 0.0683 | 1-methyl-6-[4-[4- (trifluoromethyl)phenyl]-1- piperidyl]-5H-pyrazolo[3 4-d]pyrimidin-4-one |
| I-7 | 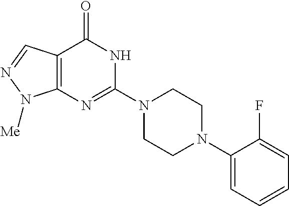 [SMILES: Cn1ncc2c(=O)[nH]c(N3CCN(c4ccccc4F)CC3)nc21] | 0.046 | 0.0412 | 6-[4-(2- fluorophenyl)piperazin-1- yl]-1-methyl-5H- pyrazolo[4 3-c]pyridin-4- one |
| I-8 | 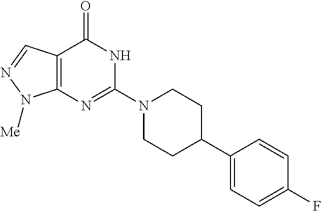 [SMILES: Cn1ncc2c(=O)[nH]c(N3CCC(c4ccc(F)cc4)CC3)nc21] | 0.042 | 0.0428 | 6-[4-(4-fluorophenyl)-1- piperidyl]-1-methyl-5H- pyrazolo[4 3-c]pyridin-4- one |
| I-9 | 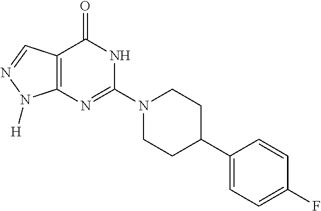 [SMILES: O=c1[nH]c(N2CCC(c3ccc(F)cc3)CC2)nc2[nH]ncc12] | 0.374 | 0.664 | 6-[4-(4-fluorophenyl)-1- piperidyl]-1 5- dihydropyrazolo[3 4- d]pyrimidin-4-one |
| I-10 | 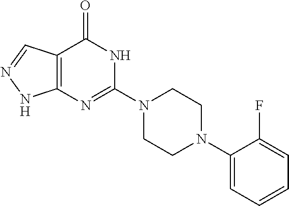 [SMILES: O=c1[nH]c(N2CCN(c3ccccc3F)CC2)nc2[nH]ncc12] | 0.147 | 0.376 | 6-[4-(2- fluorophenyl)piperazin-1- yl]-1 5-dihydropyrazolo[3 4-d]pyrimidin-4-one |
| I-11 | 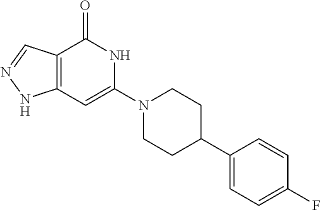 [SMILES: O=c1[nH]c(N2CCC(c3ccc(F)cc3)CC2)cc2[nH]ncc12] | 0.108 | 0.159 | 6-[4-(4-fluorophenyl)-1- piperidyl]-1 5- dihydropyrazolo[4 3- c]pyridin-4-one |
| I-12 | 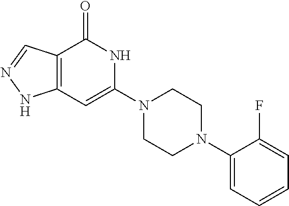 [SMILES: O=c1[nH]c(N2CCN(c3ccccc3F)CC2)cc2[nH]ncc12] | 0.105 | 0.176 | 6-[4-(2- fluorophenyl)piperazin-1- yl]-1 5-dihydropyrazolo[4 3-c]pyridin-4-one |
| I-13 | 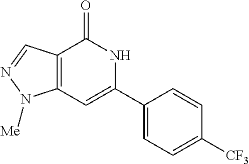 [SMILES: Cn1ncc2c(=O)[nH]c(-c3ccc(C(F)(F)F)cc3)cc21] | 0.032 | 0.0325 | 1-methyl-6-[4- (trifluoromethyl)phenyl]- 5H-pyrazolo[4 3-c]pyridin- 4-one |
| I-14 | 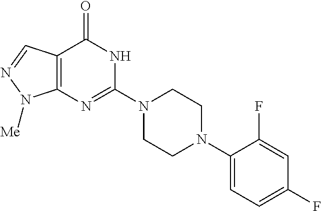 [SMILES: Cn1ncc2c(=O)[nH]c(N3CCN(c4ccc(F)cc4F)CC3)nc21] | 0.039 | 0.0471 | 6-[4-(2 4- difluorophenyl)piperazin- 1-yl]-1-methyl-5H- pyrazolo[3 4-d]pyrimidin- 4-one |
| I-15 | 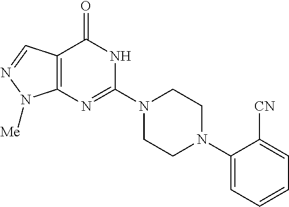 [SMILES: Cn1ncc2c(=O)[nH]c(N3CCN(c4ccccc4C#N)CC3)nc21] | 0.074 | 0.0952 | 2-[4-(1-methyl-4-oxo-5H- pyrazolo[3 4-d]pyrimidin- 6-yl)piperazin-1- yl]benzonitrile |
| I-16 | 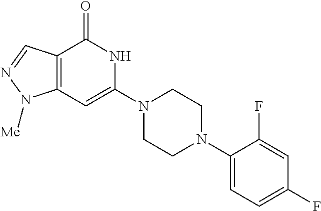 [SMILES: Cn1ncc2c(=O)[nH]c(N3CCN(c4ccc(F)cc4F)CC3)cc21] | 0.048 | 0.0474 | 6-[4-(2 4- difluorophenyl)piperazin- 1-yl]-1-methyl-5H- pyrazolo[4 3-c]pyridin-4- one |
| I-17 | 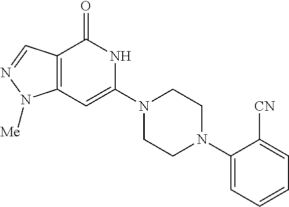 [SMILES: Cn1ncc2c(=O)[nH]c(N3CCN(c4ccccc4C#N)CC3)cc21] | 0.103 | 0.097 | 2-[4-(1-methyl-4-oxo-5H- pyrazolo[4 3-c]pyridin-6- yl)piperazin-1- yl]benzonitrile |
| I-18 | 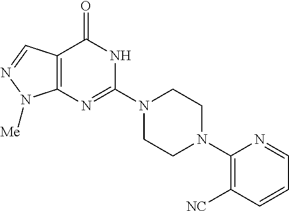 [SMILES: Cn1ncc2c(=O)[nH]c(N3CCN(c4ncccc4C#N)CC3)nc21] | 0.150 | 0.228 | 2-[4-(1-methyl-4-oxo-5H- pyrazolo[3 4-d]pyrimidin- 6-yl)piperazin-1- yl]pyridine-3-carbonitrile |
| I-19 | 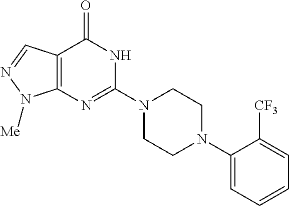 [SMILES: Cn1ncc2c(=O)[nH]c(N3CCN(c4ccccc4C(F)(F)F)CC3)nc21] | 0.027 | 0.0326 | 1-methyl-6-[4-[2- (trifluoromethyl)phenyl] piperazin-1-yl]-5H- pyrazolo[3 4-d]pyrimidin- 4-one |
| I-20 | 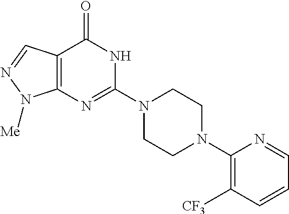 [SMILES: Cn1ncc2c(=O)[nH]c(N3CCN(c4ncccc4C(F)(F)F)CC3)nc21] | 0.031 | 0.0432 | 1-methyl-6-[4-[3- (trifluoromethyl)-2- pyridyl]piperazin-1-yl]- 5H-pyrazolo[3 4- d]pyrimidin-4-one |
| I-21 | 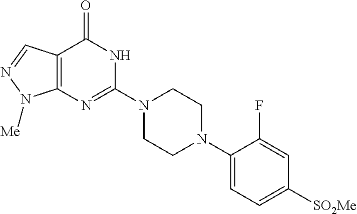 [SMILES: Cn1ncc2c(=O)[nH]c(N3CCN(c4ccc(S(C)(=O)=O)cc4F)CC3)nc21] | 0.055 | 0.0815 | 6-[4-(2-fluoro-4- methylsulfonyl- phenyl)piperazin-1-yl]-1- methyl-5H-pyrazolo[3 4- d]pyrimidin-4-one |
| I-22 | 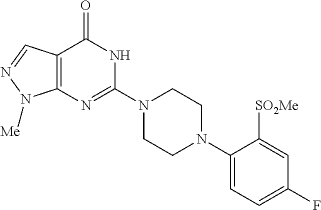 [SMILES: Cn1ncc2c(=O)[nH]c(N3CCN(c4ccc(F)cc4S(C)(=O)=O)CC3)nc21] | 0.044 | 0.055 | 6-[4-(4-fluoro-2- methylsulfonyl- phenyl)piperazin-1-yl]-1- methyl-5H-pyrazolo[3 4- d]pyrimidin-4-one |
| I-23 | 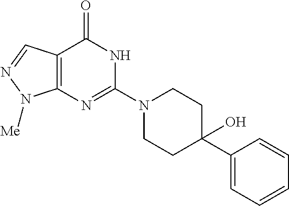 [SMILES: Cn1ncc2c(=O)[nH]c(N3CCC(O)(c4ccccc4)CC3)nc21] | 0.057 | 0.0668 | 6-(4-hydroxy-4-phenyl-1- piperidyl)-1-methyl-5H- pyrazolo[3 4-d]pyrimidin- 4-one |
| I-24 | 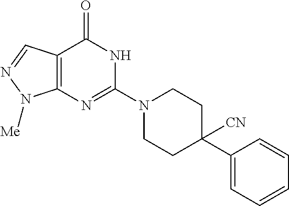 [SMILES: Cn1ncc2c(=O)[nH]c(N3CCC(C#N)(c4ccccc4)CC3)nc21] | 0.027 | 0.0333 | 1-(1-methyl-4-oxo-5H- pyrazolo[3 4-d]pyrimidin- 6-yl)-4-phenyl-piperidine- 4-carbonitrile |
| I-25 | 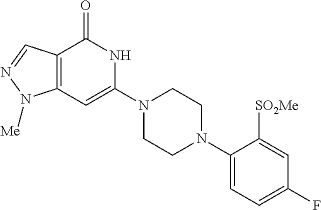 [SMILES: Cn1ncc2c(=O)[nH]c(N3CCN(c4ccc(F)cc4S(C)(=O)=O)CC3)cc21] | 0.044 | 0.0451 | 6-[4-(4-fluoro-2- methylsulfonyl- phenyl)piperazin-1-yl]-1- methyl-5H-pyrazolo[4 3- c]pyridin-4-one |
| I-26 | 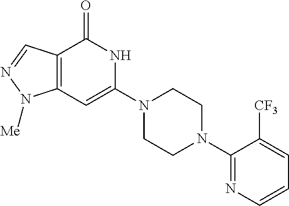 [SMILES: Cn1ncc2c(=O)[nH]c(N3CCN(c4ncccc4C(F)(F)F)CC3)cc21] | 0.029 | 0.0304 | 1-methyl-6-[4-[3- (trifluoromethyl)-2- pyridyl]piperazin-1-yl]- 5H-pyrazolo[4 3-c]pyridin- 4-one |
| I-27 | 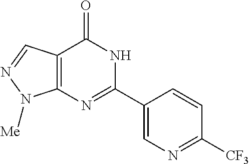 [SMILES: Cn1ncc2c(=O)[nH]c(-c3ccc(C(F)(F)F)nc3)nc21] | 0.385 | 0.428 | 1-methyl-6-[6- (trifluoromethyl)-3- pyridyl)-5H-pyrazolo[3 4- d]pyrimidin-4-one |
| I-28 | 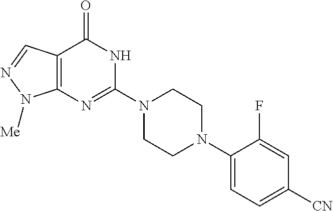 [SMILES: Cn1ncc2c(=O)[nH]c(N3CCN(c4ccc(C#N)cc4F)CC3)nc21] | 0.051 | 0.0765 | 3-fluoro-4-[4-(1-methyl-4- oxo-5H-pyrazolo[3 4- d]pyrimidin-6- yl)piperazin-1- yl]benzonitrile |
| I-29 | 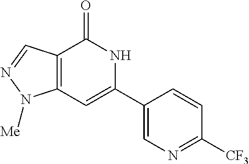 [SMILES: Cn1ncc2c(=O)[nH]c(-c3ccc(C(F)(F)F)nc3)cc21] | 0.151 | 0.104 | 1-methyl-6-[6- (trifluoromethyl)-3- pyridyl]-5H-pyrazolo[4 3- c]pyridin-4-one |
| I-30 | 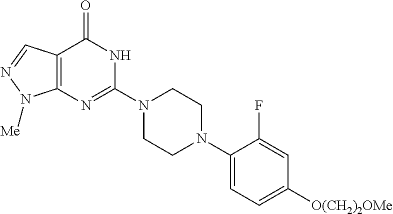 | 0.013 | 0.234 | 6-[4-[2-fluoro-4-(2- methoxyethoxy)phenyl] piperazin-1-yl]-1-methyl-5H- pyrazolo[3 4-d]pyrimidin- 4-one |
| I-31 | 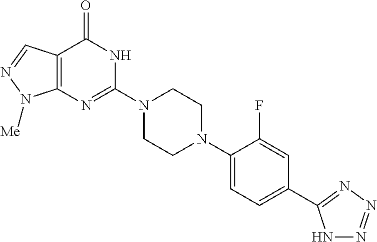 [SMILES: Cn1ncc2c(=O)[nH]c(N3CCN(c4ccc(-c5nnn[nH]5)cc4F)CC3)nc21] | 0.026 | 0.0246 | 6-[4-[2-fluoro-4-(1H- tetrazol-5- yl)phenyl]piperazin-1-yl]- 1-methyl-5H-pyrazolo[3 4- d]pyrimidin-4-one |
| I-32 | 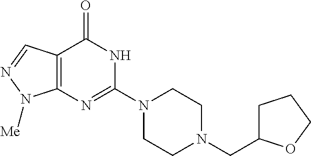 [SMILES: Cn1ncc2c(=O)[nH]c(N3CCN(CC4CCCO4)CC3)nc21] | 0.155 | 0.178 | 1-methyl-6-[4- (tetrahydrofuran-2- ylmethyl)piperazin-1-yl]- 5H-pyrazolo[3 4- d]pyrimidin-4-one |
| I-33 | 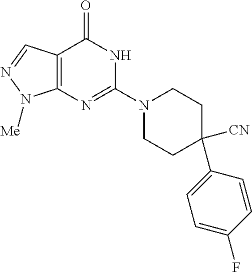 [SMILES: Cn1ncc2c(=O)[nH]c(N3CCC(C#N)(c4ccc(F)cc4)CC3)nc21] | 0.032 | 0.0217 | 4-(4-fluorophenyl)-1-(1- methyl-4-oxo-5H- pyrazolo[3 4-d]pyrimidin- 6-yl)piperidine-4- carbonitrile |
| I-34 | 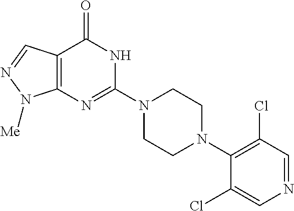 [SMILES: Cn1ncc2c(=O)[nH]c(N3CCN(c4c(Cl)cncc4Cl)CC3)nc21] | 0.029 | 0.0289 | 6-[4-(3 5-dichloro-4- pyridyl)piperazin-1-yl]-1- methyl-5H-pyrazolo[3 4- d]pyrimidin-4-one |
| I-35 | 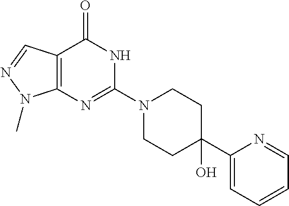 [SMILES: Cn1ncc2c(=O)[nH]c(N3CCC(O)(c4ccccn4)CC3)nc21] | 0.069 | 0.0945 | 6-[4-hydroxy-4-(2- pyridyl)-1-piperidyl)-1- methyl-5H-pyrazolo[3 4- d]pyrimidin-1-one |
| I-36 | 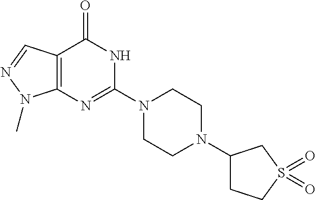 [SMILES: Cn1ncc2c(=O)[nH]c(N3CCN(C4CCS(=O)(=O)C4)CC3)nc21] | 1.935 | 1.6 | 6-[4-(1 1-dioxothiolan-3- yl)piperazin-1-yl]-1- methyl-5H-pyrazolo[3 4- d]pyrimidin-4-one |
| I-37 | 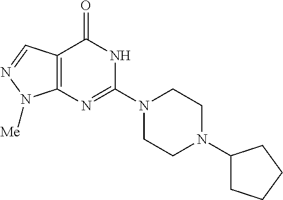 [SMILES: Cn1ncc2c(=O)[nH]c(N3CCN(C4CCCC4)CC3)nc21] | 0.165 | 0.178 | 6-(4-cyclopentylpiperazin- 1-yl)-1-methyl-5H- pyrazolo[3 4-d]pyrimidin- 4-one |
| I-38 | 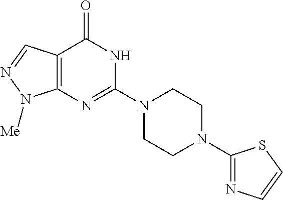 [SMILES: Cn1ncc2c(=O)[nH]c(N3CCN(c4nccs4)CC3)nc21] | 0.504 | 0.598 | 1-methyl-6-(4-thiazol-2- ylpiperazin-1-yl)-5H- pyrazolo[3 4-d]pyrimidin- 4-one |
| I-39 | 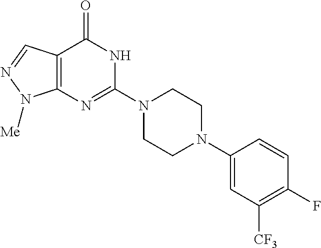 [SMILES: Cn1ncc2c(=O)[nH]c(N3CCN(c4ccc(F)c(C(F)(F)F)c4)CC3)nc21] | 0.331 | 0.303 | 6-[4-(4-fluoro-3- (trifluoromethyl)phenyl] piperazin-1-yl]-1-methyl-5H- pyrazolo[3 4-d]pyrimidin- 4-one |
| I-40 | 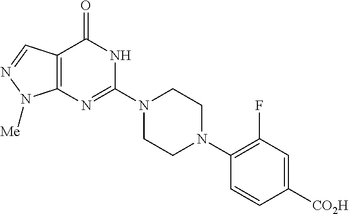 [SMILES: Cn1ncc2c(=O)[nH]c(N3CCN(c4ccc(C(=O)O)cc4F)CC3)nc21] | 0.041 | 0.0394 | 3-fluoro-4-[4-(1-methyl-4- oxo-5H-pyrazolo[3 4- d]pyrimidin-6- yl)piperazin-1-yl]benzoic acid |
| I-41 | 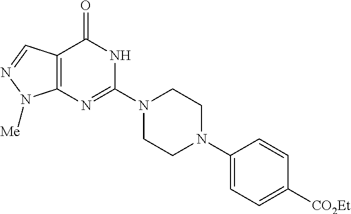 [SMILES: CCOC(=O)c1ccc(N2CCN(c3nc4c(cnn4C)c(=O)[nH]3)CC2)cc1] | 0.107 | 0.135 | ethyl 4-[4-(1-methyl-4- oxo-5H-pyrazolo[3 4- d]pyrimidin-6- yl)piperazin-1-yl]benzoate |
| I-42 | 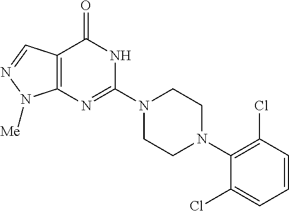 [SMILES: Cn1ncc2c(=O)[nH]c(N3CCN(c4c(Cl)cccc4Cl)CC3)nc21] | 0.024 | 0.0263 | 6-[4-(2 6- dichlorophenyl)piperazin- 1-yl]-1-methyl-5H- pyrazolo[3 4-d]pyrimidin- 4-one |
| I-43 | 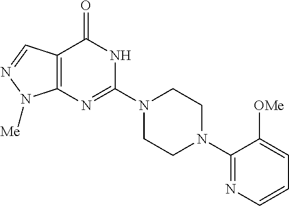 [SMILES: COc1cccnc1N1CCN(c2nc3c(cnn3C)c(=O)[nH]2)CC1] | 0.047 | 0.0553 | 6-[4-(3-methoxy-2- pyridyl)piperazin-1-yl]-1- methyl-5H-pyrazolo[3 4- d]pyrimidin-4-one |
| I-44 | 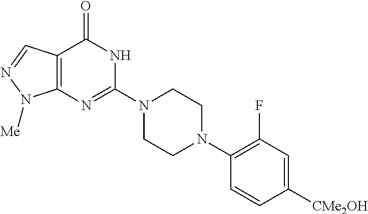 | 0.034 | 0.0953 | 6-[4-[2-fluoro-4-(1- hydroxy-1-methyl- ethyl)phenyl]piperazin-1- yl]-1-methyl-5H- pyrazolo[3 4-d]pyrimidin- 4-one |
| I-45 | 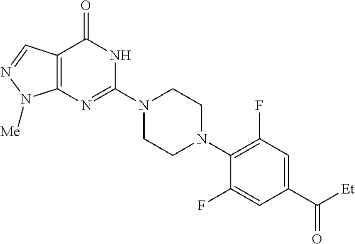 [SMILES: CCC(=O)c1cc(F)c(N2CCN(c3nc4c(cnn4C)c(=O)[nH]3)CC2)c(F)c1] | 0.028 | 0.033 | 6-[4-(2 6-difluoro-4- propanoyl- phenyl)piperazin-1-yl]-1- methyl-5H-pyrazolo[3 4- d]pyrimidin-4-one |
| I-46 | 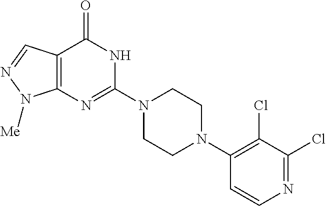 [SMILES: Cn1ncc2c(=O)[nH]c(N3CCN(c4ccnc(Cl)c4Cl)CC3)nc21] | 0.061 | 0.0939 | 6-[4-(2 3-dichloro-4- pyridyl)piperazin-1-yl]-1- methyl-5H-pyrazolo[3 4- d]pyrimidin-4-one |
| I-47 | 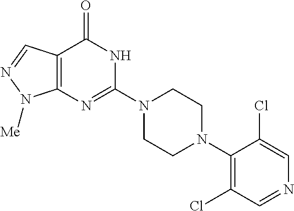 [SMILES: Cn1ncc2c(=O)[nH]c(N3CCN(c4c(Cl)cncc4Cl)CC3)nc21] | 0.026 | 0.0291 | 6-[4-(3 5-dichloro-4- pyridyl)piperazin-1-yl]-1- methyl-5H-pyrazolo[4 3- c]pyridin-4-one |
| I-48 | 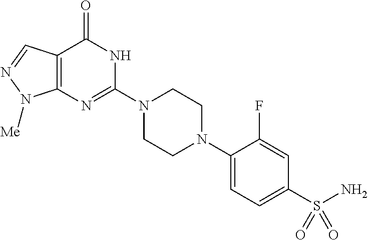 [SMILES: Cn1ncc2c(=O)[nH]c(N3CCN(c4ccc(S(N)(=O)=O)cc4F)CC3)nc21] | 0.034 | 0.0492 | 3-fluoro-4-[4-(1-methyl-4- oxo-5H-pyrazolo[3 4- d]pyrimidin-6- yl)piperazin-1- yl]benzenesulfonamide |
| I-49 | 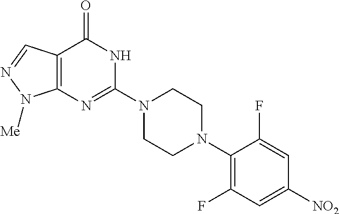 [SMILES: Cn1ncc2c(=O)[nH]c(N3CCN(c4c(F)cc([N+](=O)[O-])cc4F)CC3)nc21] | 0.034 | 0.0326 | 6-[4-(2 6-difluoro-4-nitro- phenyl)piperazin-1-yl]-1- methyl-5H-pyrazolo[3 4- d]pyrimidin-4-one |
| I-50 | 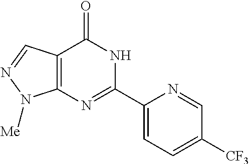 [SMILES: Cn1ncc2c(=O)[nH]c(-c3ccc(C(F)(F)F)cn3)nc21] | 0.286 | 0.325 | 1-methyl-6-[5- (trifluoromethyl)-2- pyridyl]-5H-pyrazolo[3 4- d]pyrimidin-4-one |
| I-51 | 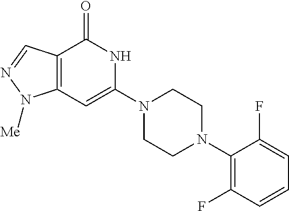 [SMILES: Cn1ncc2c(=O)[nH]c(N3CCN(c4c(F)cccc4F)CC3)cc21] | 0.025 | 0.0231 | 6-[4-(2 6- difluorophenyl)piperazin- 1-yl]-1-methyl-5H- pyrazolo[4 3-c]pyridin-4- one |
| I-52 | 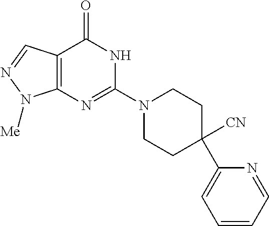 [SMILES: Cn1ncc2c(=O)[nH]c(N3CCC(C#N)(c4ccccn4)CC3)nc21] | 0.047 | 0.0545 | 1-(1-methyl-4-oxo-5H- pyrazolo[3 4-d]pyrimidin- 6-yl)-4-(2- pyridyl)piperidine-4- carbonitrile |
| I-53 | 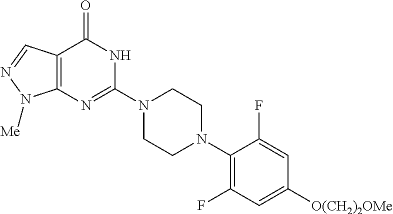 | 0.026 | 0.0193 | 6-[4-[2 6-difluoro-4-(2- methoxyethoxy)phenyl] piperazin-1-yl]-1-methyl-5H- pyrazolo[4 3-c]pyridin-4- one |
| I-54 | 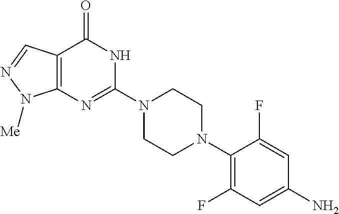 [SMILES: Cn1ncc2c(=O)[nH]c(N3CCN(c4c(F)cc(N)cc4F)CC3)nc21] | 0.021 | 0.025 | 6-[4-(4-amino-2 6- difluoro-phenyl)piperazin- 1-yl]-1-methyl-5H- pyrazolo[3 4-d]pyrimidin- 4-one |
| I-55 | 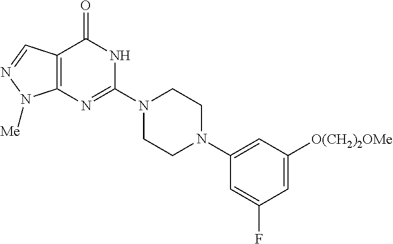 | 0.114 | 0.147 | 6-[4-[3-fluoro-5-(2- methoxyethoxy)phenyl] piperazin-1-yl]-1-methyl-5H- pyrazolo[3 4-d]pyrimidin- 4-one |
| I-56 | 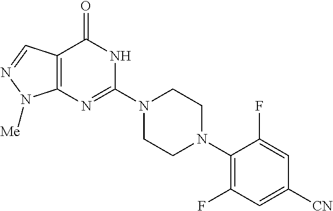 [SMILES: Cn1ncc2c(=O)[nH]c(N3CCN(c4c(F)cc(C#N)cc4F)CC3)nc21] | 0.055 | 0.0632 | 3 5-difluoro-4-[4-(1- methyl-4-oxo-5H- pyrazolo[3 4-d]pyrimidin- 6-yl)piperazin-1- yl]benzonitrile |
| I-57 | 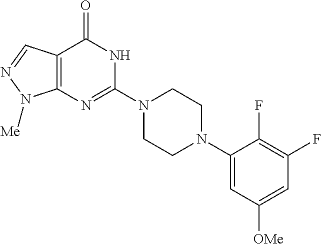 [SMILES: COc1cc(F)c(F)c(N2CCN(c3nc4c(cnn4C)c(=O)[nH]3)CC2)c1] | 0.066 | 0.0564 | 6-[4-(2 3-difluoro-5- hydroxy-phenyl)piperazin- 1-yl]-1-methyl-5H- pyrazolo[3 4-d]pyrimidin- 4-one |
| I-58 | 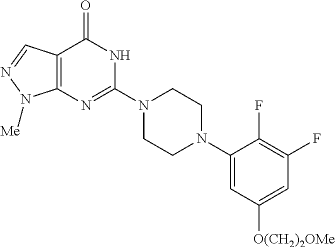 | | 0.0471 | 6-[4-(2 4- difluorophenyl)piperazin- 1-yl]-1-methyl-5H- pyrazolo[3 4-d]pyrimidin- 4-one |
| I-59 | 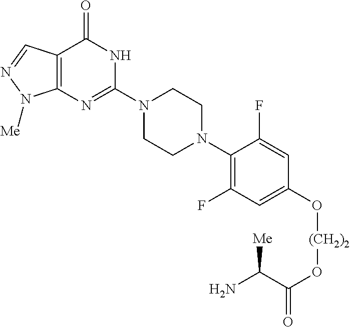 | 0.0037 | | 2-[3 5-difluoro-4-[4-(1- methyl-4-oxo-5H- pyrazolo[3 4-d]pyrimidin- 6-yl)piperazin-1- yl]phenoxy]ethyl (2S)-2- aminopropanoate |
| I-60 | 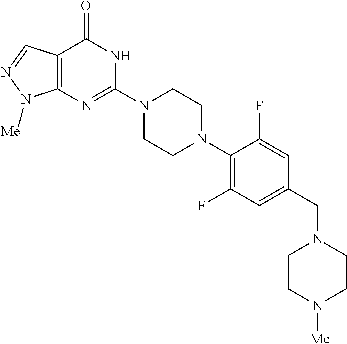 [SMILES: CN1CCN(Cc2cc(F)c(N3CCN(c4nc5c(cnn5C)c(=O)[nH]4)CC3)c(F)c2)CC1] | 0.0038 | | 6-[4-[2 6-difluoro-4-[(4- methylpiperazin-1- yl)methyl]phenyl]piperazin- 1-yl]-1-methyl-5H- pyrazolo[3 4-d]pyrimidin- 4-one |
| I-61 | 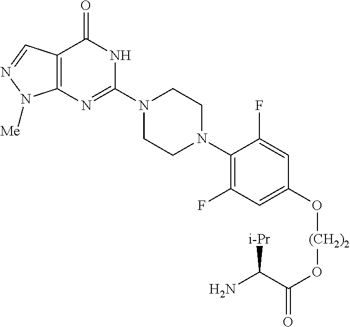 | 0.0042 | | 2-[3 5-difluoro-4-[4-(1- methyl-4-oxo-5H- pyrazolo[3 4-d]pyrimidin- 6-yl)piperazin-1- yl]phenoxy]ethyl (2S)-2- amino-3-methyl-butanoate |
| I-62 | 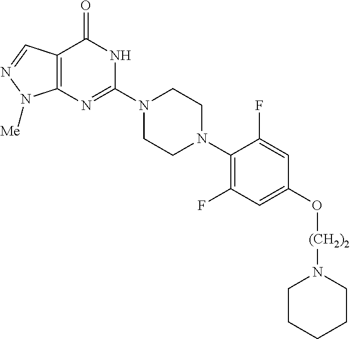 | 0.005 | | 6-{4-[2,6-Difluoro-4-(2- piperidin-1-yl-ethoxy)- phenyl]-piperazin-1-yl}-1- methyl-1,5-dihydro- pyrazolo[3,4-d]pyrimidin- 4-one |
| I-63 | 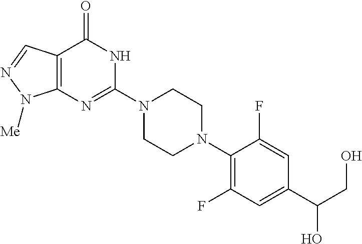 [SMILES: Cn1ncc2c(=O)[nH]c(N3CCN(c4c(F)cc(C(O)CO)cc4F)CC3)nc21] | 0.0053 | | 6-[4-[4-(1 2- dihydroxyethyl)-2 6- difluoro-phenyl]piperazin- 1-yl]-1-methyl-5H- pyrazolo[3 4-d]pyrimidin- 4-one |
| I-64 | 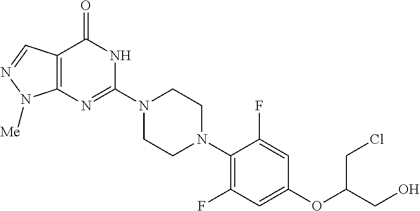 [SMILES: Cn1ncc2c(=O)[nH]c(N3CCN(c4c(F)cc(OC(CO)CCl)cc4F)CC3)nc21] | 0.0064 | | 6-[4-[4-[1-(chloromethyl)- 2-hydroxy-ethoxy]-2 6- difluoro-phenyl]piperazin- 1-yl]-1-methyl-5H- pyrazolo[3 4-d]pyrimidin- 4-one |
| I-65 | 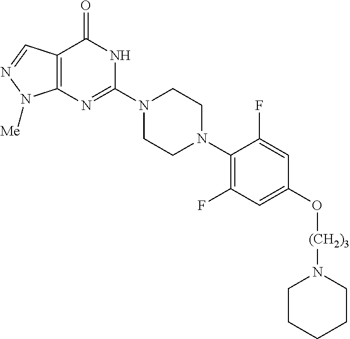 | 0.0065 | | 6-[4-[2 6-difluoro-4-(3- morpholinopropyl)phenyl] piperazin-1-yl]-1-methyl- 5H-pyrazolo[3 4- d]pyrimidin-4-one |
| I-66 | 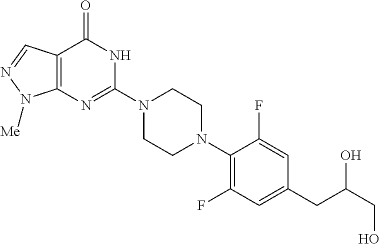 [SMILES: Cn1ncc2c(=O)[nH]c(N3CCN(c4c(F)cc(CC(O)CO)cc4F)CC3)nc21] | 0.0066 | | 6-[4-[4-(2 3- dihydroxypropyl)-2 6- difluoro-phenyl]piperazin- 1-yl]-1-methyl-5H- pyrazolo[3 4-d]pyrimidin- 4-one |
| I-67 | 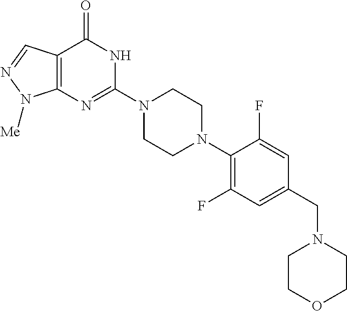 [SMILES: Cn1ncc2c(=O)[nH]c(N3CCN(c4c(F)cc(CN5CCOCC5)cc4F)CC3)nc21] | 0.0069 | | 6-[4-[2 6-difluoro-4- (morpholinomethyl)phenyl] piperazin-1-yl]-1-methyl- 5H-pyrazolo[3 4- d]pyrimidin-4-one |
| I-68 | 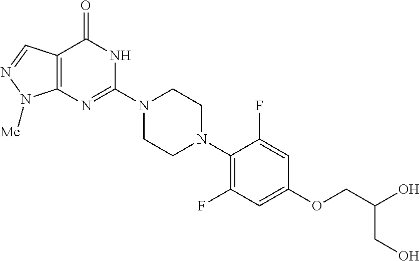 [SMILES: Cn1ncc2c(=O)[nH]c(N3CCN(c4c(F)cc(OCC(O)CO)cc4F)CC3)nc21] | 0.0070 | | 6-[4-[4-(2 3- dihydroxypropoxy)-2 6- difluoro-phenyl]piperazin- 1-yl]-1-methyl-5H- pyrazolo[3 4-d]pyrimidin- 4-one |
| I-69 | 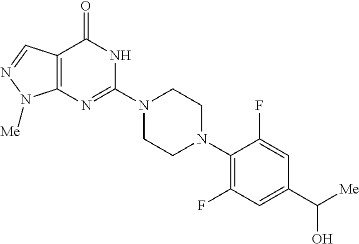 [SMILES: CC(O)c1cc(F)c(N2CCN(c3nc4c(cnn4C)c(=O)[nH]3)CC2)c(F)c1] | 0.0078 | | 6-[4-[2 6-difluoro-4-(1- hydroxyethyl)phenyl] piperazin-1-yl]-1-methyl-5H- pyrazolo[3 4-d]pyrimidin- 4-one |
| I-70 | 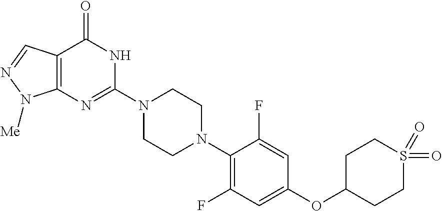 [SMILES: Cn1ncc2c(=O)[nH]c(N3CCN(c4c(F)cc(OC5CCS(=O)(=O)CC5)cc4F)CC3)nc21] | 0.0079 | | 6-[4-[4-(1 1-dioxothian-4- yl)oxy-2 6-difluoro- phenyl]piperazin-1-yl]-1- methyl-5H-pyrazolo[3 4- d]pyrimidin-4-one |
| I-71 | 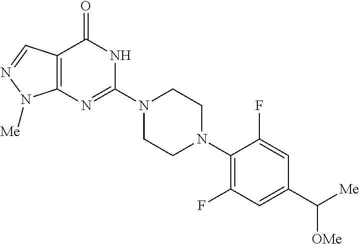 [SMILES: COC(C)c1cc(F)c(N2CCN(c3nc4c(cnn4C)c(=O)[nH]3)CC2)c(F)c1] | 0.0083 | | 6-[4-[2 6-difluoro-4-(1- methoxyethyl)phenyl] piperazin-1-yl]-1-methyl-5H- pyrazolo[3 4-d]pyrimidin- 4-one |
| I-72 | 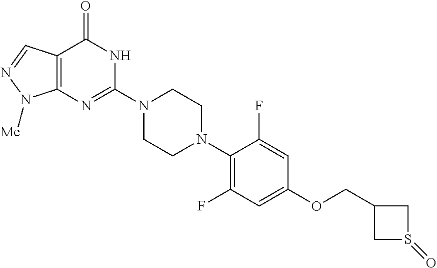 [SMILES: Cn1ncc2c(=O)[nH]c(N3CCN(c4c(F)cc(OCC5CS(=O)C5)cc4F)CC3)nc21] | 0.0086 | | 6-[4-[2 6-difluoro-4-[(1- oxothietan-3- yl)methoxy]phenyl] piperazin-1-yl]-1-methyl-5H- pyrazolo[3 4-d]pyrimidin- 4-one |
| I-73 | 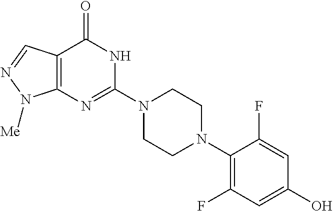 [SMILES: Cn1ncc2c(=O)[nH]c(N3CCN(c4c(F)cc(O)cc4F)CC3)nc21] | 0.0088 | | 6-[4-(2 6-difluoro-4- hydroxy-phenyl)piperazin- 1-yl]-1-methyl-5H- pyrazolo[3 4-d]pyrimidin- 4-one |
| I-74 | 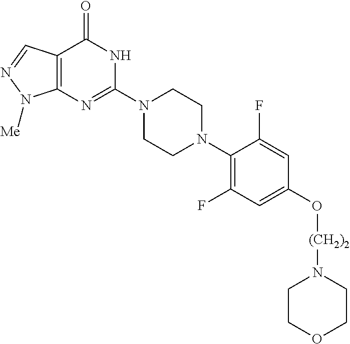 | 0.0089 | | 6-[4-[2 6-difluoro-4-(2- morpholinoethoxy)phenyl] piperazin-1-yl]-1-methyl- 5H-pyrazolo[3 4- d]pyrimidin-4-one |
| I-75 | 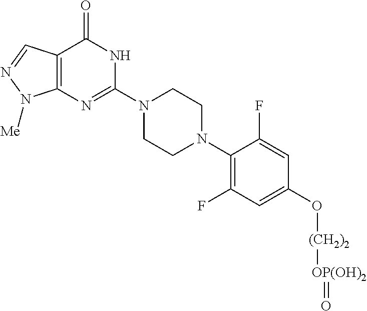 | 0.0096 | | 2-[3 5-difluoro-4-[4-(1- methyl-4-oxo-5H- pyrazolo[3 4-d]pyrimidin- 6-yl)piperazin-1- yl]phenoxy]ethyl dihydrogen phosphate |
| I-76 | 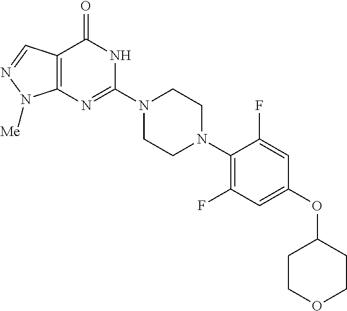 [SMILES: Cn1ncc2c(=O)[nH]c(N3CCN(c4c(F)cc(OC5CCOCC5)cc4F)CC3)nc21] | 0.0111 | | 6-[4-(2 6-difluoro-4- tetrahydropyran-4-yloxy- phenyl)piperazin-1-yl]-1- methyl-5H-pyrazolo[3 4- d]pyrimidin-4-one |
| I-77 | 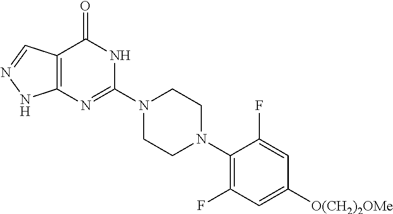 | 0.0118 | | 6-[4-[2 6-difluoro-4-(2- methoxyethoxy)phenyl] piperazin-1-yl]-1 5- dihydropyrazolo[3 4- d]pyrimidin-4-one |
| I-78 | 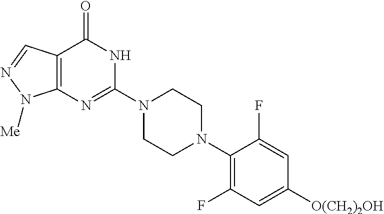 | 0.012 | | 6-[4-[2 6-difluoro-4-(2- hydroxyethoxy)phenyl] piperazin-1-yl]-1-methyl-5H- pyrazolo[3 4-d]pyrimidin- 4-one |
| I-79 | 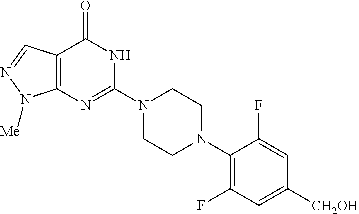 [SMILES: Cn1ncc2c(=O)[nH]c(N3CCN(c4c(F)cc(CO)cc4F)CC3)nc21] | 0.0133 | | 6-[4-[2 6-difluoro-4- (hydroxymethyl)phenyl] piperazin-1-yl]-1-methyl-5H- pyrazolo[3 4-d]pyrimidin- 4-one |
| I-80 | 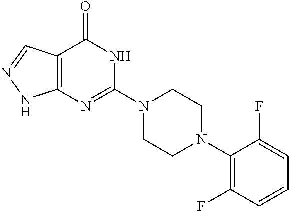 [SMILES: O=c1[nH]c(N2CCN(c3c(F)cccc3F)CC2)nc2[nH]ncc12] | 0.014 | | 6-[4-(2 6- difluorophenyl)piperazin- 1-yl]-1 5- dihydropyrazolo[3 4- d]pyrimidin-4-one |
| I-81 | 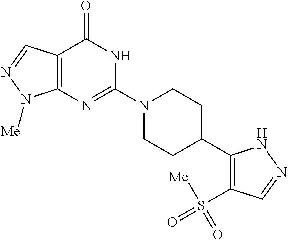 [SMILES: Cn1ncc2c(=O)[nH]c(N3CCC(c4[nH]ncc4S(C)(=O)=O)CC3)nc21] | 0.0143 | | 1-methyl-6-[4-(4- methylsulfonyl-1H- pyrazol-5-yl)-1-piperidyl]- 5H-pyrazolo[3 4- d]pyrimidin-4-one |
| I-82 | 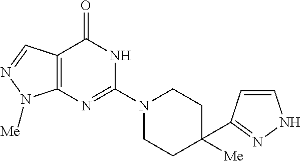 [SMILES: Cn1ncc2c(=O)[nH]c(N3CCC(C)(c4cc[nH]n4)CC3)nc21] | 0.0151 | | 1-methyl-6-[4-methyl-4- (1H-pyrazol-3-yl)-1- piperidyl]-5H-pyrazolo[3 4-d]pyrimidin-4-one |
| I-83 | 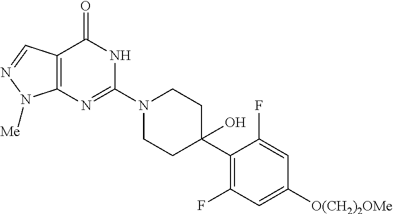 | 0.0153 | | 6-[4-[2 6-difluoro-4-(2- methoxyethoxy)phenyl]-4- hydroxy-1-piperidyl]-1- methyl-5H-pyrazolo[3 4- d]pyrimidin-4-one |
| I-84 | 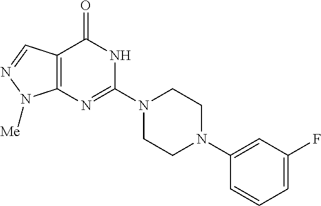 [SMILES: Cn1ncc2c(=O)[nH]c(N3CCN(c4cccc(F)c4)CC3)nc21] | 0.017 | | 6-[4-(3-fluorophenyl)-1- piperidyl]-1-methyl-5H- pyrazolo[3 4-d]pyrimidin- 4-one |
| I-85 | 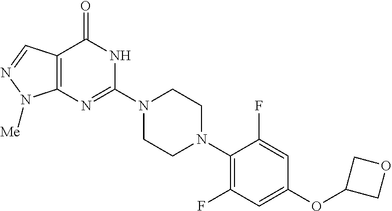 [SMILES: Cn1ncc2c(=O)[nH]c(N3CCN(c4c(F)cc(OC5COC5)cc4F)CC3)nc21] | 0.0171 | | 6-[4-[2 6-difluoro-4- (oxetan-3- yloxy)phenyl]piperazin-1- yl]-1-methyl-5H- pyrazolo[3 4-d]pyrimidin- 4-one |
| I-86 | 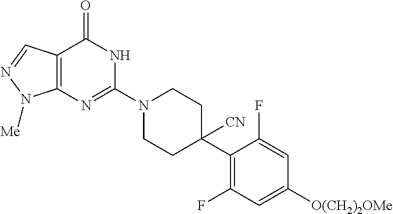 | 0.0185 | | 4-[2 6-difluoro-4-(2- methoxyethoxy)phenyl]-1- (1-methyl-4-oxo-5H- pyrazolo[3 4-d]pyrimidin- 6-yl)piperidine-4- carbonitrile |
| I-87 | 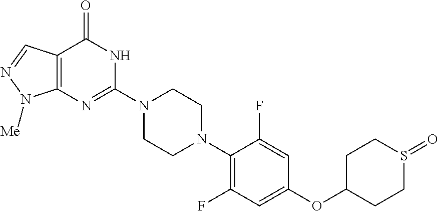 [SMILES: Cn1ncc2c(=O)[nH]c(N3CCN(c4c(F)cc(OC5CCS(=O)CC5)cc4F)CC3)nc21] | 0.0187 | | 6-[4-[2 6-difluoro-4-(1- oxothian-4-yl)oxy- phenyl]piperazin-1-yl]-1- methyl-5H-pyrazolo[3 4- d]pyrimidin-4-one |
| I-88 | 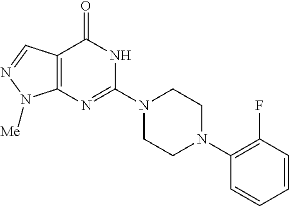 [SMILES: Cn1ncc2c(=O)[nH]c(N3CCN(c4ccccc4F)CC3)nc21] | 0.0193 | | 6-[4-(2-fluorophenyl)-1- piperidyl]-1-methyl-5H- pyrazolo[3 4-d]pyrimidin- 4-one |
| I-89 | 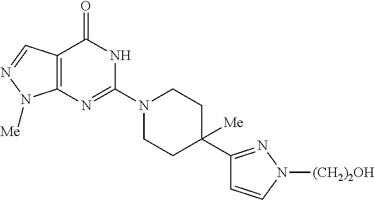 | 0.0218 | | 6-[4-[1-(2- hydroxyethyl)pyrazol-3- yl]-4-methyl-1-piperidyl]- 1-methyl-5H-pyrazolo[3 4- d]pyrimidin-4-one |
| I-90 | 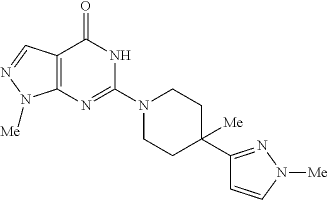 [SMILES: Cn1ccc(C2(C)CCN(c3nc4c(cnn4C)c(=O)[nH]3)CC2)n1] | 0.0236 | | 1-methyl-6-[4-methyl-4-(1- methylpyrazol-3-yl)-1- piperidyl]-5H-pyrazolo[3 4-d]pyrimidin-4-one |
| I-91 | 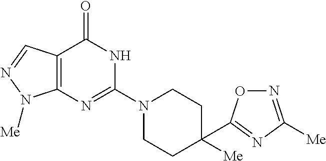 [SMILES: Cc1noc(C2(C)CCN(c3nc4c(cnn4C)c(=O)[nH]3)CC2)n1] | 0.0241 | | 1-methyl-6-[4-methyl-4-(3- methyl-1 2 4-oxadiazol-5- yl)-1-piperidyl]-5H- pyrazolo[3 4-d]pyrimidin- 4-one |
| I-92 | 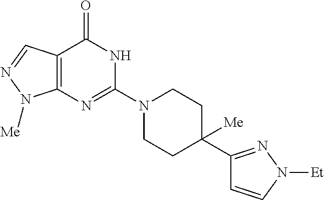 [SMILES: CCn1ccc(C2(C)CCN(c3nc4c(cnn4C)c(=O)[nH]3)CC2)n1] | 0.0245 | | 6-[4-(1-ethylpyrazol-3-yl)- 4-methyl-1-piperidyl]-1- methyl-5H-pyrazolo[3 4- d]pyrimidin-4-one |
| I-93 | 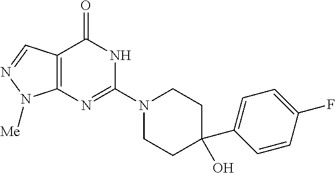 [SMILES: Cn1ncc2c(=O)[nH]c(N3CCC(O)(c4ccc(F)cc4)CC3)nc21] | 0.0246 | | 6-[4-(4-fluorophenyl)-4- hydroxy-1-piperidyl]-1- methyl-5H-pyrazolo[3 4- d]pyrimidin-4-one |
| I-94 | 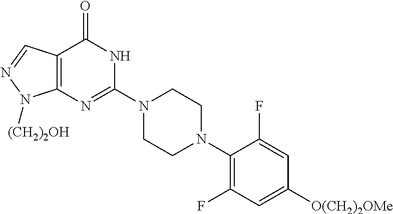 | 0.264 | | 6-[4-[2 6-difluoro-4-(2- methoxyethoxy)phenyl] piperazin-1-yl]-1-(2- hydroxyethyl)-5H- pyrazolo[3 4-d]pyrimidin- 4-one |
| I-95 | 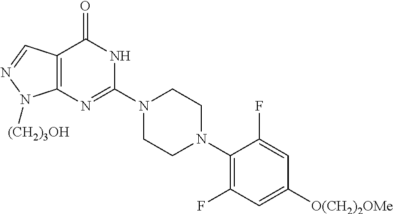 | 0.027 | | 6-[4-[2 6-difluoro-4-(2- methoxyethoxy)phenyl] piperazin-1-yl]-1-(3- hydroxypropyl)-5H- pyrazolo[3 4-d]pyrimidin- 4-one |
| I-96 | 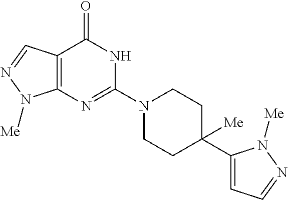 [SMILES: Cn1nccc1C1(C)CCN(c2nc3c(cnn3C)c(=O)[nH]2)CC1] | 0.0284 | | 1-methyl-6-[4-methyl-4-(2- methylpyrazol-3-yl)-1- piperidyl)-5H-pyrazolo[3 4-d]pyrimidin-4-one |
| I-97 | 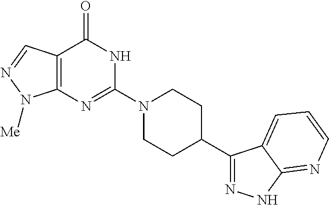 [SMILES: Cn1ncc2c(=O)[nH]c(N3CCC(c4n[nH]c5ncccc45)CC3)nc21] | 0.0319 | | 1-methyl-6-[4-(1H- pyrazolo[3 4-b]pyridin-3- yl)-1-piperidyl]-5H- pyrazolo[3 4-d]pyrimidin- 4-one |
| I-98 | 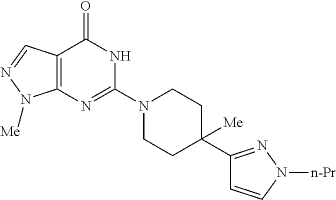 | 0.0374 | | 1-methyl-6-[4-methyl-4-(1- propylpyrazol-3-yl)-1- piperidyl]-5H-pyrazolo[3 4-d]pyrimidin-4-one |
| I-99 | 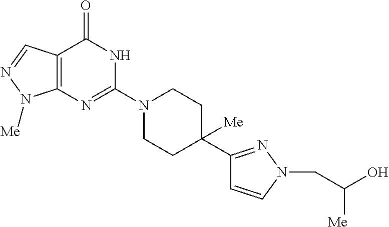 [SMILES: CC(O)Cn1ccc(C2(C)CCN(c3nc4c(cnn4C)c(=O)[nH]3)CC2)n1] | 0.0419 | | 6-[4-[1-(2- hydroxypropyl)pyrazol-3- yl]-4-methyl-1-piperidyl]- 1-methyl-5H-pyrazolo[3 4- d]pyrimidin-4-one |
| I-100 | 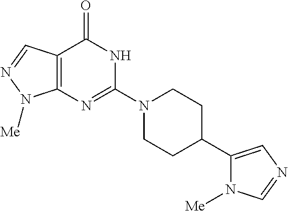 [SMILES: Cn1cncc1C1CCN(c2nc3c(cnn3C)c(=O)[nH]2)CC1] | 0.0438 | | 1-methyl-6-[4-(3- methylimidazol-4-yl)-1- piperidyl]-5H-pyrazolo[3 4-d]pyrimidin-4-one |
| I-101 | 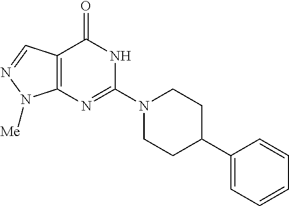 [SMILES: Cn1ncc2c(=O)[nH]c(N3CCC(c4ccccc4)CC3)nc21] | 0.0462 | | 1-methyl-6-(4-phenyl-1- piperidyl)-5H-pyrazolo[3 4-d]pyrimidin-4-one |
| I-102 | 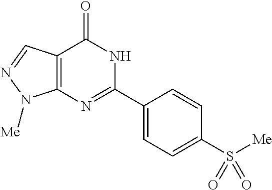 [SMILES: Cn1ncc2c(=O)[nH]c(-c3ccc(S(C)(=O)=O)cc3)nc21] | 0.0479 | | 1-methyl-6-(4- methylsulfonylphenyl)-5H- pyrazolo[3 4-d]pyrimidin- 4-one |
| I-103 | 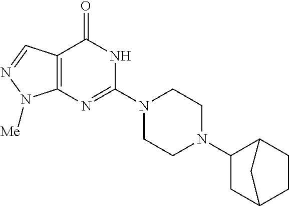 [SMILES: Cn1ncc2c(=O)[nH]c(N3CCN(C4CC5CCC4C5)CC3)nc21] | 0.0502 | | 1-methyl-6-(4-norbornan- 2-ylpiperazin-1-yl)-5H- pyrazolo[3 4-d]pyrimidin- 4-one |
| I-104 | 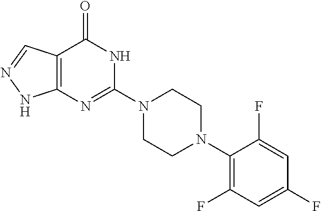 [SMILES: O=c1[nH]c(N2CCN(c3c(F)cc(F)cc3F)CC2)nc2[nH]ncc12] | 0.055 | | 6-[4-(2 4 6- trifluorophenyl)piperazin- 1-yl]-1 5- dihydropyrazolo[3 4- d]pyrimidin-4-one |
| I-105 | 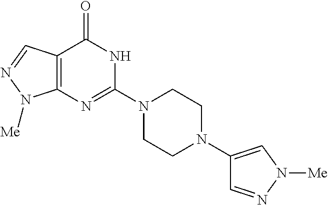 [SMILES: Cn1cc(N2CCN(c3nc4c(cnn4C)c(=O)[nH]3)CC2)cn1] | 0.0639 | | 1-methyl-6-[4-(1- methylpyrazol-4- yl)piperazin-1-yl]-5H- pyrazolo[3 4-d]pyrimidin- 4-one |
| I-106 | 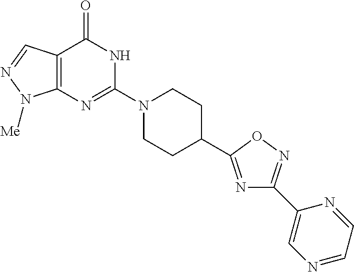 [SMILES: Cn1ncc2c(=O)[nH]c(N3CCC(c4nc(-c5cnccn5)no4)CC3)nc21] | 0.0641 | | 1-methyl-6-[4-(3-pyrazin- 2-yl-1 2 4-oxadiazol-5-yl)- 1-piperidyl]-5H-pyrazolo[3 4-d]pyrimidin-4-one |
| I-107 | 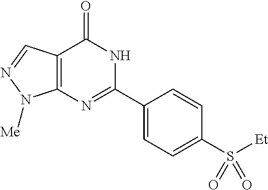 [SMILES: CCS(=O)(=O)c1ccc(-c2nc3c(cnn3C)c(=O)[nH]2)cc1] | 0.0681 | | 6-(4-ethylsulfonylphenyl)- 1-methyl-5H-pyrazolo[3 4- d]pyrimidin-4-one |
| I-108 | 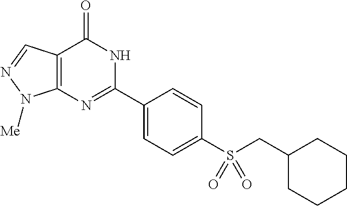 [SMILES: Cn1ncc2c(=O)[nH]c(-c3ccc(S(=O)(=O)CC4CCCCC4)cc3)nc21] | 0.0684 | | 6-[4- (cyclohexylmethylsulfonyl) phenyl]-1-methyl-5H- pyrazolo[3 4-d]pyrimidin- 4-one |
| I-109 | 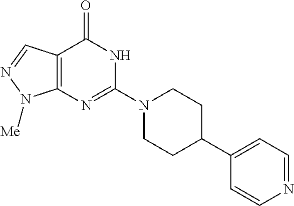 [SMILES: Cn1ncc2c(=O)[nH]c(N3CCC(c4ccncc4)CC3)nc21] | 0.0806 | | 1-methyl-6-[4-(4-pyridyl)- 1-piperidyl)-5H-pyrazolo[3 4-d]pyrimidin-4-one |
| I-110 | 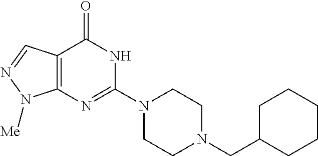 [SMILES: Cn1ncc2c(=O)[nH]c(N3CCN(CC4CCCCC4)CC3)nc21] | 0.0809 | | 6-[4- (cyclohexylmethyl) piperazin-1-yl]-1-methyl-5H- pyrazolo[3 4-d]pyrimidin- 4-one |
| I-111 | 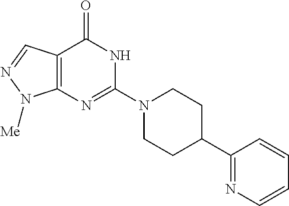 [SMILES: Cn1ncc2c(=O)[nH]c(N3CCC(c4ccccn4)CC3)nc21] | 0.0838 | | 1-methyl-6-[4-(2-pyridyl)- 1-piperidyl]-5H-pyrazolo[3 4-d]pyrimidin-4-one |
| I-112 | 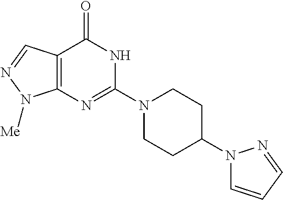 [SMILES: Cn1ncc2c(=O)[nH]c(N3CCC(n4cccn4)CC3)nc21] | 0.0883 | | 1-methyl-6-(4-pyrazol-1- yl-1-piperidyl)-5H- pyrazolo[3 4-d]pyrimidin- 4-one |
| I-113 | 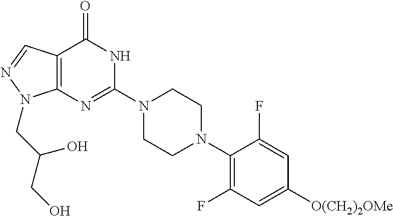 | 0.09 | | 6-[4-[2 6-difluoro-4-(2- methoxyethoxy)phenyl] piperazin-1-yl]-1-(2 3- dihydroxypropyl)-5H- pyrazolo[3 4-d]pyrimidin- 4-one |
| I-114 | 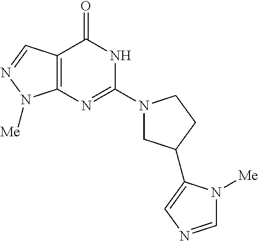 [SMILES: Cn1cncc1C1CCN(c2nc3c(cnn3C)c(=O)[nH]2)C1] | 0.0903 | | 1-methyl-6-[3-(3- methylimidazol-4- yl)pyrrolidin-1-yl]-5H- pyrazolo[3 4-d]pyrimidin- 4-one |
| I-115 | 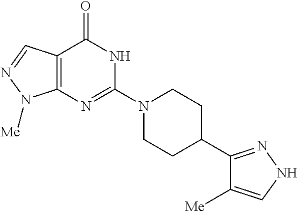 [SMILES: Cc1c[nH]nc1C1CCN(c2nc3c(cnn3C)c(=O)[nH]2)CC1] | 0.107 | | 1-methyl-6-[4-(4-methyl- 1H-pyrazol-3-yl)-1- piperidyl]-5H-pyrazolo[3 4-d]pyrimidin-4-one |
| I-116 | 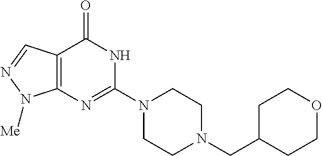 [SMILES: Cn1ncc2c(=O)[nH]c(N3CCN(CC4CCOCC4)CC3)nc21] | 0.112 | | 1-methyl-6-[4- (tetrahydropyran-4- ylmethyl)piperazin-1-yl]- 5H-pyrazolo[3 4- d]pyrimidin-4-one |
| I-117 | 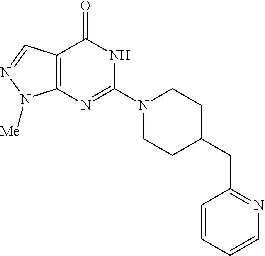 [SMILES: Cn1ncc2c(=O)[nH]c(N3CCC(Cc4ccccn4)CC3)nc21] | 0.112 | | 1-methyl-6-[4-(2- pyridylmethyl)-1- piperidyl]-5H-pyrazolo[3 4-d]pyrimidin-4-one |
| I-118 | 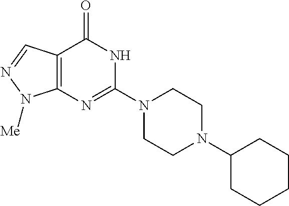 [SMILES: Cn1ncc2c(=O)[nH]c(N3CCN(C4CCCCC4)CC3)nc21] | 0.122 | | 6-(4-cyclohexylpiperazin- 1-yl)-1-methyl-5H- pyrazolo[3 4-d]pyrimidin- 4-one |
| I-119 | 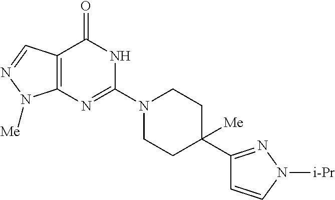 [SMILES: CC(C)n1ccc(C2(C)CCN(c3nc4c(cnn4C)c(=O)[nH]3)CC2)n1] | 0.124 | | 6-[4-(1-isopropylpyrazol- 3-yl)-4-methyl-1- piperidyl]-1-methyl-5H- pyrazolo[3 4-d]pyrimidin- 4-one |
| I-120 | 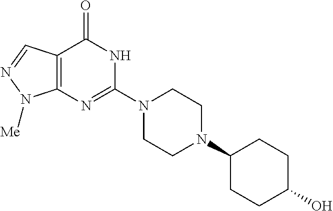 [SMILES: Cn1ncc2c(=O)[nH]c(N3CCN([C@H]4CC[C@H](O)CC4)CC3)nc21] | 0.153 | | 6-[4-(4- hydroxycyclohexyl) piperazin-1-yl]-1-methyl-5H- pyrazolo[3 4-d]pyrimidin- 4-one |
| I-121 | 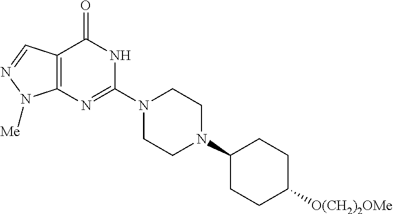 | 0.159 | | 6-[4-[4-(2- methoxyethoxy)cyclohexyl] piperazin-1-yl]-1-methyl- 5H-pyrazolo[3 4- d]pyrimidin-4-one |
| I-122 | 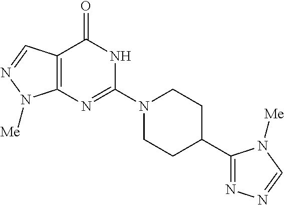 [SMILES: Cn1cnnc1C1CCN(c2nc3c(cnn3C)c(=O)[nH]2)CC1] | 0.163 | | 1-methyl-6-[4-(4-methyl-1 2 4-triazol-3-yl)-1- piperidyl]-5H-pyrazolo[3 4-d]pyrimidin-4-one |
| I-123 | 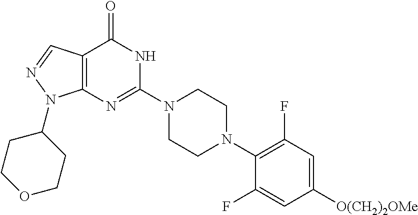 | 0.168 | | 6-[4-[2 6-difluoro-4-(2- methoxyethoxy)phenyl] piperazin-1-yl]-1- tetrahydropyran-4-yl-5H- pyrazolo[3 4-d]pyrimidin- 4-one |
| I-124 | 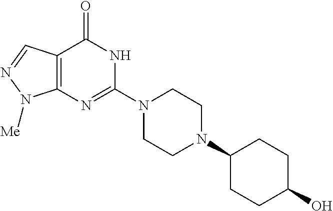 [SMILES: Cn1ncc2c(=O)[nH]c(N3CCN([C@H]4CC[C@@H](O)CC4)CC3)nc21] | 0.171 | | 6-[4-(4- hydroxycyclohexyl) piperazin-1-yl]-1-methyl-5H- pyrazolo[3 4-d]pyrimidin- 4-one |
| I-125 | 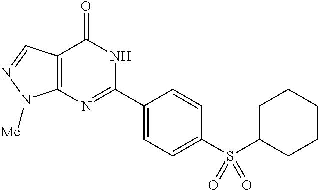 [SMILES: Cn1ncc2c(=O)[nH]c(-c3ccc(S(=O)(=O)C4CCCCC4)cc3)nc21] | 0.178 | | 6-(4- cyclohexylsulfonylphenyl)- 1-methyl-5H-pyrazolo[3 4- d]pyrimidin-4-one |
| I-126 | 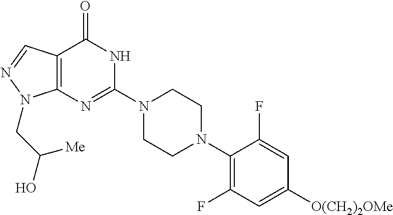 | 0.220 | | 6-[4-[2 6-difluoro-4-(2- methoxyethoxy)phenyl] piperazin-1-yl]-1-(2- hydroxypropyl)-5H- pyrazolo[3 4-d]pyrimidin- 4-one |
| I-127 | 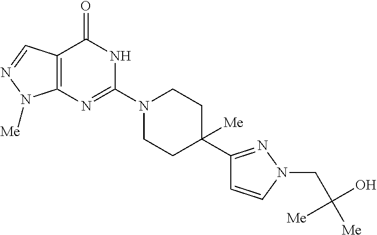 [SMILES: Cn1ncc2c(=O)[nH]c(N3CCC(C)(c4ccn(CC(C)(C)O)n4)CC3)nc21] | 0.223 | | 6-[4-[1-(2-hydroxy-2- methyl-propyl)pyrazol-3- yl)-4-methyl-1-piperidyl]- 1-methyl-5H-pyrazolo[3 4- d]pyrimidin-4-one |
| I-128 | 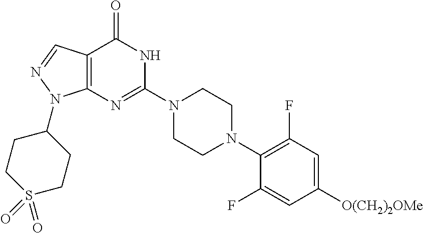 | 0.268 | | 6-[4-[2 6-difluoro-4-(2- methoxyethoxy)phenyl] piperazin-1-yl]-1-(1 1- dioxothian-4-yl)-5H- pyrazolo[3 4-d]pyrimidin- 4-one |
| I-129 | 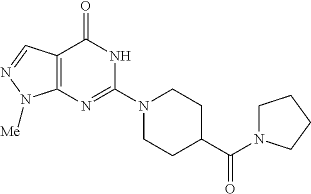 [SMILES: Cn1ncc2c(=O)[nH]c(N3CCC(C(=O)N4CCCC4)CC3)nc21] | 0.274 | | 1-methyl-6-[4-(pyrrolidine- 1-carbonyl)-1-piperidyl)- 5H-pyrazolo[3 4- d]pyrimidin-4-one |
| I-130 | 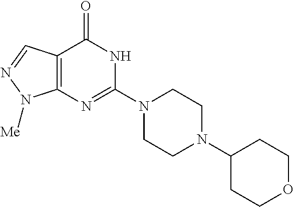 [SMILES: Cn1ncc2c(=O)[nH]c(N3CCN(C4CCOCC4)CC3)nc21] | 0.315 | | 1-methyl-6-(4- tetrahydropyran-4- ylpiperazin-1-yl)-5H- pyrazolo[3 4-d]pyrimidin- 4-one |
| I-131 | 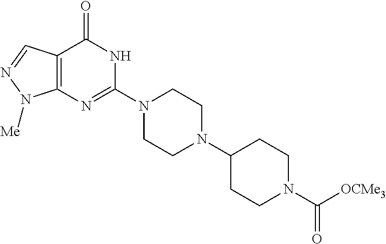 | 0.317 | | tert-butyl 4-[4-(1-methyl-4- oxo-5H-pyrazolo[3 4- d]pyrimidin-6- yl)piperazin-1- yl]piperidine-1-carboxylate |
| I-132 | 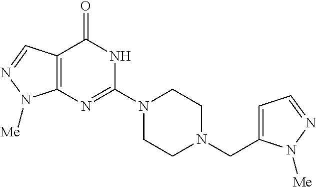 [SMILES: Cn1nccc1CN1CCN(c2nc3c(cnn3C)c(=O)[nH]2)CC1] | 0.329 | | 1-methyl-6-[4-((2- methylpyrazol-3- yl)methyl]piperazin-1-yl]- 5H-pyrazolo[3 4- d]pyrimidin-4-one |
| I-133 | 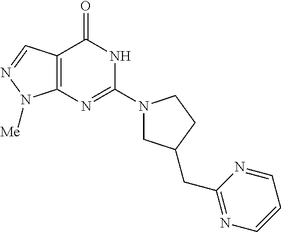 [SMILES: Cn1ncc2c(=O)[nH]c(N3CCC(Cc4ncccn4)C3)nc21] | 0.337 | | 1-methyl-6-[3-(pyrimidin- 2-ylmethyl)pyrrolidin-1- yl]-5H-pyrazolo[3 4- d]pyrimidin-4-one |
| I-134 | 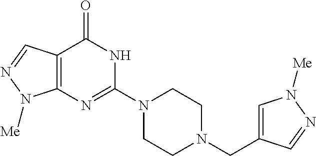 [SMILES: Cn1cc(CN2CCN(c3nc4c(cnn4C)c(=O)[nH]3)CC2)cn1] | 0.342 | | 1-methyl-6-[4-[(1- methylpyrazol-4- yl)methyl]piperazin-1-yl]- 5H-pyrazolo[3 4- d]pyrimidin-4-one |
| I-135 | 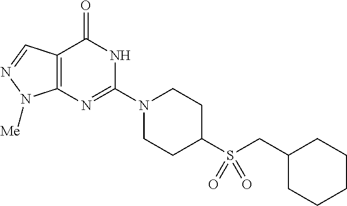 [SMILES: Cn1ncc2c(=O)[nH]c(N3CCC(S(=O)(=O)CC4CCCCC4)CC3)nc21] | 0.406 | | 6-[4- (cyclohexylmethylsulfonyl)- 1-piperidyl]-1-methyl- 5H-pyrazolo[3 4- d]pyrimidin-4-one |
| I-136 | 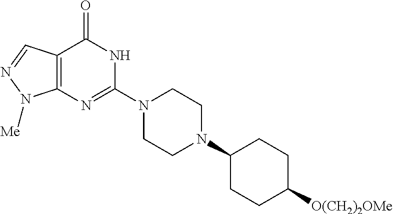 | 0.468 | | 6-[4-[4-(2- methoxyethoxy)cyclohexyl] piperazin-1-yl]-1-methyl- 5H-pyrazolo[3 4- d]pyrimidin-4-one |
| I-137 | 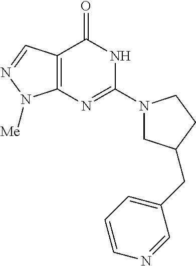 [SMILES: Cn1ncc2c(=O)[nH]c(N3CCC(Cc4cccnc4)C3)nc21] | 0.49 | | 1-methyl-6-[3-(3- pyridylmethyl)pyrrolidin- 1-yl]-5H-pyrazolo[3 4- d]pyrimidin-4-one |
| I-138 | 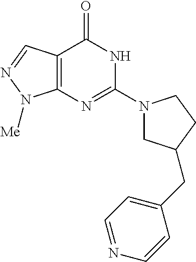 [SMILES: Cn1ncc2c(=O)[nH]c(N3CCC(Cc4ccncc4)C3)nc21] | 0.536 | | 1-methyl-6-[3-(4- pyridylmethyl)pyrrolidin- 1-yl]-5H-pyrazolo[3 4- d]pyrimidin-4-one |
| I-139 | 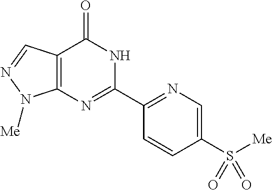 [SMILES: Cn1ncc2c(=O)[nH]c(-c3ccc(S(C)(=O)=O)cn3)nc21] | 0.547 | | 1-methyl-6-(5- methylsulfonyl-2-pyridyl)- 5H-pyrazolo[3 4- d]pyrimidin-4-one |
| I-140 | 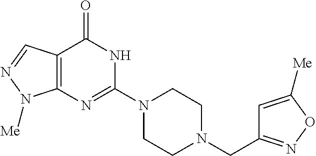 [SMILES: Cc1cc(CN2CCN(c3nc4c(cnn4C)c(=O)[nH]3)CC2)no1] | 0.566 | | 1-methyl-6-[4-[(5- methylisoxazol-3- yl)methyl]piperazin-1-yl]- 5H-pyrazolo[3 4- d]pyrimidin-4-one |
| I-141 | 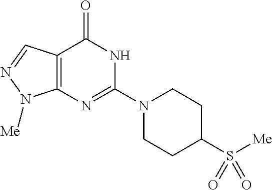 [SMILES: Cn1ncc2c(=O)[nH]c(N3CCC(S(C)(=O)=O)CC3)nc21] | 0.652 | | 1-methyl-6-(4- methylsulfonyl-1- piperidyl)-5H-pyrazolo[3 4-d]pyrimidin-4-one |
| I-142 | 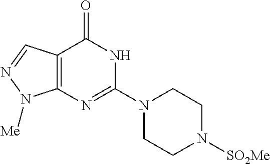 [SMILES: Cn1ncc2c(=O)[nH]c(N3CCN(S(C)(=O)=O)CC3)nc21] | 0.74 | | 1-methyl-6-(4- methylsulfonylpiperazin-1- yl)-5H-pyrazolo[3 4- d]pyrimidin-4-one |
| I-143 | 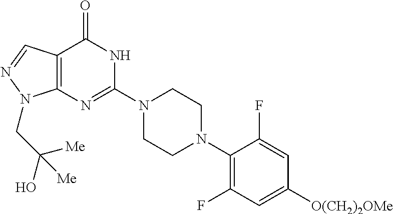 | 0.795 | | 6-[4-[2 6-difluoro-4-(2- methoxyethoxy)phenyl] piperazin-1-yl]-1-(2-hydroxy- 2-methyl-propyl)-5H- pyrazolo[3 4-d]pyrimidin- 4-one |
| I-144 | 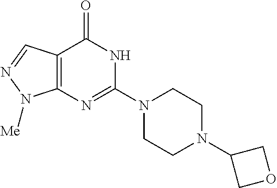 [SMILES: Cn1ncc2c(=O)[nH]c(N3CCN(C4COC4)CC3)nc21] | 0.83 | | 1-methyl-6-[4-(oxetan-3- yl)piperazin-1-yl]-5H- pyrazolo[3 4-d]pyrimidin- 4-one |

¹ Compounds I-1 to I-58 were assayed by the procedure in Example 118. Compounds I-59 to I-144 were assayed by the procedure in Example 119

General Reaction Schemes

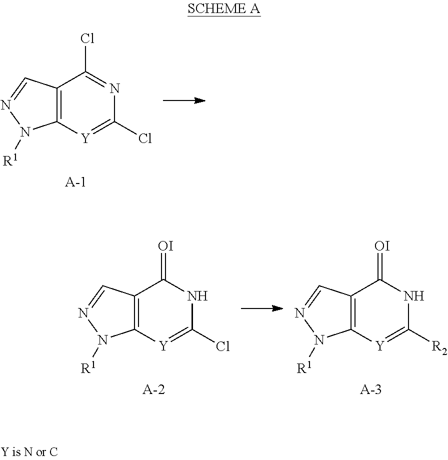

The compounds of formula I where A4 is hydrogen and where Y is carbon or nitrogen can be purchased from commercial sources.

The compound of formula I where R₁ is lower alkyl and Y is carbon can be prepared by reacting 4,6-dchloro-1H-pyrazolo[4,3-c]pyridine with a commercially available or a synthetically prepared halide of the corresponding lower alkyl derivative under basic conditions (see e.g., Chuaqui, C. E.; Huang, S.; Ioannidis, S.; Shi, J.; Su, M.; Su, Q., WO2010/038060 A1). The lower alkyl derivative may be in a protected form that may be deprotected at some point in the synthesis. The lower alkyl derivative could also be transformed through standard chemical manipulation.

The compound of formula I where R₁ is lower alkyl and Y is nitrogen can be prepared following the literature procedure (see e.g., Bursavich, M. G.; Nowak, P. W.; Malwitz, D.; Lombardi, S.; Gilbert, A. M.; Zhang, N.; Ayral-Kaloustian, S.; Anderson, J. T.; Brooijmans, N., US2010/0015141A1). The lower alkyl derivative may be in a protected form that may be deprotected at some point in the synthesis. The lower alkyl derivative could also be transformed through standard chemical manipulation.

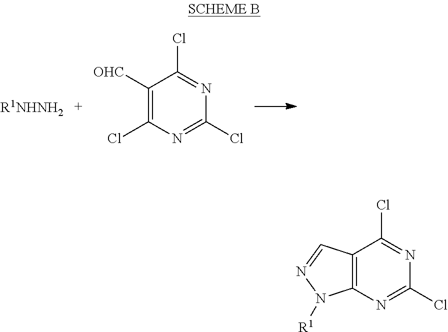

Compounds of formula I can also be prepared by reacting a substituted hydrazine derivative with 2,4,6-trichloro-pyrimidine-5-carbaldehyde.

The compound of formula A-2 where R₁ is hydrogen and Y is carbon or nitrogen can be prepared from the compound of formula A-1 where R₁ is hydrogen and Y is carbon or nitrogen by heating under basic aqueous conditions (Zhang, Z., Wallace, M. B., Feng, J., Stafford, J. A., Skene, R. J., Shi, L., Lee, B., Aertgeerts, K., Jennings, A., Xu, R., Kassel, D. B., Kaldor, S. W., Navre, M., Webb, D. R., Gwaltney, S. L, II, J. Med. Chem. 2011, 54(2):510-524).

The compound of formula A-3 where R₁ is lower alkyl, Y is carbon or nitrogen and R₂ is an appropriately substituted secondary or tertiary amino group can be prepared from the compound of formula A-2 where R₁ is lower alkyl and Y is carbon or nitrogen through nucleophilic displacement of the chloro of the compound of formula A-2 with an appropriately substituted primary or secondary amino group (see e.g., Ram, V. J., Farhanullah, Tripathi, B. K., Srivastava, A. K., Bioorg. Med. Chem. 2003 11:2439-2444). The amine reagent may be appropriately protected or functionalized such that upon displacement of the chloro the protecting group could be removed and the various functionalities could be further elaborated. The amine reagent may be commercially available or may be prepared through standard synthetic manipulation. The lower alkyl of R₁ may be in a protected form that may be deprotected at some point in the synthesis. The lower alkyl of R₁ derivative could also be transformed through standard chemical manipulation.

The compound of formula A-3 where R₁ is hydrogen, Y is carbon or nitrogen and R₂ is an appropriately substituted primary or secondary amino group can be prepared from the compound of formula A-2 where R₁ is hydrogen and Y is carbon or nitrogen through nucleophilic displacement of the chloro of the compound of formula A-2 with an appropriately substituted primary or secondary amino group (see for example, Ram, V. J., Farhanullah, Tripathi, B. K., Srivastava, A. K., Bioorg. Med. Chem. 2003 11:2439-2444). The amine reagent may be appropriately protected or functionalized such that upon displacement of the chloro the protecting group could be removed and the various functionalities could be further elaborated. The amine reagent may be commercially available or may be prepared through standard synthetic manipulation.

The compound of formula A-3 where R₁ is lower alkyl, Y is carbon or nitrogen and R₂ is aryl, substituted aryl, heteroaryl or substituted heteroaryl can be prepared from the compound of formula A-2 where R₁ is lower alkyl and Y is carbon or nitrogen through a transition metal-catalyzed coupling reaction, the Suzuki reaction, with a boronic acid or boronate ester of a aryl, substituted aryl, heteroaryl or substituted heteroaryl (see for example, Denny, W. A., Baguley, B. C., Marshall, E. S., Sutherland, H. S., WO2007/117161 A1).

The Suzuki reaction is a palladium-catalyzed coupling of a boronic acid (R—B(OH)₂) wherein R is aryl or vinyl) with an aryl or vinyl halide or triflate (WY wherein R′=aryl or vinyl; =halide or OSO₂CF₃) o afford a compound R—R′. Typical catalysts include Pd(PPh₃)₃, Pd(OAc)₂ and PdCl₂(dppf). With PdCl₂(dppf), primary alkyl borane compounds can be coupled to aryl or vinyl halide or triflate without β-elimination. Highly active catalysts have been identified (see, e.g. J. P. Wolfe et al., J. Am. Chem. Soc. 1999 121(41):9550-9561 and A. F. Littke et al., J. Am. Chem. Soc. 2000 122(17):4020-4028). The reaction can be carried out in a variety of organic solvents including toluene, THF, dioxane, 1,2-dichloroethane, DMF, PhMe, MeOH, DMSO and acetonitrile, aqueous solvents and under biphasic conditions. Reactions are typically run from about room temperature to about 150° C. Additives (e.g. CsF, KF, T10H, NaOEt and KOH) frequently accelerate the coupling. There are a large, number of parameters in the Suzuki reaction including the palladium source, ligand, additives and temperature and optimum conditions sometimes require optimization of the parameters for a given pair of reactants. A. F. Littke et al., supra, disclose conditions for Suzuki cross-coupling with arylboronic acids in high yield at RT utilizing Pd₂(dba)₃/P(tert-bu)₃ and conditions for cross-coupling of aryl- and vinyl triflates utilizing Pd(OAc)₂/P(C₆H₁₁)₃ at RT. J. P. Wolf et al., supra, disclose efficient condition for Suzuki cross-coupling utilizing Pd(OAc)₂/o-(di-tert-butylphosphino)biphenyl or o-(dicyclohexylyphosphino)biphenyl. One skilled in the art can determine optimal conditions without undue experimentation.

The lower alkyl derivative of R₁ may be in a protected form that may be deprotected at some point in the synthesis. The lower alkyl derivative of R₁ derivative could also be transformed through standard chemical manipulation.

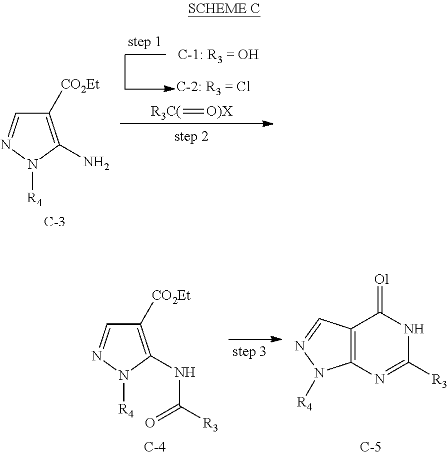

The compounds of formula C-1 where R₃ may be an aryl, substituted aryl, heteroaryl or substituted heteroaryl ring may be commercially available or able to be prepared by known synthetic methods from a variety of precursors.

The compound of formula C-2 where R₃ is aryl, substituted aryl, heteroaryl or substituted heteroaryl can be prepared from the compound of formula IV through standard synthetic methods to give the corresponding acid chloride, V (see e.g., Pellegata, R., VIIIa, I. M., Synthesis 1985 5:517-19).

The compound of formula C-3 where R₄ is lower alkyl, aryl, substituted aryl, heteroaryl or substituted heteroaryl may be commercially available or able to be prepared by known synthetic methods from a variety of precursors.

The compound of formula C-4 where R₃ is aryl, substituted aryl, heteroaryl or substituted heteroaryl and where R₄ is lower alkyl, aryl, substituted aryl, heteroaryl or substituted heteroaryl can be prepared from the compound of formula C-2 where R₃ is aryl, substituted aryl, heteroaryl or substituted heteroaryl and from the compounds of formula C-3 where R₄ is lower alkyl, aryl, substituted aryl, heteroaryl or substituted heteroaryl through nucleophilic displacement of the halide of the acid chloride of the compound of formula C-2 with the amine of the compound of formula C-3 (see for example, Werbel, L. M., Elslager, E. F., Islip, P. J., Closier, M. D., J. Med. Chem. 1977, 20(12): 1562-1569).

The compounds of formula C-6 where R₃ is aryl, substituted aryl, heteroaryl or substituted heteroaryl and where R₄ is lower alkyl, aryl, substituted aryl, heteroaryl or substituted heteroaryl can be prepared from the compound of formula C-4 where R₃ is aryl, substituted aryl, heteroaryl or substituted heteroaryl and where R₄ is lower alkyl, aryl, substituted aryl, heteroaryl or substituted heteroaryl through cyclization followed by treatment with an ammonia equivalent (see for example, Jakobsen, P., Horneman, A. M., Persson, E. Bioorg. Med. Chem. 2000 8: 2803-2812; Temple, D. L., Yevich, J. P., Covington, R. R., Hanning, C. A., Seidehamel, R. J., Mackey, H. K., Bartek, M. J., J. Med. Chem. 1979 22(5):505-510).

The groups R₃ and R₄ may be in a protected form that may be deprotected at some point in the synthesis. The groups R₃ and R₄ could also be transformed through standard chemical manipulation.

Substituted 1-(2,6-difluorophenyl)piperazines are useful intermediates for preparation of some compounds within the scope of the present invention and can be prepared from 1-[4-(2,6-difluoro-4-nitrophenyl)-1-piperazinyl]-ethanone (CASRN 1260761-78-1) as depicted in SCHEME D.

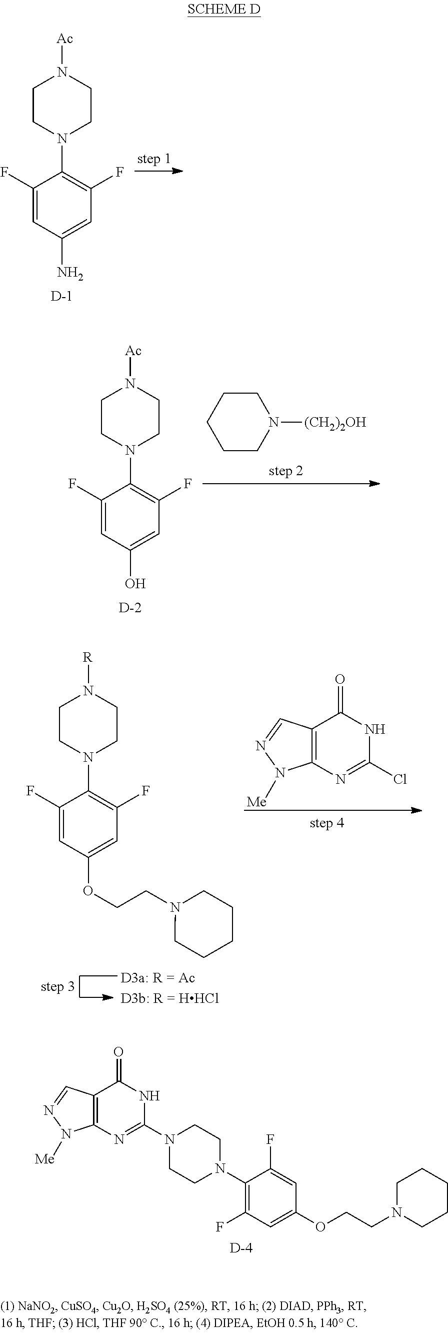

4-Methyl-4-(2-alkyl-2H-pyrazol-3-yl)-piperidines which are used to prepare compounds of the instant invention are prepare as depicted in SCHEME E.

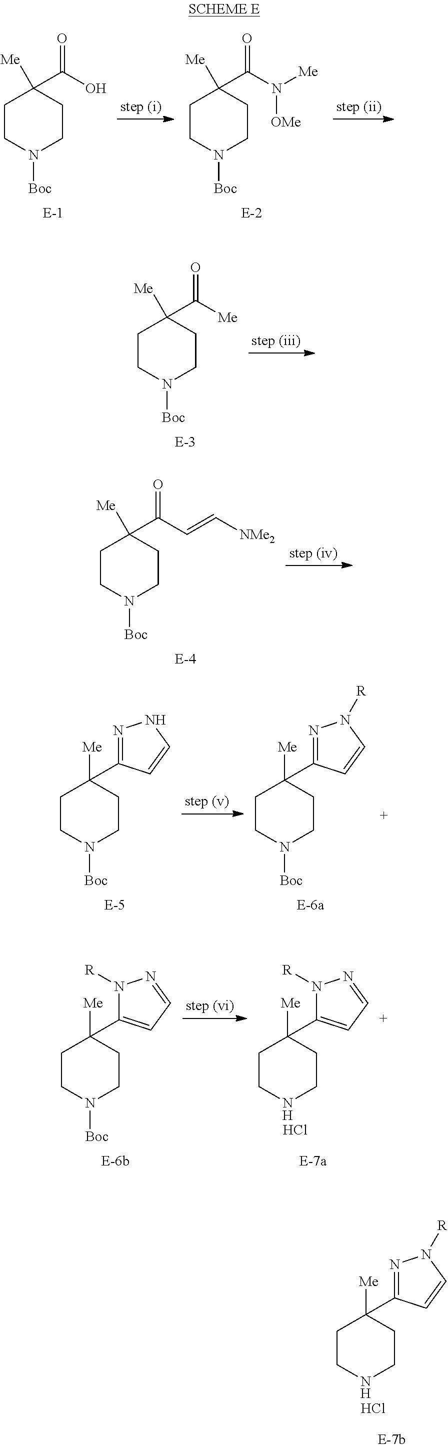

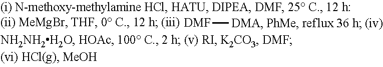

The following preparations and examples are given to enable those skilled in the art to more clearly understand and to practice the present invention. They should not be considered as limiting the scope of the invention, but merely as being illustrative and representative thereof.

Biological Activity

Determination of the activity of tankyrase activity of a compound of formula I was accomplished using the tankyrase inhibition assay exemplified in Examples 118 and 119.

Dosage & Administration

The present invention provides pharmaceutical compositions or medicaments containing the compounds of the invention and at least one therapeutically inert carrier, diluent or excipient, as well as methods of using the compounds of the invention to prepare such compositions and medicaments. In one example, compounds of formula I with the desired degree of purity may be formulated by mixing with physiologically acceptable carriers, i.e., carriers that are non-toxic to recipients at the dosages and concentrations employed into a dosage form at ambient temperature and at the appropriate pH. The pH of the formulation depends mainly on the particular use and the concentration of compound, but typically ranges anywhere from about 3 to about 8. In one example, a compound of formula I is formulated in an acetate buffer, at pH 5. In another embodiment, the compounds of formula I are sterile. The compound may be stored, for example, as a solid or amorphous composition, as a lyophilized formulation or as an aqueous solution.

Compositions are formulated, dosed, and administered in a fashion consistent with good medical practice. The term “therapeutically effective amount” denotes an amount of a compound of the present invention that, when administered to a subject, (i) treats or prevents the particular disease, condition or disorder, (ii) attenuates, ameliorates or eliminates one or more symptoms of the particular disease, condition, or disorder, or (iii) prevents or delays the onset of one or more symptoms of the particular disease, condition or disorder described herein. The therapeutically effective amount will vary depending on the particular disorder being treated, the severity of the disorder, the particular patient being treated, the clinical condition of the individual patient, the cause of the disorder, the site of delivery of the agent, the method of administration, the scheduling of administration, and other factors known to medical practitioners

The term “treating” or “treatment” of a disease state includes (1) inhibiting the disease state, i.e., arresting the development of the disease state or its clinical symptoms, or (2) relieving the disease state, i.e., causing temporary or permanent regression of the disease state or its clinical symptoms.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

Sustained-release preparations may be prepared. Suitable examples of sustained-release preparations include semipermeable matrices of solid hydrophobic polymers containing a compound of formula I, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides, copolymers of L-glutamic acid and gamma-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

A dose to treat human patients may range from about 0.1 mg to about 1000 mg of a compound of formula I. A typical dose may be about 1 mg to about 300 mg of the compound. A dose may be administered once a day (QID), twice per day (BID), or more frequently, depending on the pharmacokinetic and pharmacodynamic properties, including absorption, distribution, metabolism, and excretion of the particular compound. In addition, toxicity factors may influence the dosage and administration regimen. When administered orally, the pill, capsule, or tablet may be ingested daily or less frequently for a specified period of time. The regimen may be repeated for a number of cycles of therapy.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

For oral administration, tablets containing various excipients, such as citric acid may be employed together with various disintegrants such as starch, alginic acid and certain complex silicates and with binding agents such as sucrose, gelatin and acacia. Additionally, lubricating agents such as magnesium stearate, sodium lauryl sulfate and talc are often useful for tableting purposes. Solid compositions of a similar type may also be employed in soft and hard filled gelatin capsules. Preferred materials, therefore, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

An example of a suitable oral dosage form is a tablet containing about 25 mg, 50 mg, 100 mg, 250 mg or 500 mg of the compound of the invention compounded with about 90-30 mg anhydrous lactose, about 5-40 mg sodium croscarmellose, about 5-30 mg polyvinylpyrrolidone (PVP) K30, and about 1-10 mg magnesium stearate. The powdered ingredients are first mixed together and then mixed with a solution of the PVP. The resulting composition can be dried, granulated, mixed with the magnesium stearate and compressed to tablet form using conventional equipment. An example of an aerosol formulation can be prepared by dissolving the compound, for example 5-400 mg, of the invention in a suitable buffer solution, e.g. a phosphate buffer, adding a tonicifier, e.g. a salt such sodium chloride, if desired. The solution may be filtered, e.g., using a 0.2 micron filter, to remove impurities and contaminants.

In one embodiment, the pharmaceutical composition also includes at least one additional anti-proliferative agent.

An embodiment, therefore, includes a pharmaceutical composition comprising a compound of formula I, or a stereoisomer or pharmaceutically acceptable salt thereof. In a further embodiment includes a pharmaceutical composition comprising a compound of formula I, or a stereoisomer or pharmaceutically acceptable salt thereof, together with a pharmaceutically acceptable carrier or excipient.

The invention further provides veterinary compositions comprising at least one active ingredient as above defined together with a veterinary carrier therefore. Veterinary carriers are materials useful for the purpose of administering the composition and may be solid, liquid or gaseous materials which are otherwise inert or acceptable in the veterinary art and are compatible with the active ingredient. These veterinary compositions may be administered parenterally, orally or by any other desired route.

Combination Therapy

The compounds of formula I may be employed alone or in combination with other therapeutic agents for the treatment of a disease or disorder described herein, such as a hyperproliferative disorder (e.g., cancer). In certain embodiments, a compound of formula I is combined in a pharmaceutical combination formulation, or dosing regimen as combination therapy, with a second compound that has anti-hyperproliferative properties or that is useful for treating a hyperproliferative disorder (e.g., cancer). The second compound of the pharmaceutical combination formulation or dosing regimen preferably has complementary activities to the compound of formula I such that they do not adversely affect each other. The combination therapy may provide “synergy” and prove “synergistic”, i.e., the effect achieved when the active ingredients used together is greater than the sum of the effects that results from using the compounds separately.

The combination therapy may be administered as a simultaneous or sequential regimen. When administered sequentially, the combination may be administered in two or more administrations. The combined administration includes co-administration, using separate formulations or a single pharmaceutical formulation, and consecutive administration in either order, wherein preferably there is a time period while both (or all) active agents simultaneously exert their biological activities.

Suitable dosages for any of the above co-administered agents are those presently used and may be lowered due to the combined action (synergy) of the newly identified agent and other chemotherapeutic agents or treatments.

Combination therapies according to the present invention thus comprise the administration of at least one compound of formula I, or a stereoisomer, geometric isomer, tautomer, metabolite, or pharmaceutically acceptable salt and the use of at least one other cancer treatment method. The amounts of the compound(s) of formula I and the other pharmaceutically active chemotherapeutic agent(s) and the relative timings of administration will be selected in order to achieve the desired combined therapeutic effect.

Articles of Manufacture

In another embodiment of the invention, an article of manufacture, or “kit”, containing materials useful for the treatment of the diseases and disorders described above is provided. In one embodiment, the kit comprises a container comprising a compound of formula I, or a stereoisomer, tautomer, or pharmaceutically acceptable salt thereof. The kit may further comprise a label or a package insert on or associated with the container. The term “package insert” is used to refer to instructions customarily included in commercial packages of therapeutic products, that contain information about the indications, usage, dosage, administration, contraindications and/or warnings concerning the use of such therapeutic products. Suitable containers include, for example, bottles, vials, syringes, blister pack, etc. The container may be formed from a variety of materials such as glass or plastic. The container may hold a compound of formula I or a formulation thereof which is effective for treating the condition and may have a sterile access port (for example, the container may be an intravenous solution bag or a vial having a stopper pierceable by a hypodermic injection needle). At least one active agent in the composition is a compound of formula I. Alternatively, or additionally, the article of manufacture may further comprise a second container comprising a pharmaceutically diluent, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

In another embodiment, the kits are suitable for the delivery of solid oral forms of a compound of formula I, such as tablets or capsules. Such a kit can include a number of unit dosages. An example of such a kit is a “blister pack”. Blister packs are well known in the packaging industry and are widely used for packaging pharmaceutical unit dosage forms.

According to one embodiment, a kit may comprise (a) a first container with a compound of formula I contained therein; and optionally (b) a second container with a second pharmaceutical formulation contained therein, wherein the second pharmaceutical formulation comprises a second compound with anti-hyperproliferative activity. Alternatively, or additionally, the kit may further comprise a third container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The following examples illustrate the preparation and biological evaluation of compounds within the scope of the invention. These examples and preparations which follow are provided to enable those skilled in the art to more clearly understand and to practice the present invention. They should not be considered as limiting the scope of the invention, but merely as being illustrative and representative thereof.

General Conditions

Compounds of the invention can be made by a variety of methods depicted in the illustrative synthetic reactions described below in the Examples section.

The starting materials and reagents used in preparing these compounds generally are either available from commercial suppliers, such as Aldrich Chemical Co., or are prepared by methods known to those skilled in the art following procedures set forth in references such as Fieser and Fieser's Reagents for Organic Synthesis; Wiley & Sons: New York, 1991, Volumes 1-15; Rodd's Chemistry of Carbon Compounds, Elsevier Science Publishers, 1989, Volumes 1-5 and Supplementals; and Organic Reactions, Wiley & Sons: New York, 1991, Volumes 1-40. It should be appreciated that the synthetic reaction schemes shown in the Examples section are merely illustrative of some methods by which the compounds of the invention can be synthesized, and various modifications to these synthetic reaction schemes can be made and will be suggested to one skilled in the art having referred to the disclosure contained in this application.

The starting materials and the intermediates of the synthetic reaction schemes can be isolated and purified if desired using conventional techniques, including but not limited to, filtration, distillation, crystallization, chromatography, and the like. Such materials can be characterized using conventional means, including physical constants and spectral data.

Unless specified to the contrary, the reactions described herein are typically conducted under an inert atmosphere at atmospheric pressure at a reaction temperature range of from about −78° C. to about 150° C., often from about 0° C. to about 125° C., and more often and conveniently at about room (or ambient) temperature, e.g., about 20° C.

Preparative reverse-phase high-pressure liquid chromatography (RP HPLC) was performed using one of the following systems: (A). a Waters Delta prep 4000 pump/controller, a 486 detector set at 215 nm, and a LKB Ultrorac fraction collector; or (B). a Sciex LC/MS system with a 150 EX single quad mass spec, a Shimadzu LC system, a LEAP autoinjector, and a Gilson fraction collector. The sample was dissolved in a mixture of acetonitrile/20 mM aqueous ammonium acetate or acetonitrile/water/TFA, applied on a Pursuit C-18 20×100 mm column and eluted at 20 mL/min with a linear gradient of 10%-90% B, where (A): 20 mM aqueous ammonium acetate (pH 7.0) and (B): acetonitrile or (A): water with 0.05% TFA and (B): acetonitrile with 0.05% TFA.

Flash chromatography was performed using standard silica gel chromatography, pre-packed silica columns (Analogix) with an Analogix BSR pump system or AnaLogix IntelliFlash Automated systems. Reactions heated in a microwave were performed using the Biotage Initiator 60 microwave or the CEM Explore microwave

### PREPARATIVE EXAMPLES

### Intermediate A

### 6-Chloro-1-methyl-1,5-dihydro-pyrazolo[3,4-d]pyrimidin-4-one

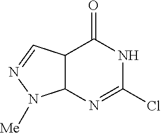 [SMILES: CN1N=CC2C(=O)NC(Cl)=NC21]

A sealed reaction vessel was charged with 4,6-dichloro-1-methyl-1H-pyrazolo[3,4-d]pyrimidine (US 20100015141 A1, 2.2 g, 10.8 mmol) and a 2M aqueous sodium hydroxide solution (50 mL). The vessel was sealed, and the reaction was heated to 70° C. behind a blast shield and stirred for 30 min. The resulting clear solution was transferred with water, brought to pH 6-7 with a 2M aqueous hydrochloric solution and concentrated in vacuo. The remaining solids were filtered and rinsed with ethanol (=300 mL), and the filtrate was concentrated in vacuo onto Celite®. Flash chromatography (40 g silica gel column, 1-10% methanol:methylene chloride) afforded 6-chloro-1-methyl-1,5-dihydro-pyrazolo[3,4-d]pyrimidin-4-one as a white solid (1.45 g, 72.5%). ¹H NMR (300 MHz, DMSO-d₆) δ ppm 3.87 (s, 3H) 8.06 (s, 1H) 13.18 (br. s., 1H). LC-MS calcd. for C₆H₆ClN₄O [(M+H)⁺] 185, obsd. 184.9.

### Intermediate B

### 6-Chloro-1,5-dihydro-pyrazolo[3,4-d]pyrimidin-4-one

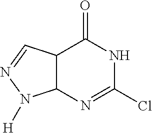 [SMILES: O=C1NC(Cl)=NC2NN=CC12]

A solution of 4,6-dichloro-1H-pyrazolo[3,4-d]pyrimidine (400 mg, 2.12 mmol) and a 2M aqueous sodium hydroxide solution (60 mL) in 1,4-dioxane (5 mL) was heated to reflux for 1.5 h. At this time, the resulting mixture was poured onto iced water, acidified to pH 6.5 with a 6M aqueous hydrochloric acid solution and extracted with ethyl acetate. The combined organic extracts were washed with a saturated aqueous sodium chloride solution, dried over sodium sulfate, filtered and concentrated in vacuo. The resulting residue was triturated with acetonitrile and ether to afford 6-chloro-1,5-dihydro-pyrazolo[3,4-d]pyrimidin-4-one. LC-MS calcd. for C₅H₃ClN₄O [(M+H)⁺] 171, obsd. 170.9.

### Intermediate C

### 6-Chloro-1-methyl-1,5-dihydro-pyrazolo[4,3-c]pyridin-4-one

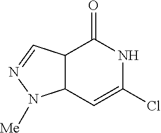 [SMILES: CN1N=CC2C(=O)NC(Cl)=CC21]

A solution of 4,6-dichloro-1H-pyrazolo[4,3-c]pyridine (1.00 g, 5.32 mmol) in tetrahydrofuran (23 mL) cooled to 0° C. under nitrogen was treated with 60% dispersion of sodium hydride in mineral oil (0.40 g, 10 mmol). The reaction mixture was stirred at 0° C. for 10 min. At this time, the reaction was treated with methyl iodide (1.51 g, 10.6 mmol) and stirred for 1 h at 0° C. The ice bath was then removed, and the mixture was stirred at room temperature overnight. At this time, the reaction was quenched with a saturated aqueous ammonium hydroxide solution and then was concentrated in vacuo. The resulting mixture was extracted with ethyl acetate (2×50 mL). The combined organics were washed with a saturated aqueous sodium chloride solution (25 mL), dried over magnesium sulfate, filtered, and concentrated in vacuo. Flash chromatography (40 g silica gel column, 10-50% ethyl acetate/hexanes) afforded 4,6-dichloro-1-methyl-1H-pyrazolo[4,3-c]pyridine (563.9 mg, 52.5%) as an off-white solid. ¹H NMR (400 MHz, DMSO-d₆) □ ppm 4.07 (s, 3H) 8.01 (d, J=1.00 Hz, 1H) 8.38 (d, J=1.00 Hz, 1H) and 4,6-dichloro-2-methyl-2H-pyrazolo[4,3-c]pyridine (356.9 mg, 33.2%) as an off-white solid ¹H NMR (400 MHz, DMSO-d₆) □ ppm 4.22 (d, J=0.50 Hz, 3H) 7.76 (d, J=1.00 Hz, 1H) 8.76-8.99 (m, 1H).

A microwave reaction vial was charged with 4,6-dichloro-1-methyl-1H-pyrazolo[4,3-c]pyridine (80 mg, 0.39 mmol) and a 2M aqueous sodium hydroxide solution (5 mL). The vial was sealed and heated in the microwave at 140° C. for 30 min. At this time, the resulting mixture was acidified to pH 6.5 with a 6M aqueous hydrochloric acid solution and then concentrated in vacuo. The residue was diluted with ethanol. The solids were removed by filtration and the filtrate was concentrated in vacuo. Flash chromatography (15/1 methylene chloride/methanol) afforded 6-chloro-1-methyl-1,5-dihydro-pyrazolo[4,3-c]pyridin-4-one (69 mg, 94.9%). LC-MS calcd. for C₇H₆ClN₃O [(M+H)⁺] 184, obsd. 183.9.

### Intermediate D

### 6-Chloro-1,5-dihydro-pyrazolo[4,3-c]pyridin-4-one

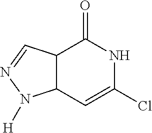 [SMILES: O=C1NC(Cl)=CC2NN=CC12]

A solution of 4,6-dichloro-1H-pyrazolo[4,3-c]pyridine (200 mg, 1.06 mmol) and a 2M aqueous sodium hydroxide solution (30 mL) in 1,4-dioxane (4 mL) was heated to reflux for 3 days. At this time, the resulting mixture was poured onto ice water and then acidified to pH 6.5 with a 6M aqueous hydrochloric acid solution. This solution was concentrated in vacuo. The resulting residue was diluted with ethanol. The insoluble material was removed by filtration. The filtrate was concentrated in vacuo to afford 6-chloro-1,5-dihydro-pyazrolo[4,3-c]pyridin-4-one. LC-MS calcd. for C₆H₄ClN₃O [(M+H)⁺] 170, obsd. 169.9.

### Intermediate E

### 1-(4-Fluoro-3-trifluoromethyl-phenyl)-piperazine

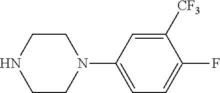 [SMILES: Fc1ccc(N2CCNCC2)cc1C(F)(F)F]

A mixture of piperazine-1-carboxylic acid tert-butyl ester (184 mg, 0.98 mmol), 4-bromo-1-fluoro-2-trifluoromethyl-benzene (200 mg, 0.81 mmol), cesium carbonate (373.6 mg, 1.14 mmol), palladium(II) acetate (9.15 mg, 0.041 mmol) and 2,2′-bis(diphenylphosphino)-1,1′-binaphthyl (38.1 mg, 0.61 mmol) in toluene (2 mL) was heated to 100° C. for 24 h. At this time, the reaction was cooled to room temperature, diluted with ethyl acetate, filtered and concentrated in vacuo. Flash chromatography (20 g column, 0-10% ethyl acetate/hexanes) afforded 4-(4-fluoro-3-trifluoromethyl-phenyl)-piperazine-1-carboxylic acid tert-butyl ester (300 mg, quant.) as a white solid. ¹H NMR (300 MHz, Chloroform-d) □ ppm 1.49 (s, 9H) 2.93-3.24 (m, 4H) 3.49-3.74 (m, 4H) 6.92-7.19 (m, 3H). LC-MS calcd. for C₁₆H₂₀F₄N₂O₂ [(M+H)⁺] 349, obsd. 348.1.

A solution of 4-(4-fluoro-3-trifluoromethyl-phenyl)-piperazine-1-carboxylic acid tert-butyl ester (3.67 g, 10.5 mmol) in methylene chloride (39 mL) was treated with trifluoroacetic acid (4 mL). The resulting yellow solution was stirred at room temperature for 30 min and then was heated to reflux for 5 h. At this time, the reaction was concentrated in vacuo. The resulting residue was dissolved in water (100 mL), treated with a 5N aqueous sodium hydroxide solution until the solution was basic, and then extracted with diethyl ether (2×) and methylene chloride (2×). The combined organics were dried over sodium sulfate, filtered and concentrated in vacuo to afford 1-(4-fluoro-3-trifluoromethyl-phenyl)-piperazine as a yellow solid (2.3 g, 87.9%). ¹H NMR (300 MHz, Chloroform-d) δ ppm 2.98-3.18 (m, 8H) 6.97-7.17 (m, 3H). LC-MS calcd. for C₁₁H₁₃F₄N₂ [(M+H)⁺] 249, obsd. 249.2.

### Intermediate F

### 1-(2,6-Difluoro-4-nitro-phenyl)-piperazine

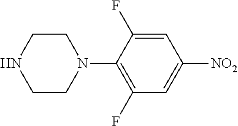 [SMILES: O=[N+]([O-])c1cc(F)c(N2CCNCC2)c(F)c1]

A solution of 1,2,3-trifluoro-5-nitrobenzene (0.34 g, 224 μL, 1.92 mmol) in acetonitrile (3.6 mL) was treated with piperazine (0.41 g, 4.76 mmol). The reaction solution was warmed to 60° C., where it stirred for 30-45 min. The reaction was concentrated in vacuo and partitioned between ethyl acetate (50 mL) and water (25 mL). The organics were washed with a saturated aqueous sodium chloride solution (25 mL), dried over magnesium sulfate, filtered and rinsed with ethyl acetate, and concentrated in vacuo. Flash chromatography (1% methanol/methylene chloride) afforded 1-(2,6-difluoro-4-nitrophenyl)piperazine (381.6 mg, 81.7%) as a yellow solid. ¹H NMR (300 MHz, DMSO-d₆) δ ppm 2.71-2.87 (m, 4H) 3.13-3.28 (m, 4H) 7.78-8.18 (m, 2H). LC-MS calcd. for C₁₀H₁₂F₂N₃O₂ [(M+H)⁺] 244, obsd. 244.0.

### Intermediate G

### 1-(3-fluoro-5-(2-methoxyethoxy)phenyl)piperazine

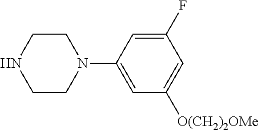

A mixture of 2-bromo-1,3-difluoro-5-(2-methoxyethoxy)benzene (534 mg, 2 mmol), piperazine (482 mg, 5.6 mmol), sodium 2-methylpropan-2-olate (125 mg, 1.3 mmol), 2,2′-bis(diphenylphosphino)-1,1′-binaphthyl (33.6 mg, 54.0 μmol) and tris(dibenzylideneacetone)dipalladium(0) (16.5 mg, 18.0 μmol) in toluene (5 mL) was heated to 130° C. for 4 d. At this time, the reaction mixture was poured into water (20 mL) and extracted with methylene chloride (3×50 mL). The combined organic layers were washed with a saturated aqueous sodium chloride solution, dried over sodium sulfate, filtered and concentrated in vacuo to afford 1-(3-fluoro-5-(2-methoxyethoxy)phenyl)piperazine (145.3 mg, 26.7%) as a brown oil. The material was used without further purification. ¹H NMR (400 MHz, CHLOROFORM-d) □ 7.29 (s, 1H), 6.23-6.30 (m, 2H), 6.16 (td, J=2.10, 10.35 Hz, 1H), 4.04-4.19 (m, 2H), 3.66-3.86 (m, 2H), 3.47 (s, 3H), 3.14 (dd, J=3.76, 6.27 Hz, 4H), 2.96-3.07 (m, 4H). LC-MS calcd. for C₁₃H₂₀FN₂O₂ [(M+H)⁺] 255, obsd. 254.9. Rₜ=3.09 min.

### Intermediate H

### 3,4-difluoro-5-(piperazin-1-yl)phenol

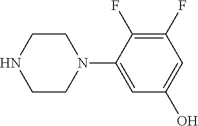 [SMILES: Oc1cc(F)c(F)c(N2CCNCC2)c1]

A mixture of 3,4,5-trifluorophenol (1 g, 6.75 mmol), piperazine (2.33 g, 27 mmol) in N-methyl-2-pyrrolidone (4 mL) was heated to 130° C. The reaction was stirred at 130° C. over the weekend. At this time, the reaction mixture was poured onto water (20 mL) and was extracted with methylene chloride (3×50 mL). The combined organic layers were washed with a saturated aqueous sodium chloride solution, dried over sodium sulfate, filtered and concentrated in vacuo to afford 3,4-difluoro-5-(piperazin-1-yl)phenol (0.76 g, 52.5%) as an off-white powder. ¹H NMR 400 MHz, DMSO-d₆) □ 9.64 (br. s., 1H), 6.29 (ddd, J=2.76, 6.02, 12.05 Hz, 1H), 6.18 (td, J=2.13, 4.27 Hz, 1H), 2.75-2.95 (m, 8H). LC-MS calcd. for C₁₀H₁₃F₂N₂O [(M+H)⁺] 215, obsd. 214.8. Rₜ=2.28 min.

### Intermediate I

### 1-[2,6-Difluoro-4-(2-methoxy-ethoxy)-phenyl]-piperazine

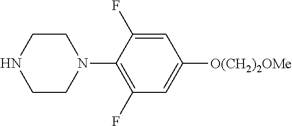

A mixture of 4-bromo-3,5-difluoro-phenol (0.4 g, 1.91 mmol), 1-bromo-2-methoxyethane (0.80 g, 5.74 mmol), and potassium carbonate (1.07 g, 7.66 mmol) in acetone (10 mL) was heated to 60° C. overnight. The reaction mixture was filtered and concentrated in vacuo. Flash chromatography (20% ethyl acetate/hexane) afforded 2-bromo-1,3-difluoro-5-(2-methoxy-ethoxy)-benzene (0.44 g, 86.1%) as a pale yellow oil. ¹H NMR (400 MHz, DMSO-d₆) □ ppm 3.29 (s, 3H) 3.59-3.71 (m, 2H) 4.07-4.19 (m, 2H) 6.89-7.06 (m, 2H)

A solution of 2-bromo-1,3-difluoro-5-(2-methoxy-ethoxy)-benzene (242 mg, 0.91 mmol), piperazine (390 mg, 4.53 mmol), sodium 2-methylpropan-2-olate (131 mg, 1.36 mmol), 2,2′-bis(diphenylphosphino)-1,1′-binaphthyl (28.2 mg, 45.3 and tris(dibenzylideneacetone)dipalladium(0) (10.4 mg, 18.1 μmol) in toluene (3 mL) was heated to 110° C. overnight. The reaction mixture was quenched with water and concentrated in vacuo. Flash chromatography (20/1 methylene chloride/methanol with 1% triethylamine) afforded 1-[2,6-difluoro-4-(2-methoxy-ethoxy)-phenyl]-piperazine (88 mg, 35.7%) as a brown oil. ¹H NMR (376 MHz, Chloroform-d) □ ppm 2.66-2.75 (m, 4H) 2.75-2.84 (m, 4H) 3.17-3.21 (m, 3H) 3.45-3.52 (m, 2H) 3.79-3.86 (m, 2H) 6.40 (d, J=−11.04 Hz, 2H), LC-MS calcd. for C₁₃H₁₉F₂N₂O₂ [(M+H)⁺] 273, obsd. 273.0.

### Intermediate L

### 1-[4-(2,6-difluoro-4-hydroxyphenyl)piperazin-1-yl]ethan-1-one

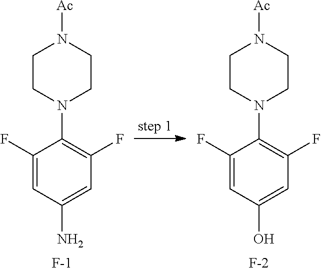

Step 1: To a stirred solution of 1-[4-(4-amino-2,6-difluorophenyl)piperazin-1-yl]ethan-1-one (25.5 g, 99.90 mmol, 1.00 equiv) in 25% sulfuric acid (100 mL) at 0° C. was added dropwise a solution of NaNO₂ (7.7 g, 111.59 mmol, 1.12 equiv). The reaction mixture was stirred at 0° C. for 1 h. The above solution was added to a suspension of CuSO₄ (16 g, 100.00 mmol, 1.00 equiv) and Cu₂O (15 g, 104.83 mmol, 1.05 equiv) in 100 mL of water at 0° C. and the resulting solution was stirred overnight at RT. The solution was adjusted pH 9 with solid sodium carbonate and then extracted with EtOAc (3×100 mL). The combined organic extracts were washed with water (3×100 mL) and brine (2×100 mL), dried (Na₂SO₄) over anhydrous sodium sulfate, filtered and concentrated under vacuum. The residue was purified by SiO₂ chromatography eluting with 5% MeOH in DCM to afford 1 g (4%) of 1-[4-(2,6-difluoro-4-hydroxyphenyl)piperazin-1-yl]ethan-1-one as an off-white solid. LCMS (LCMS45, ESI): RT=0.76 min, m/z=256.0 [M+H]⁺.

### Intermediate M

### tert-Butyl 4-(2,6-Difluoro-4-formyl-phenyl)-piperazine-1-carboxylate

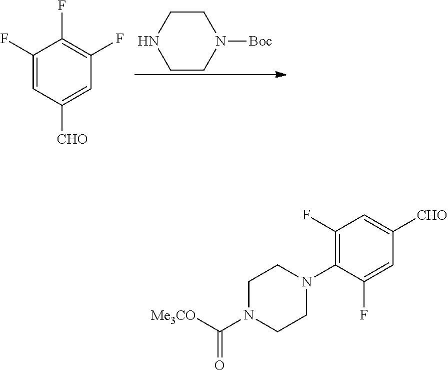

A mixture of 3,4,5-trifluorobenzaldehyde (1.6 g, 9.99 mmol, 1.00 equiv), tert-butyl piperazine-1-carboxylate (1.86 g, 9.99 mmol, 1.00 equiv) and K₂CO₃ (2.76 g, 19.97 mmol, 2.00 equiv). in DMSO (30 mL) was stirred at 120° C. for 10 h. The resulting mixture was cooled to RT and concentrated under vacuum. The residue was purified by SiO₂ chromatography eluting with 10% EtOAc/petroleum ether to afford 1.2 g (37%) of tert-butyl 4-(2,6-difluoro-4-formylphenyl)piperazine-1-carboxylate as an off-white solid. TLC: R_{f}=0.5, petroleum ether/ethyl acetate=2:1.

### Intermediate M

### 1-[4-(4-Bromo-2,6-difluoro-phenyl)-piperazin-1-yl]-ethanone

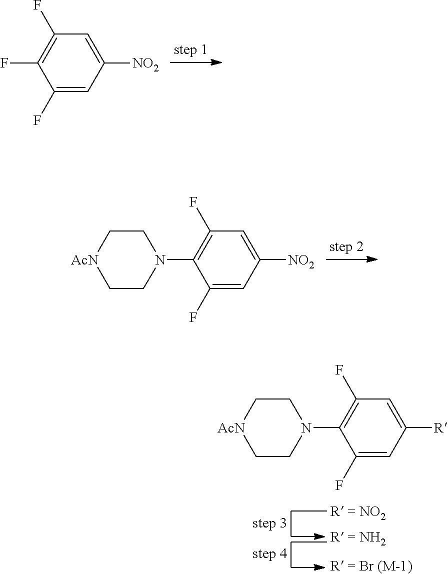

Reaction of acetyl-piperazine and 3,4,5-trifluoronitrobenzene (MeCN) afforded 1-[4-(2,6-Difluoro-4-nitro-phenyl)-piperazin-1-yl]-ethanone which is reduced to the corresponding amine (Zn, NH₄Cl, MeOH, H₂O, Subjecting the amine to a Sandmeyer reaction (NaNO₂, HBr, CuBr,MeOH, H₂O) afforded the title compound.

### Intermediate N

### 1-[4-(4-hydroxy-2,6-difluoro-phenyl)-piperazin-1-yl]-ethanone

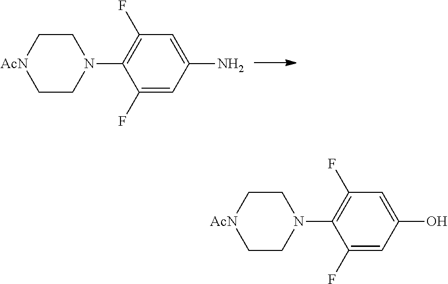

Reaction of tert-butyl 4-(4-amino-2,6-difluoro-phenyl)-piperazine-1-carboxylate from Example M under Sandermeyer conditions (H₂SO₄, NaNO₂, CuSO₄, Cu₂O.5H₂O) affords tert-butyl 4-(4-hydroxy-2,6-difluoro-phenyl)-piperazine-1-carboxylate

### Example 1

### 1-Methyl-6-(4-thiazol-2-yl-piperazin-1-yl)-1,5-dihydro-pyrazolo[3,4-d]pyrimidin-4-one (I-38)

A sealed tube apparatus was charged with 6-chloro-1-methyl-1H-pyrazolo[3,4-d]pyrimidin-4(5H)-one (30 mg, 163 μmol, Eq: 1.0) and ethanol (460 μL) and was treated with 1-thiazol-2-yl-piperazine (60.5 mg, 358 μmol, Eq: 2.2) and DIPEA (67.2 mg, 90.6 μL, 520 μmol, Eq: 3.2). The tube was sealed and heated to 100° C., where it stirred overnight. The reaction was allowed to cool to room temperature, diluted with methylene chloride and methanol, and concentrated in vacuo onto Celite. The crude material was purified by flash chromatography (AnaLogix IntelliFlash 280, 12 g silica gel column, 1-3 methylene chloride/methanol) to yield I-methyl-6-(4-thiazol-2-yl-piperazin-1-yl)-1,5-dihydro-pyrazolo[3,4-d]pyrimidin-4-one as a white solid (46.4 mg, 90.0%). ¹H NMR (400 MHz, DMSO-d₆) δ ppm 3.44-3.56 (m, 4H) 3.74 (s, 3H) 3.76-3.85 (m, 4H) 6.90 (d, J=3.76 Hz, 1H) 7.20 (d, J=3.76 Hz, 1H) 7.79 (s, 1H) 11.05 (s, 1H). LC-MS calcd. for C₁₃H₁₆N₇OS [(1\441)⁺] 318, obsd. 317.8.

In an analogous manner the following compounds were synthesized:

### Example 2

### 6-[4-(4-Fluoro-3-trifluoromethyl-phenyl)-piperazin-1-yl]-1-methyl-1,5-dihydro-pyrazolo[3,4-d]pyrimidin-4-one (I-39)

From 6-chloro-1-methyl-1,5-dihydro-pyrazolo[3,4-d]pyrimidin-4-one (Intermediate A) and 1-(4-fluoro-3-trifluoromethyl-phenyl)piperazine (Intermediate E): 6-[4-(4-fluoro-3-trifluoromethyl-phenyl)-piperazin-1-yl]-1-methyl-1,5-dihydro-pyrazolo[3,4-d]pyrimidin-4-one was obtained a white solid (59.2 mg, 91.9%). ¹H NMR (400 MHz, DMSO-d₆) δ ppm 3.22-3.30 (m, 4H) 3.74 (s, 3H) 3.76-3.86 (m, 4H) 7.24 (dd, J=5.90, 2.89 Hz, 1H) 7.27-7.42 (m, 2H) 7.79 (s, 1H) 11.03 (s, 1H). LC-MS calcd. for C₁₇H₁₅F₄N₆O [(M−H)⁻] 395, obsd. 394.9.

### Example 3

### 4-[4-(1-Methyl-4-oxo-4,5-dihydro-1H-pyrazolo[3,4-d]pyrimidin-6-yl)-piperazin-1-yl]-benzoic acid ethyl ester (I-41)

From 6-chloro-1-methyl-1,5-dihydro-pyrazolo[3,4-d]pyrimidin-4-one (Intermediate A) and 4-(piperazin-1-yl)-benzoic acid ethyl ester: 4-[4-(1-methyl-4-oxo-4,5-dihydro-1H-pyrazolo[3,4-d]pyrimidin-6-yl)-piperazin-1-yl]-benzoic acid ethyl ester was obtained a white solid (66.8 mg, 89.8%). ¹H NMR (400 MHz, DMSO-d₆) δ ppm 1.29 (t, J=7.03 Hz, 3H) 3.40-3.50 (m, 4H) 3.74 (s, 3H) 3.77-3.85 (m, 4H) 4.24 (q, J=7.19 Hz, 2H) 7.03 (d, J=9.03 Hz, 2H) 7.69-7.96 (m, 3H) 11.03 (s, 1H). LC-MS calcd. for C₁₉H₂₃N₆O₃ [(M+H)⁺] 383, obsd. 383.0.

### Example 4

### 6-[4-(2,4-Difluoro-phenyl)-piperazin-1-yl]-1-methyl-1,5-dihydro-pyrazolo[3,4-d]pyrimidin-4-one (I-16)

A microwave reaction vial was charged with 6-chloro-1-methyl-1,5-dihydro-pyrazolo[3,4-d]pyrimidin-4-one (Intermediate A) (37 mg, 0.2 mmol), difluorophenyl)piperazine (79.5 mg, 0.41 mmol), and DIPEA (77.7 mg, 0.60 mmol) in ethanol (2 mL). The vial was sealed and heated in the microwave at 140° C. for 20 min. At this time, the resulting mixture was concentrated in vacuo. Flash chromatography (20/1 methylene chloride/methanol) afforded 6-[4-(2,4-difluoro-phenyl)-piperazin-1-yl]-1-methyl-1,5-dihydro-pyrazolo[3,4-d]pyrimidin-4-one (64 mg, 92.2%). ¹H NMR (400 MHz, DMSO-d₆) δ ppm 2.94-3.10 (m, 4H) 3.74 (s, 3H) 3.76-3.88 (m, 4H) 7.01 (td, J=8.16, 2.76 Hz, 1H) 7.12 (td, J=9.35, 6.15 Hz, 1H) 7.23 (ddd, J=12.30, 9.16, 2.89 Hz, 1H) 7.79 (s, 1H) 11.01 (s, 1H). LC-MS calcd. for C₁₆H₁₇F₂N₆O [(M+H)⁺] 347, obsd. 347.0.

In an analogous manner the following compounds were synthesized following the above procedure:

### Example 5

### 6-[4-(2-Fluoro-phenyl)-piperazin-1-yl]-1-methyl-1,5-dihydro-pyrazolo[3,4-d]pyrimidin-4-one (I-3)

From 6-chloro-1-methyl-1,5-dihydro-pyrazolo[3,4-d]pyrimidin-4-one (Intermediate A) and 1-(2-fluorophenyl)piperazine: 6-[4-(2-fluoro-phenyl)-piperazin-1-yl]-1-methyl-1,5-dihydro-pyrazolo[3,4-d]pyrimidin-4-one was obtained as a white solid (45 mg, 93.7%). ¹HNMR (400 MHz, DMSO-d₆) δ ppm 11.0 (s, 1H), 7.80 (s, 1H), 7.22-6.99 (m, 4H), 3.89-3.80 (m, 4H), 3.78 (s, 3H), 3.15 3.05 (m, 4H). LC-MS calcd. for C₁₆H₁₈FN₆O [(M+H)⁺] 329, obsd. 329.0.

### Example 6

### 2-[4-(1-Methyl-4-oxo-4,5-dihydro-1H-pyrazolo[3,4-d]pyrimidin-6-yl)-piperazin-1-yl]-benzonitrile (I-15)

From 6-chloro-1-methyl-1,5-dihydro-pyrazolo[3,4-d]pyrimidin-4-one (Intermediate A) and 2-(piperazin-1-yl)benzonitrile: 2-[4-(1-methyl-4-oxo-4,5-dihydro-1H-pyrazolo[3,4-d]pyrimidin-6-yl)-piperazin-1-yl]-benzonitrile was obtained as a white solid (33 mg, 90.8%). ¹H NMR (400 MHz, DMSO-d₆) □ ppm 3.16-3.28 (m, 4H) 3.74 (s, 3H) 3.79-3.88 (m, 4H) 7.13 (td, J=7.59, 0.88 Hz, 1H) 7.21 (d, J=8.03 Hz, 1H) 7.62 (ddd, J=8.53, 7.28, 1.76 Hz, 1H) 7.74 (dd, J=7.78, 1.51 Hz, 1H) 7.78 (s, 1H) 11.02 (br. s., 1H). LC-MS calcd. for C₁₇H₁₈N₇O [(M+H)⁺] 336, obsd. 336.0.

### Example 7

### 3-Fluoro-4-[4-(1-methyl-4-oxo-4,5-dihydro-1H-pyrazolo[3,4-d]pyrimidin-6-yl)-piperazin-1-yl]-benzonitrile (I-28)

From 6-chloro-1-methyl-1,5-dihydro-pyrazolo[3,4-d]pyrimidin-4-one (Intermediate A) and 3-fluoro-4-(piperazin-1-yl)benzonitrile: 3-fluoro-4-[4-(1-methyl-4-oxo-4,5-dihydro-1H-pyrazolo[3,4-d]pyrimidin-6-yl)-piperazin-1-yl]-benzonitrile was obtained as a white solid (24 mg, 17.9%). ¹H NMR (400 MHz, DMSO-d₆) □ ppm 3.22-3.31 (m, 4H) 3.74 (s, 3H) 3.76-3.87 (m, 4H) 7.18 (t, J=8.78 Hz, 1H) 7.60 (dd, J=8.53, 2.01 Hz, 1H) 7.75 (dd, J=13.30, 1.76 Hz, 1H) 7.79 (s, 1H) 11.03 (s, 1H). LC-MS calcd. for C₁₇H₂₇FN₇O [(M+H)⁺] 354, obsd. 354.0.

### Example 8

### 6-{4-[2-Fluoro-4-(2-methoxy-ethoxy)-phenyl]-piperazin-1-yl}-1-methyl-1,5-dihydro-pyrazolo[3,4-d]pyrimidin-4-one (I-30)

From 6-chloro-1-methyl-1,5-dihydro-pyrazolo[3,4-d]pyrimidin-4-one (Intermediate A) and 1-(2-fluoro-4-(2-methoxyethoxy)phenyl)piperazine (Intermediate G): 6-{4-[2-fluoro-4-(2-methoxy-ethoxy)-phenyl]-piperazin-1-yl}-1-methyl-1,5-dihydro-pyrazolo[3,4-d]pyrimidin-4-one was obtained as a white solid (80 mg, 91.7%). ¹H NMR (400 MHz, DMSO-d₆) □ ppm 2.90-3.07 (m, 4H) 3.30 (s, 3H) 3.58-3.67 (m, 2H) 3.74 (s, 3H) 3.76-3.85 (m, 4H) 4.01-4.10 (m, 2H) 6.73 (dt, J=8.91, 1.44 Hz, 1H) 6.86 (dd, J=13.93, 2.89 Hz, 1H) 7.02 (dd, J=10.04, 9.03 Hz, 1H) 7.79 (s, 1H) 10.99 (s, 1H). LC-MS calcd. for C₁₉H₂₄FN₆O₃ [(M+H)⁺] 403, obsd. 403.1.

### Example 9

### 6-[4-(3-Methoxy-pyridin-2-yl)-piperazin-1-yl]-1-methyl-1,5-dihydro-pyrazolo[3,4-d]pyrimidin-4-one (I-43)

From 6-chloro-1-methyl-1,5-dihydro-pyrazolo[3,4-d]pyrimidin-4-one (Intermediate A) and 1-(3-methoxypyridin-2-yl)piperazine dihydrochloride: 6-[4-(3-methoxy-pyridin-2-yl)-piperazin-1-yl]-1-methyl-1,5-dihydro-pyrazolo[3,4-d]pyrimidin-4-one was obtained as a white solid (64 mg, 98.9%). ¹HNMR (400 MHz, DMSO-d₆) □ ppm 3.34-3.40 (m, 4H) 3.73 (s, 3H) 3.74-3.79 (m, 4H) 3.82 (s, 3H) 6.93 (dd, J=7.91, 4.89 Hz, 1H) 7.28 (dd, J=8.03, 1.25 Hz, 1H) 7.78 (s, 1H) 7.80 (dd, J=4.77, 1.51 Hz, 1H) 10.96 (s, 1H). LC-MS calcd. for C₁₆H₂₀N₇O₂ [(M+H)⁺] 342, obsd. 342.1.

### Example 10

### 6-{4-[2,6-Difluoro-4-(2-methoxy-ethoxy)-phenyl]-piperazin-1-yl}-1-methyl-1,5-dihydro-pyrazolo[3,4-d]pyrimidin-4-one (I-1)

From 6-chloro-1-methyl-1,5-dihydro-pyrazolo[3,4-d]pyrimidin-4-one (Intermediate A) and 1-(2,6-difluoro-4-(2-methoxyethoxy)phenyl)piperazine (Intermediate I): 6-{4-[2,6-difluoro-4-(2-methoxy-ethoxy)-phenyl]-piperazin-1-yl}-1-methyl-1,5-dihydro-pyrazolo[3,4-d]pyrimidin-4-one was obtained as a white powder (610.8 mg, 96.9%). ¹H NMR (400 MHz, DMSO-d₆) □□ 10.97 (s, 1H), 7.67-7.94 (m, 1H), 6.56-6.83 (m, 2H), 4.01-4.15 (m, 2H), 3.71-3.82 (m, 7H), 3.60-3.67 (m, 2H), 3.30 (s, 3H), 3.05-3.12 (m, 4H). LC-MS calcd. for C₁₉H₂₂F₂N₆O₃ [(M+H)⁺] 421, obsd. 421.1.

### Example 11

### 3-Fluoro-4-[4-(1-methyl-4-oxo-4,5-dihydro-1H-pyrazolo[3,4-d]pyrimidin-6-yl)-piperazin-1-yl]-benzenesulfonamide (I-48)

From 6-chloro-1-methyl-1,5-dihydro-pyrazolo[3,4-d]pyrimidin-4-one (Intermediate A) and 3-fluoro-4-(piperazin-1-yl)benzenesulfonamide: 3-fluoro-4-[4-(1-methyl-4-oxo-4,5-dihydro-1H-pyrazolo[3,4-d]pyrimidin-6-yl)-piperazin-1-yl]-benzenesulfonamide was obtained as a white solid (55 mg, 49.8%). ¹H NMR (400 MHz, DMSO-d₆) δ ppm 3.17-3.25 (m, 4H) 3.74 (s, 3H) 3.77-3.89 (m, 4H) 7.21 (t, J=8.66 Hz, 1H) 7.33 (s, 2H) 7.48-7.61 (m, 2H) 7.79 (s, 1H) 11.02 (s, 1H). LC-MS calcd. for C₁₆H₁₈FN₇O₃S [(M)⁺] 407, obsd. 407.9.

### Example 12

### 6-[4-(4-Fluoro-phenyl)-piperidin-1-yl]-1-methyl-1,5-dihydro-pyrazolo[3,4-d]pyrimidin-4-one (I-5)

From 6-chloro-1-methyl-1,5-dihydro-pyrazolo[3,4-d]pyrimidin-4-one (Intermediate A) and 4-(4-fluorophenyl)piperidine hydrochloride: 6-[4-(4-fluoro-phenyl)-piperidin-1-yl]-1-methyl-1,5-dihydro-pyrazolo[3,4-d]pyrimidin-4-one was obtained as a white solid (40 mg, 90.2%). ¹H NMR (400 MHz, DMSO-d₆) δ ppm 1.5-1.7 (m, 2H) 1.8 (d, J=11.29 Hz, 2H) 2.8 (t, J=12.05 Hz, 1H) 3.0 (t, J=12.05 Hz, 2H) 3.7 (s, 3H) 4.5 (d, J=13.30 Hz, 2H) 7.1 (t, J=8.91 Hz, 2H) 7.3 (dd, J=8.53, 5.77 Hz, 2H) 7.8 (s, 1H) 10.9 (br. s., 1H). LC-MS calcd. for C₁₇H₁₉FN₅O [(M+H)⁺] 328, obsd. 328.1.

### Example 13

### 1-Methyl-6-[4-(4-trifluoromethyl-phenyl)-piperidin-1-yl]-1,5-dihydro-pyrazolo[3,4-d]pyrimidin-4-one (I-6)

From 6-chloro-1-methyl-1,5-dihydro-pyrazolo[3,4-d]pyrimidin-4-one (Intermediate A) and 4-(4-(trifluoromethyl)phenyl)piperidine hydrochloride: 1-methyl-6-[4-(4-trifluoromethyl-phenyl)-piperidin-1-yl]-1,5-dihydro-pyrazolo[3,4-d]pyrimidin-4-one was obtained as a white solid (9.0 mg, 48.9%). LC-MS calcd. for C₁₈H₁₉F₃N₅O [(M+H)⁺] 378, obsd. 378.1.

### Example 14

### 4-(4-Fluoro-phenyl)-1-(1-methyl-4-oxo-4,5-dihydro-1H-pyrazolo[3,4-d]pyrimidin-6-yl)-piperidine-4-carbonitrile (I-33)

From 6-chloro-1-methyl-1,5-dihydro-pyrazolo[3,4-d]pyrimidin-4-one (Intermediate A) and 4-(4-fluorophenyl)piperidine-4-carbonitrile hydrochloride: 4-(4-fluoro-phenyl)-1-(1-methyl-4-oxo-4,5-dihydro-1H-pyrazolo[3,4-d]pyrimidin-6-yl)-piperidine-4-carbonitrile was obtained as a white solid (76.4 mg, 69.4%). ¹H NMR (400 MHz, DMSO-d₆) δ ppm 2.08 (td, J=12.92, 3.76 Hz, 2H) 2.24 (d, J=13.55 Hz, 2H) 3.22 (t, J=12.17 Hz, 2H) 3.74 (s, 3H) 4.61 (d, J=14.31 Hz, 2H) 7.16-7.39 (m, 2H) 7.53-7.68 (m, 2H) 7.80 (s, 1H) 11.04 (s, 1H). LC-MS calcd. for C₁₈H₁₈FN₆O [(M+H)⁺] 353, obsd. 353.0.

### Example 15

### 1′-(1-Methyl-4-oxo-4,5-dihydro-1H-pyrazolo[3,3-d]pyrimidin-6-yl)-2′,3′,5′,6′-tetrahydro-1′H-[2,4′]bipyridinyl-4′-carbonitrile (I-52)

From 6-chloro-1-methyl-1,5-dihydro-pyrazolo[3,4-d]pyrimidin-4-one (Intermediate A) and 4-(pyridin-2-yl)piperidine-4-carbonitrile dihydrochloride: 1′-(1-methyl-4-oxo-4,5-dihydro-1H-pyrazolo[3,3-d]pyrimidin-6-yl)-2′,3′,5′,6′-tetrahydro-1′H-[2,4′]bipyridinyl-4′-carbonitrile was obtained as a white solid (38 mg, 65.4%). ¹H NMR (400 MHz, DMSO-d₆) δ ppm 2.07-2.33 (m, 4H) 3.20-3.31 (m, 2H) 3.74 (s, 3H) 4.56 (d, J=14.56 Hz, 2H) 7.42 (ddd, J=7.53, 4.77, 1.00 Hz, 1H) 7.66 (dt, J=8.03, 1.00 Hz, 1H) 7.79 (s, 1H) 7.91 (td, J=7.78, 1.76 Hz, 1H) 8.62 (ddd, J=4.77, 1.76, 1.00 Hz, 1H) 11.07 (s, 1H). LC-MS calcd. for C₁₇H₁₈N₇O [(M+H)⁺] 336, obsd. 336.1.

### Example 16

### 1-Methyl-6-[4-(tetrahydro-furan-2-ylmethyl)-piperazin-1-yl]-1,5-dihydro-pyrazolo[3,4-d]pyrimidin-4-one (I-32)

From 6-chloro-1-methyl-1,5-dihydro-pyrazolo[3,4-d]pyrimidin-4-one (Intermediate A) and 1-((tetrahydrofuran-2-yl)methyl)piperazine: 1-methyl-6-[4-(tetrahydro-furan-2-ylmethyl)-piperazin-1-yl]-1,5-dihydro-pyrazolo[3,4-d]pyrimidin-4-one was obtained as a white solid (48 mg, 92.1%). ¹H NMR (300 MHz, DMSO-d₆) δ ppm 1.49 (dd, J=19.97, 7.35 Hz, 1H) 1.64-2.08 (m, 2H) 2.19-2.69 (m, 11H) 3.51-3.67 (m, 3H) 3.68-3.81 (m, 2H) 3.95 (d, J=6.59 Hz, 1H) 7.77 (s, 1H) 10.85 (s, 1H). LC-MS calcd. for C₁₅H₂₃N₆O₂ [(M+H)⁺] 319, obsd. 319.1.

### Example 17

### 6-[4-(3,5-Dichloro-pyridin-4-yl)-piperazin-1-yl]-1-methyl-1,5-dihydro-pyrazolo[3,4-d]pyrimidin-4-one (I-34)

From 6-chloro-1-methyl-1,5-dihydro-pyrazolo[3,4-d]pyrimidin-4-one (Intermediate A) and 1-(3,5-dichloropyridin-4-yl)piperazine: 6-[4-(3,5-dichloro-pyridin-4-yl)-piperazin-1-yl]-1-methyl-1,5-dihydro-pyrazolo[3,4-d]pyrimidin-4-one was obtained as a white solid (57.6 mg, 92.6%). ¹H NMR (300 MHz, DMSO-d₆) δ ppm 3.38 (d, J=4.33 Hz, 4H) 3.75 (s, 3H) 3.81 (br. s., 4H) 7.80 (s, 1H) 8.49 (s, 2H) 11.01 (d, J=12.62 Hz, 1H). LC-MS calcd. for C₁₅H₁₆Cl₂N₇O [(M+H)⁺] 380, obsd. 380.0.

### Example 18

### 3-Fluoro-4-[4-(1-methyl-4-oxo-4,5-dihydro-1H-pyrazolo[3,4-d]pyrimidin-6-yl)-piperazin-1-yl]-benzoic acid (I-40)

From 6-chloro-1-methyl-1,5-dihydro-pyrazolo[3,4-d]pyrimidin-4-one (Intermediate A) and 3-fluoro-4-(piperazin-1-yl)benzoic acid: 3-fluoro-4-[4-(1-methyl-4-oxo-4,5-dihydro-1H-pyrazolo[3,4-d]pyrimidin-6-yl)-piperazin-1-yl]-benzoic acid was obtained as a white solid (200 mg, 99.1%). ¹H NMR (300 MHz, DMSO-d₆) δ ppm 3.25 (br. s., 4H) 3.75 (s, 3H) 3.83 (br. s., 4H) 7.16 (d, J=8.85 Hz, 1H) 7.53-7.76 (m, 2H) 7.80 (s, 1H). LC-MS calcd. for C₁₇H₁₈FN₆O₃ [(M+H)⁺] 373, obsd. 373.1.

### Example 19

### 6-[4-(2,6-Dichloro-phenyl)-piperazin-1-yl]-1-methyl-1,5-dihydro-pyrazolo[3,4-d]pyrimidin-4-one (I-42)

From 6-chloro-1-methyl-1,5-dihydro-pyrazolo[3,4-d]pyrimidin-4-one (Intermediate A) and 1-(2,6-dichlorophenyl)piperazine hydrochloride: 6-[4-(2,6-dichloro-phenyl)-piperazin-1-yl]-1-methyl-1,5-dihydro-pyrazolo[3,4-d]pyrimidin-4-one was obtained as a white solid (94.2 mg, 92.3%). ¹H NMR (300 MHz, DMSO-d₆) δ ppm 3.16-3.26 (m, 4H) 3.73 (s, 3H) 3.79 (br. s., 4H) 7.11-7.29 (m, 1H) 7.45 (d, J=7.91 Hz, 2H) 7.78 (s, 1H) 10.88-11.08 (m, 1H).

### Example 20

### 6-[4-(2,6-Difluoro-4-propionyl-phenyl)-piperazin-1-yl]-1-methyl-1,5-dihydro-pyrazolo[3,4-d]pyrimidin-4-one (I-45)

From 6-chloro-1-methyl-1,5-dihydro-pyrazolo[3,4-d]pyrimidin-4-one (Intermediate A) and 1-(3,5-difluoro-4-(piperazin-1-yl)phenyl)propan-1-one: 6-[4-(2,6-difluoro-4-propionyl-phenyl)-piperazin-1-yl]-1-methyl-1,5-dihydro-pyrazolo[3,4-d]pyrimidin-4-one was obtained as a white solid (36.2 mg, 93.3%). ¹H NMR (300 MHz, DMSO-d₆) δ ppm 1.06 (t, J=7.16 Hz, 3H) 2.99 (q, J=6.97 Hz, 2H) 3.31 (s, 4H) 3.66-3.89 (m, 7H) 7.62 (d, J=10.74 Hz, 2H) 7.79 (s, 1H) 10.99 (s, 1H). LC-MS calcd. for C₁₉H₂₁F₂N₆O₂ [(M+H)⁺] 403, obsd. 403.0.

### Example 21

### 6-[4-(2,3-Dichloro-pyridin-4-yl)-piperazin-1-yl]-1-methyl-1,5-dihydro-pyrazolo[3,4-d]pyrimidin-4-one (I-46)

From 6-chloro-1-methyl-1,5-dihydro-pyrazolo[3,4-d]pyrimidin-4-one (Intermediate A) and 1-(2,3-dichloropyridin-4-yl)piperazine hydrochloride: 6-[4-(2,3-dichloro-pyridin-4-yl)-piperazin-1-yl]-1-methyl-1,5-dihydro-pyrazolo[3,4-d]pyrimidin-4-one was obtained as a white solid (74.8 mg, 72.2%). ¹H NMR (300 MHz, DMSO-d₆) δ ppm 3.29 (br. s., 4H) 3.74 (s, 3H) 3.82 (br. s., 4H) 7.16 (d, J=5.65 Hz, 1H) 7.80 (s, 1H) 8.19 (d, J=5.46 Hz, 1H) 11.04 (s, 1H). LC-MS calcd. for C₁₅H₁₆Cl₂N₇O [(M+H)⁺] 380, obsd. 380.0.

### Example 22

### 2-[4-(1-Methyl-4-oxo-4,5-dihydro-1H-pyrazolo[3,4-d]pyrimidin-6-yl)-piperazin-1-yl]-nicotinonitrile (I-18)

From 6-chloro-1-methyl-1,5-dihydro-pyrazolo[3,4-d]pyrimidin-4-one (Intermediate A) and 2-(piperazin-1-yl)nicotinonitrile: 2-[4-(1-methyl-4-oxo-4,5-dihydro-1H-pyrazolo[3,4-d]pyrimidin-6-yl)-piperazin-1-yl]-nicotinonitrile was obtained as a white solid (70.8 mg, 75.9%). ¹H NMR (400 MHz, DMSO-d₆) δ ppm 3.68-3.77 (m, 7H) 3.78-3.85 (m, 4H) 6.83-7.10 (m, 1H) 7.79 (s, 1H) 8.11 (dd, J=7.65, 1.88 Hz, 1H) 8.44 (dd, J=4.77, 1.76 Hz, 1H) 10.97 (s, 1H). LC-MS calcd. for C₁₆H₁₇N₈O [(M+H)⁺] 337, obsd. 337.0.

### Example 23

### 1-Methyl-6-[4-(3-trifluoromethyl-pyridin-2-yl)-piperazin-1-yl]-1,5-dihydro-pyrazolo[3,4-d]pyrimidin-4-one (I-20)

From 6-chloro-1-methyl-1,5-dihydro-pyrazolo[3,4-d]pyrimidin-4-one (Intermediate A) and 1-(3-(trifluoromethyl)pyridin-2-yl)piperazine: 1-methyl-6-[4-(3-trifluoromethyl-pyridin-2-yl)-piperazin-1-yl]-1,5-dihydro-pyrazolo[3,4-d]pyrimidin-4-one was obtained as a white solid (80.8 mg, 73.8%). ¹H NMR (400 MHz, DMSO-d₆) δ ppm 3.23-3.30 (m, 4H) 3.74 (s, 3H) 3.75-3.82 (m, 4H) 7.24 (dd, J=7.53, 5.02 Hz, 1H) 7.79 (s, 1H) 8.11 (dd, J=8.03, 1.76 Hz, 1H) 8.55 (dd, J=4.89, 1.38 Hz, 1H) 10.99 (s, 1H). LC-MS calcd. for C₁₆H₁₇F₃N₇O [(M+H)⁺] 380, obsd. 380.0.

### Example 24

### 6-[4-(2-Fluoro-4-methanesulfonyl-phenyl)-piperazin-1-yl]-1-methyl-1,5-dihydro-pyrazolo[3,4-d]pyrimidin-4-one (I-21)

From 6-chloro-1-methyl-1,5-dihydro-pyrazolo[3,4-d]pyrimidin-4-one (Intermediate A) and 1-(2-fluoro-4-(methylsulfonyl)phenyl)piperazine: 6-[4-(2-fluoro-4-methanesulfonyl-phenyl)-piperazin-1-yl]-1-methyl-1,5-dihydro-pyrazolo[3,4-d]pyrimidin-4-one was obtained as a white solid (99.1 mg, 88.1%). ¹H NMR (400 MHz, DMSO-d₆) δ ppm 3.20 (s, 3H) 3.24-3.31 (m, 4H) 3.74 (s, 3H) 3.79-3.86 (m, 4H) 7.26 (t, J=8.66 Hz, 1H) 7.59-7.74 (m, 2H) 7.79 (s, 1H) 11.03 (s, 1H). LC-MS calcd. for C₁₇H₂₀FN₆O₃S [(M+H)⁺] 407, obsd. 407.0.

### Example 25

### 6-[4-(4-Fluoro-2-methanesulfonyl-phenyl)-piperazin-1-yl]-1-methyl-1,5-dihydro-pyrazolo[3,4-d]pyrimidin-4-one (I-22)

From 6-chloro-1-methyl-1,5-dihydro-pyrazolo[3,4-d]pyrimidin-4-one (Intermediate A) and 1-(4-fluoro-2-(methylsulfonyl)phenyl)piperazine: 6-[4-(4-fluoro-2-methanesulfonyl-phenyl)-piperazin-1-yl]-1-methyl-1,5-dihydro-pyrazolo[3,4-d]pyrimidin-4-one was obtained as a white solid (80.8 mg, 73.8%). ¹H NMR (400 MHz, DMSO-d₆) δ ppm 3.02 (br. s., 4H) 3.40-3.50 (m, 3H) 3.55-4.10 (m, 7H) 7.55-7.68 (m, 2H) 7.72 (dd, J=8.91, 4.89 Hz, 1H) 7.79 (s, 1H) 10.98 (s, 1H). LC-MS calcd. for C₁₇H₂₀FN₆O₃S [(M+H)⁺] 407, obsd. 407.0.

### Example 26

### 6-(4′-Hydroxy-3′,4′,5′,6′-tetrahydro-2′H-[2,4′]bipyridinyl-1′-yl)-1-methyl-1,5-dihydro-pyrazolo[3,4-d]pyrimidin-4-one (I-35)

From 6-chloro-1-methyl-1,5-dihydro-pyrazolo[3,4-d]pyrimidin-4-one (Intermediate A) and 4-(pyridin-2-yl)piperidin-4-ol: 6-(4′-hydroxy-3′,4′,5′,6′-tetrahydro-2′H-[2,4]bipyridinyl-1′-yl)-1-methyl-1,5-dihydro-pyrazolo[3,4-d]pyrimidin-4-one was obtained as a white solid (43.5 mg, 82.0%). ¹H NMR (300 MHz, DMSO-d₆) δ ppm 1.58 (d, J=14.69 Hz, 2H) 2.01-2.28 (m, 2H) 3.26-3.47 (m, 2H) 3.71 (s, 3H) 4.33 (d, J=11.68 Hz, 2H) 5.42 (s, 1H) 7.24 (ddd, J=7.44, 4.80, 1.32 Hz, 1H) 7.64-7.87 (m, 3H) 8.47 (d, J=3.96 Hz, 1H) 10.88 (s, 1H). LC-MS calcd. for C₁₆H₁₉N₆O₂ [(M+H)⁺] 327, obsd. 326.9.

### Example 27

### 6-[4-(1,1-Dioxo-tetrahydro-1λ*6*-thiophen-3-yl)-piperazin-1-yl]-1-methyl-1,5-dihydro-pyrazolo[3,4-d]pyrimidin-4-one (I-36)

From 6-chloro-1-methyl-1,5-dihydro-pyrazolo[3,4-d]pyrimidin-4-one (Intermediate A) and 1-(1,1-dioxo-tetrahydro-1λ*6*-thiophen-3-yl)-piperazine: 6-[4-(1,1-dioxo-tetrahydro-1λ*6*-thiophen-3-yl)-piperazin-1-yl]-1-methyl-1,5-dihydro-pyrazolo[3,4-d]pyrimidin-4-one was obtained as a white solid (51.9 mg, 89.7%). ¹H NMR (300 MHz, DMSO-d₆) δ ppm 1.87-2.09 (m, 1H) 2.34 (d, J=12.81 Hz, 1H) 2.42-2.66 (m, 5H) 2.89-3.14 (m, 2H) 3.16-3.42 (m, 2H) 3.64 (t, J=4.99 Hz, 4H) 3.72 (s, 3H) 7.77 (s, 1H) 10.91 (s, 1H). LC-MS calcd. for C₁₄H₂₁N₆O₃S [(M+H)⁺] 353, obsd. 353.0.

### Example 28

### 6-(4-Cyclopentyl-piperazin-1-yl)-1-methyl-1,5-dihydro-pyrazolo[3,4-d]pyrimidin-4-one (I-37)

From 6-chloro-1-methyl-1,5-dihydro-pyrazolo[3,4-d]pyrimidin-4-one (Intermediate A) and 1-cyclopentylpiperazine: 6-(4-cyclopentyl-piperazin-1-yl)-1-methyl-1,5-dihydro-pyrazolo[3,4-d]pyrimidin-4-one was obtained as a white solid (44.0 mg, 87.5%). ¹H NMR (400 MHz, DMSO-d₆) δ ppm 1.26-1.68 (m, 6H) 1.78 (br. s., 2H) 2.38-2.48 (m, 5H) 3.55-3.66 (m, 4H) 3.71 (s, 3H) 7.76 (s, 1H) 10.86 (s, 1H). LC-MS calcd. for C₁₅H₂₃N₆O [(M+H)⁺] 303, obsd. 303.1.

### Example 29

### 6-[4-(2,6-Difluoro-phenyl)-piperazin-1-yl]-1-methyl-1,5-dihydro-pyrazolo[3,4-d]pyrimidin-4-one (I-2)

From 6-chloro-1-methyl-1,5-dihydro-pyrazolo[3,4-d]pyrimidin-4-one (Intermediate A) and 1-(2,6-difluorophenyl)piperazine 2,2,2-trifluoroacetate: 6-[4-(2,6-difluoro-phenyl)-piperazin-1-yl]-1-methyl-1,5-dihydro-pyrazolo[3,4-d]pyrimidin-4-one was obtained as an off-white solid (51.5 mg, 88.5%). ¹H NMR (300 MHz, DMSO-d₆) δ ppm 3.18 (br. s., 4H) 3.70-3.81 (m, 7H) 6.96-7.20 (m, 3H) 7.79 (s, 1H) 10.97 (s, 1H). LC-MS calcd. for C₁₆H₁₇F₂N₆O [(M+H)⁺] 347, obsd. 347.0.

### Example 30

### 6-[4-(2,6-Difluoro-4-nitro-phenyl)-piperazin-1-yl]-1-methyl-1,5-dihydro-pyrazolo[3,4-d]pyrimidin-4-one (I-49)

From 6-chloro-1-methyl-1,5-dihydro-pyrazolo[3,4-d]pyrimidin-4-one (Intermediate A) and 1-(2,6-difluoro-4-nitro-phenyl)-piperazine (Intermediate F): 6-[4-(2,6-difluoro-4-nitro-phenyl)-piperazin-1-yl]-1-methyl-1,5-dihydro-pyrazolo[3,4-d]pyrimidin-4-one was obtained a yellow solid (91.1 mg, 84.6%). ¹H NMR (400 MHz, DMSO-d₆) δ ppm 3.42 (br. s., 4H) 3.74 (s, 3H) 3.75-3.83 (m, 4H) 7.79 (s, 1H) 7.94-8.09 (m, 2H) 11.00 (s, 1H). LC-MS calcd. for C₁₆H₁₆F₂N₇O₃ [(M+H)⁺] 392, obsd. 392.0.

### Example 31

### 1-Methyl-6-[4-(2-trifluoromethyl-phenyl)-piperazin-1-yl]-1,5-dihydro-pyrazolo[3,4-d]pyrimidin-4-one (I-19)

A mixture of 6-chloro-1-methyl-1,5-dihydro-pyrazolo[3,4-d]pyrimidin-4-one (Intermediate A) (40 mg, 0.22 mmol), 1-(2-trifluoromethyl-phenyl)-piperazine (60 mg, 0.26 mmol), and DIPEA (76 μL, 0.433 mmol) in ethanol (2 mL) was heated to 150° C. for 15 min in a microwave reactor. The resulting precipitate was collected by filtration, washed with methanol and air dried to afford 1-methyl-6-[4-(2-trifluoromethyl-phenyl)-piperazin-1-yl]-1,5-dihydro-pyrazolo[3,4-d]pyrimidin-4-one (58 mg, 70.7%) as a white solid. ¹H NMR (300 MHz, DMSO-d₆) δ ppm 10.99 (s, 1H), 7.80 (s, 1H), 7.72-7.59 (m, 3H), 7.38 (t, 1H), 3.80 (t, 4H), 3.78 (s, 3H), 2.95 (t, 4H). LC-MS calcd. for C₁₇H₁₈F₃N₆O [(M+H)+] 379, obsd. 379.0.

In an analogous manner the following compounds were synthesized following the above procedure:

### Example 32

### 6-(4-Hydroxy-4-phenyl-piperidin-1-yl)-1-methyl-1,5-dihydro-pyrazolo[3,4-d]pyrimidin-4-one (I-23)

From 6-chloro-1-methyl-1,5-dihydro-pyrazolo[3,4-d]pyrimidin-4-one (Intermediate A) and 4-phenyl-piperidin-4-ol: 6-(4-hydroxy-4-phenyl-piperidin-1-yl)-1-methyl-1,5-dihydro-Pyrazolo[3,4-d]pyrimidin-4-one was obtained as a white solid. ¹H NMR (300 MHz, DMSO-d₆) δ ppm 10.82 (s, 1H), 7.76 (s, 1H), 7.51 (dd, 2H), 7.32 (t, 2H), 7.2 (t, 1H), 5.15 (s, 1H), 4.39 (d, 2H), 3.70 (s, 3H), 3.31 (t, 2H), 1.95 (t, 2H), 1.65 (t, 2H). LC-MS calcd. for C₁₇H₂₀N₅O₂ [(M+H)⁺] 326, obsd. 326.0.

### Example 33

### 1-(1-Methyl-4-oxo-4,5-dihydro-1H-pyrazolo[3,4-d]pyrimidin-6-yl)-4-phenyl-piperidine-4-carbonitrile (I-24)

From 6-chloro-1-methyl-1,5-dihydro-pyrazolo[3,4-d]pyrimidin-4-one (Intermediate A) and 4-phenylpiperidine-4-carbonitrile hydrochloride: 1-(1-methyl-4-oxo-4,5-dihydro-1H-pyrazolo-[3,4d]pyrimidin-6-yl)-4-phenyl-piperidine-4-carbonitrile was obtained as a white solid. ¹H NMR (300 MHz, DMSO-d₆) δ ppm 11.05 (s, 1H), 7.81 (s, 1H), 7.59 (dd, 2H), 7.49 (t, 2H), 7.40 (t, 1H), 4.61 (d, 2H), 3.75 (s, 3H), 3.25 (t, 2H), 2.22 (d, 2H), 2.09 (t, 2H). LC-MS calcd. for C₁₈H₁₉N₆O [(M+H)⁺] 335, obsd. 334.9.

### Example 34

### 3,5-Difluoro-4-[4-(1-methyl-4-oxo-4,5-dihydro-1H-pyrazolo[3,4-d]pyrimidin-6-yl)-piperazin-1-yl]-benzonitrile (I-56)

From 6-chloro-1-methyl-1,5-dihydro-pyrazolo[3,4-d]pyrimidin-4-one (Intermediate A) and 3,5-difluoro-4-(piperazin-1-yl)benzonitrile trifluoromethyl acetate salt, 3,5-difluoro-4-[4-(1-methyl-4-oxo-4,5-dihydro-1H-pyrazolo[3,4-d]pyrimidin-6-yl)-piperazin-1-yl]-benzonitrile was obtained as a white powder (35.6 mg, 88.5%). ¹H NMR (400 MHz, DMSO-d₆) δ ppm 10.98 (br. s., 1H), 7.80 (s, 1H), 7.73 (d, J=9.79 Hz, 2H), 3.75-3.80 (m, 4H), 3.74 (s, 3H), 3.34-3.37 (m, 4H). LC-MS calcd. for C₁₇H₁₆F₂N₇O [(M+H)⁺] 372, obsd. 372.0. Rₜ=3.91 min.

### Example 35

### 6-{4-[3-Fluoro-5-(2-methoxy-ethoxy)-phenyl]-piperazin-1-yl}-1-methyl-1,5-dihydro pyrazolo[3,4-d]pyrimidin-4-one (I-55)

From 6-chloro-1-methyl-1,5-dihydro-pyrazolo[3,4-d]pyrimidin-4-one (Intermediate A) and 1-(3-fluoro-5-(2-methoxyethoxy)phenyl)piperazine (Intermediate G), 6-{4-[3-fluoro-5-(2-methoxy-ethoxy)-phenyl]-piperazin-1-yl}-1-methyl-1,5-dihydro pyrazolo[3,4-d]pyrimidin-4-one was obtained as a light yellow solid (4.6 mg, 42.2%). ¹H NMR (400 MHz, DMSO-d₆) δ ppm 9.72 (br. s, 1H), 7.80 (s, 1H), 6.42 (d, J=9.80 Hz, 1H), 6.33 (br. s, 1), 6.26 (d, J=9.80 Hz, 1H), 4.06-4.10 (m, 2H), 3.75-3.80 (m, 4H), 3.75 (s, 3H), 3.25-3.30 (m, 4H). LC-MS calcd. for C₁₉H₂₄FN₆O₃ [(M+H)⁺] 403, obsd. 403.1. Rₜ=3.90 min.

### Example 36

### 6-[4-(2,3-Difluoro-5-hydroxy-phenyl)-piperazin-1-yl]-1-methyl-1,5-dihydro-pyrazolo[3,4-d]pyrimidin-4-one (I-57)

From 6-chloro-1-methyl-1,5-dihydro-pyrazolo[3,4-d]pyrimidin-4-one (Intermediate A) and 3,4-difluoro-5-(piperazin-1-yl)phenol (Intermediate H), 6-[4-(2,3-difluoro-5-hydroxy-phenyl)-piperazin-1-yl]-1-methyl-1,5-dihydro-pyrazolo[3,4-d]pyrimidin-4-one was obtained as a white powder (58.8 mg, 81.1%). ¹H NMR (400 MHz, DMSO-d₆) δ ppm 9.72 (br. s, 1H), 7.80 (s, 1H), 6.30-6.38 (m, 1H), 6.21-6.23 (m, 1H), 3.75-3.85 (m, 4H), 3.72 (s, 3H), 3.05-3.10 (m, 4H). LC-MS calcd. for C₁₆H₁₇F₂N₆O₂ [(M+H)⁺] 363, obsd. 363.0. Rₜ=3.80 min.

### Example 37

### 6-[4-(4-Amino-2,6-difluoro-phenyl)-piperazin-1-yl]-1-methyl-1,5-dihydro-pyrazolo[3,4-d]pyrimidin-4-one (I-54)

A solution of 6-(4-(2,6-difluoro-4-nitrophenyl)piperazin-1-yl)-1-methyl-1H-pyrazolo[3,4-d]pyrimidin-4(5H)-one (80 mg, 204 μmol) (Example 30) in ethanol (150 mL), glacial acetic acid (50 mL) and 1,4-dioxane (50 mL) was exposed to a H-Cube reaction system (20 Bar/45° C.). The crude reaction mixture was concentrated in vacuo. Reverse phase chromatography (CombiFlash Rf system, C18 column, 20-100% acetonitrile in water) afforded 6-[4-(4-amino-2,6-difluoro-phenyl)-piperazin-1-yl]-1-methyl-1,5-dihydro-pyrazolo[3,4-d]pyrimidin-4-one (35.6 mg, 48.2%) as a white powder. ¹H NMR (400 MHz, DMSO-d₆) δ ppm 10.98 (br. s., 1H), 7.80 (s, 1H), 6.17 (d, J=9.79 Hz, 2H), 5.52 (br. s, 2H), 3.74 (s, 3H), 3.70-3.74 (m, 4H), 3.00-3.05 (m, 4H). LC-MS calcd. for C₁₆H₁₈F₂N₇O [(M+H)⁺] 362, obsd. 362.1. Rₜ=2.83 min.

### Example 38

### 6-[4-(4-Fluoro-phenyl)-piperidin-1-yl]-1,5-dihydro-pyrazolo[3,4-d]pyrimidin-4-one (I-11)

A mixture of 6-chloro-1,5-dihydro-pyrazolo[3,4-d]pyrimidin-4-one (Intermediate B) (29 mg, 0.17 mmol), DIPEA (223 mg, 1.72 mmol), and 4-(4-fluorophenyl)piperidine hydrochloride (91 mg, 0.42 mmol) in ethanol (0.5 mL) was heated at 140° C. in a sealed tube for 1.5 h. At this time, the resulting mixture was concentrated in vacuo. Flash chromatography (15/1 methylene chloride/methanol) afforded 6-[4-(4-fluoro-phenyl)-piperidin-1-yl]-1,5-dihydro-pyrazolo[3,4-d]pyrimidin-4-one (30 mg, 56.3%) as a white solid. ¹H NMR (400 MHz, DMSO-d₆) δ ppm 1.51-1.69 (m, 2H) 1.79 (d, J=11.29 Hz, 2H) 2.82 (t, J=12.05 Hz, 1H) 2.99 (t, J=12.30 Hz, 2H) 4.46 (d, J=12.80 Hz, 2H) 6.98-7.18 (m, 2H) 7.19-7.42 (m, 2H) 7.77 (s, 1H) 10.86 (br. s., H) 12.89 (br. s., 1H). LC-MS calcd. for C₁₆H₁₆FN₅O [(M)⁺] 314, obsd. 313.9.

In an analogous manner the following compound was synthesized following the above procedure:

### Example 39

### 6-[4-(2-Fluoro-phenyl)-piperazin-1-yl]-1,5-dihydro-pyrazolo[3,4-d]pyrimidin-4-one (I-10)

From 6-chloro-1,5-dihydro-pyrazolo[3,4-d]pyrimidin-4-one (Intermediate B) and 1-(2-fluoro-phenyl)-piperazine: 6-[4-(2-fluoro-phenyl)-piperazin-1-yl]-1,5-dihydro-pyrazolo[3,4-d]pyrimidin-4-one was obtained as a white solid (23 mg, 43%). ¹H NMR (400 MHz, DMSO-d₆) δ ppm 2.99-3.14 (m, 4H) 3.76 (br. s., 4H) 6.83-7.30 (m, 4H) 7.79 (s, 1H) 11.00 (br. s., 1H) 12.98 (br. s., 1H). LC-MS calcd. for C₁₅H₁₆FN₆O [(M+H)⁺] 315, obsd. 315.0.

### Example 40

### 6-[4-(4-Fluoro-phenyl)-piperidin-1-yl]-1-methyl-1,5-dihydro-pyrazolo[4,3-c]pyridin-4-one (I-8)

A mixture of 6-chloro-1-methyl-1,5-dihydro-pyrazolo[4,3-c]pyridin-4-one (25 mg, 0.135 mmol) (Intermediate C), DIPEA (371 mg, 2.87 mmol), and 4-(4-fluorophenyl)piperidine hydrochloride (58.4 mg, 0.271 mmol) was heated at 140° C. in a sealed tube overnight. Flash chromatography (15/1 methylene chloride/methanol) afforded 6-[4-(4-fluoro-phenyl)-piperidin-1-yl]-1-methyl-1,5-dihydro-pyrazolo[4,3-c]pyridin-4-one (30 mg, 70.3%) as a white solid. ¹H NMR (400 MHz, DMSO-d₆) δ ppm 1.61-1.96 (m, 4H) 2.62-2.89 (m, 3H) 3.70-3.87 (m, 5H) 5.79 (s, 1H) 7.13 (t, J=8.91 Hz, 2H) 7.31 (dd, J=8.53, 5.77 Hz, 2H) 7.79 (s, 1H) 10.70 (s, 1H). LC-MS calcd. for C₁₈H₂₀FN₄O [(M+H)⁺] 327, obsd. 327.1.

In an analogous manner the following compounds were synthesized following the above procedure:

### Example 41

### 6-[4-(2-Fluoro-phenyl)-piperazin-1-yl]-1-methyl-1,5-dihydro-pyrazolo[4,3-c]pyridin-4-one (I-7)

From 6-chloro-1-methyl-1,5-dihydro-pyrazolo[4,3-c]pyridin-4-one (Intermediate C) and 1-(2-fluorophenyl)piperazine: 6-[4-(2-fluoro-phenyl)-piperazin-1-yl]-1-methyl-1,5-dihydro-pyrazolo[4,3-c]pyridin-4-one was obtained as a white solid (27 mg, 75.7%). ¹H NMR (400 MHz, DMSO-d₆) δ ppm 3.1 (d, J=4.52 Hz, 4H) 3.8 (s, 3H) 5.9 (s, 1H) 7.0-7.0 (m, 1H) 7.1-7.2 (m, 3H) 7.8 (s, 1H) 10.8 (s, 1H). LC-MS calcd. for C₁₇H₁₉FN₅O [(M+H)⁺] 328, obsd. 328.0.

### Example 42

### 6-[4-(2,4-Difluoro-phenyl)-piperazin-1-yl]-1-methyl-1,5-dihydro-pyrazolo[4,3-c]pyridin-4-one (I-14)

From 6-chloro-1-methyl-1,5-dihydro-pyrazolo[4,3-c]pyridin-4-one (Intermediate C) and 1-(2,4-difluorophenyl)piperazine: 6-[4-(2,4-difluoro-phenyl)-piperazin-1-yl]-1-methyl-1,5-dihydro-pyrazolo[4,3-c]pyridin-4-one was obtained as a white solid (46 mg, 90.6%). ¹H NMR (400 MHz, DMSO-d₆) δ ppm 3.01-3.12 (m, 4H) 3.27-3.33 (m, 4H) 3.83 (s, 3H) 5.85 (s, 1H) 6.96-7.07 (m, 1H) 7.13 (td, J=9.41, 5.77 Hz, 1H) 7.23 (ddd, J=12.36, 9.10, 2.89 Hz, 1H) 7.82 (d, J=0.75 Hz, 1H) 10.81 (s, 1H). LC-MS calcd. for C₁₇H₁₈F₂N₅O [(M+H)⁺] 346, obsd. 346.0.

### Example 43

### 2-[4-(1-Methyl-4-oxo-4,5-dihydro-1H-pyrazolo[4,3-c]pyridin-6-yl)-piperazin-1-yl]-benzonitrile (I-17)

From 6-chloro-1-methyl-1,5-dihydro-pyrazolo[4,3-c]pyridin-4-one (Intermediate C) and 2-(piperazin-1-yl)benzonitrile: 2-[4-(1-methyl-4-oxo-4,5-dihydro-1H-pyrazolo[4,3-c]pyridin-6-yl)-piperazin-1-yl]-benzonitrile was obtained as a white solid (20 mg, 90.6%). ¹H NMR (400 MHz, DMSO-d₆) δ ppm 3.27 (d, J=5.02 Hz, 4H) 3.31-3.39 (m, 4H) 3.84 (s, 3H) 5.91 (s, 1H) 7.14 (td, J=7.53, 1.00 Hz, 1H) 7.24 (d, J=8.03 Hz, 1H) 7.63 (ddd, J=8.53, 7.28, 1.51 Hz, 1H) 7.74 (dd, J=7.78, 1.51 Hz, 1H) 7.82 (d, J=0.50 Hz, 1H) 10.83 (br. s., 1H). LC-MS calcd. for C₁₈H₁₉N₆O [(M+H)⁺] 335, obsd. 335.1.

### Example 44

### 6-[4-(4-Fluoro-2-methylsulfonyl-phenyl)-piperazin-1-yl]-1-methyl-1,5-dihydro-pyrazolo[4,3-c]pyridin-4-one (I-25)

From 6-chloro-1-methyl-1,5-dihydro-pyrazolo[4,3-c]pyridin-4-one (Intermediate C) and 1-(4-fluoro-2-(methylsulfonyl)phenyl)piperazine: 6-[4-(4-fluoro-2-methylsulfonyl-phenyl)-piperazin-1-yl]-1-methyl-1,5-dihydro-pyrazolo[4,3-c]pyridin-4-one was obtained as a white solid (40 mg, 72.5%). ¹H NMR (400 MHz, DMSO-d₆) δ ppm 3.07 (br. s., 4H) 3.25-3.39 (m, 4H) 3.45 (s, 3H) 3.84 (s, 3H) 5.89 (s, 1H) 7.56-7.70 (m, 2H) 7.75 (dd, J=8.66, 4.89 Hz, 1H) 7.82 (d, J=0.75 Hz, 1H) 10.79 (s, 1H). LC-MS calcd. for C₁₈H₂₁FN₅O₃S [(M+H)⁺] 406, obsd. 406.0.

### Example 45

### 1-Methyl-6-[4-(3-trifluoromethyl-pyridin-2-yl)-piperazin-1-yl]-1,5-dihydro-pyrazolo[4,3-c]pyridin-4-one (I-26)

From 6-chloro-1-methyl-1,5-dihydro-pyrazolo[4,3-c]pyridin-4-one (Intermediate C) and 1-(3-(trifluoromethyl)pyridin-2-yl)piperazine: 1-methyl-6-[4-(3-trifluoromethyl-pyridin-2-yl)-piperazin-1-yl]-1,5-dihydro-pyrazolo[4,3-c]pyridin-4-one was obtained as a white solid (36 mg, 69.9%). ¹H NMR (400 MHz, DMSO-d₆) δ ppm 3.33 (s, 6H) 3.38-3.51 (m, 2H) 3.83 (s, 3H) 5.88 (s, 1H) 7.14-7.34 (m, 1H) 7.82 (d, J=0.75 Hz, 1H) 8.11 (dd, J=7.91, 1.63 Hz, 1H) 8.56 (dd, J=4.77, 1.00 Hz, 1H) 10.81 (s, 1H). LC-MS calcd. for C₁₇H₁₈F₃N₆O [(M+H)⁺] 379, obsd. 379.1.

### Example 46

### 6-[4-(3,5-Dichloro-pyridin-4-yl)-piperazin-1-yl]-1-methyl-1,5-dihydro-pyrazolo[4,3-c]pyridin-4-one (I-47)

From 6-chloro-1-methyl-1,5-dihydro-pyrazolo[4,3-c]pyridin-4-one (Intermediate C) and 1-(3,5-dichloropyridin-4-yl)piperazine: 6-[4-(3,5-dichloro-pyridin-4-yl)-piperazin-1-yl]-1-methyl-1,5-dihydro-pyrazolo[4,3-c]pyridin-4-one was obtained as a white solid (46 mg, 92.8%). ¹H NMR (400 MHz, DMSO-d₆) δ ppm 3.30 (d, J=4.77 Hz, 4H) 3.38-3.50 (m, 4H) 3.84 (s, 3H) 5.90 (s, 1H) 7.83 (d, J=0.75 Hz, 1H) 8.49 (s, 2H) 10.84 (s, 1H). LC-MS calcd. for C₁₆H₁₆Cl₂N₆O [(M)⁺] 379, obsd. 379.0.

### Example 47

### 6-[4-(2,6-Fluoro-phenyl)-piperazin-1-yl]-1-methyl-1,5-dihydro-pyrazolo[4,3-c]pyridin-4-one (I-51)

A mixture of 6-chloro-1-methyl-1,5-dihydro-pyrazolo[4,3-c]pyridin-4-one (Intermediate C) (28 mg, 0.15 mmol), DIPEA (127 mg, 0.98 mmol) and 1-(2,6-difluorophenyl)piperazine trifluoroacetic acid salt (150 mg, 0.48 mmol) in ethanol (0.2 mL) was heated at 140° C. in a sealed tube for 3 days. At this time, the resulting mixture was concentrated in vacuo. Flash chromatography (15/1 methylene chloride/methanol) afforded 6-[4-(2,6-fluoro-phenyl)-piperazin-1-yl]-1-methyl-1,5-dihydro-pyrazolo[4,3-c]pyridin-4-one (47 mg, 89.2%) as a white solid. ¹H NMR (400 MHz, DMSO-d₆) δ ppm 3.25 (d, J=5.02 Hz, 8H) 3.84 (s, 3H) 5.86 (s, 1H) 6.95-7.23 (m, 3H) 7.82 (d, J=0.75 Hz, 1H) 10.80 (s, 1H). LC-MS calcd. for C₁₇H₁₈F₂N₅O [(M+H)⁺] 346, obsd. 346.0.

In an analogous manner the following compound was synthesized following the above procedure:

### Example 48

### 6-{4-[2,6-Difluoro-4-(2-methoxy-ethoxy)-phenyl]-piperazin-1-yl}-1-methyl-1,5-dihydro-pyrazolo[4,3-c]pyridin-4-one (I-53)

From 6-chloro-1-methyl-1,5-dihydro-pyrazolo[4,3-c]pyridin-4-one (Intermediate C) and 1-(2,6-difluoro-4-(2-methoxyethoxy)phenyl)piperazine (Intermediate I): 6-{4-[2,6-difluoro-4-(2-methoxy-ethoxy)-phenyl]-piperazin-1-yl}-1-methyl-1,5-dihydro-pyrazolo[4,3-c]pyridin-4-one was obtained as a white solid (56 mg, 66.3%). ¹H NMR (400 MHz, DMSO-d₆) δ ppm 3.13 (br. s., 4H) 3.24 (br. s., 4H) 3.29 (s, 3H) 3.57-3.69 (m, 2H) 3.83 (s, 3H) 4.08 (dd, J=5.27, 3.51 Hz, 2H) 5.84 (s, 1H) 6.74 (d, J=11.29 Hz, 2H) 7.81 (s, 1H) 10.77 (s, 1H). LC-MS calcd. for C₂₀H₂₄F₂N₅O₃ [(M+H)⁺] 420, obsd. 420.1.

In an analogous manner the following compounds were synthesized following the above procedure:

### Example 49

### 6-[4-(4-Fluoro-phenyl)-piperidin-1-yl]-1,5-dihydro-pyrazolo[4,3-c]pyridin-4-one (I-9)

From 6-chloro-1,5-dihydro-pyrazolo[4,3-c]pyridin-4-one (Intermediate D) and 4-(4-fluorophenyl)piperidine hydrochloride: 6-[4-(4-fluoro-phenyl)-piperidin-1-yl]-1,5-dihydro-pyrazolo[4,3-c]pyridin-4-one was obtained as a white solid (25 mg, 46.8%). ¹H NMR (400 MHz, DMSO-d₆) δ ppm 1.60-1.77 (m, 2H) 1.77-1.90 (m, 2H) 2.60-2.85 (m, 3H) 3.72 (d, J=12.05 Hz, 2H) 5.60 (s, 1H) 7.01-7.20 (m, 2H) 7.22-7.43 (m, 2H) 7.83 (s, 1H) 10.69 (br. s., 1H) 12.78 (s, 1H). LC-MS calcd. for C₁₇H₁₈FN₄O [(M+H)⁺] 313, obsd. 313.0.

### Example 50

### 6-[4-(2-Fluoro-phenyl)-piperazin-1-yl]-1,5-dihydro-pyrazolo[4,3-c]pyridin-4-one (I-12)

From 6-chloro-1,5-dihydro-pyrazolo[4,3-c]pyridin-4-one (Intermediate D) and 1-(2-fluorophenyl)piperazine: 6-[4-(2-fluoro-phenyl)-piperazin-1-yl]-1,5-dihydro-pyrazolo[4,3-c]pyridin-4-one was obtained as a white solid (30 mg, 56%). ¹H NMR (400 MHz, DMSO-d₆) δ ppm 3.13 (d, J=4.77 Hz, 4H) 3.28 (br. s., 4H) 5.64 (s, 1H) 6.92-7.27 (m, 4H) 7.86 (s, 1H) 10.79 (br. s., 1H) 12.87 (br. s., 1H). LC-MS calcd. for C₁₆H₁₇FN₅O [(M+H)⁺] 314, obsd. 314.0.

### Example 51

### 1-Methyl-6-(6-trifluoromethyl-pyridin-3-yl)-1,5-dihydro-pyrazolo[3,4-d]pyrimidin-4-one (I-27)

6-Chloro-1-methyl-1H-pyrazolo[3,4-d]pyrimidin-4(5H)-one (75 mg, 0.41 mmol), 6-(trifluoromethyl)pyridin-3-ylboronic acid (155 mg, 0.81 mmol), 2M aqueous sodium carbonate (609 μL, 1.22 mmol), DMF and ethanol were mixed in a microwave vessel, degassed and flushed with nitrogen three times. Tetrakis(triphenylphosphine)palladium(0) (47 mg, 0.04 mmol) was added. The vessel was sealed, degassed and flushed with nitrogen three times. The resulting mixture was heated at 140° C. for 15 min via microwave. The mixture was diluted with EtOAc (200 mL) and water (100 mL). The organic phase was separated, washed with saturated ammonium chloride and brine, dried over sodium sulfate, filtered and evaporated in vacuo. The resulted yellow solid was sonicated with EtOAc (10 mL) for 10 minutes. The solid was collected by filtration, washed with EtOAc, and air dried to yield 1-methyl-6-(6-trifluoromethyl-pyridin-3-yl)-1,5-dihydro-pyrazolo[3,4-d]pyrimidin-4-one (91 mg, 76%) as a white solid. ¹H NMR (300 MHz, DMSO-d₆) δ ppm 12.76 (s, 1H), 9.42 (s, 1H), 8.75 (d, 1H), 8.20-8.10 (m, 2H), 3.99 (s, 3H). LC-MS calcd. for C₁₂H₉F₃N₅O [(M+H)⁺] 296, obsd. 296.0.

### Example 52

### 1-Methyl-6-(4-trifluoromethyl-phenyl)-1,5-dihydro-pyrazolo[4,3-c]pyridin-4-one (I-13)

A microwave reaction vial was charged with 6-chloro-1-methyl-1,5-dihydro-pyrazolo[4,3-e]pyridin-4-one (Intermediate C) (10 mg, 0.05 mmol), 4-(trifluoromethyl)phenylboronic acid (20.7 mg, 0.11 mmol), tetrakis(triphenylphosphine)palladium(0) (3.15 mg, 0.003 mmol), and a 2M aqueous sodium carbonate solution (0.08 mL) in ethanol (1 mL). The vial was sealed and then heated in the microwave at 140° C. for 10 min. At this time, the resulting mixture was filtered through a pad of Celite® and then was concentrated in vacuo. Flash chromatography (20/1 methylene chloride/methanol) afforded 1-methyl-6-(4-trifluoromethyl-phenyl)-1,5-dihydro-pyrazolo[4,3-c]pyridin-4-one (15 mg, 93.9%) as a white solid. ¹H NMR (400 MHz, DMSO-d₆) δ ppm 4.0 (s, 3H) 7.1 (s, 1H) 7.9 (d, J=8.28 Hz, 2H) 8.0 (d, J=8.03 Hz, 2H) 8.1 (s, 1H) 11.4 (s, 1H). LC-MS calcd. for C₁₄H₁₁F₃N₃O [(M+H)⁺] 294, obsd. 294.0.

In an analogous manner the following compound was synthesized following the above procedure:

### Example 53

### 1-Methyl-6-(6-trifluoromethyl-pyridin-3-yl)-1,5-dihydro-pyrazolo[4,3-c]pyridin-4-one (I-29)

From 6-chloro-1-methyl-1,5-dihydro-pyrazolo[4,3-c]pyridin-4-one (Intermediate C) and 6-(trifluoromethyl)pyridin-3-ylboronic acid: 1-methyl-6-(6-trifluoromethyl-pyridin-3-yl)-1,5-dihydro-pyrazolo[4,3-c]pyridin-4-one was obtained as a as a white solid. ¹H NMR (400 MHz, DMSO-d₆) δ ppm 4.02 (s, 3H) 7.24 (d, J=0.75 Hz, 1H) 8.05-8.12 (m, 2H) 8.46 (dd, J=8.03, 2.01 Hz, 1H) 9.16 (d, J=2.26 Hz, 1H) 11.55 (br. s., 1H). LC-MS calcd. for C₁₃H₁₀F₃N₄O [(M+H)⁺] 295, obsd. 295.0.

### Example 54

### 1-Methyl-6-(4-trifluoromethyl-phenyl)-1,5-dihydro-pyrazolo[3,4-d]pyrimidin-4-one (I-4)

A microwave reaction vial was charged with 6-chloro-1-methyl-1,5-dihydro-pyrazolo[3,4-d]pyrimidin-4-one (Intermediate A) (30 mg, 0.163 mmol), 4-(trifluoromethyl)phenylboronic acid (40.1 mg, 0.211 mmol), tetrakis(triphenylphosphine)palladium(0) (9.39 mg, 0.008 mmol), and a 2M aqueous sodium carbonate solution (0.24 mL) in ethanol (2 mL). The vial was sealed and heated in the microwave at 140° C. for 10 min. The resulting mixture was filtered through a pad of Celite® and concentrated in vacuo. Flash chromatography (20/1 methylene chloride/methanol) afforded 1-methyl-6-(4-trifluoromethyl-phenyl)-1,5-dihydro-pyrazolo[3,4-d]pyrimidin-4-one (35 mg, 73.2%) as a white solid. ¹H NMR (400 MHz, DMSO-d₆) δ ppm 4.0 (s, 3H) 7.9 (d, J=8.28 Hz, 2H) 8.1 (s, 1H) 8.4 (d, J=8.28 Hz, 2H) 12.6 (br. s., 1H). LC-MS calcd. for C₁₃H₁₀F₃N₄O [(M+H)⁺] 295, obsd. 295.0.

### Example 55

### 6-{4-[2-Fluoro-4-(1-hydroxy-1-methyl-ethyl)-phenyl]-piperazin-1-yl}-1-methyl-1,5-dihydro-pyrazolo[3,4-d]pyrimidin-4-one (1.44)

A microwave reaction vial was charged with 3-fluoro-4-[4-(1-methyl-4-oxo-4,5-dihydro-1H-pyrazolo[3,4-d]pyrimidin-6-yl)-piperazin-1-yl]-benzoic acid (180 mg, 483 μmol) and methanol (2.4 mL). This mixture was treated with concentrated sulfuric acid (2 drops). The vial was capped, and the reaction was heated at 75° C. overnight. At this time, the reaction was diluted with methanol and methylene chloride and concentrated in vacuo onto Celite®. Flash chromatography (10 g silica gel column, 1-10% methanol/methylene chloride) afforded methyl 3-fluoro-4-(4-(1-methyl-4-oxo-4,5-dihydro-1H-pyrazolo[3,4-d]pyrimidin-6-yl)piperazin-1-yl)benzoate as a light brown gum (200 mg, 107%). ¹H NMR (300 MHz, DMSO-d₆) δ ppm 3.26 (br. s., 4H) 3.74 (s, 3H) 3.82 (s, 7H) 7.16 (t, J=8.76 Hz, 1H) 7.57-7.77 (m, 2H) 7.79 (s, 1H). LC-MS calcd. for C₁₈H₂₀FN₆O₃ [(M+H)⁺] 387, obsd. 386.9.

A microwave reaction vial was charged with methyl 3-fluoro-4-(4-(1-methyl-4-oxo-4,5-dihydro-1H-pyrazolo[3,4-d]pyrimidin-6-yl)piperazin-1-yl)benzoate (50 mg, 129 μmol) and tetrahydrofuran (1.1 ml) under nitrogen was treated dropwise over 10 min with methyl magnesium bromide (3M, 220 μL, 660 μmol). The reaction was stirred at room temperature for 4 h. At this time, the reaction was quenched with methanol (2 mL) and then was concentrated in vacuo onto Celite®. Flash chromatography (10 g silica gel column, 1-10% methanol/methylene chloride) afforded 6-{4-[2-fluoro-4-(1-hydroxy-1-methyl-ethyl)-phenyl]-piperazin-1-yl}-1-methyl-1,5-dihydro-pyrazolo[3,4-d]pyrimidin-4-one as a light brown solid (16.1 mg, 32.2%). ¹H NMR (300 MHz, DMSO-d₆) δ ppm 1.39 (s, 6H) 3.05 (br. s., 4H) 3.74 (s, 3H) 3.81 (br. s., 4H) 5.02 (s, 1H) 6.89-7.09 (m, 1H) 7.10-7.29 (m, 2H) 7.78 (s, 1H) 10.95 (d, J=14.13 Hz, 1H). LC-MS calcd. for C₁₉H₂₄FN₆O₂ [(M+H)⁺] 387, obsd. 386.9ple 56

### 1-Methyl-6-(5-trifluoromethyl-pyridin-2-yl)-1,5-dihydro-pyrazolo[3,4-d]pyrimidin-4-one (I-50)

A solution of 5-(trifluoromethyl)picolinic acid (320 mg, 1.67 mmol) in anhydrous methylene chloride (10 mL) cooled to 0° C. was treated with a solution of oxalyl chloride (2M in methylene chloride, 2.51 mL, 5.02 mmol) and tetrahydrofuran (8 mL). The reaction was stirred at 0° C. for 2 h and was then concentrated in vacuo. The resulting residue was azeotroped with toluene (1×30 mL). A solution of the resulting residue in anhydrous tetrahydrofuran (5 mL) was added to a solution of ethyl 5-amino-1-methyl-1H-pyrazole-4-carboxylate (283 mg, 1.67 mmol) in tetrahydrofuran (5 mL) cooled to 0° C. The reaction mixture was then treated with pyridine (1 mL) and was allowed to warm to room temperature where it was stirred for 2 h. At this time, the reaction was diluted with ethyl acetate (200 mL), washed with water (2×100 mL) and a saturated aqueous sodium chloride solution (2×100 mL), dried over sodium sulfate, filtered and concentrated in vacuo. Flash chromatography (40 g silica gel column, 0-50% ethyl acetate/hexanes) afforded ethyl 1-methyl-5-(5-(trifluoromethyl)picolinamido)-1H-pyrazole-4-carboxylate (450 mg, 78.5%) as a white solid. LC-MS calcd. for C₁₄H₁₄F₃N₄O₃ [(M+H)⁺] 343, obsd. 343.0.

A mixture of ethyl 1-methyl-5-(5-(trifluoromethyl)picolinamido)-1H-pyrazole-4-carboxylate (197 mg, 0.58 mmol) and triphenylphosphine (453 mg, 1.78 mmol) in dry acetonitrile (5 mL) and carbon tetrachloride (266 mg, 1.73 mmol) was stirred at room temperature over the weekend. At this time, the reaction was treated with excess ammonium acetate and heated at 110° C. overnight in a sealed vial. The reaction was then diluted with ethyl acetate (150 mL), washed with water (2×50 mL) and a saturated aqueous sodium chloride solution (2×50 mL), dried over sodium sulfate, filtered and concentrated in vacuo. Flash chromatography (40 g silica gel column, 0-5% methanol/methylene chloride followed by 24 g silica gel column, 10-60% ethyl acetate/hexanes) afforded 1-methyl-6-(5-trifluoromethyl-pyridin-2-yl)-1,5-dihydro-pyrazolo[3,4-d]pyrimidin-4-one (68 mg, 40%) as a white solid. ¹H NMR (300 MHz, DMSO-d₆) δ ppm 11.95 (s, 1H), 9.15 (s, 1H), 8.62 (d, 1H), 8.55 (d, 1H), 8.15 (s, 1H), 4.05 (s, 3H). LC-MS calcd. for C₁₂H₉F₃N₅O [(M+H)⁺] 296, obsd. 295.8.

### Example 57

### 6-{4-[2-Fluoro-4-(1H-tetrazol-5-yl)-phenyl]-piperazin-1-yl}-1-methyl-1,5-dihydro-pyrazolo[3,4-d]pyrimidin-4-one (I-31)

A mixture of 3-fluoro-4-(4-(1-methyl-4-oxo-4,5-dihydro-1H-pyrazolo[3,4-d]pyrimidin-6-yl)piperazin-1-yl)benzonitrile (70 mg, 0.198 mmol), sodium azide (38.6 mg, 0.59 mmol) and ammonium chloride (31.8 mg, 0.59 mmol) in DMF (2 mL) was heated at 110° C. for 2 days. At this time, the reaction mixture was quenched with water and then extracted with ethyl acetate. The aqueous layer was concentrated in vacuo. Flash chromatography (20% methanol/methylene chloride with 0.2% triethylamine) afforded 6-{4-[2-fluoro-4-(1H-tetrazol-5-yl)-phenyl]-piperazin-1-yl}-1-methyl-1,5-dihydro-pyrazolo[3,4-d]pyrimidin-4-one (25 mg, 31.8%) as a white solid. ¹H NMR (400 MHz, DMSO-d₆) δ ppm 3.24 (br. s., 4H) 3.75 (s, 3H) 3.84 (br. s., 4H) 7.29 (t, J=8.91 Hz, 1H) 7.66-7.90 (m, 3H) 11.04 (s, 1H). LC-MS calcd. for C₁₇H₁₈FN₀O [(M+H)⁺] 397, obsd. 397.0.

### Example 58

### 1-methyl-6-[3-(3-pyridylmethyl)pyrrolidin-1-yl]-5H-pyrazolo[3,4-d]pyrimidin-4-one (I-137)

From 6-chloro-1-methyl-1,5-dihydro-pyrazolo[3,4-d]pyrimidin-4-one (Intermediate A) and 3-(pyrrolidin-3-ylmethyl)pyridine: 1-methyl-6-[3-(3-pyridylmethyl)pyrrolidin-1-yl]-5H-pyrazolo[3,4-d]pyrimidin-4-one (CASRN 1018827-45-6) was obtained a white solid (17.6 mg, 30%). ¹H NMR (400 MHz, DMSO-d₆) δ 10.47 (d, J=30.5 Hz, 1H), 8.48 (dd, J=2.3, 0.9 Hz, 1H), 8.44 (dd, J=4.8, 1.7 Hz, 1H), 7.71 (s, 1H), 7.70-7.65 (m, 1H), 7.34 (ddd, J=7.8, 4.8, 0.8 Hz, 1H), 3.68 (m, 5H), 3.44 (ddd, J=10.8, 8.5, 7.1 Hz, 1H), 3.20 (dd, J=10.8, 7.8 Hz, 1H), 2.74 (d, J=7.5 Hz, 2H), 2.60-2.54 (m, 1H), 2.05-1.93 (m, 1H), 1.66 (dq, J=12.2, 8.5 Hz, 1H). LC-MS calcd. for C16H18N6O [(M+H)⁺] 311.3, obsd. 311.2.

### Example 59

### 1-methyl-6-[3-(pyrimidin-2-ylmethyl)pyrrolidin-1-yl]-5H-pyrazolo[3,4-d]pyrimidin-4-one (I-133)

From 6-chloro-1-methyl-1,5-dihydro-pyrazolo[3,4-d]pyrimidin-4-one (Intermediate A) and 2-(pyrrolidin-3-ylmethyl)pyrimidine (CASRN 1316224-83-5) was obtained 1-methyl-6-[3-(pyrimidin-2-ylmethyl)pyrrolidin-1-yl]-5H-pyrazolo[3,4-d]pyrimidin-4-one as a white solid (13.8 mg, 25%). ¹H NMR (400 MHz, DMSO-d₆) δ 8.76 (d, J=4.9 Hz, 2H), 7.70 (s, 1H), 7.37 (t, J=4.9 Hz, 1H), 3.80-3.60 (m, 5H), 3.54-3.38 (m, 1H), 3.22 (dd, J=10.9, 7.7 Hz, 1H), 3.02 (d, J=7.4 Hz, 2H), 2.82 (dq, J=14.7, 7.4, 7.0 Hz, 1H), 2.07 (dtd, J=13.6, 6.9, 4.2 Hz, 1H), 1.72 (dq, J=12.2, 8.3 Hz, 1H). LC-MS calcd. for C15H17N7O [(M+H)⁺1312.3, obsd. 312.2.

### Example 60

### 1-methyl-6,4-(3-methylimidazol-4-yl)-1-piperidyl]-5H-pyrazolo[3,4-d]pyrimidin-4-one (I-100)

From 6-chloro-1-methyl-1,5-dihydro-pyrazolo[3,4-d]pyrimidin-4-one (Intermediate A) and 4-(1-methyl-1H-imidazol-5-yl)piperidine was obtained 1-methyl-6-[4-(3-methylimidazol-4-yl)-1-piperidyl]-5H-pyrazolo[3,4-d]pyrimidin-4-one as a white solid (32.9 mg, 58%). ¹H NMR (400 MHz, DMSO-d₆) δ 7.74 (s, 1H), 7.47 (d, J=1.1 Hz, 1H), 6.69-6.61 (m, 1H), 4.50 (d, J=13.3 Hz, 2H), 3.72 (s, 3H), 3.59 (s, 3H), 3.04 (td, J=13.0, 2.4 Hz, 2H), 2.88 (tt, J=11.9, 3.7 Hz, 1H), 2.00-1.87 (m, 2H), 1.50 (qd, J=12.5, 3.9 Hz, 2H). LC-MS calcd. for C15H19N7O [(M+H)⁺] 314.1, obsd. 314.2.

### Example 61

### 1-methyl-6-[3-(4-pyridylmethyl)pyrrolidin-1-yl]-5H-pyrazolo[3,4-d]pyrimidin-4-one (I-138)

From 6-chloro-1-methyl-1,5-dihydro-pyrazolo[3,4-d]pyrimidin-4-one (Intermediate A) and 4-(pyrrolidin-3-ylmethyl)pyridine (CASRN 1316223-46-7) was obtained 1-methyl-6-[3-(4-pyridylmethyl)pyrrolidin-1-yl]-5H-pyrazolo[3,4-d]pyrimidin-4-one as a white solid (18.0 mg, 32%). ¹H NMR (400 MHz, DMSO-d₆) δ 8.53-8.45 (m, 2H), 7.71 (s, 1H), 7.33-7.25 (m, 2H), 3.74-3.57 (m, 5H), 3.51-3.37 (m, 1H), 3.17 (dd, J=10.8, 7.8 Hz, 1H), 2.74 (d, J=7.5 Hz, 2H), 2.65-2.52 (m, 1H), 2.07-1.93 (m, 1H), 1.66 (dq, J=12.2, 8.5 Hz, 1H). LC-MS calcd., for C16H18N6O [(M+H)⁺] 311.1, obsd. 311.1.

### Example 62

### 1-methyl-6-[4-(4-methyl-1H-pyrazol-3-yl)-1-piperidyl]-5H-pyrazolo[3,4-d]pyrimidin-4-one (I-115)

From 6-chloro-1-methyl-1,5-dihydro-pyrazolo[3,4-d]pyrimidin-4-one (Intermediate A) and 4-(4-methyl-1H-pyrazol-3-yl)piperidine (CASRN 1316223-49-0) was obtained 1-methyl-6-[4-(4-methyl-1H-pyrazol-3-yl)-1-piperidyl]-5H-pyrazolo[3,4-d]pyrimidin-4-one as a white solid (12.9 mg, 23%). ¹H NMR (400 MHz, DMSO-d₆) δ 12.62 11.65 (m, 1H), 7.75 (s, 1H), 7.69-6.81 (m, 1H), 4.86 4.21 (m, 2H), 3.72 (s, 3H), 3.12 2.96 (m, 2H), 2.96 2.83 (m, 1H), 1.98 (s, 3H), 1.88 1.54 (m, 4H). LC-MS calcd. for C15H19N7O [(M+H)⁺] 314.1, obsd. 314.2.

### Example 63

### 1-methyl-6-[4-(2-pyridylmethyl)-1-piperidyl]-5H-pyrazolo[3,4-d]pyrimidin-4-one (I-117)

From 6-chloro-1-methyl-1,5-dihydro-pyrazolo[3,4-d]pyrimidin-4-one (Intermediate A) and 2-(piperidin-4-ylmethyl)pyridine (CASRN 1316218-40-2) was obtained 1-methyl-6-[4-(2-pyridylmethyl)-1-piperidyl]-5H-pyrazolo[3,4-d]pyrimidin-4-one as a white solid (11.4 mg, 20%). ¹H NMR (400 MHz, DMSO-d₆) δ 10.63 (s, 1H), 8.49 (ddd, J=4.9, 1.9, 0.9 Hz, 1H), 7.73 (s, 1H), 7.69 (td, J=7.6, 1.9 Hz, 1H), 7.27 7.16 (m, 2H), 4.41-4.32 (m, 2H), 3.70 (s, 3H), 2.95 2.82 (m, 2H), 2.67 (d, J=7.1 Hz, 2H), 2.04 (ddq, J=15.0, 7.6, 4.2 Hz, 1H), 1.61 (dd, J=13.5, 3.6 Hz, 2H), 1.28 1.15 (m, 2H). LC-MS calcd. for C17H20N6O [(M+H)⁺] 325.4, obsd. 325.2.

### Example 64

### 6-[4-(4-fluorophenyl)-4-hydroxy-1-piperidyl]-1-methyl-5H-pyrazolo[3,4-d]pyrimidin-4-one (I-93)

From 6-chloro-1-methyl-1,5-dihydro-pyrazolo[3,4-d]pyrimidin-4-one (Intermediate A) and 4-(4-fluorophenyl)piperidin-4-ol (CASRN 3888-65-1) was obtained 6-[4-(4-fluorophenyl)-4-hydroxy-1-piperidyl]-1-methyl-5H-pyrazolo[3,4-d]pyrimidin-4-one as a white solid (12.7 mg, 21%). ¹H NMR (400 MHz, DMSO-d₆) δ 7.75 (s, 1H), 7.57 7.45 (m, 2H), 7.19 7.04 (m, 2H), 5.21 (s, 1H), 4.42 4.28 (m, 2H), 3.71 (s, 3H), 3.29 (m, 2H), 1.94 (td, J=13.1, 4.4 Hz, 2H), 1.65 (d, J=13.4 Hz, 2H). LC-MS calcd. for C17H18FN5O2 [(M+H)⁺] 344.3, obsd. 344.2.

### Example 65

### 1-methyl-6-[4-(4-methyl-124-triazol-3-yl)-1-piperidyl]-5H-pyrazolo[3,4-d]pyrimidin-4-one (I-122)

From 6-chloro-1-methyl-1,5-dihydro-pyrazolo[3,4-d]pyrimidin-4-one (Intermediate A) and 4-(4-methyl-4H-1,2,4-triazol-3-yl)piperidine (CASRN 297172-18-0) was obtained 1-methyl-6-[4-(4-methyl-124-triazol-3-yl)-1-piperidyl]-5H-pyrazolo[3,4-d]pyrimidin-4-one as a white solid (25.9 mg, 46%). ¹H NMR (400 MHz, DMSO-d₆) δ 10.86 (s, 1H), 8.34 (s, 1H), 7.76 (s, 1H), 4.42 (dt, J=13.4, 3.6 Hz, 2H), 3.73 (s, 3H), 3.64 (s, 3H), 3.22-3.07 (m, 3H), 1.94 (dd, J=13.6, 3.7 Hz, 2H), 1.73 (qd, J=11.5, 3.8 Hz, 2H). LC-MS calcd. for C14H18N8O [(M+H)⁺] 315.3, obsd. 315.2.

### Example 66

### 1-methyl-6-[4-(3-pyrazin-2-yl-124-oxadiazol-5-yl)-1-piperidyl]-5H-pyrazolo[3,4-d]pyrimidin-4-one (I-106)

From 6-chloro-1-methyl-1,5-dihydro-pyrazolo[3,4-d]pyrimidin-4-one (Intermediate A) and 5-(piperidin-4-yl)-3-(pyrazin-2-yl)-1,2,4-oxadiazole (CASRN 849925-00-4 was obtained 1-methyl-6-[4-(3-pyrazin-2-yl-124-oxadiazol-5-yl)-1-piperidyl]-5H-pyrazolo[34-d]pyrimidin-4-one as a white solid (11.2 mg, 16%). ¹H NMR (400 MHz, DMSO-d₆) δ 9.25 (dd, J=4.0, 1.4 Hz, 1H), 8.90-8.83 (m, 2H), 7.76 (s, 1H), 4.39 (dt, J=13.6, 4.0 Hz, 2H), 3.73 (s, 3H), 3.53 (td, J=6.8, 3.3 Hz, 1H), 3.30-3.19 (m, 2H), 2.19 (dd, J=13.6, 3.7 Hz, 2H), 1.87 (qd, J=11.1, 3.8 Hz, 2H). LC-MS calcd. for C17H17N9O2 [(M+H)⁺] 380.1, obsd. 380.2.

### Example 67

### 1-methyl-6-(4-phenyl-1-piperidyl)-5H-pyrazolo[3,4-d]pyrimidin-4-one (I-101)

From 6-chloro-1-methyl-1,5-dihydro-pyrazolo[3,4-d]pyrimidin-4-one (Intermediate A) and 4-phenylpiperidine (CASRN 771-99-3) was obtained 1-methyl-6-(4-phenyl-1-piperidyl)-5H-pyrazolo[3,4-d]pyrimidin-4-one as a white solid (14.4 mg, 26%). ¹H NMR (400 MHz, DMSO-d₆) δ 7.75 (s, 1H), 7.36-7.12 (m, 4H), 7.22-7.14 (m, 1H), 4.56 (d, J=13.4 Hz, 2H), 3.72 (s, 3H), 3.01 (t, J=12.0 Hz, 2H), 2.88-2.76 (m, 1H), 1.84 (d, J=13.1 Hz, 2H), 1.63 (qd, J=12.7, 4.0 Hz, 2H). LC-MS calcd. for C17H19N5O [(M+H)⁺] 310.3, obsd. 310.2.

### Example 68

### 6-[4-(3-fluorophenyl)-1-piperidyl]-1-methyl-5H-pyrazolo[3,4-d]pyrimidin-4-one (I-84)

From 6-chloro-1-methyl-1,5-dihydro-pyrazolo[3,4-d]pyrimidin-4-one (Intermediate A) and 4-(3-fluorophenyl)piperidine (CASRN 104774-88-1) was obtained 6-[4-(3-fluorophenyl)-1-piperidyl]-1-methyl-5H-pyrazolo[3,4-d]pyrimidin-4-one as a white solid (8.9 mg, 15%). ¹H NMR (400 MHz, DMSO-d₆) δ 10.41 (s, 1H), 7.75 (s, 1H), 7.39-7.28 (m, 1H), 7.18-7.07 (m, 2H), 7.06-6.96 (m, 1H), 4.61-4.52 (m, 2H), 3.72 (s, 3H), 3.00 (td, J=13.0, 2.5 Hz, 2H), 2.86 (tt, J=12.3, 3.8 Hz, 1H), 1.85 (d, J=12.1 Hz, 2H), 1.64 (qd, J=12.7, 4.0 Hz, 2H). LC-MS calcd. for C17H18N5O [(M+H)⁺] 328.1, obsd. 328.2.

### Example 69

### 1-methyl-6-[4-(4-pyridyl)-1-piperidyl]-5H-pyrazolo[3,4-d]pyrimidin-4-one (I-109)

From 6-chloro-1-methyl-1,5-dihydro-pyrazolo[3,4-d]pyrimidin-4-one (Intermediate A) and 4-(piperidin-4-yl)pyridine ((CASRN 581-45-3) was obtained 1-methyl-6-[4-(4-pyridyl)-1-piperidyl]-5H-pyrazolo[3,4-d]pyrimidin-4-one as a white solid (5.0 mg, 9%). ¹H NMR (400 MHz, DMSO-d₆) δ 8.50 8.43 (m, 2H), 7.76 (s, 1H), 7.33 7.26 (m, 2H), 4.55 (dd, J=12.9, 3.8 Hz, 2H), 3.72 (s, 3H), 3.03 (td, J=13.0, 2.6 Hz, 2H), 2.85 (m, 1H), 1.91 1.78 (m, 2H), 1.80-1.58 (m, 2H). LC-MS calcd. for C16H18N6O [(M−1-H)⁺] 311.1, obsd. 311.2.

### Example 70

### 6-[4-(2-fluorophenyl)-1-piperidyl]-1-methyl-5H-pyrazolo[3,4-d]pyrimidin-4-one (I-88)

From 6-chloro-1-methyl-1,5-dihydro-pyrazolo[3,4-d]pyrimidin-4-one (Intermediate A) and 4-(2-fluorophenyl)piperidine (CASRN 180161-17-5) was obtained 6-[4-(2-fluorophenyl)-1-piperidyl]-1-methyl-5H-pyrazolo[3,4-d]pyrimidin-4-one as a white solid (8.8 mg, 15%). ¹H NMR (400 MHz, DMSO-d₆) δ 10.61 (s, 1H), 7.76 (s, 1H); 7.34 (td, J=7.7, 1.8 Hz, 1H), 7.30-7.21 (m, 1H), 7.21 7.09 (m, 2H), 4.60 4.51 (m, 2H), 3.72 (s, 3H), 3.19 2.99 (m, 3H), 1.86-1.76 (m, 2H), 1.70 (qd, J=12.5, 3.9 Hz, 2H). LC-MS calcd. for C17H18FN5O [(M+H)⁺] 328.1, obsd. 328.2.

### Example 71

### 1-methyl-6-[4-methyl-4-(1H-pyrazol-3-yl)-1-piperidyl]-5H-pyrazolo[3,4-d]pyrimidin-4-one (I-82)

From 6-chloro-1-methyl-1,5-dihydro-pyrazolo[3,4-d]pyrimidin-4-one (Intermediate A) and 4-methyl-4-(1H-pyrazol-3-yl)piperidine (CASRN 1316224-68-6) was obtained 1-methyl-6-[4-methyl-4-(1H-pyrazol-3-yl)-1-piperidyl]-5H-pyrazolo[3,4-d]pyrimidin-4-one as a white solid (12.0 mg, 21%). NMR (400 MHz, DMSO-d₆) δ 13.14 11.98 (m, 1H), 11.22 9.85 (m, 1H), 7.72 (s, 1H), 7.66 7.31 (m, 1H), 6.15 (s, 1H), 4.09 3.75 (m, 1H), 3.51 3.31 (m, 2H), 2.20-1.96 (m, 1H), 1.60 (t, J=9.5 Hz, 1H), 1.22 (s, 3H). LC-MS calcd. for C15H19N7O [(M+H)⁺] 314.1, obsd. 314.1.

### Example 72

### 1-methyl-6-[4-(4-methylsulfonyl-1H-pyrazol-5-yl)-1-piperidyl]-5H-pyrazolo[3,4-d]pyrimidin-4-one (I-81)

From 6-chloro-1-methyl-1,5-dihydro-pyrazolo[3,4-d]pyrimidin-4-one (Intermediate A) and 4-(4-(methylsulfonyl)-1H-pyrazol-5-yl)piperidine was obtained 1-methyl-6-[4-(4-methylsulfonyl-1H-pyrazol-5-yl)-1-piperidyl]-5H-pyrazolo[3,4-d]pyrimidin-4-one as a white solid (18.3 mg, 27%). ¹H NMR (400 MHz, DMSO-d₆) δ 8.03 (s, 1H), 7.74 (s, 1H), 4.54 (d, J=13.3 Hz, 2H), 3.72 (s, 3H), 3.40 (ddd, J=11.9, 8.1, 3.9 Hz, 1H), 3.19 (s, 3H), 3.02 (t, J=12.8 Hz, 2H), 1.91 (d, J=12.3 Hz, 2H), 1.73 (qd, J=12.6, 4.1 Hz, 2H). LC-MS calcd. for C15H19N7O3S [(M+H)⁺] 378.1, obsd. 378.1.

### Example 73

### 1-methyl-6-[4-methyl-4-(3-methyl-1 2 4-oxadiazol-5-yl)-1-piperidyl]-5H-pyrazolo[3,4-d]pyrimidin-4-one (I-91)

From 6-chloro-1-methyl-1,5-dihydro-pyrazolo[3,4-d]pyrimidin-4-one (Intermediate A) and 3-methyl-5-(4-methylpiperidin-4-yl)-1,2,4-oxadiazole (CASRN 1316227-37-8) was obtained 1-methyl-6-[4-methyl-4-(3-methyl-1 2 4-oxadiazol-5-yl)-1-piperidyl]-5H-pyrazolo[3,4-d]pyrimidin-4-one as a white solid (24.9 mg, 42¹H NMR (400 MHz, DMSO-d₆) δ 10.87 (s, 1H), 7.75 (s, 1H), 4.03 (dt, J=14.2, 4.6 Hz, 2H), 3.71 (s, 3H), 3.37 3.23 (m, 2H), 2.34 (s, 3H), 2.21-2.13 (m, 2H), 1.75 (ddd, J=13.7, 9.8, 3.7 Hz, 2H), 1.37 (s, 3H). LC-MS calcd. for C15H19N7O2 [(M+H)⁺] 330.1, obsd. 330.2.

### Example 74

### 1-methyl-6-[4-(1H-pyrazolo[3,4-b]pyridin-3-yl)-1-piperidyl]-5H-pyrazolo[3,4-d]pyrimidin-4-one (I-97)

From 6-chloro-1-methyl-1,5-dihydro-pyrazolo[3,4-d]pyrimidin-4-one (Intermediate A) and 3-(piperidin-4-yl)-1H-pyrazolo[3,4-b]pyridine (CASRN 1185192-81-7) was obtained 1-methyl-6-[4-(1H-pyrazolo[3,4-b]pyridin-3-yl)-1-piperidyl]-5H-pyrazolo[3,4-d]pyrimidin-4-one as a white solid (10.8 mg, 17%). ¹H NMR (400 MHz, DMSO-d₆) δ 13.26 (s, 1H), 8.51 8.43 (m, 1H), 8.31 (dd, J=8.1, 1.6 Hz, 1H), 7.74 (s, 1H), 7.14 (dd, J=8.0, 4.5 Hz, 1H), 4.51 (d, J=13.4 Hz, 2H), 3.72 (s, 3H), 3.38 (m, 1H), 3.17 (t, J=12.3 Hz, 2H), 2.07 (d, J=12.3 Hz, 2H), 1.92-1.78 (m, 2H). LC-MS calcd. for C17H18N8O [(M+H)⁺] 351.1, obsd. 351.2.

### Example 75

### 1-methyl-6-[4-(2-pyridyl)-1-piperidyl]-5H-pyrazolo[3,4-d]pyrimidin-4-one (I-111)

From 6-chloro-1-methyl-1,5-dihydro-pyrazolo[3,4-d]pyrimidin-4-one (Intermediate A) and 2-(piperidin-4-yl)pyridine (CASRN 30532-37-7) was obtained 1-methyl-6-[4-(2-pyridyl)-1-piperidyl]-5H-pyrazolo[3,4-d]pyrimidin-4-one as a white solid (20.7 mg, 37%). ¹H NMR (400 MHz, DMSO-d₆) δ 10.49 (s, 1H), 8.49 (ddd, J=4.8, 1.9, 0.9 Hz, 1H), 7.76 (s, 1H), 7.72 (td, J=7.7, 1.9 Hz, 1H), 7.31 (dt, J=7.9, 1.0 Hz, 1H), 7.21 (ddd, J=7.5, 4.8, 1.1 Hz, 1H), 4.52 (d, J=13.3 Hz, 2H), 3.72 (s, 3H), 3.14 2.92 (m, 3H), 1.90 (dd, J=13.7, 3.6 Hz, 2H), 1.73 (qd, J=12.5, 4.0 Hz, 2H). LC-MS calcd. for C16H18N6O [(M+H)⁺] 311.1, obsd. 311.2.

### Example 76

### 1-methyl-6-[3-(3-methylimidazol-4-yl)pyrrolidin-1-yl]-5H-pyrazolo[3,4-d]pyrimidin-4-one (I-114)

From 6-chloro-1-methyl-1,5-dihydro-pyrazolo[3,4-d]pyrimidin-4-one (Intermediate A) and 1-methyl-5-(pyrrolidin-3-yl)-1H-imidazole was obtained 1-methyl-6-[3-(3-methylimidazol-4-yl)pyrrolidin-1-yl]-5H-pyrazolo[3,4-d]pyrimidin-4-one as a white solid (8.5 mg, 16%). ¹H NMR (400 MHz, DMSO-d₆) δ 7.77 7.72 (m, 1H), 7.54 (dd, J=1.1, 0.5 Hz, 1H), 6.75 (t, J=1.0 Hz, 1H), 4.02 (dd, J=10.3, 7.2 Hz, 1H), 3.78 3.68 (m, 4H), 3.67 3.37 (m, 6H), 2.42-2.29 (m, 1H), 2.00 (dq, J=12.1, 8.6 Hz, 1H). LC-MS calcd. for C14H17N7O [(M+H)⁺] 300.1.1, obsd. 300.2.

### Example 77

### 1-methyl-6-[4-(pyrrolidine-1-carbonyl)-1-piperidyl]-5H-pyrazolo[3,4-d]pyrimidin-4-one (I-129)

From 6-chloro-1-methyl-1,5-dihydro-pyrazolo[3,4-d]pyrimidin-4-one (Intermediate A) and piperidin-4-yl(pyrrolidin-1-yl)methanone (CASRN 35090-95-0) was obtained 1-methyl-6-[4-(pyrrolidine-1-carbonyl)-1-piperidyl]-5H-pyrazolo[3,4-d]pyrimidin-4-one as a white solid (12.8 mg, 22%). ¹H NMR (400 MHz, DMSO-d₆) δ 7.75 (s, 1H), 4.38 (d, J=13.4 Hz, 2H), 3.71 (s, 3H), 3.50 (t, J=6.7 Hz, 2H), 3.27 (t, J=6.9 Hz, 2H), 3.00 (td, J=12.9, 2.6 Hz, 2H), 2.72 (tt, J=11.2, 3.9 Hz, 1H), 1.94 1.83 (m, 2H), 1.83 1.68 (m, 4H), 1.54 (qd, J=12.3, 4.0 Hz, 2H). LC-MS calcd. for C16H22N6O2 [(M+H)⁺] 331.1.1, obsd. 331.2.

### Example 78

### 6-[4-[2 6-difluoro-4-[(4-methylpiperazin-1-yl)methyl]phenyl]piperazin-1-yl]-1-methyl-5H-pyrazolo[3,4-d]pyrimidin-4-one (I-60)

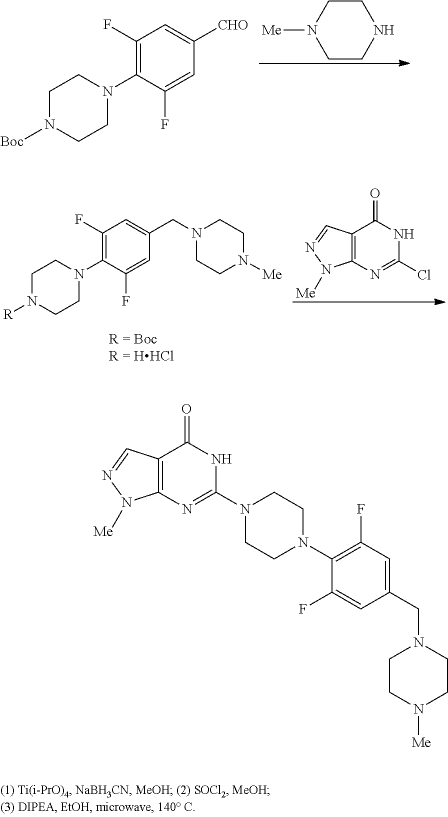

Step 1: To a solution of tert-butyl 4-(2,6-difluoro-4-formylphenyl)piperazine-1-carboxylate (500 mg, 1.53 mmol, 1.00 equiv) in methanol (10 mL) was added 1-methylpiperazine (307 mg, 3.07 mmol, 2.00 equiv) and Ti(i-PrO)₄ (690 mg, 3.07 mmol, 2.00 equiv). The reaction mixture was stirred overnight at RT then NaBH₃CN (193.2 mg, 3.07 mmol, 2.00 equiv) was added. The resulting solution was stirred at RT for 2 h then concentrated under vacuum to remove the excess MeOH. The resulting solution was quenched by the addition of 100 mL of sat'd. aq. NH₄Cl. The solid was removed by filtration. The filtrate was extracted with 2×200 mL of EtOAc. The combined organic layers was washed with 1×150 ml, of brine, dried over anhydrous sodium sulfate and concentrated under vacuum. The residue was purified by SiO₂ chromatography eluting with DCM/MeOH (10:1) to afford 250 mg (40%) of tert-butyl 4-[2,6-difluoro-4-[(4-methylpiperazin-1-yl)methyl]phenyl]piperazine-1-carboxylate as a yellow oil. LCMS (LCMS19, ESI): RT=1.27 min, m/z=411.0 [M+H]⁺

Step 2: To a solution of 1-[[3,5-difluoro-4-(piperazin-1-yl)phenyl]methyl]-4-methylpiperazine hydrochloride (250 mg, 0.72 mmol, 1.00 equiv) in MeOH (20 mL) at 0° C. was added thionyl chloride (2 mL) dropwise. The resulting solution was stirred overnight at RT then concentrated under vacuum. The crude solid was triturated with 100 mL of EtOAc then collected by filtration to give 230 mg of crude 1-[[3,5-difluoro-4-(piperazin-1-yl)phenyl]methyl]-4-methylpiperazine trihydrochloride as a light yellow solid. LCMS (LCM, ESI): RT=0.59 min, m/z=311.1 [M+H]⁺.

Step 3: A 8 mL tube was charged with 1-[2,6-difluoro-4-[(4-methylpiperazin-1-yl)methyl]phenyl]piperazine trihydrochloride (230 mg, 0.55 mmol, 1.00 equiv), DIFA (191 mg, 1.48 mmol, 2.69 equiv) and 6-chloro-1-methyl-1H,4H,5H-pyrazolo[3,4-d]pyrimidin-4-one (137 mg, 0.74 mmol, 1.34 equiv) in EtOH (2 mL), sealed and irradiated in a microwave for 30 min at 140° C. The resulting mixture was cooled to RT and concentrated under vacuum. The residue was first purified by SiO2 chromatography eluting with DCM/MeOH (20:1). The partially purified product was repurified by Prep-HPLC with the following conditions (Prep-HPLC-005): Column, XBridge Prep C18 OBD Column, 5 um, 19*150 mm; mobile phase, water with 10 mmol NH₄HCO₃ and MeCN (25.0% MeCN up to 47.0% in 10 min, up to 95.0% in 1 min, hold 95.0% in 1 min, down to 32.0% in 2 min); Detector, UV 254/220 nm to give 78.2 mg (26%) of 6-(4-[2-fluoro-6-methyl-4-[(4-methylpiperazin-1-yl)methyl]phenyl]piperazin-1-yl)-1-methyl-1H,4H,5H-pyrazolo[3,4-d]pyrimidin-4-one as a white solid. LCMS (LCMS 15, ESI): RT=1.37 min, m/z=459.3 [M+H]⁺. ¹H NMR (400 MHz, DMSO-d₆,) δ: 10.95 (s, 1H), 7.78 (s, 1H), 7.00-6.95 (m, 2H), 3.76-3.73 (m, 7H), 3.40 (s, 2H), 3.20-3.12 (m, 4H), 2.50-2.20 (m, 8H), 2.15 (s, 3H).

### Example 79

### 6-{4-[2,6-Difluoro-4-(2-piperidin-1-yl-ethoxy)-phenyl]-piperazin-1-yl}-1-methyl-1,5-dihydro-pyrazolo[3,4-d]pyrimidin-4-one (I-62)

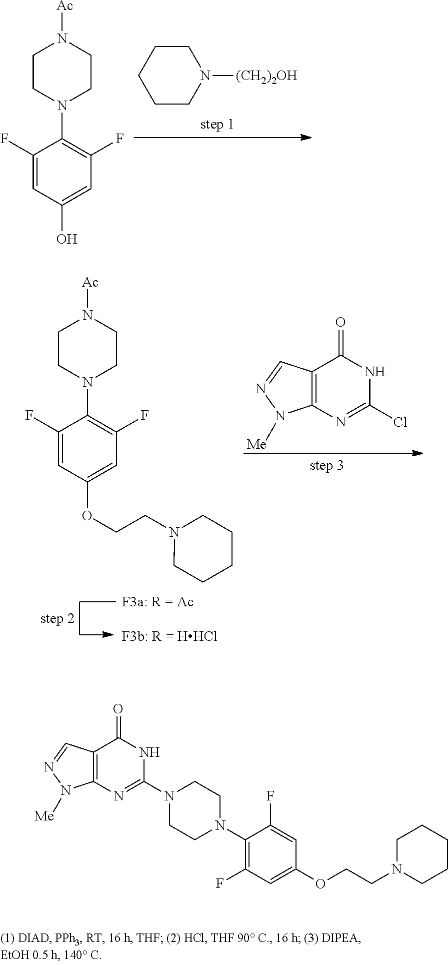

Step 1: To a stirred solution of 1-[4-(2,6-difluoro-4-hydroxyphenyl)piperazin-1-yl]ethan-1-one (256 mg, 1.00 mmol, 1.00 equiv), 2-(piperidin-1-yl)ethan-1-ol (150 mg, 1.16 mmol, 1.16 equiv) and PPh₃ (400 mg, 1.53 mmol, 1.53 equiv) in anhydrous THF (10 mL) was added DIAD (300 mg, 1.48 mmol, 1.49 equiv) dropwise under nitrogen at RT. The resulting solution was then stirred overnight at RT. The resulting mixture was concentrated under vacuum and the residue was triturated in ether for 30 min. The mixture was filtered to remove triphenylphosphine oxide and the filtrate was concentrated under vacuum. The residue was purified by SiO₂ chromatography eluted with 6% MeOH/DCM to afford 0.22 g (60%) of 1-(4-[2,6-difluoro-4-[2-(piperidin-1-yl)ethoxy]phenyl]piperazin-1-yl)ethan-1-one as an off-white solid. TLC: R_{f}=0.8; DCM/MeOH=20:1.

Step 2: To a solution of 1-(4-[2,6-difluoro-4-[2-(piperidin-1-yl)ethoxy]phenyl]piperazin-1-yl)ethan-1-one (220 mg, 0.60 mmol, 1.00 equiv) in THF (4 mL) was added 6M HCl (1 mL). The reaction mixture was stirred overnight at 90° C. The mixture was cooled to RT then concentrated under vacuum to give 0.25 g of crude 1-[2,6-difluoro-4-[2-(piperidin-1-yl)ethoxy]phenyl]piperazine dihydrochloride as an off-white solid. TLC: R_{f}=0.2, DCM/MeOH=1:5.

Step 3: A 25 mL tube was charged with a solution of 6-chloro-1-methyl-1H-pyrazolo[3,4-d]pyrimidin-4-ol (300 mg, 1.63 mmol, 1.00 equiv), 1-[2,6-difluoro-4-[2-(piperidin-1-yl)ethoxy]phenyl]piperazine (582 mg, 1.79 mmol, 1.10 equiv) and DIPEA (630 mg, 4.88 mmol, 3.00 equiv) in EtOH (10 mL), sealed and irradiated in a microwave reactor at 140° C. for 30 min. The reaction mixture was cooled to RT then the solid was collected by filtration and dried under vacuum to yield 64.7 mg (8%) of 1-60 as a white solid. LCMS (LCMS34, ESI): RT=1.84 min; m/z=474.0 [M+1]⁺. ¹HNMR (300 MHz, DMSO-d₆) δ: 10.95 (s, 1H), 7.78 (s, 1H), 6.71 (d, J=11.4 Hz, 2H), 4.02 (t, J=5.8 Hz, 2H), 3.75-3.73 (m, 7H), 3.09-3.07 (m, 4H), 2.59 (t, J=5.5 Hz, 2H), 2.43-2.41 (m, 4H), 1.52-1.47 (m, 4H), 1.38-1.36 (m, 2H).

6-[4-[2,6-difluoro-4-(3-morpholinopropyl)phenyl]piperazin-1-yl]-1-methyl-5H-pyrazolo[3,4-d]pyrimidin-4-one (I-65) was prepared analogously except in step 1,2-(piperidin-1-yl)ethan-1-ol was replaced with 2-(piperidin-1-yl)propan-1-ol: ¹HNMR (300 MHz, DMSO-d₆) δ ppm 10.94 (s, 1H), 7.78 (s, 1H), 6.71 (d, J=11.4 Hz, 2H), 4.09-4.05 (m, 1H), 3.75-3.73 (m, 7H), 3.58-3.55 (m, 4H), 3.07-3.06 (m, 4H), 2.67-2.63 (m, 2H), 2.50-2.43 (m, 4H).

6-[4-[2,6-difluoro-4-(2-morpholinoethoxy)phenyl]piperazin-1-yl]-1-methyl-5H-pyrazolo[3,4-d]pyrimidin-4-one (I-74) was prepared analogously except in step 1,2-(piperidin-1-yl)ethan-1-ol was replaced with 2-(piperidin-1-yl)propan-1-ol: ¹HNMR (300 MHz, DMSO-d₆) δ ppm 10.90 (s, 1H), 7.77 (s, 1H), 6.92 (d, J=10.5 Hz, 2H), 3.75-3.73 (m, 7H), 3.57-3.55 (m, 4H), 3.13-3.12 (m, 4H), 2.57-2.54 (m, 2H), 2.32-2.22 (m, 6H), 1.72-1.67 (m, 2H)

### Example 80

### 6-[4-[2,6-difluoro-4-(2-hydroxyethoxy)phenyl]piperazin-1-yl]-1-methyl-5H-pyrazolo[3 4-d]pyrimidin-4-one (I-78)

4-bromo-3,5-difluoro-phenol was treated with ethylene glycol, urea, zinc oxide and Na₂CO₃ to afford 2-(4-bromo-3,5-difluoro-phenoxy)-ethanol which was subsequently converted to the corresponding benzyl ether (benzyl bromide, NaH, DMF) to afford 5-(2-benzyloxy-ethoxy)-2-bromo-1,3-difluoro-benzene. Palladium-catalyzed amination with 1-boc-piperazine (Pd(OAc)₂, 2,2′-bis(diphenylphosphino)-1,1′-binaphthyl, NaO-tert-Bu) afforded tert-butyl 4-[4-(2-benzyloxy-ethoxy)-2,6-difluoro-phenyl]-piperazine-1-carboxylate.

Deprotection of the carbamate and benzyl ether and condensation of the resulting amine with 6-chloro-1-methyl-1,5-dihydro-pyrazolo[3,4-d]pyrimidin-4-one (Intermediate A) afforded the title compound.

### Example 81

### 6-[4-[4-(1 2-dihydroxyethyl)-2 6-difluoro-phenyl]piperazin-1-yl]-1-methyl-5H-pyrazolo[3,4-d]pyrimidin-4-one (I-63)

Palladium/zinc-mediated displacement of bromine in 1-[4-(4-bromo-2,6-difluoro-phenyl)-piperazin-1-yl]-ethanone the vinyl Grignard (ZnCl₂, Pd(PPh₃)₄, THF, 60° C., 4 h) afforded 1-[4-(2,6-difluoro-4-vinyl-phenyl)-piperazin-1-yl]-ethanone which is treated with OsO₄ ((N-methylmorpholine oxide, H₂O, THF RT, 18 h) and subsequently deacetylated (HCl) to afford 1-(3,5-difluoro-4-piperazin-1-yl-phenyl)-ethane-1,2-diol. Condensation of the resulting amine with 6-chloro-1-methyl-1,5-dihydro-pyrazolo[3,4-d]pyrimidin-4-one (Intermediate A) afforded the title compound.

### Example 82

### 2-[3,5-difluoro-4-[4-(1-methyl-4-oxo-5H-pyrazolo[3,4-d]pyrimidin-6-yl)piperazin-1-yl]phenoxy]ethyl (2S)-2-amino-3-methyl-butanoate (I-61)

Step 1: To a stirred mixture of (2S)-2-amino-3-methylbutanoic acid (3 g, 25.61 mmol, 1.00 equiv) and K₂CO₃ (7.08 g, 51.23 mmol, 2.00 equiv) in water (20 mL) and THF (30 mL) at 0° C. was added dropwise a solution of di-tert-butyl dicarbonate (8.4 g, 38.49 mmol, 1.50 equiv) in THF (10 mL). The resulting mixture was stirred at 25° C. for 2 h. Water (50 mL) was added and the resulting mixture was extracted with DCM (3×100 mL). The combined organic layers were dried (Na₂SO₄), filtered and concentrated under vacuum to give 3 g of crude (2S)-2-[[(tert-butoxy)carbonyl]amino]-3-methylbutanoic acid as a colorless solid: LC-MS calcd for C₁₀H₁₉NO₄ [(M+H)⁺] 218, obsd.218.0.

Step 2: A mixture of (2S)-2-[[(tert-butoxy)carbonyl]amino]-3-methylbutanoic acid (321 mg, 1.48 mmol, 3.00 equiv), 6-[4-[2,6-difluoro-4-(2-hydroxyethoxy)phenyl]piperazin-1-yl]-1-methyl-1H-pyrazolo[3,4-d]pyrimidin-4-ol (I-78, 200 mg, 0.49 mmol, 1.00 equiv), EDC.HCl (123 mg, 0.64 mmol, 1.30 equiv), DIPEA (127 mg, 0.98 mmol, 2.00 equiv) and DMAP (30 mg, 0.25 mmol, 0.50 equiv) in DMF (5 mL) was stirred at 25° C. for 10 h. The resulting mixture was concentrated under vacuum and the residue was purified by SiO2 chromatography eluting with DCM/MeOH (30:1) to afford 100 mg (34%) of 2-[3,5-difluoro-4-(4-[4-hydroxy-1-methyl-1H-pyrazolo[3,4-d]pyrimidin-6-yl]piperazin-1-yl)phenoxy]ethyl (2S)-2-[[(tert-butoxy)carbonyl]amino]-3-methylbutanoate as a light yellow solid: LC-MS calcd for C28H37F2N7O6 [(M+H)⁺] 606, obsd.606.0.

Step 3: Thionyl chloride (2 mL) was added dropwise to MeOH (5 mL) with stirring at 0° C. 2-[3,5-Difluoro-4-(4-[1-methyl-4-oxo-1H,4H,5H-pyrazolo[3,4-d]pyrimidin-6-yl]piperazin-1-yl)phenoxy]ethyl (2S)-2-[[(tert-butoxy)carbonyl]amino]-3-methylbutanoate (100 mg, 0.17 mmol, 1.00 equiv) was then added and the resulting solution was stirred at 25° C. for 2 h. The resulting mixture was concentrated under vacuum. The crude product was purified by CombiFlash Prep-MPLC with the following conditions (IntelFlash-1): Column, C18; mobile phase, H₂O:CH₃CN=9:1 increasing to H₂O:CH₃CN=1:1 within 14 min; Detector, UV 254 nm to give 19.9 mg (22%) of 2-[3,5-difluoro-4-(4-[1-methyl-4-oxo-1H,4H,5H-pyrazolo[3,4-d]pyrimidin-6-yl]piperazin-1-yl)phenoxy]ethyl (2S)-2-amino-3-methylbutanoate hydrochloride as a white solid.

¹H NMR (400 MHz, DMSO-d₆) δ ppm 10.90 (s, 1H), 8.48-8.47 (m, 2H), 7.79 (s, 1H), 6.77-6.76 (m, 2H), 6.66-6.00 (m, 2H), 4.61-4.59 (m, 1H), 4.45-4.41 (m, 1H), 4.25-4.23 (m, 2H), 3.98-3.95 (m, 1H), 3.75-3.73 (m, 7H), 3.08 (s, 4H), 2.21-2.13 (m, 1H), 1.00-0.94 (m, 6H)

### Example 83

### 2-[3,5-difluoro-4-[4-(1-methyl-4-oxo-5H-pyrazolo[3,4-d]pyrimidin-6-yl)piperazin-1-yl]phenoxy]ethyl dihydrogen phosphate (I-75)

Step 1: Sodium hydride (230 mg, 5.75 mmol, 2.92 equiv, 60%) was added to a solution of 6-[4-[2,6-difluoro-4-(2-hydroxyethoxy)phenyl]piperazin-1-yl]-1-methyl-1H,4H,5H-pyrazolo[3,4-d]pyrimidin-4-one (I-78, 800 mg, 1.97 mmol, 1.00 equiv) in anhydrous THF (30 mL) at 0° C. The resulting mixture was warmed to 25° C. and stirred for 1 h. Dibenzyl [[bis-(benzyloxy)phosphoryl]oxy]phosphonate (3.18 g, 5.91 mmol, 3.00 equiv) was then added in portions at 25° C. The resulting solution was stirred overnight at 25° C. The reaction mixture was diluted with 50 mL of ethyl acetate then washed with H₂O (2×30 mL) and brine (2×30 mL). The organic layer was dried (Na₂SO₄), filtered and concentrated under vacuum to afford 0.6 g (46%) of crude dibenzyl 2-[3,5-difluoro-4-(4-[1-methyl-4-oxo-1H,4H,5H-pyrazolo[3,4-d]pyrimidin-6-yl]piperazin-1-yl)phenoxy]ethyl phosphate as a white solid: LC-MS calcd for C₃₂H₃₃F₂N₆O₆P [(M+H)⁺] 666, obsd. 666.0.

Step 2: To a mixture of dibenzyl 2-[3,5-difluoro-4-(4-[4-hydroxy-1-methyl-1H-pyrazolo[3,4-d]pyrimidin-6-yl]piperazin-1-yl)phenoxy]ethyl phosphate (500 mg, 0.75 mmol, 1.00 equiv) and 10% palladium on carbon (0.1 g) in MeOH (100 mL) was added a solution of NaHCO₃ (190 mg) in 15 mL of water. The mixture was stirred under 1 atmosphere of hydrogen at RT for 1 h. The catalyst was removed by filtration and the filtrate was concentrated under vacuum. The residue was partially purified on a C18 column eluting with water/MeOH (4/6). The product was re-purified by Prep-HPLC with the following conditions (Prep-HPLC-005): Column, XBridge Prep C18 OBD, 5 um, 19×150 mm; mobile phase, water with 10 mmol NH₄HCO₃ and MeCN (32.0% MeCN up to 60.0% in 10 min, up to 95.0% in 1 min, hold 95.0% in 1 min, down to 32.0% in 2 min); detector, UV 254/220 nm to give 0.057 g (16%) of 1-75 as a solid: ¹H NMR (300 MHz, D₂O) δ ppm 7.80 (s, 1H), 6.54 (d, J=13.2 Hz, 2H), 4.15-4.05 (m, 4H), 3.81-3.75 (m, 4H), 3.75 (s, 3H), 3.21-3.09 (m, 4H). LC-MS calcd for C₁₈H₂₁F₂N₆O₆P [(M+H)⁺] 486, obsd. 487.0.

### Example 84

### 6-[4-[4-(2 3-dihydroxypropyl)-2 6-difluoro-phenyl]piperazin-1-yl]-1-methyl-5H-pyrazolo[3,4-d]pyrimidin-4-one (I-66)

Palladium-mediate reaction of vinyl-tributyl tin (Pd(PPh₃)₄, DMF, 100° C. 18 h) and intermediate M afforded 1-[4-(4-allyl-2,6-difluoro-phenyl)-piperazin-1-yl]-ethanone. Osmium-catalyzed dihydroxylation as described in Example 83, hydrolysis of the amide NaOH(H2O, 100° C.) and condensation with Intermediate A afforded the title compound.

### Example 85

### 6-[4-[2,6-difluoro-4-(morpholinomethyl)phenyl]piperazin-1-yl]-1-methyl-5H-pyrazolo[3 4-d]pyrimidin-4-one (I-67)

The title compound was prepared in analogously with the procedure in Example 78 except in step 1, N-methyl-piperazine was replaced with morpholine to afford the title compound.

### Example 86

### 6-[4-[4-(2 3-dihydroxypropoxy)-2 6-difluoro-phenyl]piperazin-1-yl]-1-methyl-5H-pyrazolo[3,4-d]pyrimidin-4-one (I-68)

Intermediate L is alkylated with allyl bromide (K₂CO₃, DMF, 40° C., 18 h) and dihydroxylated as described in Example 83. Deacetylation and condensation with Intermediate A afforded the title compound.

### Example 87

### 6-[4-[2,6-difluoro-4-(1-hydroxyethyl)phenyl]piperazin-1-yl]-1-methyl-5H-pyrazolo[3,4-d]pyrimidin-4-one (I-69) and 6-[4-[2,6-difluoro-4-(1-methoxyethyl)phenyl]piperazin-1-yl]-1-methyl-5H-pyrazolo[3,4-d]pyrimidin-4-one (I-71)

Addition of MeLi to Intermediate M afforded tert-butyl 4-[2,6-difluoro-4-(1-hydroxyethyl)-phenyl]-piperazine-1-carboxylate. Deprotection (HCl/MeOH) affords a mixture of the desired alcohol (10%) and the corresponding methyl ether (80%) which were separated and each condensed with Intermediate A to afford the title compounds.

I-69: ¹HNMR (400 MHz, DMSO-d₆) δ ppm 10.62 (s, 1H), 7.78 (s, 1H), 7.04-6.98 (m, 2H), 5.31 (d, J=4.4 Hz, 1H), 4.68-4.65 (m, 1H), 3.77-3.73 (m, 7H), 3.17-3.12 (m, 4H), 1.30 (d, J=6.8 Hz, 3H).

I-70: ¹HNMR (400 MHz, DMSO-d₆) δ ppm 10.96 (s, 1H), 7.79 (s, 1H), 6.90-6.86 (m, 2H), 4.71-4.70 (m, 1H), 3.75-3.73 (m, 7H), 3.32-3.10 (m, 8H), 2.23-2.13 (m, 4H).

### Example 88

### 6-[4-[2,6-difluoro-4-[(1-oxothietan-3-yl)methoxy]phenyl]piperazin-1-yl]-1-methyl-5H-pyrazolo[3,4-d]pyrimidin-4-one (I-72)

3-Thietane-carboxaldehyde (CASRN 87373-79-3) can be reduced with NaBH₄ to afford thietan-3-yl-methanol which can be treated with Intermediate N under Mitsunobu conditions which will afford tert-butyl 4-[2,6-difluoro-4-(thietan-3-ylmethoxy)-phenyl]-piperazine-1-carboxylate. Deprotection of the Boc protecting group can be accomplished with HCl/MeOH and the piperazine nitrogen condensed with Intermediate A to afford 6-{4-[2,6-difluoro-4-(thietan-3-ylmethoxy)-phenyl]-piperazin-1-yl}-1,5-dihydro-pyrazolo[3,4-d]pyrimidin-4-one. Oxidation of the sulfur to the sulfoxide will afford the title compound.

### Example 89

### 6-[4-[4-(1 1-dioxothian-4-yl)oxy-2 6-difluoro-phenyl]piperazin-1-yl]-1-methyl-5H-pyrazolo[3,4-d]pyrimidin-4-one (I-70)

Tetrahydrothiopyran-4-ol (CASRN 29683-23-6) can be converted to the corresponding tosylate and reacted with Intermediate N under Mitsunobu conditions which will afford tert-butyl 4-[2,6-difluoro-4-(thietan-3-ylmethoxy)-phenyl]-piperazine-1-carboxylate. Deprotection of the Boc protecting group can be accomplished with HCl/MeOH and the piperazine nitrogen condensed with Intermediate A to afford 6-{4-[2,6-difluoro-4-(thietan-3-ylmethoxy)-phenyl]-piperazin-1-yl}-1,5-dihydro-pyrazolo[3,4-d]pyrimidin-4-one. Oxidation of the sulfide to the sulfone will afford the title compound: ¹HNMR (400 MHz, DMSO-d₆) δ ppm 10.96 (s, 1H), 7.79 (s, 1H), 6.90-6.86 (m, 2H), 4.71-4.70 (m, 1H), 3.75-3.73 (m, 7H), 3.32-3.10 (m, 8H), 2.23-2.13 (m, 4H).

6-[4-[2 6-difluoro-4-(1-oxothian-4-yl)oxy-phenyl]piperazin-1-yl]-1-methyl-5H-pyrazolo[3,4-d]pyrimidin-4-one (I-87) is prepared analogously except in the final step the sulfide is oxidized to the sulfoxide: ¹HNMR (400 MHz, DMSO-d₆) δ ppm 7.78 (s, 1H), 6.84-6.81 (m, 2H), 4.68-4.67 (m, 1H), 3.74-3.73 (m, 7H), 3.30-3.29 (m, 1H), 2.92-2.90 (m, 2H), 2.70-2.67 (m, 2H), 2.35-2.29 (m, 2H), 1.86-1.82 (m, 1H)

6-[4-(2 6-difluoro-4-tetrahydropyran-4-yloxy-phenyl)piperazin-1-yl]-1-methyl-5H-pyrazolo[3,4-d]pyrimidin-4-one (I-76) is prepared analogously be replacing thietan-3-yl-methanol with 4-hydroxy-tetrahydropyran: ¹HNMR (300 MHz, DMSO-d₆) δ ppm 10.97 (s, 1H), 7.80 (s, 1H), 6.81-6.82 (d, J=3.6 Hz, 2H), 4.58-4.57 (m, 1H), 3.87-3.74 (m, 10H), 3.48-3.47 (m, 2H), 3.09 (s, 1H), 1.98-1.95 (m, 2H), 1.61-1.57 (m, 2H)

### Example 90

### 6-[4-(2 6-difluoro-4-hydroxy-phenyl)piperazin-1-yl]-1-methyl-5H-pyrazolo[3,4-d]pyrimidin-4-one

Condensation of Intermediate L and Intermediate A will afford the title compound: ¹H-NMR (300 MHz, DMSO-d₆) δ ppm 10.96 (s, 1H), 10.11 (s, 1H), 7.78 (s, 1H), 6.44 (d, J=11.1 Hz, 2H), 3.73-3.19 (m, 7H), 3.03-3.01 (m, 4H).

6-[4-(2 6-difluorophenyl)piperazin-1-yl]-1 5-dihydropyrazolo[3,4-d]pyrimidin-4-one (I-80) was prepared analogously except Intermediate L was replaced with 1-(2,6)-difluoro-phenyl)-piperazine (CASRN 255893-56-2): DH-NMR (300 MHz, DMSO-d₆) δ ppm 12.96 (s, 1H), 10.96 (s, 1H), 7.81 (s, 1H), 7.15-7.02 (m, 3H), 3.71-3.69 (m, 4H), 3.23-3.12 (m, 41-1)

### Example 91

### 6-[4-[2,6-difluoro-4-(2-methoxyethoxy)phenyl]piperazin-1-yl]-1 5-dihydropyrazolo[3,4-d]pyrimidin-4-one (I-77)

The title compound is prepared by condensation of Intermediate D and Intermediate L (DIPEA, EtOH) to afford the title compound: ¹H-NMR (300 MHz, DMSO-d₆) δ ppm 12.95 (s, 1H), 10.94 (s, 1H), 7.79 (s, 1H), 6.75 (d, J=11.1 Hz, 2H), 4.09-4.06 (t, J=4.4 Hz, 2H), 3.72-3.62 (m, 4H), 3.64-3.61 (m, 2H), 3.29 (s, 3H), 3.09-3.01 (m, 4H).

### Example 92

### 6-[4-[2,6-difluoro-4-(hydroxymethyl)phenyl]piperazin-1-yl]-1-methyl-5H-pyrazolo[3,4-d]pyrimidin-4-one (I-79))

Intermediate M could be reduced to the primary alcohol with NaBH4 and deprotected with HCl/dioxane to afford (3,5-difluoro-4-piperazin-1-yl-phenyl)-methanol which was condensed with intermediate A to afford the title compound: ¹H NMR (300 MHz, DMSO-d₆) δ ppm 10.99 (s, 1H), 7.81 (s, 1H), 6.98-7.02 (d, J=10.2 Hz, 2H), 5.36 (t, J=5.7 Hz, 1H), 4.46-4.44 (d, J=6.0 Hz, 2H), 3.78-3.75 (m, 7H), 3.16-3.17 (m, 4H).7

### Example 93

### 6-[4-[2,6-difluoro-4-(2-methoxyethoxy)phenyl]-4-hydroxy-1-piperidyl]-1-methyl-5H-pyrazolo[3,4-d]pyrimidin-4-one (I-83)

Step 1: To a stirred solution of 2-bromo-1,3-difluoro-5-(2-methoxyethoxy)benzene (3 g, 11.23 mmol, 1.00 equiv) in ether (100 mL) maintained under nitrogen at −78° C. was added dropwise a 2.5M solution of n-butyllithium (4.98 mL, 1.10 equiv) in hexane. The resulting solution was stirred at −78° C. for 2 h. A solution of tert-butyl 4-oxopiperidine-1-carboxylate (2.67 g, 13.40 mmol, 1.20 equiv) in ether (20 mL) was added dropwise at −78° C. The resulting solution was stirred for 2 h while warmed slowly to 25° C. The reaction was quenched by the addition of 50 mL of water and then extracted with Et₂O (3×50 mL). The combined organic layers were dried (Na₂SO₄), filtered and concentrated under vacuum to afford 5 g of crude tert-butyl 4-[2,6-difluoro-4-(2-methoxyethoxy)phenyl]-4-hydroxypiperidine-1-carboxylate as light yellow oil: LC-MS calcd. for C₁₉H₂₇F₂NO₅ [(M+H)⁺] 388, obsd. 388.2.

Step 2: To a stirred solution of tert-butyl 4-[2,6-difluoro-4-(2-methoxyethoxy)phenyl]-4-hydroxypiperidine-1-carboxylate (5 g, 12.91 mmol, 1.00 equiv) in EtOAc (400 mL) at 0° C. was added dropwise a 3M HCl (8 mL, 2.00 equiv) solution. The reaction mixture was stirred at 25° C. overnight and then concentrated under vacuum to give 3 g of crude 4-[2,6-difluoro-4-(2-methoxyethoxy)phenyl]piperidin-4-ol hydrochloride as a white solid: LC-MS calcd for C₁₄H₂₀ClF₂NO₃ [(M+H)⁺] 288, obsd. 288.2.

Step 3: A 20 mL tube was charged with a mixture of 6-chloro-1-methyl-1H,4H,5H-pyrazolo[3,4-d]pyrimidin-4-one (173 mg, 0.94 mmol, 1.00 equiv), DIPEA (606.8 mg, 4.70 mmol, 5.00 equiv) and 4-[2,6-difluoro-4-(2-methoxyethoxy)phenyl]piperidin-4-ol hydrogen chloride (270 mg, 0.94 mmol, 1.00 equiv) in EtOH (10 mL), the tube was flushed with N₂, sealed and irradiated in a microwave at 140° C. for 30 min. The reaction mixture was cooled to 25° C. and then concentrated under vacuum. The crude product (200 mg) was purified by Prep-HPLC with the following conditions (Pre-HPLC-006 (Waters)): Column, XSelect CSH Prep C18 OBD Column, 5 μm, 19×150 mm; mobile phase, Water with 10 mmol NH₄HCO₃ and CH₃CN (5.0% CH₃CN up to 30.0% in 10 min, up to 95.0% in 1 min, hold 95.0% in 2 min, down to 5.0% in 2 min); Detector, UV 254/220 nm to give 90 mg (22%) of 6-[4-[2,6-difluoro-4-(2-methoxyethoxy)phenyl]-4-hydroxypiperidin-1-yl]-1-methyl-1H,4H,5H-pyrazolo[3,4-d]pyrimidin-4-one as a yellow solid: ¹H NMR (300 MHz, DMSO-d₆) δ ppm 10.78 (s, 1H), 7.75 (s, 1H), 6.69-6.60 (m, 2H), 5.38 (s, 1H), 4.24-4.19 (m, 2H), 4.11-4.08 (m, 2H), 3.71 (s, 3H), 3.63-3.60 (m, 2H), 3.42-3.40 (m, 2H), 3.28 (s, 3H), 2.09-2.06 (m, 4H). LC-MS calcd for C₁₄H₂₀ClF₂NO₃ [(M+H)⁺] 436, obsd. 436.1

### Example 94

### 6-[4-[2 6-difluoro-4-(oxetan-3-yloxy)phenyl]piperazin-1-yl]-1-methyl-5H-pyrazolo[3,4-d]pyrimidin-4-one (I-85)

Condensation of 3-(4-methylbenzenesulfonate)-3-oxetanol (CASRN 26272-83-3) and 4-bromo-3,5-difluorophenol under Mirsunobu conditions (PPh₃, DIAD, DCM, RT) afforded 3-(4-bromo-3,5-difluoro-phenoxy)-oxetane which was condensed tert-butyl piperazine-1-carboxylate utilizing palladium coupling described in Intermediate I. Removal of the boc (TFA/DCM) and condensation with Intermediate A (DIPEA, EtOH, 140° C.) affords the title compound: ¹H NMR (300 MHz, DMSO-d₆) δ ppm 10.96 (s, 1H), 7.80 (s, 1H), 6.61 (d, J=10.8 Hz, 2H), 5.30-5.26 (m, 1H), 4.95-4.91 (m, 2H), 4.54-4.50 (m, 2H), 3.76-3.74 (m, 7H), 3.10-3.09 (m, 4H).

### Example 95

### 4-[2,6-difluoro-4-(2-methoxyethoxy)phenyl]-1-(1-methyl-4-oxo-5H-pyrazolo[3,4-d]pyrimidin-6-yl)piperidine-4-carbonitrile (I-86)

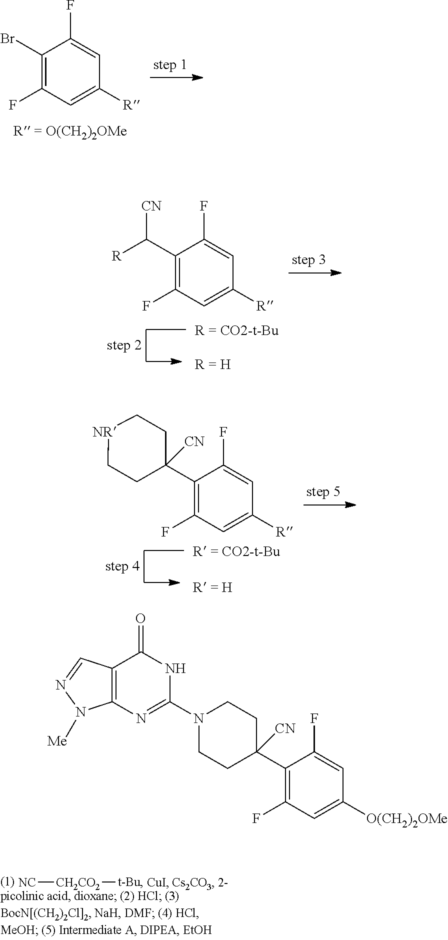

1-[2,6-Difluoro-4-(2-methoxy-ethoxy)-phenyl]-cyclohexanecarbonitrile was prepared as herein described and condensed with intermediate A to afford the title compound: ¹H-NMR (300 MHz, DMSO-d₆) δ ppm 10.92 (s, 1H), 7.78 (s, 1H), 6.87-6.83 (m, 2H), 4.55-4.50 (m, 2H), 4.15 (t, J=4.4 Hz, 2H), 3.73 (s, 3H), 3.63 (t, J=4.4 Hz, 2H), 3.31-3.19 (m, 5H), 2.41-2.23 (m, 4H).

### Example 96

### 1-methyl-6-[4-methyl-4-(1-methylpyrazol-3-yl)-1-piperidyl]-5H-pyrazolo[3,4-d]pyrimidin-4-one (I-90)

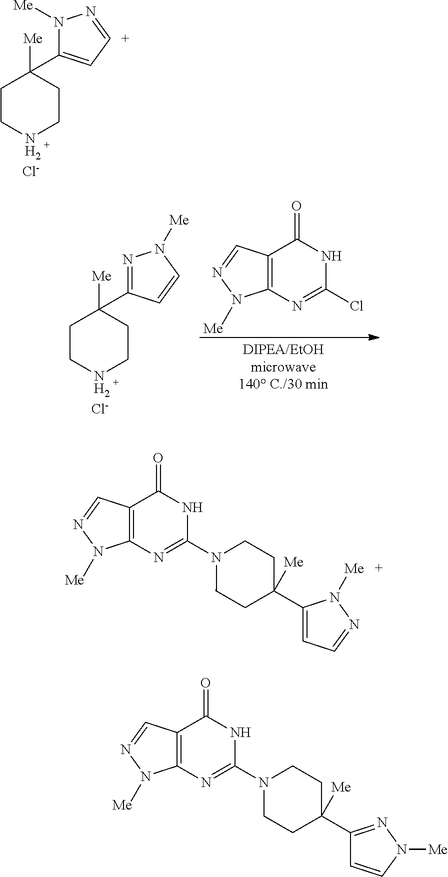

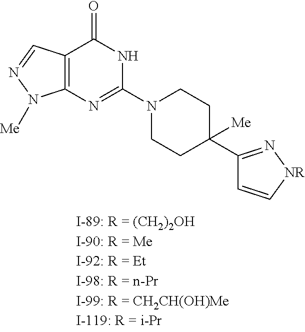

Step 1: A solution of 1-[(tert-butoxy)carbonyl]-4-methylpiperidine-4-carboxylic acid (CASRN 189321-63-9, 10 g, 41.10 mmol, 1.00 equiv), HATU (18.7 g, 49.21 mmol, 1.20 equiv) and DIPEA (26.6 g, 206.20 mmol, 5.00 equiv) in DMF (100 mL) was stirred RT for 30 min. Methoxy(methyl)amine hydrochloride (4.8 g, 49.21 mmol, 1.20 equiv) was then added and the resulting solution was stirred overnight at 25° C. The reaction was quenched by the addition of 200 mL of water and then extracted with EtOAc (3×100 mL). The combined organic layers were washed with brine (3×50 mL), dried (Na₂SO₄), filtered and concentrated under vacuum. The residue was purified by SiO₂ chromatography eluting with EtOAc/petroleum ether (25/75) to afford 10 g (85%) of tert-butyl 4-[methoxy(methyl)carbamoyl]-4-methylpiperidine-1-carboxylate as a light yellow oil: LC-MS calcd for C₁₄H₂₆N₂O₄ [(M+Na)⁺] 309, obsd. 309.1

Step 2: To a stirred solution of tert-butyl 4-[methoxy(methyl)carbamoyl]-4-methylpiperidine-1-carboxylate (10.4 g, 36.32 mmol, 1.00 equiv) in anhydrous THF (200 mL) maintained under nitrogen at 0° C. was added dropwise a 3.0M solution of MeMgBr (56 4 mL, 4.00 equiv) in ether. The resulting solution was stirred overnight at 25° C. then quenched by the addition of 300 mL of sat'd. aq. NH₄Cl. The resulting mixture was extracted with EtOAc (3×150 mL). The combined organic layers were washed with brine (3×50 mL), dried (Na₂SO₄), filtered and concentrated under vacuum to afford 8.5 g (97%) of crude tert-butyl 4-acetyl-4-methylpiperidine-1-carboxylate as a colorless oil. TLC: Rf=0.5; ethyl acetate/petroleum ether=1:2.

Step 3: To a solution of tert-butyl 4-acetyl-4-methylpiperidine-1-carboxylate (10 g, 41.44 mmol, 1.00 equiv) in toluene (300 mL) was added DMF-DMA (49.6 g, 416.81 mmol, 10.00 equiv). The reaction mixture was stirred at 115° C. for 48 h. The resulting solution was cooled to RT and then concentrated under vacuum. The residue was diluted with 100 mL of EtOAc then washed with brine (3×20 mL). The organic layer was dried (Na₂SO₄) and concentrated in vacuum. The residue was purified by SiO₂ chromatography eluting with petroleum ether/EtOAc (1/1) to afford 4.6 g (37%) of tert-butyl 4-[(2E)-3-(dimethylamino)prop-2-enoyl]-4-methylpiperidine-1-carboxylate as a colorless oil. TLC: R_{f}=0.2; ethyl acetate/petroleum ether=1:2.

Step 4: A solution of tert-butyl 4-[(2E)-3-(dimethylamino)prop-2-enoyl]-4-methylpiperidine-1-carboxylate (4.6 g, 15.52 mmol, 1.00 equiv) and NH₂NH₂.H₂O (3.9 g, 77.60 mmol, 5.00 equiv) in EtOH (150 mL) was refluxed for 2 h. The resulting mixture was cooled to RT then concentrated under vacuum. The residue was diluted with 150 mL of EtOAc then washed with 2×20 mL of brine, dried (Na₂SO₄), filtered and concentrated in vacuum. The residue was purified by SiO₂ chromatography eluting with EtOAc/petroleum ether (1/4) to yield 3.2 g (78%) of tert-butyl 4-methyl-4-(1H-pyrazol-3-yl)piperidine-1-carboxylate as a colorless oil: LC-MS calcd for C₁₄H₂₃N₃O₂ [(M+H)⁺] 266, obsd. 266.1.

Step 5: Sodium hydride (226 mg, 5.65 mmol, 5.00 equiv) was added in portions to a stirred solution of tert-butyl 4-methyl-4-(1H-pyrazol-3-yl)piperidine-1-carboxylate (300 mg, 1.13 mmol, 1.00 equiv) in anhydrous THF (15 mL) at 0° C. The reaction mixture was stirred for at 0° C. 1 h. Iodomethane (344 mg, 2.42 mmol, 2.00 equiv) was then added at 0° C. and the resulting solution was stirred overnight at 25° C. The reaction was quenched with 20 mL of water and then extracted with EtOAc (3×20 mL). The combined organic layers were washed with brine (3×10 mL), dried (Na₂SO₄), filtered and concentrated under vacuum to afford 260 mg (82%) of a mixture of tert-butyl 4-methyl-4-(1-methyl-1H-pyrazol-3-yl)piperidine-1-carboxylate and tert-butyl 4-methyl-4-(1-methyl-1H-pyrazol-5-yl)piperidine-1-carboxylate as a colorless oil: LC-MS calcd for C₁₅H₂₅N₃O₂ [(M+H)⁺] 280, obsd. 280.1

Step 6: The mixture pyrazoles from step 5 (260 mg, 0.93 mmol, 1.00 equiv) was dissolved in a saturated solution of hydrogen chloride in MeOH1 (20 mL). The reaction mixture was stirred overnight at 25° C. then concentrated under vacuum to afford a crude mixture of 220 mg of 4-methyl-4-(1-methyl-1H-pyrazol-3-yl)piperidine hydrochloride and 4-methyl-4-(1-methyl-1H-pyrazol-5-yl)piperidine hydrochloride as a colorless oil which were used in the next step with further purification: LC-MS calcd for C₁₀H₁₇N₃ [(M+H)⁺] 180, obsd. 180.1

Step 7: A solution of 4-methyl-4-(1-methyl-1H-pyrazol-3-yl)piperidine hydrochloride and 4-methyl-4-(1-methyl-1H-pyrazol-5-yl)piperidine hydrochloride (110 mg, 0.51 mmol, 1.00 equiv), 6-chloro-1-methyl-1H,4H,5H-pyrazolo[3,4-d]pyrimidin-4-one (94.2 mg, 0.51 mmol, 1.00 equiv) and DIPEA (330 mg, 2.55 mmol, 5.01 equiv) in EtOH (3 mL) was irradiated in a microwave for 30 min at 140° C. The reaction mixture was cooled RT then concentrated under vacuum. The residue was first purified by SiO2 chromatography eluting with EtOAc/petroleum ether (90/100). The product (300 mg) was re-purified by prep-HPLC with the following conditions (Pre-HPLC-006(Waters)): Column, XSelect CSH Prep C18 OBD Column, 5 m, 19×150 mm; mobile phase, water with 10 mmol NH₄HCO₃ and CH₃CN (10.0% CH₃CN up to 27.0% in 10 min then up to 95.0% in 1 min, hold 95.0% in 1 min, down to 10.0% in 2 min); detector, UV 254/220 nm to afford 86.3 mg (50%) of 1-methyl-6-[4-methyl-4-(1-methyl-1H-pyrazol-3-yl)piperidin-1-yl]-1H,4H,5H-pyrazolo[3,4-d]pyrimidin-4-one (I-96) as a white solid and 46.2 mg (38%) of 1-methyl-6-[4-methyl-4-(1-methyl-1H-pyrazol-5-yl)piperidin-1-yl]-1H,4H,5H-pyrazolo[3,4-d]pyrimidin-4-one (I-90) as a white solid.

I-89: ¹H-NMR (300 MHz, DMSO-d₆) δ ppm 7.72 (s, 1H), 7.59 (s, 1H), 6.14 (s, 1H), 4.81 (t, J=5.4 Hz, 1H), 4.08 (t, J=5.4 Hz, 2H), 3.95-3.91 (m, 2H), 3.72-3.67 (m, 5H), 3.38-3.29 (m, 2H), 2.10-2.05 (m, 2H), 1.56 (d, J=5.4 Hz, 2H), 1.20 (s, 3H).

I-90: ¹H NMR (400 MHz, DMSO-d₆) δ ppm 10.79 (s, 1H), 7.74 (s, 1H), 7.57 (d, J=2.4 Hz, 1H), 6.14 (d, J=2.4 Hz, 1H), 3.95-3.91 (m, 2H), 3.78 (s, 3H), 3.70 (s, 3H), 3.37-3.30 (m, 2H), 2.10-2.06 (m, 2H), 1.60-1.53 (m, 2H), 1.19 (s, 3H).

I-92: ¹H-NMR (300 MHz, DMSO-d₆) δ ppm 10.79 (s, 1H), 7.74 (s, 1H), 7.62 (s, 1H), 6.14 (s, 1H), 4.10-4.04 (m, 2H), 3.97-3.94 (m, 2H), 3.70 (s, 3H), 3.36-3.29 (m, 2H), 2.10-2.06 (m, 2H), 1.59-1.57 (m, 2H), 1.36 (t, J=5.4 Hz, 3H), 1.20 (s, 3H).

I-98 ¹H NMR (400 MHz, DMSO-d₆) δ ppm 10.70 (s, 1H), 7.74 (s, 1H), 7.60 (d, J=2.0 Hz, 1H), 6.14 (d, J=2.4 Hz, 1H), 4.01-3.94 (m, 4H), 3.70 (s, 3H), 3.29-3.26 (m, 2H), 2.11-2.09 (m, 2H), 1.78-1.69 (m, 2H), 1.59-1.53 (m, 2H), 1.19 (s, 3H), 0.79 (t, J=4.5 Hz, 3H).

I-99: ¹H-NMR (300 MHz, DMSO-d₆,) δ ppm 7.74 (s, 1H), 7.57 (d, J=1.5 Hz, 1H), 6.14 (d, J=1.5 Hz, 1H), 4.85 (d, J=2.1 Hz, 1H), 3.99-3.91 (m, 5H), 3.70 (s, 3H), 3.32-3.25 (m, 2H), 2.11-2.07 (m, 2H), 1.59-1.53 (m, 2H), 1.20 (s, 3H), 0.98 (d, J=4.0 Hz, 3H).

I-119: ¹HNMR (400 MHz, DMSO-d₆) δ ppm 10.70 (s, 1H), 7.74 (s, 1H), 7.64 (d, J=2.8 Hz, 1H), 6.14 (d, J=3.2 Hz, 1H), 4.44-4.37 (m, 1H), 3.96-3.91 (m, 2H), 3.70 (s, 3H), 3.33-3.29 (m, 2H), 2.11-2.09 (m, 2H), 1.60-1.52 (m, 2H), 1.37 (d, J=9.2 Hz, 6H), 1.20 (s, 3H).

### Example 97

### 1-methyl-6-[4-methyl-4-(2-methylpyrazol-3-yl)-1-piperidyl]-5H-pyrazolo[3,4-d]pyrimidin-4-one (I-96)

The title compound was isolated by separation of the crude reaction mixture in step 7 of example 96: ¹H NMR (400 MHz, DMSO-d₆) δ ppm 10.84 (s, 1H), 7.75 (s, 1H), 7.28 (d, J=2.0 Hz, 1H), 6.11 (d, J=2.0 Hz, 1H), 3.90 (s, 3H), 3.71 (s, 3H), 3.69-3.64 (m, 4H), 2.07-2.01 (m, 2H), 1.83-1.77 (m, 2H), 1.33 (s, 3H).

### Example 98

### 6-[4-[2,6-difluoro-4-(2-methoxyethoxy)phenyl]piperazin-1-yl]-1-(2-hydroxyethyl)-5H-pyrazolo[3,4-d]pyrimidin-4-one (I-94)

Step 1: To a stirred solution of 2,4,6-trichloropyrimidine-5-carbaldehyde (300 mg, 1.42 mmol, 1.00 equiv) in EtOH (15 mL) maintained under nitrogen at −78° C. was added TEA (289 mg, 2.86 mmol, 2.00 equiv) and 2-hydrazinylethan-1-ol (109 mg, 1.43 mmol, 1.00 equiv). The reaction mixture was stirred for 30 min at −78° C. then concentrated under vacuum. The residue was purified by SiO2 chromatography with EtOAc/petroleum ether (1:1) to afford 130 mg (39%) of 2-[4,6-dichloro-1H-pyrazolo[3,4-d]pyrimidin-1-yl]ethan-1-01 as a off-white solid: LC-MS calcd for C₇H₆Cl₂N₄O [(M−1-H)⁺] 233, obsd 233.0.

Step 2: A mixture of 2-[4,6-dichloro-1H-pyrazolo[3,4-d]pyrimidin-1-yl]ethan-1-ol (130 mg, 0.56 mmol, 1.00 equiv) in 1M NaOH (5.5 mL, 10.00 equiv) and water (10 mL) was refluxed for 1 h. The reaction mixture was cooled to RT and the pH was adjusted to 6 with 1M HCl. The mixture was concentrated under vacuum and the residue was purified by SiO₂ chromatography eluting with 20% DCM/MeOH to afford 100 mg (84%) of 6-chloro-1-(2-hydroxyethyl)-1H,4H,5H-pyrazolo[3,4-d]pyrimidin-4-one as an off-white solid: LC-MS calcd for C₇H₇ClN₄O₂ [(M+H)⁺] 215, obsd 215.0.

Step 3: A 10 mL tube was charged with 6-chloro-1-(2-hydroxyethyl)-1H,4H,5H-pyrazolo[3,4-d]pyrimidin-4-one (100 mg, 0.47 mmol, 1.00 equiv), DIPEA (60 mg, 0.46 mmol, 1.00 equiv) and 1-[2,6-difluoro-4-(2-methoxyethoxy)phenyl]piperazine (127 mg, 0.47 mmol, 1.00 equiv) in EtOH (3 mL), sealed and irradiated in a microwave for 30 min at 140° C. The resulting mixture was cooled to RT and concentrated under vacuum. The residue was dissolved in 5 mL of DCM and the product was precipitated by dilution with 50 mL of ethyl acetate. The product was collected by filtration to afford 104 mg (50%) of 1-94 as an off-white solid. ¹H NMR (300 MHz, DMSO-d₆) δ ppm 10.64 (s, 1H), 7.79 (s, 1H), 6.76 (s, 1H), 6.72 (s, 1H), 4.86-4.83 (m, 1H), 4.16-4.06 (m, 4H), 3.75-3.72 (m, 6H), 3.63-3.61 (m, 2H), 3.29 (s, 3H), 3.08-3.02 (m, 4H). LC-MS calcd for C₂₀H₂₄F₂N₆O₄ [(M+H)⁴] 451, obsd 451.1.

### Example 99

### 1-methyl-6-(4-methylsulfonylphenyl)-5H-pyrazolo[3,4-d]pyrimidin-4-one 1-(I-102), 6-(4-ethylsulfonylphenyl)-1-methyl-5H-pyrazolo[3,4-d]pyrimidin-4-one (I-107), 6-(4-cyclohexylsulfonylphenyl)-1-methyl-5H-pyrazolo[3,4-d]pyrimidin-4-one (I-125) and 6-[4-(cyclohexylmethylsulfonyl)phenyl]-1-methyl-5H-pyrazolo[3,4-d]pyrimidin-4-one (I-108)

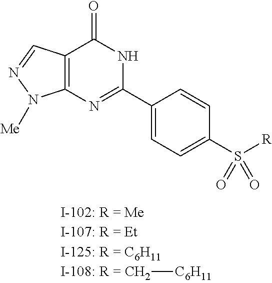

The title compounds were prepared in accord with the procedure described in Example 54 except 4-trifluorophenyl boronic acid was replaced with B[4-methansulfonylphenyl]-boronic acid (CASRN 149104-88-1), B[4-ethanesulfonylphenyl]-boronic acid (CASRN 352530-24-6) and B-[4-cyclohexanesulfonylphenyl]-boronic acid (CASRN 1236189-74-4) to afford the title compounds.

I-108 was prepared analogously from B-[4-(cyclohexylmethylsulfonyl)phenyl]-boronic acid which was prepared by borolation of (4-bromophenyl)(cyclohexylmethyl)sulfane (n-BuLi, B(O-i-Pr)₃, THF) condensation with Intermediate A (Pd(PPh₃)₄, Cs₂CO₃, dioxane, H₂O) and then oxidation (MCPBA, DCM) to the sulfoxide.

I-102: ¹H-NMR (400 MHz, DMSO-d₆) δ ppm 12.60 (s, 1H), 8.38 (d, J=8.0 Hz, 2H), 8.12-8.09 (m, 3H), 3.99 (s, 3H), 3.32 (s, 3H)

I-107 1H-NMR (400 MHz, DMSO-d₆) δ ppm 12.63 (s, 1H), 8.38 (d, J=8.4 Hz, 2H), 8.13 (s, 1H), 8.05 (d, J=8.0 Hz, 2H), 3.99 (s, 3H), 3.43-3.37 (m, 2H), 1.11 (t, J=7.2 Hz, 3H)

I-108: 1H-NMR (400 MHz, DMSO-d₆) δ ppm 12.63 (s, 1H), 8.38 (d, J=8.4 Hz, 2H), 8.13 (s, 1H), 8.07 (d, J=8.0 Hz, 2H), 3.99 (s, 3H), 3.31 (d, J=8.0 Hz, 2H), 1.79-1.77 (m, 3H), 1.63-1.54 (m, 3H), 1.23-1.02 (m, 5H)

I-125 1H-NMR (400 MHz, DMSO-d₆) δ ppm 12.61 (s, 1H), 8.38 (d, J=8.4 Hz, 2H), 8.13 (s, 1H), 8.00 (d, J=8.4 Hz, 2H), 3.99 (s, 3H), 3.33-3.29 (s, 1H), 1.91-1.89 (m, 2H), 1.78-1.75 (m, 2H), 1.61-1.58 (m, 1H), 1.32-1.19 (m, 4H), 1.11-1.08 (m, 1H)

### Example 100

### 1-methyl-6-(4-norbornan-2-ylpiperazin-1-yl)-5H-pyrazolo[3,4-d]pyrimidin-4-one (I-103)

The title compound was prepared by condensation of 1-bicyclo[2.2.1]hep-2-yl-piperazine (CASRN 1365836-29-8) and Intermediate A to afford the title compound: ¹H-NMR (400 MHz, DMSO-d₆) δ ppm 7.77 (s, 1H), 3.72 (s, 3H), 3.67-3.58 (m, 4H), 2.51-2.30 (m, 5H), 2.23-2.15 (m, 2H), 1.74-1.67 (m, 2H), 1.46-1.15 (m, 5H), 1.20 (m, 1H), 0.89 (s, 1H).

### Example 101

### 6-[4-(2 46-trifluorophenyl)piperazin-1-yl]-1 5-dihydropyrazolo[3,4-d]pyrimidin-4-one (I-104)

The title compound was prepared by condensation of Intermediate B and 4-(2,4,6-trifluorophenyl)-piperazine (CASRN 223513-17-5) to afford the title compound: ¹H-NMR (300 MHz, DMSO-d₆) δ ppm 12.96 (s, 1H), 10.96 (s, 1H), 7.79 (s, 1H), 7.22-7.13 (m, 2H), 3.70-3.64 (m, 4H), 3.15-3.08 (m, 4H).

### Example 102

### 1-methyl-6-[4-(1-methylpyrazol-4-yl)piperazin-1-yl]-5H-pyrazolo[3,4-d]pyrimidin-4-one (I-105)

The title compound was prepared by condensation of 1-(1-methyl-1H-pyrazol-4-yl)piperazine (CASRN 1174207-79-4) and Intermediate A: ¹H-NMR (300 MHz, DMSO-d₆) δ ppm 10.81 (s, 1H), 7.76 (s, 1H), 7.31 (s, 1H), 7.20 (s, 1H), 3.77-3.73 (m, 10H), 2.89 (t, J=5.0 Hz, 4H).

### Example 103

6-[4-(cyclohexylmethyl)piperazin-1-yl]-1-methyl-5H-pyrazolo[3,4-d]pyrimidin-4-one (I-110), 1-methyl-6-[4-(tetrahydropyran-4-ylmethyl)piperazin-1-yl]-5H-pyrazolo[3,4-d]pyrimidin-4-one (I-116), 6-(4-cyclohexylpiperazin-1-yl)-1-methyl-5H-pyrazolo[3,4-d]pyrimidin-4-one (I-118), 6-[4-(trans-4-hydroxycyclohexyl)piperazin-1-yl]-1-methyl-5H-pyrazolo[3,4-d]pyrimidin-4-one (I-120), 6-[4-(4-cis-hydroxycyclohexyl)piperazin-1-yl]-1-methyl-5H-pyrazolo[3,4-d]pyrimidin-4-one (I-124), 1-methyl-6-(4-tetrahydropyran-4-ylpiperazin-1-yl)-5H-pyrazolo[3,4-d]pyrimidin-4-one (I-130), 1-methyl-6-[4-[(5-methylisoxazol-3-yl)methyl]piperazin-1-yl]-5H-pyrazolo[3,4-d]pyrimidin-4-one (I-140), 1-methyl-6-[4-(oxetan-3-yl)piperazin-1-yl]-5H-pyrazolo[3,4-d]pyrimidin-4-one (I-144).

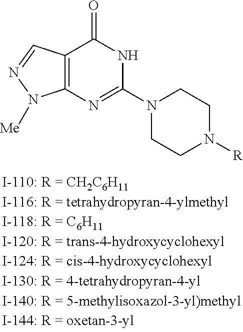

The title compounds were prepared by condensation of Intermediate A with 1-(cyclohexylmethyl)-piperazine (CASRN 57184-23-3), 1-[(tetrahydro-2H-pyran-4-yl)methyl]-piperazine (CASRN 787518-60-9), 1-cyclohexyl-piperazine (CASRN 17766-28-8), trans-4-(1-piperazinyl-cyclohexanol (CASRN 223605-18-3), cis-4-(1-piperazinyl-cyclohexanol (CASRN 223605-17-2), 1-(tetrahydro-2H-pyran-4-yl)piperazine (CASRN 398137-19-4), 1-[(5-methyl-3-isoxazolyl)methyl]piperazine (CASRN 073850-51-6) and 1-(3-oxetanyl)piperazine (CASDN 1254115-23-5).

I-110: ¹H-NMR (300 MHz, DMSO-d₆) δ ppm 10.86 (s, 1H), 7.76 (s, 1H), 3.71 (s, 3H), 3.68-3.58 (m, 4H), 2.39-2.27 (m, 4H), 2.09 (d, J=6.0 Hz, 2H), 1.75-1.65 (m, 4H), 1.53-1.48 (m, 1H), 1.27-1.24 (m, 4H), 0.91-0.76 (m, 2H).

I-116: ¹H-NMR (300 MHz, DMSO-d₆) δ ppm 7.82 (s, 1H), 3.97-3.92 (m, 2H), 3.79 (s, 3H), 3.73-3.70 (m, 4H), 3.48-3.40 (m, 2H), 2.56-2.52 (m, 4H), 2.28 (d, J=4.6 Hz, 2H), 1.90-1.83 (m, 1H), 1.75-1.71 (m, 2H), 1.36-1.18 (m, 2H)

I-118: ¹H-NMR (300 MHz, DMSO-d₆) δ ppm 7.86 (s, 1H), 3.92-3.91 (m, 6H), 3.82-3.74 (m, 3H), 3.88-3.08 (m, 4H), 2.79-2.55 (m, 1H), 2.09-2.04 (m, 2H), 1.94-1.86 (m, 2H), 1.78-1.62 (m, 2H), 1.40-1.12 (m, 6H).

I-124: ¹H-NMR (400 MHz, CD₃OD) δ ppm 7.82 (s, 1H), 3.90-3.30 (m, 1H), 3.80 (s, 3H), 3.72-3.70 (m, 4H), 2.73-2.70 (m, 4H), 2.41-2.36 (m, 1H), 1.88-1.84 (m, 2H), 1.79-1.73 (m, 2H), 1.68-1.64 (m, 2H), 1.62-1.50 (m, 2H).

I-103: ¹H-NMR (300 MHz, CD₃OD) δ ppm 7.83 (s, 1H), 4.07-3.98 (m, 2H), 3.84 (s, 3H), 3.80-3.71 (m, 4H), 3.46-3.38 (m, 2H), 2.77-2.68 (m, 4H), 2.57-2.49 (m, 1H), 1.89-1.86 (m, 2H), 1.64-1.58 (m, 2H)

I-140: ¹H-NMR (300 MHz, CD₃OD) δ ppm 7.82 (s, 1H), 6.19 (s, 1H), 3.79 (s, 3H), 3.70 (t, J=5.0 Hz, 4H), 3.64 (s, 2H), 2.59 (t, J=5.1 Hz, 4H), 2.43 (s, 3H)

I-144: 1H¹H-NMR (300 MHz, CD₃OD) δ ppm 7.84 (s, 1H), 4.75-4.63 (m, 4H), 3.84-3.75 (m, 7H), 3.61-3.52 (m, 1H), 2.49-2.46 (t, J=5.4 Hz, 4H).

### Example 104

### 1-methyl-6-(4-pyrazol-1-yl-1-piperidyl)-5H-pyrazolo[3,4-d]pyrimidin-4-one (I-112)

The title compound was prepared by reacting intermediate S\A with 4-(1H-pyrazol-1-yl)piperidine: ¹HNMR (300 MHz, DMSO-d₆) δ ppm 10.94 (s, 1H), 7.80-7.78 (m, 2H), 7.44-7.43 (d, J=1.2 Hz, 2H), 6.24-6.23 (m, 1H), 4.51-4.46 (m, 3H), 3.73 (s, 3H), 3.17-3.10 (m, 2H), 2.09-1.85 (m, 4H).

### Example 105

### 6-[4-[2,6-difluoro-4-(2-methoxyethoxy)phenyl]piperazin-1-yl]-1-(2 3-dihydroxypropyl)-5H-pyrazolo[3,4-d]pyrimidin-4-one (I-113)

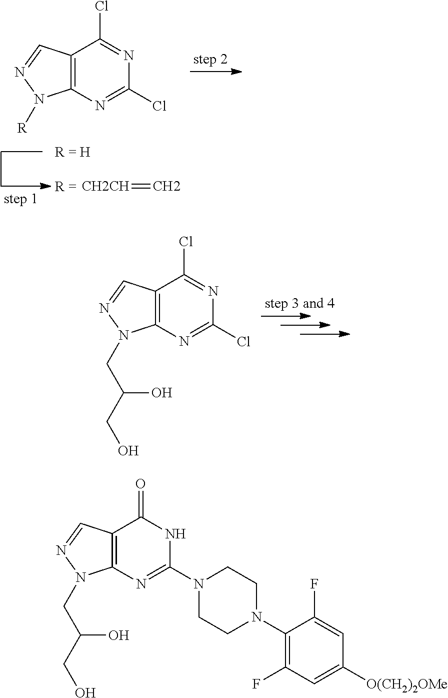

¹H NMR (400 MHz, DMSO-d₆) δ ppm 10.95 (s, 1H), 7.80 (s, 1H), 6.75 (s, 1H), 6.72 (s, 1H), 4.85 (d, J=4.8 Hz, 1H), 4.64 (s, 1H), 4.08-4.03 (m, 4H), 3.96-3.95 (m, 1H), 3.74-3.71 (m, 4H), 3.63-3.60 (m, 2H), 3.46-3.35 (m, 2H), 3.28 (s, 3H), 3.18-3.02 (m, 4H)

### Example 106

### 6-[4-[2,6-difluoro-4-(2-methoxyethoxy)phenyl]piperazin-1-yl]-1-tetrahydropyran-4-yl-5H-pyrazolo[3,4-d]pyrimidin-4-one (I-123) and 6-[4-[2,6-difluoro-4-(2-methoxyethoxy)phenyl]piperazin-1-yl]-1-(1 1-dioxothian-4-yl)-5H-pyrazolo[3 4-d]pyrimidin-4-one (I-128)

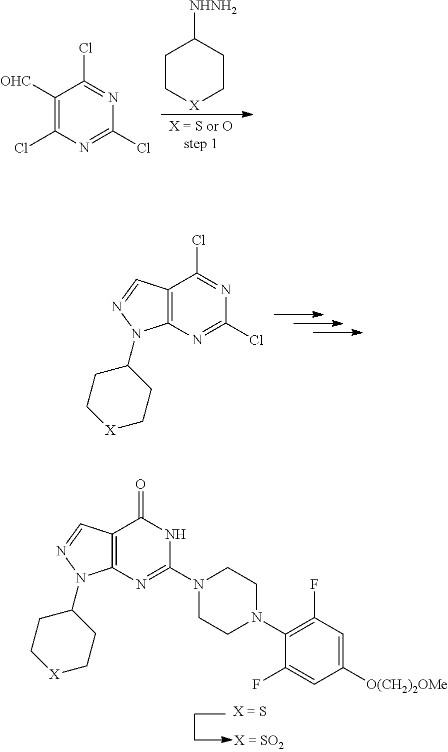

Tetrahydro-2H-pyran-4-yl-hydrazine and tetrahydro-2H-thiopyran-4-yl-hydrazine were each condensed (DIPEA, EtOH) with 2,4,6-trichloro-pyrimidine-5-carbaldehyde to afford 4,6-dichloro-1-(tetrahydro-pyran-4-yl)-1H-pyrazolo[3,4-d]pyrimidine and 4,6-dichloro-1-(tetrahydro-thiopyran-4-yl)-1H-pyrazolo[3,4-d]pyrimidine respectively. Hydrolysis (aqueous NaOH) affords -chloro-1-(tetrahydro-2H-pyran-4-yl)-1H-pyrazolo[3,4-d]pyrimidin-4(5H)— one and 6-chloro-1-(tetrahydro-2H-thiopyran-4-yl)-1H-pyrazolo[3,4-d]pyrimidin-4 (5H)-one which can be condensed with intermediate I to afford 6-(4-(2,6-difluoro-4-(2-methoxyethoxy)phenyl)piperazin-1-yl)-1-(tetrahydro-2H-pyran-4-yl)-1H-pyrazolo[3,4-d]pyrimidin-4(5H)-one (I-123) and 6-(4-(2,6-difluoro-4-(2-methoxyethoxy)phenyl)piperazin-1-yl)-1-(tetrahydro-2H-thiopyran-4-yl)-1H-pyrazolo[3,4-d]pyrimidin-4 (5H)-one. The latter is oxidized to the corresponding sulfone (I-128).

I-123: ¹H NMR (300 MHz, DMSO-d₆) δ ppm 10.96 (s, 1H), 7.81 (s, 1H), 6.78-6.69 (m, 2H), 4.69-4.59 (m, 1H), 4.10-4.07 (m, 2H), 3.99-3.94 (m, 2H), 3.74-3.71 (m, 4H), 3.64-3.61 (m, 2H), 3.46 (t, J=11.1 Hz, 2H), 3.29 (s, 3H), 3.12-3.02 (m, 4H), 2.16-2.02 (m, 2H), 1.82-1.76 (m, 2H)

I-128: ¹H-NMR (300 MHz, DMSO-d₆) δ ppm 7.84 (s, 1H), 6.75 (s, 1H), 6.72 (s, 1H), 4.91-4.89 (m, 1H), 4.09-4.06 (t, J=4.2 Hz, 2H), 3.81-3.74 (m, 4H), 3.64-3.61 (t, J=4.5 Hz, 2H), 3.58-3.32 (m, 2H), 3.29 (s, 3H), 3.11-3.27 (m, 2H), 3.12-3.07 (m, 4H), 2.59-2.48 (m, 2H), 2.38-2.10 (m, 2H)

### Example 107

### 6-[4-[2 6-difluoro-4-(2-methoxyethoxy)phenyl]piperazin-1-yl]-1-(2-hydroxypropyl)-5H-pyrazolo[3,4-d]pyrimidin-4-one (I-126)

Condensation (DIPEA, EtOH) of 1-hydrazinyl-2-popanol (CASRN 18501-20-7) and 2,4,6-trichloro-pyrimidine-5-carbaldehyde affords 1-(4,6-dichloro-3a,7a-dihydro-1H-pyrazolo[3,4-d]pyrimidin-1-yl)propan-2-ol which is subjected to basic hydrolysis (NaOH) and condensation (DIPEA, EtOH) with Intermediate I to afford the title compound: ¹H NMR (300 MHz, DMSO-d₆) δ ppm 11.96 (s, 1H), 7.80 (s, 1H), 6.78-6.70 (m, 2H), 4.84 (d, J=4.5 Hz, 1H), 4.12-4.06 (m, 4H), 3.97-3.92 (m, 1H), 3.74-3.71 (m, 4H), 3.64-3.61 (m, 2H), 3.29 (s, 3H), 3.08-3.02 (m, 4H), 1.02 (d, J=6.0 Hz, 3H).

### Example 108

### 6-[4-[2,6-difluoro-4-(2-methoxyethoxy)phenyl]piperazin-1-yl]-1-(2-hydroxy-2-methyl-propyl)-5H-pyrazolo[3,4-d]pyrimidin-4-one (I-143)

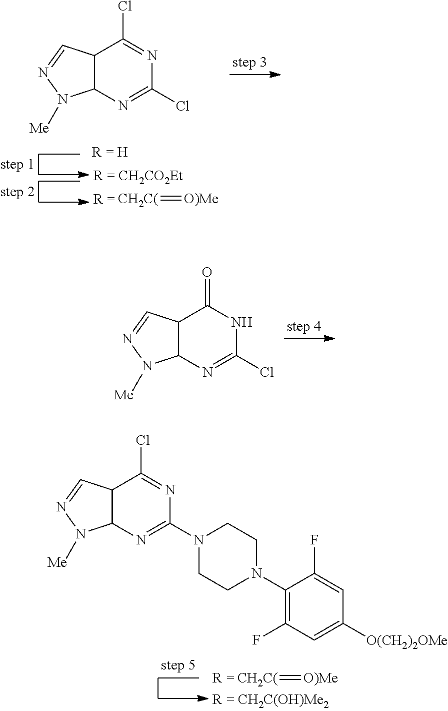

Condensation (DIAD, PPh₃, THF) of 4,6-dichloro-3a,7a-dihydro-1H-pyrazolo[3,4-d]pyrimidine and ethyl hydroxyacetate affords ethyl 2-(4,6-dichloro-3a,7a-dihydro-1H-pyrazolo[3,4-d]pyrimidin-1-yl)acetate which afforded the ketone, 1-(4,6-dichloro-3a,7a-dihydro-1H-pyrazolo[3,4-d]pyrimidin-1-yl)propan-2-one, when treated with MeMgBr/THF at −60° C. Hydrolysis (1M NaOH) and condensation with Intermediate I (DIPEA, EtOH) affords 6-(4-(2,6-difluoro-4-(2-methoxyethoxy)phenyl)piperazin-1-yl)-1-(2-oxopropyl)-5,7a-dihydro-1H-pyrazolo[3,4-d]pyrimidin-4(3aH)-one which again treated with MeMgBr/THF to afford the title compound: ¹H NMR (300 MHz, DMSO-d₆) δ ppm 10.94 (s, 1H), 7.80 (s, 1H), 6.74 (s, 1H), 6.68 (s, 1H), 4.70 (s, 1H), 4.08-4.03 (m, 4H), 3.73-3.71 (m, 4H), 3.63-3.59 (m, 2H), 3.28 (s, 3H), 3.07-3.02 (m, 4H), 1.09 (s, 6H).

### Example 109

### 1-methyl-6-(4-methylsulfonylpiperazin-1-yl)-5H-pyrazolo[3,4-d]pyrimidin-4-one (I-142)

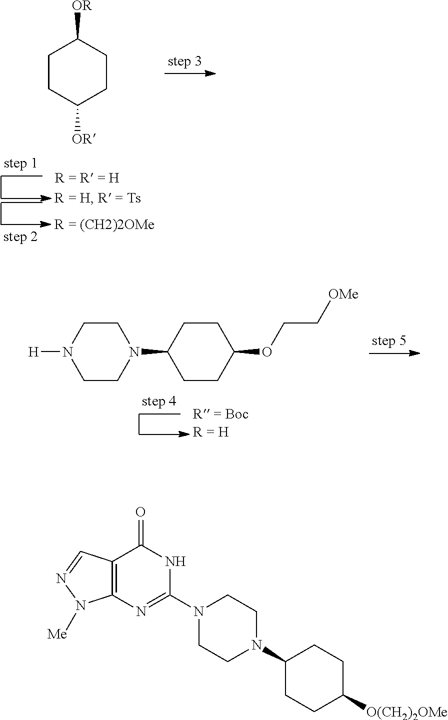

The title compound was prepared by condensation (DIPEA/EtOH) of 1-(methylsulfonyl)-piperazine (CASRN 55776-43-2) and Intermediate A: ¹H-NMR (400 MHz, DMSO-d₆) δ ppm 11.07 (s, 1H), 7.80 (s, 1H), 3.78-3.74 (m, 7H), 3.18 (t, J=4.4 Hz, 4H), 2.91 (s, 3H).

### Example 110

### 1-methyl-6-(4-methylsulfonyl-1-piperidyl)-5H-pyrazolo[3,4-d]pyrimidin-4-one (I-141)

The title compound was prepared by condensation (DIPEA/EtOH) of 4-methylsulfonyl-piperidine (CASRN 290328-55-1) and Intermediate A: ¹H-NMR (400 MHz, DMSO-d₆) δ ppm 10.97 (s, 1H), 7.77 (s, 1H), 4.49 (d, J=16.0 Hz, 2H), 3.73 (s, 3H), 3.43-3.35 (m, 1H), 3.03-2.95 (m, 5H), 2.09 (d, J=11.2 Hz, 2H), 1.65-1.55 (m, 2H).

### Example 111

### 1-methyl-6-(5-methylsulfonyl-2-pyridyl)-5H-pyrazolo[3,4-d]pyrimidin-4-one

2-Bromo-5-(methylthio)-pyridine (CASRN 134872-23-4) is converted to an organozinc compound (n-BuLi, ZnCl₂) and condensed with Intermediate A (Pd₂(dpa)₃/dppf) and the resulted adducted oxidized to the corresponding sulfone (MCPBA) to afford the title compound: ¹H NMR (400 MHz, DMSO-d₆) δ ppm 11.95 (s, 1H), 9.22 (d, J=1.6 Hz, 1H), 8.67 (d, J=8.0 Hz, 1H), 8.58 (dd, J=8.4 Hz, J=2.4 Hz, 1H), 8.16 (s, 1H), 4.03 (s, 3H), 3.44 (s, 3H).

### Example 112

### 6-(4-((cis)-4-(2-methoxyethoxy)cyclohexyl)piperazin-1-yl)-1-methyl-1H-pyrazolo[3,4-d]pyrimidin-4(5H)-one (I-136) and 6-(4-((trans)-4-(2-methoxyethoxy)cyclohexyl)piperazin-1-yl)-1-methyl-1H-pyrazolo[3,4-d]pyrimidin-4(5H)-one (I-121)

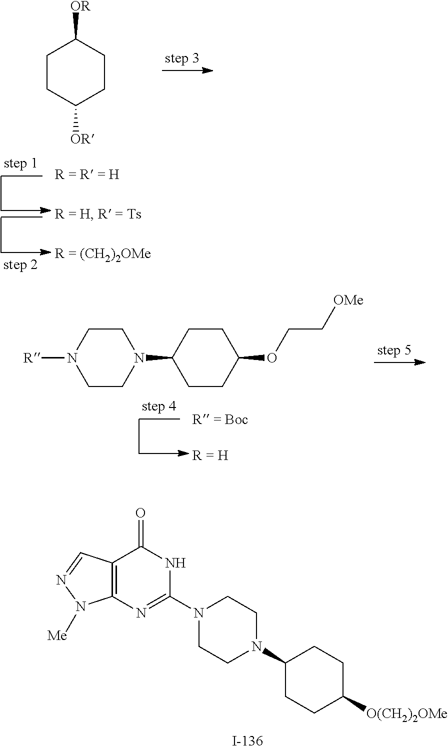

1-((1s,4s)-4-(2-methoxyethoxy)cyclohexyl)piperazine was prepared from trans-1,2 cyclohexanediol by monotosylation and O-alkylation (Br(Ch₂)₂—OMe, NaH, DMF), displacement of the tosyl group with i-Boc-piperazine and deprotection (HCl/MeOH). Condensation of the resulting compound with Intermediate A ((DIPEA/EtOH) affords I-136: ¹H-NMR (300 MHz, DMSO-d₆) δ ppm 7.76 (s, 1H), 3.71 (s, 3H), 3.61 (s, 4H), 3.47-3.41 (m, 6H), 3.26-3.23 (m, 4H), 2.81 (s, 1H), 2.50 (s, 1H), 2.27-2.23 (m, 1H), 1.83-1.79 (m, 2H), 1.57-1.37 (m, 6H).

By analogy a-1,4-cyclohexanediol p-toluenesulfonate (CASRN 132961-64-9) affords I-121: ¹H-NMR (300 MHz, DMSO-d₆) δ ppm 10.81 (s, 1H), 7.76 (s, 1H), 3.71 (s, 3H), 3.62-3.59 (m, 4H), 3.52-3.49 (m, 2H), 3.42-3.38 (m, 2H), 3.23 (s, 3H), 3.18-3.17 (m, 1H), 2.75-2.54 (m, 4H), 2.40-2.20 (m, 1H), 2.01-1.97 (m, 2H), 1.82-1.80 (m, 2H), 1.10-1.27 (m, 4H).

### Example 113

### 1-methyl-6-[4-[(2-methylpyrazol-3-yl)methyl]piperazin-1-yl]-5H-pyrazolo[3,4-d]pyrimidin-4-one (I-132)

The title compound is prepared by the condensation (DIPEA, EtOH) of Intermediate A and 1[(1-methyl-1H-pyrazol-5-yl)methyl]-piperazine (CASRN 1172340-74-7): ¹H-NMR (300 MHz, CD₃OD) δ ppm 7.82 (s, 1H), 7.39 (d, J=1.8 Hz, 1H), 6.24 (d, J=1.8 Hz, 1H), 3.91 (s, 3H), 3.79 (s, 3H), 3.73-3.70 (t, J=5.0 Hz, 4H), 3.64 (s, 2H), 2.59-2.55 (t, J=5.0 Hz, 4H).

### Example 114

### 1-methyl-6-[4-[(1-methylpyrazol-4-yl)methyl]piperazin-1-yl]-5H-pyrazolo[3,4-d]pyrimidin-4-one (I-134)

The title compound is prepared by the condensation (DIPEA, EtOH) of Intermediate A and 1-[(1-methyl-1H-pyrazol-4-yl)methyl]-piperazine (CASRN 1001757-59-0): ¹H-NMR (300 MHz, CD₃OD) δ ppm 7.82 (s, 1H), 7.58 (s, 1H), 7.45 (s, 1H), 3.88 (s, 3H), 3.79 (s, 3H), 3.71 (t, J=4.7 Hz, 4H), 3.55 (s, 2H), 2.65-2.55 (m, 4H).

### Example 115

### 6-[4-(cyclohexylmethylsulfonyl)-1-piperidyl]-1-methyl-5H-pyrazolo[3,4-d]pyrimidin-4-one (I-135)

tert-Butyl 4-mercapto-1-piperidinecarboxylate was alkylated with (bromomethyl)cyclohexane (KOH, MeOH), oxidized to the sulfone (MCPBA) and deprotected by catalytic hydrogenolysis (Pd/C, H₂, MeOH). Condensation of the resulting amine with Intermediate A affords the title compound: ¹H-NMR (400 MHz, DMSO-d6) δ ppm 10.96 (s, 1H), 7.77 (s, 1H), 4.48 (d, J=13.6 Hz, 2H), 3.72 (s, 3H), 3.45-3.32 (m, 1H), 3.03-2.97 (m, 4H), 2.07 (d, J=11.6 Hz, 2H), 1.97-1.84 (m, 3H), 1.67-1.55 (m, 5H), 1.33-1.20 (m, 2H), 1.20-1.02 (m, 3H)

### Example 116

### 6-[4-[4-[1-(chloromethyl)-2-hydroxy-ethoxy]-2 6-difluoro-phenyl]piperazin-1-yl]-1-methyl-5H-pyrazolo[3,4-d]pyrimidin-4-one (I-64)

Step 1: Thionyl chloride (0 2 mL) was added to MeOH (1 mL) at 25° C. and then stirred for 15 min. tert-Butyl 4-[2,6-difluoro-4-(oxetan-3-yloxy)phenyl]piperazine-1-carboxylate (10 mg, 0.03 mmol, 1.00 equiv) was then added and the resulting solution was stirred at 25° C. for 30 min. The reaction mixture was concentrated under vacuum to afford 5 mg (60%) of 3-chloro-2-[3,5-difluoro-4-(piperazin-1-yl)phenoxy]propan-1-ol as a white solid. LC-MS calcd for C₁₈H₂₅ClF₂N₂O₄ [(M+H)⁺] 307, obsd 307.0.

Step 2: A 5 mL tube was charged with 6-chloro-1-methyl-1H-pyrazolo[3,4-d]pyrimidin-4-ol (5 mg, 0.03 mmol, 1.00 equiv), 3-chloro-2-[3,5-difluoro-4-(piperazin-1-yl)phenoxy]propan-1-ol (10 mg, 0.03 mmol, 1.20 equiv) and DIPEA (10.8 mg, 0.08 mmol, 3.08 equiv) in EtOH (2 mL), sealed and irradiated in a microwave for 20 min at 140° C. The reaction mixture was cooled to RT and the precipitated product was collected by filtration to afford 9.6 mg (78%) of 6-(4-[4-[(1-chloro-3-hydroxypropan-2-yl)oxy]-2,6-difluorophenyl]piperazin-1-yl)-1-methyl-1H-pyrazolo[3,4-d]pyrimidin-4-ol as a white solid: ¹H NMR (300 MHz, DMSO-d₆) δ ppm 10.96 (s, 1H), 7.80 (s, 1H), 6.80 (d, J=11.4 Hz, 2H), 5.10-5.00 (m, 1H), 4.55-4.51 (m, 1H), 3.77-3.75 (m, 9H), 3.63-3.61 (m, 7H), 3.10-3.09 (m, 4H).

LC-MS called for C₁₉H₂₁ClF₂N₆O₃ [(M+H)⁺] 455, obsd 455.1.

¹H NMR (300 MHz, DMSO-d₆) δ ppm 10.96 (s, 1H), 7.80 (s, 1H), 6.80 (d, J=11.4 Hz, 2H), 5.10-5.00 (m, 1H), 4.55-4.51 (m, 1H), 3.77-3.75 (m, 9H), 3.63-3.61 (m, 7H), 3.10-3.09 (m, 4H).

### Example 117

### 6-[4-[2 6-difluoro-4-(2-methoxyethoxy)phenyl]piperazin-1-yl]-1-(3-hydroxypropyl)-5H-pyrazolo[3,4-d]pyrimidin-4-one (I-95)

Step 1: To a stirred solution of 4,6-dichloro-1H-pyrazolo[3,4-d]pyrimidine (600 mg, 3.17 mmol, 1.00 equiv), PPh₃ (2.1 g, 8.01 mmol, 2.50 equiv) and propane-1,3-diol (364 mg, 4.78 mmol, 1.50 equiv) in THF (50 mL) at 0° C. was added dropwise DD (1.6 g, 7.91 mmol, 2.50 equiv) over a 5 min period. The resulting solution was stirred for 1 h at 25° C. and then diluted with 10 mL of water and 100 then dried over anhydrous sodium sulfate and mL of DCM. The organic layer was washed with 1×100 mL of water and 1×100 mL of brine, dried (Na₂SO₄), filtered and concentrated under vacuum. The residue was purified by SiO2 chromatography eluting with 50% EtOAc/petroleum ether to afford 700 mg (42%) of 3-[4,6-dichloro-1H-Pyrazolo[3,4-d]pyrimidin-1-yl]propan-1-ol as a light yellow solid: LC-MS calcd for C₈H₈Cl₂N₄O [(M+H)⁺] 247, obsd 247.0.

3-[4,6-Dichloro-1H-pyrazolo[3,4-d]pyrimidin-1-yl]propan-1-ol was converted to the title compound in analogy with steps 2 and 3 of example 98: The final product was purified by SiO2 chromatography eluting with DCM/MeOH (97/3) to afford 51.2 mg (48%) of 6-[4-[2,6-difluoro-4-(2-methoxyethoxy)phenyl]piperazin-1-yl]-1-(3-hydroxypropyl)-1H,4H,5H-pyrazolo[3,4-d]pyrimidin-4-one as an off-white solid: ¹H NMR (400 MHz, DMSO-d₆) δ ppm 10.95 (s, 1H), 7.79 (s, 1H), 6.75 (s, 1H), 6.71 (s, 1H), 4.50 (t, J=4.8 Hz, 1H), 4.17-4.06 (m, 4H), 3.73-3.72 (m, 4H), 3.63-3.61 (m, 2H), 3.43-3.40 (m, 2H), 3.29 (s, 3H), 3.08-3.04 (m, 4H), 1.94-1.89 (m, 2H). LC-MS calcd for C₂₁H₂₆F₂N₆O₄ [(M+H)⁺] 465, obsd.465.3.

### Example 118

μHTS-TNKS-IWR2 TR-FRET Binding Assay (10 μL/well in BD1536-well plate, a single point)

Reagents and Stock Solutions

Tankyrase 1 (TNKS1): 184.3 μM=5.2 mg/mL His6-TNKS1, MW=28.2 KDa (construct: 1088-1327, 1266M) in 20 mM Tris pH 8, 150 mM NaCl, 10% glycerol, and 0.5 mM TCEP. Alternatively, His6-tankyrase 2 (construct: 934-1166) (His6-TNKS2) or His6-PARP1 (full length) can be substituted for His6-TNKS 1.

Biotin-4-((1S,2R,6S,7R)-3,5-Dioxo-4-aza-tricyclo[5.2.1.0*2,6]dec-8-en-4-yl)-N-(4-methyl-quinolin-8-yl)-benzamide (Biotin-IWR2): 10 mM Biotin-IWR2 stock in DMSO, stored at −20° C.

Positive control: 10 mM 2-(4-Trifluoromethyl-phenyl)-3,5,7,8-tetrahydro-thiopyrano[4,3-d]pyrimidin-4-one (XAV939) in DMSO, stored at −20° C.

Eu-Streptavidin: 38.1 μM (2.1 mg/mL) Eu-SA (Bio# Eu-2212, Lot# N 18001-BDH02)

APC-anti-His Ab: 8.50 μM SL-APC, 8.26 μM anti-6His antibody-SureLight APC (Columia Bioscience, Catalog Number D3-1711, Lot Number N01010-AAH04)

Assay plate: BD 1536-well, clear/black plate (Catalog Number 353255)

NP-40: 10% NP-40 solution (PIERCE, Catalog Number 28324, Lot Number 97101671)

Assay Buffer Preparation

Assay buffer 1a (AB1a) for TNKS dilution: 50 mM Tris, pH 7.4, 100 mM sodium chloride solution, 1 mM magnesium chloride solution, 1 mM DL-dithiothreitol solution, 0.2 mg/mL bovine serum albumin solution, 0.025% NP-40.

Assay buffer 1b (AB1b) for Biotin-IWR2 dilution: 50 mM Tris, pH 7.4, 100 mM sodium chloride solution, 1 mM magnesium chloride solution, 1 mM DL-dithiothreitol solution, 0.2 mg/mL bovine serum albumin solution, 0.05% NP-40

Assay buffer 1c (AB1c) for compound dilution: 50 mM Tris, pH 7.4, 100 mM sodium chloride solution, 1 mM magnesium chloride solution, 1 mM DL-dithiothreitol solution, 0.2 mg/mL bovine serum albumin solution

Assay buffer 2 (AB2) for Eu/APC: 50 mM Tris, pH 7.4, 100 mM sodium chloride solution, 1 mM magnesium chloride solution, 0.2 mg/mL bovine serum albumin solution

Reagent Stock Solution Preparation

Prepare Biotinylated IWR2 stock solution (3.33× stock) for TOTL and compound wells: 200 nM Biotin-IWR2 in 5% DMSO/AB1b buffer

Prepare BLANK well stock solution: 5% DMSO/AB1b buffer

Prepare POSITIVE CONTROL well stock solution (3.33× stock): 200 nM XAV939 in 200 nM Biotin-IWR2/5% DMSO/AB1b buffer

Prepare TNKS 1 stock solution (5× stock): 300 nM TNKS in AB1a buffer. (Alternatively, use TNKS2 or PARP1 stock solutions.)

Prepare Eu/APC stock solution (5× stock): 3.5 nM Eu-SA/50 nM APC-His6Ab in AB2 buffer.

Assay Procedure

Compound Preparations:

Add 25 μL/well 1.5% DMSO/AB1c buffer in each compound well to the compound concentration at 74 μM in 8.8% DMSO/AB1c buffer or in the 2 μL DMSO CONTROL wells (BLANK, TOTAL and POSITIVE wells) in the compound plate. Transfer 3 μL/well of above solution (solution 1,2,3) to an empty assay plate (BD1536-well plate) as follows:
TOTAL and cpd wells: Solution 1 (Biotin-IWR2):
BLANK wells: Solution 2 (No Biotin-IWR2):
POSITIVE CONTROL wells: Solution 3 (Biotin-IWR2+XAV939)

Transfer 3 μL/well of the above diluted compound solutions or compound dilution buffer to the above assay plate. Add 2 μL/well of 300 nM TNKS stock solution (4) to every well in the above assay plate. Centrifuge the assay plate at 2100 rpm for 2 min. Incubate the assay plate at 26° C. for 30 minutes. Add 2 μL/well 3.5 nMEu/50 nM APC solution (5) to every well in the above assay plate. Centrifuge the assay plate at 2100 rpm for 2 min. Incubate the assay plate at 26° C. for 60 min. Read the assay plate immediately at excitation wavelength of 330 nM and emission wavelength of 615 and 665 nM in time resolved fluorescence mode.

Final Assay Conditions

Biotin-IWR2: 60 nM

TNKS: 60 nM

Eu-SA: 0.7 nM

APC-His Ab: 10 nM

XAV939 (+ve control): 60 nM at ˜70% Inhibition

General Library compounds: 22.23 μM in 4% DMSO

Representative compound data for assays are listed in Table I. Values are in μM.

### Example 119

### Tankyrase 1 Assay

Inhibition of the Wnt stimulated TCF transcriptional activity by tankyrase inhibitors was determined utilizing a HEK293-TS 112 TCF reporter cell line. A Wnt-responsive luciferase reporter named TOPbrite was constructed by cloning the enhancer element of Super8xTOPFlash containing eight TCF/LEF binding sites into the pGL4.28 vector (Promega) upstream of the minimal promoter element, and selecting for hygromycin B resistance (50 μg/ml). Cells were seeded into 384-well plates in the presence of 0.5 ug/mL of Wnt3A at a density of 20,000 cells per well in 25 ul of F:12 DMEM media supplemented with 10% FBS and 2 mM Glutamax. Cells seeded without the addition of Wnt3A were used as background signal. Compounds of various concentrations were added to cells and incubated at 37 degrees with 5% CO2 for 16 hours. The assay was terminated with the addition of Promega Dual Glo kit per manufacturer's instructions. A ratio of TOPbrite Firefly Luciferase and SV40 Renillla Luciferase was calculated and the background from the neutral wells was subtracted yielding the final normalized measurement of TCF transcriptional activity. Compound IC₅₀s were determined by four-parameter curve fitting using GeneData software.

### Example 120

Pharmaceutical compositions of the subject Compounds for administration via several routes can be prepared as described in this Example.

Composition for Oral Administration (A)

| Ingredient | % wt./wt. |
|---|---|
| Active ingredient | 20.0% |
| Lactose | 79.5% |
| Magnesium stearate | 0.5% |

The ingredients are mixed and dispensed into capsules containing about 100 mg each; one capsule would approximate a total daily dosage.

Composition for Oral Administration (B)

| Ingredient | % wt./wt. |
|---|---|
| Active ingredient | 20.0% |
| Magnesium stearate | 0.5% |
| Crosscarmellose sodium | 2.0% |
| Lactose | 76.5% |
| PVP (polyvinylpyrrolidine) | 1.0% |

The ingredients are combined and granulated using a solvent such as methanol. The formulation is then dried and formed into tablets (containing about 20 mg of active compound) with an appropriate tablet machine.

Composition for Oral Administration (C)

| Ingredient | % wt./wt. | |
|---|---|---|
| Active compound | 1.0 | g |
| Fumaric acid | 0.5 | g |
| Sodium chloride | 2.0 | g |
| Methyl paraben | 0.15 | g |
| Propyl paraben | 0.05 | g |
| Granulated sugar | 25.5 | g |
| Sorbitol (70% solution) | 12.85 | g |
| Veegum K (Vanderbilt Co.) | 1.0 | g |
| Flavoring | 0.035 | ml |
| Colorings | 0.5 | mg |
| Distilled water | q.s. to 100 | ml |

The ingredients are mixed to form a suspension for oral administration.

Parenteral Formulation (D)

| Ingredient | % wt./wt. |
|---|---|
| Active ingredient | 0.25 g |
| Sodium Chloride | qs to make isotonic |
| Water for injection to | 100 ml |

The active ingredient is dissolved in a portion of the water for injection. A sufficient quantity of sodium chloride is then added with stirring to make the solution isotonic. The solution is made up to weight with the remainder of the water for injection, filtered through a 0.2 micron membrane filter and packaged under sterile conditions.

Suppository Formulation (E)

| Ingredient | % wt./wt. |
|---|---|
| Active ingredient | 1.0% |
| Polyethylene glycol 1000 | 74.5% |
| Polyethylene glycol 4000 | 24.5% |

The ingredients are melted together and mixed on a steam bath, and poured into molds containing 2.5 g total weight.

Topical Formulation (F)

| Ingredients | grams |
|---|---|
| Active compound | 0.2-2 |
| Span 60 | 2 |
| Tween 60 | 2 |
| Mineral oil | 5 |
| Petrolatum | 10 |
| Methyl paraben | 0.15 |
| Propyl paraben | 0.05 |
| BHA (butylated hydroxy | 0.01 |
| anisole) | |
| Water | q.s. 100 |

All of the ingredients, except water, are combined and heated to about 60° C. with stirring. A sufficient quantity of water at about 60° C. is then added with vigorous stirring to emulsify the ingredients, and water then added q.s. about 100 g.

The features disclosed in the foregoing description, or the following claims, expressed in their specific forms or in terms of a means for performing the disclosed function, or a method or process for attaining the disclosed result, as appropriate, may, separately, or in any combination of such features, be utilized for realizing the invention in diverse forms thereof.

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

The patents, published applications, and scientific literature referred to herein establish the knowledge of those skilled in the art and are hereby incorporated by reference in their entirety to the same extent as if each was specifically and individually indicated to be incorporated by reference. Any conflict between any reference cited herein and the specific teachings of this specifications shall be resolved in favor of the latter. Likewise, any conflict between an art-understood definition of a word or phrase and a definition of the word or phrase as specifically taught in this specification shall be resolved in favor of the latter.

We claim:

1. A compound of the formula (I) 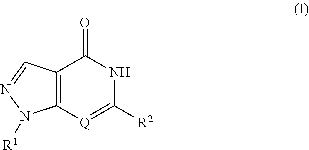
wherein
Q and X are independently in each occurrences N or CH;
R¹ is selected from the group consisting of hydrogen, C₁₋₆ alkyl, C₁₋₆ hydroxyalkyl, C₁₋₆-dihydroxyalkyl, 1,1-dioxothian-4-yl or tetrahydropyran-4-yl;
R² is 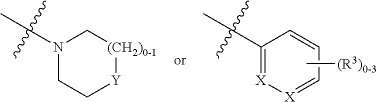
Y is selected from the group consisting of CR⁴R⁵ or NR⁴ wherein R⁵ is hydrogen, C₁₋₆ alkyl, —OH or —CN;
R³ is selected from the group consisting of (i) hydrogen, (ii) C₁₋₆ alkyl, (iii) C₁₋₆ haloalkyl, (iv) halogen, (v) C₁₋₆ alkoxy, (vi) S(O)₂R^{3a} wherein R^{1a} is C₁₋₆ alkyl, C₃₋₆ cycloalkyl, C₁₋₃ alkyl-C₃₋₆ cycloalkyl or NH₂ or (vii) CONR^{3b}R^{3c} wherein R^{3b} and R^{3c} are independently hydrogen, C₁₋₃ alkyl or R^{3b} and R^{3c} together with the nitrogen to which they are attached form a cyclic amine
R⁴ is selected from the group consisting of:
(i) hydrogen,
(ii) C₁₋₆ alkyl,
(iii) C₁₋₆ haloalkyl optionally substituted with hydroxyl,
(iv) C₃₋₇ cycloalkyl
(v) C₃₋₇cycloalkyl-C₁₋₃ alkyl,
(vi) C₅₋₁₀ bicycloalkyl, 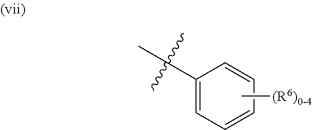
wherein each R⁶ is independently selected from the group consisting of:
(a) C₁₋₆ alkyl,
(b) C₁₋₆ haloalkyl optionally substituted with hydroxyl,
(c) C₁₋₆ hydroxyalkyl,
(d) C₁₋₆-dihydroxyalkyl,
(e) C₁₋₃ alkoxy-C₁₋₃ alkyl,
(f) C₃₋₇ cycloalkyl,
(g) C₁₋₆ acyl,
(h) halo,
(i) cyano,
(j) NO₂,
(k) carboxyl,
(l) C₁₋₆ alkoxycarbonyl,
(m) CO NR^{4b}R^{4c} wherein R^{4b} and R^{4e} are independently hydrogen, C₁₋₆ alkyl or R^{4b} and R^{4c} together with the nitrogen atom to which they are attached are a cyclic amine,
(n) —S(O)₂R^{4a} wherein R^{4a} is C₁₋₆ alkyl, C₃₋₆ cycloalkyl, C₁₋₃ alkyl-C₃₋₆ cycloalkyl or NH₂,
(o) NR^{4b}R^{4c},
(p) OR^{4d} wherein R^{4d} is selected from the group consisting of (i) hydrogen, (ii) C₁₋₆ alkyl, (iii) C₁₋₃ alkoxy-C₁₋₃ alkyl, (iv) C₁₋₆ hydroxyalkyl said hydroxalkyl further optionally substituted with halogen, (v) C₁₋₆ dihydroxyalkyl, (vi) (alkylene)₂₋₆NR^{4e}R^{4f} wherein R^{4e} and R^{4f} are independently hydrogen or C₁₋₆ alkyl or R^{4e} and R^{4f} together with the nitrogen to which they are attached form a cyclic amine optionally containing another heteroatom selected from NR^{4g}, O or S(O)₀₋₂ wherein R^{4g} is hydrogen or C₁₋₃ alkyl, (vii) oxetanyl, (viii) tetrahydropyranyl, (ix) 1,1-dioxothianyl, (x) (1-oxothietan-3-yl)methyl and (xi) (alkylene)₂₋₆OR^{4h} wherein R^{4h} is C(O)CH(NH₂)R⁴ⁱ wherein R⁴ⁱ C₁₋₆ alkyl or P(═O)(OH)₂;
(q) heterocyclyl-C₁₋₃ alkyl wherein said heterocycle is piperidine, morpholine, piperazine or 4-methyl-piperazine;
(r) 1H-tetrazol-5-yl, and,
(s) 1,1-dioxothiolan-3-yl;
(viii) heteroaryl
(ix) heteroaryl-C₁₋₃ alkyl
(x) heterocyclyl;
(xi) heterocyclyl C₁₋₃ alkyl;
and wherein:
each said cycloalkyl is optionally substituted by one to three hydroxyl or C₁₋₃ alkoxy-C₁₋₆ alkoxy;
each said heteroaryl is optionally further substituted with C₁₋₆ alkyl, C₁₋₃ hydroxyalkyl, C₁₋₆ haloalkyl, halogen or C₁₋₆ alkylsulfonyl;
each said heterocycle is selected from tetrahydropyran-4-yl, tetrahydrofuran-2-yl, oxetan-3-yl, 1,1-dioxo-tetrahydrothiophenyl, 1-Boc-piperidinyl, piperidin-4-yl, 1-methyl-piperidin-4-yl, 1-Boc-piperazin-4-yl; 1-methyl-piperazin-4-yl or piperazin-4-yl;
or a pharmaceutically acceptable salt thereof.

2. The compound according to claim 1 wherein:
R¹ is hydrogen or C₁₋₆ alkyl;
R² is 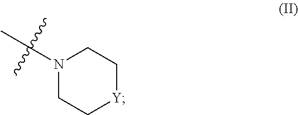
and,
Y is NR⁴ or CR⁵R⁴.

3. The compound of claim 2 wherein Y is NR⁴ and Q is N.

4. The compound of claim 2 wherein Y is NR⁴ and Q is CH.

5. The compound of claim 2 wherein Y is CR⁵R⁴ and Q is N.

6. The compound of claim 2 wherein Y is CR⁵R⁴ and Q is CH.

7. The compound of any of claim 3, 4, 5 or 6 wherein R⁴ is optionally substituted phenyl.

8. The compound of claim 7 wherein R⁵, when present, is hydrogen and R⁴ is 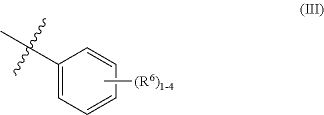
wherein one R⁶ is independently selected from the group consisting of (c) C₁₋₆ hydroxyalkyl, (d) C₁₋₆-dihydroxyalkyl, (q) heterocyclyl C₁₋₃ alkyl and (p) OR^{4d} wherein R^{4d} is selected from the group consisting of (iii) C₁₋₃ alkoxy-C₁₋₃ alkyl, (iv) C₁₋₆ hydroxyalkyl said hydroxyalkyl further optionally substituted with halogen, (v) C₁₋₆ dihydroxyalkyl, (vi) (alkylene)₂₋₆NR^{4e}R^{4f} wherein R^{4e} and R^{4f} are independently hydrogen or C₁₋₆ alkyl or R^{4e} and R^{4f} together with the nitrogen to which they are attached form a cyclic amine optionally containing another heteroatom selected from NR^{4g}, O or S(O)₀₋₂ wherein R^{4g} is hydrogen or C₁₋₃ alkyl, (vii) oxetanyl, (viii) tetrahydropyranyl, (ix) 1,1-dioxothianyl, (x) (1-oxothietan-3-yl)methyl and (xi) (alkylene)₂₋₆OR^{4h} wherein R^{4h} is C(O)CH(NH₂)R⁴ⁱ or P(═O)(OH)₂ wherein R⁴ⁱ C₁₋₆ alkyl and wherein said phenyl is further optionally substituted by one or two halogens.

9. The compound according to claim 8 wherein Q and X are N and R⁴ is:

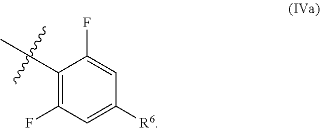

10. The compound of either of claim 7 wherein R⁴ is:

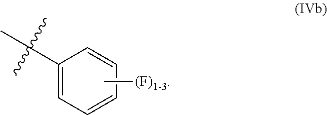

11. The compound of any of claim 3, 4, 5 or 6 wherein R⁴ is optionally substituted pyridinyl and R⁵, when present, is hydrogen or C₁₋₆ alkyl.

12. The compound of any of claim 3, 4, 5 or 6 wherein R⁴ is optionally substituted heteroaryl and R⁵, when present, is hydrogen or C₁₋₆ alkyl.

13. The compound of claim 12 wherein said optionally substituted heteroaryl is selected from the group consisting of (a) pyridinyl, (b) pyrimidinyl, (c) thiazolyl, (d) isothiazolyl, (e) oxazolyl, (f) isoxazole, (g) imidazolyl, (h) pyrazolyl, (i) 1,2,4-triazolyl, (j) 3-(pyrazinyl)-1,2,4-oxadiazolyl and (k) 1,2,4-oxadiazolyl.

14. The compound of any of claim 1 wherein:
R¹ is hydrogen or C₁₋₆ alkyl;
R² is

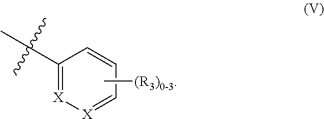

15. The compound of claim 15 wherein where each X is CH.

16. The compound of claim 15 wherein where one X is N and the other X is CH.

17. The compound according to any of claim 16 or 17 wherein each R³ is independently selected from the group consisting of C₁₋₆ alkyl, C₁₋₆ haloalkyl, halo, cyano, C₁₋₆ alkylsulfonyl, and OR^{4d} wherein R^{4d} is selected from the group consisting of (i) C₁₋₆ alkyl (ii) C₁₋₃ alkoxy-C₁₋₃ alkyl, (iii) C₁₋₆ hydroxyalkyl and (iv) C₁₋₆-dihydroxyalkyl.

18. The compound of claim 1 selected from the compounds I-1 to I-144 in Table 1.

19. A method of inhibiting tankyrase 1 and/or tankyrase 2 by contacting either or both with a compound of claim 1.

20. A method for treating cancer by administering to a patient in need thereof a therapeutically active amount of a compound of claim 1.

21. The method of claim 20 wherein the cancer is colorectal cancer.

22. The use of a compound according to claim 1 for the preparation of a medicament for the treatment of cancer.

23. A composition containing a compound according to claim 1 and at least one pharmaceutically acceptable carrier, diluent or excipient.
